(19) United States
(12) Patent Application Publication
(10) Pub. No.: US 2023/0133209 A1
(43) Pub. Date: May 4, 2023

(54) CELL CLASSIFIER CIRCUITS AND METHODS OF USE THEREOF

(71) Applicant: Eidgenössische Technische Hochschule Zürich, Zürich (CH)

(72) Inventors: Yaakov Benenson, Binningen (CH); Bartolomeo Angelici, Basel (CH)

(73) Assignee: Eidgenössische Technische Hochschule Zürich, Zürich (CH)

(21) Appl. No.: 17/918,147

(22) PCT Filed: Apr. 14, 2021
(86) PCT No.: PCT/IB2021/000246

§ 371 Date: Oct. 11, 2022

Related U.S. Application Data
(60) Provisional application No. 63/009,736, filed on Apr. 14, 2020.

(51) Int. Cl. C12N 15/52; C12N 9/22; C12N 15/86; A61P 35/00

(52) U.S. Cl. CPC C12N 15/52; C12N 9/22; C12N 15/86; A61P 35/00; C12N 2750/14143; C12N 2310/20

(57) ABSTRACT

Disclosed herein are contiguous DNA sequences encoding highly compact multi-input genetic logic gates for precise in vivo cell targeting, and methods of treating disease using a combination of in vivo delivery and such contiguous DNA sequences.

### FIELD

Disclosed herein are contiguous DNA sequences encoding highly compact multi-input genetic logic gates for precise in vivo cell targeting, and methods of treating disease using a combination of in vivo delivery and such contiguous DNA sequences.

### BACKGROUND

Gene therapy is on the rise as a next generation therapeutic option for genetic disease and cancer. However, current gene therapy vectors are plagued by low efficacy, high toxicity, and long developmental timelines to generate therapeutic leads. One reason for these drawbacks is insufficiently tight control of therapeutic gene expression in the gene therapy vector which leads to gene expression (i) in unintended cell types and tissues or (ii) at either insufficient or too-high dosage. In other words, precise control of gene expression, both in terms of gene product dosage (i.e., the number of protein molecules per cell) and cell type-restricted expression remains an open challenge in gene therapy.

### SUMMARY

Research in biomolecular computing and synthetic biology has long sought to enable new types of therapeutic approaches based on: (i) multi-input sensing of molecular disease indicators; (ii) a molecular level computation to determine the intensity of the therapeutic response; and (iii) the potentiation of a therapy in situ in a highly precise and coordinated fashion. Described herein are cell classifier gene circuits that enable precise identification of heterogeneous cell types via complex logical integration of multiple cellular inputs. Also described herein are methods of using the classifier gene circuits to treat disease. Cancer has been considered a class of diseases that would benefit most from cell classifier approaches due to tumor similarity to healthy cells, tumor heterogeneity, and its dissemination both at primary and secondary loci. The studies described herein support the notion that multi-input gene circuits for precise cell targeting are an ideal avenue for the next generation of gene therapies.

As such, in some aspects the disclosure relates to contiguous polynucleic acid molecules. In some embodiments, the contiguous polynucleic acid molecule comprises: a) a first cassette encoding a first RNA whose expression is operably linked to a transactivator response element, wherein the first RNA comprises: (i) a nucleic acid sequence of an output; and (ii) a target site for a miRNA listed in TABLE 1 or a combination thereof; and b) a second cassette encoding a second RNA, wherein the second RNA comprises a nucleic acid sequence of a transactivator; wherein the transactivator of the second cassette, when expressed as a protein, binds and transactivates the transactivator response element of the first cassette.

In some embodiments, the first RNA comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the first RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the first RNA comprises a 5′ UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the second RNA further comprises a target site for a microRNA listed in TABLE 1 or a combination thereof.

In some embodiments, wherein the second RNA further comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the second RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the second RNA comprises a 5′ UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, at least one miRNA target site of the first cassette and at least one miRNA target site of the second cassette are identical nucleic acid sequences or are different sequences regulated by the same miRNA.

In some embodiments, the first RNA and the second RNA each comprises a let-7c target site.

In some embodiments, the transactivator response element comprises a nucleic acid sequence listed in TABLE 3 or a combination thereof.

In some embodiments, expression of the second RNA is operably linked to a transcription factor response element. In some embodiments, the transcription factor response element comprises a nucleic acid sequence listed in TABLE 4 or a combination thereof.

In some embodiments, the transactivator binds and transactivates the transactivator response element independently.

In some embodiments, expression of the first RNA is operably linked to a transcription factor response element. In some embodiments, the transcription factor response element comprises a nucleic acid sequence listed in TABLE 4 or a combination thereof.

In some embodiments, the transactivator binds and transactivates the transactivator response element only in the presence of a transcription factor bound to the transcription factor response element.

In some embodiments, the first cassette and/or the second cassette comprises a promoter element. In some embodiments, the promoter element comprises a nucleic acid sequence listed in TABLE 5 or a combination thereof. In some embodiments, the promoter element comprises a mammalian promoter or promoter fragment.

In some embodiments: the first cassette comprises, from 5′ to 3′: (i) an upstream regulatory component comprising the transactivator response element and the transcription factor response element; (ii) the nucleic acid sequence encoding the output; and (iii) a downstream component comprising a let-7c target site; and the second cassette comprises, from 5′ to 3′: (i) an upstream regulatory component comprising a transcription factor response element; (ii) the nucleic acid sequence encoding the transactivator; and (iii) a downstream component comprising a let-7c target site.

In some embodiments, the transcription factor response element of the first cassette and the transcription factor response element of the second cassette consist of identical nucleic acid sequences.

In some embodiments, the transcription factor response element of the first cassette and the transcription factor response element of the second cassette consist of different nucleic acid sequences.

In some embodiments, the first cassette and/or the second cassette comprises two or more transcription factor response elements.

In some embodiments, the first cassette and/or the second cassette comprises two different transcription factor response elements.

In some embodiments, the upstream regulatory component of the first cassette comprises a promoter element. In some embodiments, the promoter element comprises a mammalian promoter or promoter fragment.

In some embodiments, the upstream regulatory component of the second cassette comprises a promoter element. In some embodiments, the promoter element comprises a mammalian promoter or promoter fragment.

In some embodiments, the first cassette and the second cassette are in a convergent orientation. In some embodiments, first cassette and the second cassette are in a divergent orientation. In some embodiments, the first cassette and the second cassette are in a head-to-tail orientation.

In some embodiments, the first cassette and/or the second cassette is flanked by an insulator.

In some embodiments, the transactivator of the second cassette is tTA, rtTA, PIT-RelA, PIT-VP16, ET-VP16, ET-RelA, NarLc-VP16, or NarLc-RelA.

In some embodiments, the transactivator of the second cassette comprises a nucleic acid sequence listed in TABLE 2.

In some embodiments, the output is a protein or an RNA molecule. In some embodiments, the output is a therapeutic. In some embodiments, the output is a fluorescent protein, a cytotoxin, an enzyme catalyzing a prodrug activation, an immunomodulatory protein and/or RNA, a DNA-modifying factor, cell-surface receptor, a gene expression-regulating factor, a kinase, an epigenetic modifier, and/or a factor necessary for vector replication, and/or a sequence encoding an antigen polypeptide of a pathogen. In some embodiments, the output is the thymidine kinase enzyme from human simplex herpes virus 1 (HSV-TK). In some embodiments, the immunomodulatory protein and/or RNA is a cytokine or a colony stimulating factor. In some embodiments, the DNA-modifying factor is a gene encoding a protein intended to correct a genetic defect, a DNA-modifying enzyme, and/or a component of a DNA-modifying system. In some embodiments, the DNA-modifying enzyme is a site-specific recombinase, homing endonuclease, or a protein component of a CRISPR/Cas DNA modification system. In some embodiments, the gene expression-regulating factor is a protein capable of regulating gene expression or a component of a multi-component system capable of regulating gene expression.

In some embodiments, the contiguous polynucleic acid molecule comprising a nucleic acid sequence listed in TABLE 6.

In some embodiments, the contiguous polynucleic acid molecule comprises a cassette encoding an RNA whose expression is operably linked to a transactivator response element, wherein the RNA comprises: (i) a nucleic acid sequence of an output; (ii) a nucleic acid sequence of a transactivator; and (iii) a target site for a miRNA listed in TABLE 1 or a combination thereof; wherein the transactivator, when expressed as a protein, binds and transactivates the transactivator response element.

In some embodiments, the first RNA comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the RNA further comprises a nucleic acid sequence of a polycistronic expression element separating the nucleic acid sequences of the output and the transactivator.

In some embodiments, the RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the RNA comprises a 5′UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

In some embodiments, the RNA comprises a let-7c target site.

In some embodiments, the transactivator response element comprises a nucleic acid sequence listed in TABLE 3 or a combination thereof.

In some embodiments, the transactivator binds and transactivates the transactivator response element independently.

In some embodiments, the expression of the RNA is operably linked to a transactivator response element and a transcription factor response element. In some embodiments, the transcription factor response element comprises a nucleic acid sequence listed in TABLE 4 or a combination thereof.

In some embodiments, the transactivator binds and transactivates the transactivator response element only in the presence of a transcription factor bound to the transcription factor response element.

In some embodiments, the cassette comprises a promoter element. In some embodiments, the promoter element comprises a nucleic acid sequence listed in TABLE 5 or a combination thereof. In some embodiments, the promoter element comprises a mammalian promoter or promoter fragment.

In some embodiments, the contiguous polynucleic acid molecule comprises, from 5′ to 3′: (i) an upstream regulatory component comprising the transactivator response element and the transcription factor response element; (ii) the nucleic acid sequence encoding the output and the transactivator; and (iii) a downstream component comprising a let-7c target site.

In some embodiments, the upstream regulatory component in (i) comprises a promoter element. In some embodiments, the promoter element comprises a mammalian promoter or promoter fragment.

In some embodiments, the transactivator of at least one cassette is tTA, rtTA, PIT-RelA, PIT-VP16, ET-VP16, ET-RelA, NarLc-VP16, or NarLc-RelA.

In some embodiments, the output is a protein or an RNA molecule. In some embodiments, the output is a therapeutic protein or RNA molecule. In some embodiments, the output is a fluorescent protein, a cytotoxin, an enzyme catalyzing a prodrug activation, an immunomodulatory protein and/or RNA, a DNA-modifying factor, cell-surface receptor, a gene expression-regulating factor, a kinase, an epigenetic modifier, and/or a factor necessary for vector replication, and/or a sequence encoding an antigen polypeptide of a pathogen. In some embodiments, the output is the thymidine kinase enzyme from human simplex herpes virus 1 (HSV-TK). In some embodiments, the immunomodulatory protein and/or RNA is a cytokine or a colony stimulating factor. In some embodiments, the DNA-modifying factor is a gene encoding a protein intended to correct a genetic defect, a DNA-modifying enzyme, and/or a component of a DNA-modifying system. In some embodiments, the DNA-modifying enzyme is a site-specific recombinase, homing endonuclease, or a protein component of the CRISPR/Cas system. In some embodiments, the gene expression-regulating factor is a protein capable of regulating gene expression or a component of a multi-component system capable of regulating gene expression.

In other aspects, the disclosure relates to vectors comprising a contiguous polynucleic acid described herein.

In other aspects, the disclosure relates to engineered viral genomes comprising a contiguous polynucleic acid described herein. In some embodiments, the engineered viral genome is derived from an adeno-associated virus (AAV) genome, a lentivirus genome, an adenovirus genome, a herpes simplex virus (HSV) genome, a Vaccinia virus genome, a poxvirus genome, a Newcastle Disease virus (NDV) genome, a Coxsackievirus genome, a rheovirus genome, a measles virus genome, a Vesicular Stomatitis virus (VSV) genome, a Parvovirus genome, a Seneca valley viral genome, a Maraba virus genome or a common cold virus genome.

In other aspects, the disclosure relates to virions comprising an engineered viral genome disclosed herein. In some embodiments, the virion comprises an AAV-DJ, AAV8, AAV6, or AAV-B1 capsid.

In other aspects, the disclosure relates to methods of stimulating a cell-specific event in a population of cells. In some embodiments, a method of stimulating a cell-specific event in a population of cells comprises contacting a population of cells with a contiguous polynucleic acid molecule described herein, a vector described herein, an engineered viral genome described herein, or a virion described herein, wherein the population of cells comprises at least one target cell type and one or more non-target cell types, wherein the target cell type(s) and the non-target cell types differ in levels and/or activity of one or more endogenous miRNAs, such that the levels and/or activity of the one or more endogenous miRNAs are at least two times higher in each of the two or more non-target cells relative to each of the target cells; and wherein the cell-specific event is regulated by expression levels of the output in the cells of the population of cells.

In some embodiments, at least a subset of the target cells and at least a subset of the non-target cells differ in levels or activity of an endogenous transcription factor, wherein the contiguous nucleic acid molecule further comprises a transcription factor response element corresponding to the endogenous transcription factor.

In some embodiments, at least a subset of the target cells and at least a subset of the non-target cells differ in levels or activity of a promoter fragment, wherein the contiguous nucleic acid molecule further comprises this promoter fragment.

In other aspects, the disclosure relates to methods of diagnosing a disease or condition. In some embodiments, a method of diagnosing a disease or a condition comprising administering a contiguous polynucleic acid molecule described herein, a vector described herein, an engineered viral genome described herein, or a virion described herein to a subject exhibiting one or more signs or symptoms associated with a disease or condition, wherein the levels of the output indicates the presence or absence of the disease and or condition.

In some embodiments, the disease is cancer. In some embodiments, the cancer is hepatocellular carcinoma (HCC), metastatic colorectal cancer, a metastatic tumor in the liver, breast cancer, lung cancer, retinoblastoma, and glioblastoma.

In other aspects, the disclosure relates to methods of treating a disease or a condition. In some embodiments, a method of treating a disease or a condition comprising administering a contiguous polynucleic acid molecule described herein, a vector described herein, an engineered viral genome described herein, or a virion described herein to a subject having the disease or condition.

In some embodiments, the method further comprises administering a prodrug, optionally wherein the prodrug is ganciclovir, optionally wherein the contiguous polynucleic acid molecule comprises a nucleic acid sequence listed in TABLE 6.

In some embodiments, the disease is cancer. In some embodiments, the cancer is hepatocellular carcinoma (HCC), metastatic colorectal cancer, a metastatic tumor in the liver, breast cancer, lung cancer, retinoblastoma, and glioblastoma.

In some aspects, the disclosure relates to method for use in a method of stimulating a cell-specific event. In some embodiments, a composition for use in a method of stimulating a cell-specific event in a population of cells comprises contacting a population of cells with a contiguous polynucleic acid molecule described herein, a vector described herein, an engineered viral genome described herein, or a virion described herein, wherein the population of cells comprises at least one target cell type and one or more non-target cell types, wherein the target cell type(s) and the non-target cell types differ in levels and/or activity of one or more endogenous miRNAs, such that the levels and/or activity of the one or more endogenous miRNAs are at least two times higher in each of the two or more non-target cells relative to each of the target cells; and wherein the cell-specific event is regulated by expression levels of the output in the cells of the population of cells.

In some embodiments, at least a subset of the target cells and at least a subset of the non-target cells differ in levels or activity of an endogenous transcription factor, wherein the contiguous nucleic acid molecule further comprises a transcription factor response element corresponding to the endogenous transcription factor.

In some embodiments, at least a subset of the target cells and at least a subset of the non-target cells differ in levels or activity of a promoter fragment, wherein the contiguous nucleic acid molecule further comprises this promoter fragment.

In other aspects, the disclosure relates to compositions for use in a method of diagnosing a disease or condition. In some embodiments, a composition for use in a method of diagnosing a disease or a condition comprises administering a contiguous polynucleic acid molecule described herein, a vector described herein, an engineered viral genome described herein, or a virion described herein to a subject exhibiting one or more signs or symptoms associated with a disease or condition, wherein the levels of the output indicates the presence or absence of the disease and or condition.

In some embodiments, the disease is cancer. In some embodiments, the cancer is hepatocellular carcinoma (HCC), metastatic colorectal cancer, a metastatic tumor in the liver, breast cancer, lung cancer, retinoblastoma, and glioblastoma.

In other aspects, the disclosure relates to compositions for use in a method of treating a disease or condition. In some embodiments, composition for use in a method of treating a disease or a condition comprising administering a contiguous polynucleic acid molecule described herein, a vector described herein, an engineered viral genome described herein, or a virion described herein to a subject having the disease or condition.

In some embodiments, the method further comprises administering a prodrug, optionally wherein the prodrug is ganciclovir, optionally wherein the contiguous polynucleic acid molecule comprises a nucleic acid sequence listed in TABLE 6.

In some embodiments, the disease is cancer. In some embodiments, the cancer is hepatocellular carcinoma (HCC), metastatic colorectal cancer, a metastatic tumor in the liver, breast cancer, lung cancer, retinoblastoma, and glioblastoma.

In other aspects, the disclosure relates to methods of stimulating a cell-specific event in a population of cells. In some embodiments, a method of stimulating a cell-specific event in a population of cells comprises contacting the population of cells with the contiguous polynucleic acid molecule or a composition comprising said contiguous polynucleic aid molecule, wherein: a) the population of cells comprises at least one target cell type and two or more non-target cell types, wherein the target cell type(s) and the non-target cell types differ in levels of one or more endogenous miRNAs, such that the levels of the one or more endogenous miRNAs are at least two times higher in at least a subset of the non-target cells, such as at least two and optionally each of the two or more non-target cells, relative to each of the target cells; and b) the contiguous polynucleic acid molecule comprises: (i) a first cassette encoding a RNA whose expression is operably linked to a transactivator response element, wherein the first RNA comprises: a nucleic acid sequence of an output; and one or more miRNA target sites corresponding to the one or more endogenous miRNAs; and (ii) a second cassette encoding a second RNA, wherein the second RNA comprises a nucleic acid sequence of a transactivator; wherein the transactivator of the second cassette, when expressed as a protein, binds and transactivates the transactivator response element of the first cassette; and wherein the cell-specific event is regulated by expression levels of the output in the cells of the population of cells. In some embodiments, the contiguous polynucleic acid molecule comprises a nucleic acid sequence listed in TABLE 6.

In some embodiments, a method of stimulating a cell-specific event in a population of cells comprising contacting the population of cells with the contiguous polynucleic acid molecule or a composition comprising said contiguous polynucleic aid molecule, wherein: a) the population of cells comprises at least one target cell type and two or more non-target cell types, wherein the target cell type(s) and the non-target cell types differ in levels of one or more endogenous miRNAs, such that the levels of the one or more endogenous miRNAs are at least two times higher in at least a subset of the non-target cells, such as at least two and optionally each of the two or more non-target cells, relative to each of the target cells; and b) the contiguous polynucleic acid molecule comprises a cassette encoding a mRNA whose expression is operably linked to a transactivator response element, wherein the RNA comprises: a nucleic acid sequence of an output; a nucleic acid sequence of a transactivator; and one or more miRNA target sites corresponding to the one or more endogenous miRNAs; and wherein the transactivator, when expressed as a protein, binds and transactivates the transactivator response element of the cassette; and wherein the cell-specific event is regulated by expression levels of the output in the cells of the population of cells.

In some embodiments, a composition comprising the contiguous polynucleic aid molecule comprises a vector comprising the contiguous polynucleic acid, an engineered viral genome comprising the contiguous polynucleic acid, or a virion comprising the polynucleic acid.

In some embodiments, the endogenous miRNA is selected from the miRNAs listed in TABLE 1 or a combination of miRNAs listed in TABLE 1. In some embodiments, the endogenous miRNA is selected from the group consisting of let-7c, let-7a, let-7b, let-7d, let-7e, let-7f, let-7g, let-7i, miR-22, miR-26b, miR-122, miR-208a, miR-208b, miR-1, miR-217, miR-216a, or a combination thereof.

In some embodiments, at least a subset of the target cells and at least a subset of the non-target cells differ in levels or activity of an endogenous transcription factor, wherein the contiguous nucleic acid molecule further comprises a transcription factor response element corresponding to the endogenous transcription factor.

In some embodiments, at least a subset of the target cells and at least a subset of the non-target cells differ in levels or activity of a promoter fragment, wherein the contiguous nucleic acid molecule further comprises this promoter fragment.

In some embodiments, the target cells are tumor cells and the cell-specific event is tumor cell death. In some embodiments, the tumor cell death is mediated by immune targeting through the expression of activating receptor ligands, specific antigens, stimulating cytokines or any combination thereof.

In some embodiments, the target cells are senescent cells and the cell-specific event is senescent cell death.

In some embodiments, the method further comprises contacting the population of cells with prodrug or a non-toxic precursor compound that is metabolized by the output into a therapeutic or a toxic compound.

In some embodiments, output expression ensures the survival of the target cell population while the non-target cells are eliminated due to lack of output expression and in the presence of an unrelated and unspecific cell death-inducing agent.

In some embodiments, the target cells comprise a particular phenotype of interest such that output expression is limited to the cells of this particular phenotype.

In some embodiments, the target cells are a cell type of choice and the cell-specific event is the encoding of a novel function, through the expression of a gene naturally absent or inactive in the cell type of choice.

In some embodiments, the population of cells comprises a multicellular organism. In some embodiments, the multicellular organism is an animal. In some embodiments, the animal is a human.

In some embodiments, the population of cells is contacted ex-vivo. In some embodiments, the population of cells is contacted in-vivo.

In other aspects, the disclosure relates to contiguous polynucleic acid molecules. In some embodiments, a contiguous polynucleic acid molecule comprises: a) a first cassette encoding a first RNA whose expression is operably linked to a transactivator response element, wherein the first RNA comprises: (i) a nucleic acid sequence of an output; and (ii) a target site for a miRNA, wherein said miRNA is highly expressed and/or active in at least two different healthy tissues of a mammal and is expressed at low level in one or more types of target cells; b) a second cassette encoding a second RNA, wherein the second RNA comprises a nucleic acid sequence of a wherein the transactivator of the second cassette, when expressed as a protein, binds and transactivates the transactivator response element of the first cassette.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1N. Translation of a multi-plasmid circuit architecture to a viral vector. FIG. 1A. Schematics of genetic arrangements. Divergent (top) and convergent (bottom) arrangements were made; two variants were made for each, using different variants of the auxiliary transactivator PIT (divergent: D-P2: PIT=PIT::RelA; D-PV: PIT=PIT::VPI6; convergent: C-P2: PIT=PIT::RelA; C-PV: PIT=PIT::VPI6). FIG. 1B. Testing of backbone DNA performance using transient transfections and ectopic input expression in HeLa cells. Bars in each grouping, from left to right: C-P2, D-P2, C-PV, D-PV. FIG. 1C. Evaluation of constructs' response to endogenous inputs in HuH-7 and HeLa cells. Bars in each grouping, from left to right: C-P2, D-P2, C-PV, D-PV. FIG. 1D. Schematics of constructs incorporating miRNA targets as robust Off switches, illustrated using the miR-424 target sequence. Divergent (top) and convergent (bottom) arrangements were made; two variants were made for each, using different variants of the auxiliary transactivator PIT (divergent: D-P2: PIT=PIT::RelA-T424; D-PV: PIT=PIT::VPI6-T424; convergent: C-P2: PIT=PIT::RelA-T424; C-PV: PIT=PIT::VPI6-T424). FIG. 1E. Validation of the AND-gate component of the logic program in HeLa cells via ectopic expression of TF inputs. Bars in each grouping, from left to right: C-P2-T424, D-P2-T424, C-PV-T424, D-PV-T424. FIG. 1F. Evaluation of circuit response to endogenous transcriptional inputs in HuH-7 and HeLa cells. The order of bars is identical to FIG. 1E. FIG. 1G. A complete evaluation of the three-input program encoded on the divergent orientation in HeLa cells using ectopic input delivery. The input combination with only miR-424 present was not evaluated due to obvious futility, given the lack of expression in the absence of all inputs and the fact that miR-424 is a negative regulator. Bars in each grouping, from left to right: D-P2-T424, D-PV-T424. FIG. 1H. Functionality of the miRNA switch in the presence of inducing TF inputs. Circuit output is tested in HuH-7 cells with and without ectopic transfection of miR-424 mimic (indicated under X axis). The order of bars is identical to FIG. 1G. FIG. 1I. Evaluation of circuits harboring miR-126 target with respect to their repressibility in the presence of endogenously expressed inducing TF inputs. The order of bars is identical to FIG. 1G. FIG. 1J. Evaluation of the miRNA target effect on cell classification performance with two HCC cells lines and HeLa cells as a negative control. Bars in each grouping, from left to right: D-P2, D-PV, D-P2-T424, D-PV-T424, C-PV-T126, D-PV-T126. FIG. 1K. Evaluation of the circuit panel, with and without miRNA sensors incorporated, packaged into DJ-pseudotyped AAV vectors, in HCC cell lines HepG2 and HuH-7. HeLa and HCT-116 cell lines are used as counter samples. Bars in each grouping, from left to right: CMV, D-P2, D-PV, D-P2-T424, D-PV-T424, C-PV-T126, D-PV-T126. FIG. 1L. In vitro evaluation of a panel of miRNAs for their capacity to distinguish healthy primary hepatocytes from HCC cell lines. Bars in each grouping, from left to right: TFF5, T424, T126, T122. FIGS. 1M-1N. The exploration of different miRNA target arrangements and their impact on the magnitude of output repression. FIG. 1M. Schematics of the different constructs and their shorthand notations. FIG. 1N. Output generated in the HepG2 cells (no miR-122 expression) and HuH-7 cells (intermediate level of miR-122 expression). Bars in each grouping, from left to right: HepG2, Huh-7. Abbreviations: ITR: internal terminal repeat of AAV2; pA: an SV40 polyadenylation signal (convergent orientation), hGH next to mCherry and SV40 pA next to PIT genes in divergent orientation; Cherry: a sequence encoding an mCherry fluorescent protein; TATA: a minimal TATA box (Angelici et al., 2016); HNF1 RE: a response element binding HNF1A and HNF1B; PIT RE: a response element binding PIT::RelA and PIT::VP16 transactivator; SOX RE: a DNA sequence that binds SOX9 and SOX10 transcription factors, and possibly other transcription factors from the SOX family SOX1-SOX15, SOX17, SOX18, SOX21, SOX30, and SRY; PIT: pristinomycin-inducible transactivator (Fussenegger et al., 2000), which stands either for PIT:RelA or PIT::VP16 fusion. Chart designs: The normalized expression of the output mCherry is shown on Y axis.

FIGS. 2A-2F. Pilot evaluation of specificity and efficacy in the orthotopic mouse model of HCC. FIG. 2A. In vitro validation of cell classification capacity of the chosen circuit packaged into DJ-pseudotyped viral vector. FIG. 2B. In vitro cell elimination by the circuit with HSV-TK output, compared to the constitutive control vector. Schematics of the circuits employed here are shown above the bar charts. For every cell line or primary hepatocytes, the dose-response to ganciclovir (X axis) is measured in the presence of a constitutive HSV-TK vector, the circuit, and with GCV alone. Cell viability MTS readouts are shown on Y axis. FIG. 2C. The progression of tumor load in tumor-bearing mice, shown for different experimental arms of the pilot experiment (n=2), as indicated in the panel. FIG. 2D. Tumor load in the liver at termination, quantified by luminescence, the images on the left are superpositions of livers (grayscale) and the bioluminescent signal. FIG. 2E. Quantitative analysis of the tumor load in the livers post-termination. FIG. 2F. The correlation between tumor load soon after inoculation, and the tumor load at termination. The two mice from the treatment arm are represented by two red dots.

FIGS. 3A-3F. Identification of a selective and broadly-applicable miRNA input for the tumor-targeting program. FIG. 3A. The schematics of cell profiling and ranking of miRNA candidates based on their high expression in healthy liver and low expression in the HCC samples. FIG. 3B. The schematics of functional validation of the pre-selected miRNA inputs. A reporter viral vector is created for every input, and every vector is delivered to every sample of interest (one by one) to evaluate the biological activity of the inputs. FIG. 3C. The results of the functional evaluation of a miRNA panel in two HCC cell lines and primary healthy hepatocytes. Low reporter expression corresponds to high miRNA activity. FF5 is a control target. FIG. 3D. The correlation between the miRNA expression count identified in the NGS profiling experiment (Dastor et al., 2018) and the functional response of selected miRNA sensors. The trend line is fit to a repressor Hill function. FIG. 3E. The quantified expression of a panel of miRNA reporter vectors in different mouse organs, following systemic delivery. Expression of different reporters in the same organ (indicated above a chart) is grouped together. The bar shading indicates in which organ the reporter was expected to respond based on literature analysis and profiling data. The values are normalized to the control vector bearing TFF5 target; with that, it is clear that this target is responding to cryptic inputs in vivo and many reporters result in output values above 1. FIG. 3F. Representative images of reporter expression in various organs. The name of the reporter is indicated on the left. The cerulean panel shows the expression of constitutive mCerulean internal control. The Cherry panel shows the residual expression of the mCherry reporter, furnished with the indicated miRNA target.

FIGS. 4A-4C. Validation of circuit specificity in vitro. FIG. 4A. The panel of control constructs used to evaluate a circuit's mechanism of action. The abbreviations are the same as in FIGS. 1A, 1D and 1M. FIG. 4B. Mapping C.TF-AND sub-circuit response to endogenous inputs in 10 cell lines and primary hepatocytes. For every cell line, the log-transformed output of the feedback-amplified sensor for SOX9/10 and HNF1A/B, normalized to the constitutive output in these cells, is shown respectively on X and Y axis. The output of the C.TF-AND circuit is shown on Z axis. FIG. 4C. Mapping HCC.V2 circuit response in 10 cell lines and primary hepatocytes. Log-transformed output of the C.TF-AND circuit and log-transformed C.let-7c reporter circuit response magnitude are plotted on axes X and Y, while the output of the complete circuit in every cell line is shown on axis Z. All values for a given cell type are normalized to constitutive expression in that cell type.

FIGS. 5A-5D. In vivo characterization of circuit targeting specificity. FIG. 5A. Output of selected sub-programs, control vector, the full program, and background, obtained using B1-pseudotyped AAV vectors in various organs. The values are obtained by quantitative image analysis. FIG. 5B. Images of tissue slices representing different organs, showing the expression of mCherry from different vectors as indicated. The Phase image and the mCherry channel are shown. Two different exposures are used to represent pancreas slices, to reflect the large dynamic range of the mCherry change. FIG. 5C. Expression of mCherry output from HCC.V2 circuit in the tumor and in the organs of HepG2-tumor bearing mice. The tumor is stably transduced with mCitrine and is showing in the Yellow fluorescent channel. FIG. 5D. Quantitative analysis of mCherry expression in the tumor and various organs of tumor-bearing mice, obtained using image processing.

FIGS. 6A-6B. In vitro efficacy of the circuit and controls in two HCC cell lines and primary hepatocytes. FIG. 6A. Dose-response to GCV in the absence of any AAV vector (squares), in the presence of a constitutive HSV-TK expression cassette (triangles) or the complete circuit (circles). Cell viability measured using MTS assay is shown on Y axis. Schematic representations of the circuits and their IDs are shown on top. FIG. 6B. The sensitivity of HuH-7 cell line to different vector dosage of the constitutive HSV-TK cassette and the two different tumor targeting programs. Top chart, comparison between the two circuit variants; bottom, the comparison between the constitutive vector and the second circuit variant.

FIGS. 7A-7F. Efficacy of HCC-targeting circuit in orthotopic mouse model. FIG. 7A. The schematics of tumor establishment and treatment regimen. FIG. 7B. Tumor load over time in various experimental arms. Tumor load, measured via in vivo whole-body bioluminescence, is imaged over time. For each animal, the load is normalized to the load on the day before initiating GCV injection regimen. FIG. 7C. A spider plot showing tumor load development for individual animals in the main experimental arms, normalized to the tumor load on the day before initiating GCV injection regimen. FIG. 7D. Representative images of whole-body luminescence of individual animals from a number of experimental arms. FIG. 7E. Images of individual livers and the tumor load in the liver measured by whole-organ bioluminescence at termination for a number of experimental arms. FIG. 7F. Quantification of the tumor load in FIG. 7E.

FIGS. 8A-8C. In vivo evaluation of AAV-B1 tumor transduction. FIG. 8A. Output of control vector, C.TF-AND subprogram and the full program packaged in DJ-pseudotyped AAV vectors are compared to the output of the full circuit packaged in B1-pseudotuped AAV vectors in liver and HepG2-tumors. The tumor is stably transduced with mCitrine and is showing in the Yellow fluorescent channel. FIG. 8B. Quantification of HCC.V2 driven output level (mCherry) in the tumor upon AAV-DJ and AAV-B1 delivery. The values are obtained by quantitative image analysis. FIG. 8C. Output from HCC.V2 circuit delivered by B1-pseudotyped AAV in core section of a large tumor nodule.

FIGS. 9A-9B. Rational design of optimized circuit combining multiple liver protective miRNAs. FIG. 9A. Schematics of candidate circuits (HCC.V3) that combine strong miR-let7c and weak miR-122 repression. The strong miR-let7c repression is obtained by using the target configuration describe in HCC.V2. The repression strength elicited by miR-122 can be tuned by varying the number, arrangement or sequence of the miRNA targets. Depicted are shown 3 different strategies to reduce miR-122 repression levels compared to HCC.V1: (i) use of a perfect miR-122 target (T-122*) only on the transactivator branch of the circuit; (ii) double repression of the transactivator and the output using miR-122 targets with imperfect complementarity (T-122*); or (iii) a mixed approach that relies on perfect target to repress the transactivator and imperfect miRNA targets to repress the output. The candidate that maximizes the repression in liver lines while minimizing the loss of expression in a panel of HCC cell lines (HUH-7 in particular) is selected. Each candidate is tested in both possible miRNA targets relative positioning variants. FIG. 9B. Example of imperfect miR-122 target (T-122*) derived from the conserved UTR region of an endogenous gene (P4HA1) regulated by miR-122 (SEQ ID NOS: 305 and 306, top and bottom respectively). Targets with imperfect complementarity are obtained either by using sequence occurring in endogenous genes or by introducing random mutations in the region flanking the miRNA seed sequence. Both approaches will be used to create a selection of targets with different dose-response profiles.

### DETAILED DESCRIPTION

One of the promises of molecular computing (Benenson, 2012) and synthetic biology (Weber and Fussenegger, 2012) has been the rational design of “smart” therapies (Benenson et al., 2004) that sense and respond to disease-related cues in complex fashion and in real time, resulting in precise and “on demand” therapeutic actuation. In order to deliver on this promise, three separate challenges are addressed. First, a disease mechanism is sufficiently understood in order to design blueprints for a therapeutically relevant sense-compute-respond cascades. In particular, relevant inputs are identified and the program that would result in the most efficacious and the least toxic response preferably is determined. Second, robust synthetic biology platforms capable of implementing these therapeutic cascades exist, or are developed de novo for the purpose. Third, these platforms are adapted to clinically-relevant therapeutic modalities. Among the latter, cell and gene therapies have been identified as the most suitable for the clinical translation of synthetic gene circuits, given the fact that both of these modalities enable, and often require, the incorporation of engineered genetic payload.

Addressing all three challenges narrows down the field of potential medical indications to develop the approach in the translational setting. One line of work has focused on cell-based implants, where the genetically modified cells are able to sense a particular disease-related cue in blood circulation and secrete a molecular agent with therapeutic properties in response. In this line of work, the cell implant serves as a sentinel and a “factory” that senses organismal disease state and produces a therapy that affects the entire organism in response (Auslander et al., 2014; Tastanova et al., 2018; Ye et al., 2017). A second line of research has built on the CAR-T cell therapy approach and augmented these cells with multi-input combinatorial sensing properties, in order to improve their specificity toward cancer cells expressing combinations of surface antigens, and reduce on-target, off-tumor effects (Cho et al., 2018; Kloss et al., 2013; Roybal et al., 2016; Zah et al., 2016).

Synthetic biology applications in the field of gene therapy have also shown initial success in animal disease models. A hybrid approach, combining a set of lentiviral vectors addressing ovarian cancer cells and expressing immunomodulators in these cells, and engineered T-cells, showed efficacy in a mouse model of ovarian metastasis to the peritoneal cavity. Cell targeting was implemented as a miRNA sponge-enabled AND gate between two promoters whose combination was shown to be tumor specific (Nissim et al., 2017). In another recent work, an oncolytic adenovirus was engineered to replicate based on a multi-input logical control of its life cycle and showed efficacy upon intratumoral injection into a subcutaneous tumor (Huang et al., 2019).

The main added value of synthetic gene circuits for gene and cell therapies arises from the sophisticated approaches to “program” the therapeutic response, that is, regulate the specificity, the timing, and the dosage of the therapeutic actuation in a predetermined fashion, potentially in a dynamic manner and in combination with various feedback regulatory motifs (Angelici et al., 2016; Xie et al., 2011). However, furnishing a known therapeutic transgene with a gene circuit regulating its expression may not necessarily be better than a more established approaches that often use a constitutively-driven or tissue-specific promoter-driven therapeutic gene packaged into a viral vector that additionally possesses a degree of organ or cell type specificity via its capsid (Al-Zaidy et al., 2019; Landegger et al., 2017; Scholl et al., 2016). Alternatively, viral vectors can be injected directly into the tissue or organ of interest (Juttner et al., 2019; Nelson et al., 2016), reducing the diversity of cell types that need to be specifically addressed. Indeed, the majority of approved therapies, including clinically approved CAR-T cells (June et al., 2018) and many gene therapies (Keeler and Flotte, 2019), engineered based on this approach, show satisfactory efficacy and safety profiles. Thus, a burden is on the synthetic biology community to prove this advantage.

Cancer is a disease that has tremendous potential to benefit from therapies powered by synthetic biology. Even narrowly defined cancers are heterogenous disease, both between patient groups and even between individual tumors in the same patient (Dagogo-Jack and Shaw, 2018). Tumors in a patient are often spread between primary and metastatic loci, making intratumoral injection possible only for a subset of cases. Lastly, anti-tumor therapies are very toxic, meaning that their activation in non-tumor cells will lead to often dramatic adverse effects. Together, the requirement to address a complex, heterogeneous cell population precisely, combined with the need to deliver the agent systemically to address a spread population of tumors, suggests that the use of synthetic biology approaches can be beneficial.

Disclosed herein are contiguous polynucleic acid molecules that encode classifier gene circuits compatible with commonly used gene therapy viral and non-viral vectors. Also disclosed herein are methods of implementing complex multi-input control over the expression of an output (i.e., gene of interest) in a population of cells. These methods include gene therapies for the diagnosis and treatment of diseases such as cancer (e.g., hepatocellular carcinoma (HCC)).

### I. Compositions of Contiguous Polynucleic Acid Molecules

In some aspects, the disclosure relates to contiguous polynucleic acid molecules comprising a gene circuit. As used herein, the term “contiguous polynucleic acid molecule” refers to a single, continuous nucleic acid molecule (i.e., a single-stranded polynucleic acid molecule) or two complementary continuous nucleic acid molecules (i.e., a double-stranded polynucleic acid molecule comprising two complementary strands). In some embodiments, the contiguous polynucleic acid is an RNA (e.g., single-stranded or double-stranded). In some embodiments, the contiguous polynucleic acid is a DNA (e.g., single-stranded or double-stranded). In some embodiments, the contiguous polynucleic acid is a DNA:RNA hybrid.

A contiguous polynucleic acid described herein comprises a gene circuit that is encoded one or more expression cassettes. As used herein, the terms “expression cassette” and “cassette” are used interchangeably and refer to a polynucleic acid comprising: (i) a nucleic acid sequence encoding an RNA (e.g., comprising the nucleic acid sequence of an output and/or a transactivator); and (ii) a nucleic acid sequence that regulates expression levels of the RNA (e.g., a transactivator response element, a transcription factor response element, a minimal promoter, and/or a promoter element).

In some embodiments, a contiguous polynucleic acid molecule comprises a gene circuit consisting of a single cassette. In other embodiments, a contiguous polynucleic acid molecule comprises a gene circuit comprising two or more cassettes.

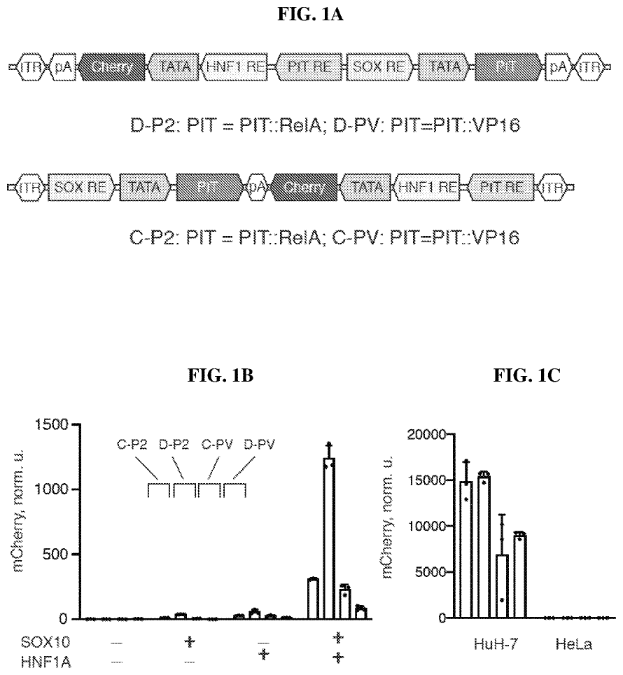

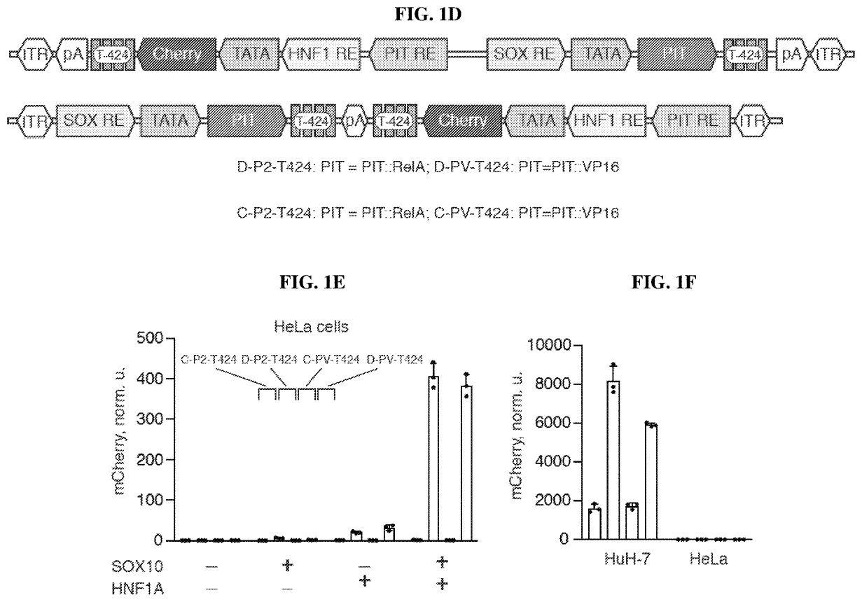

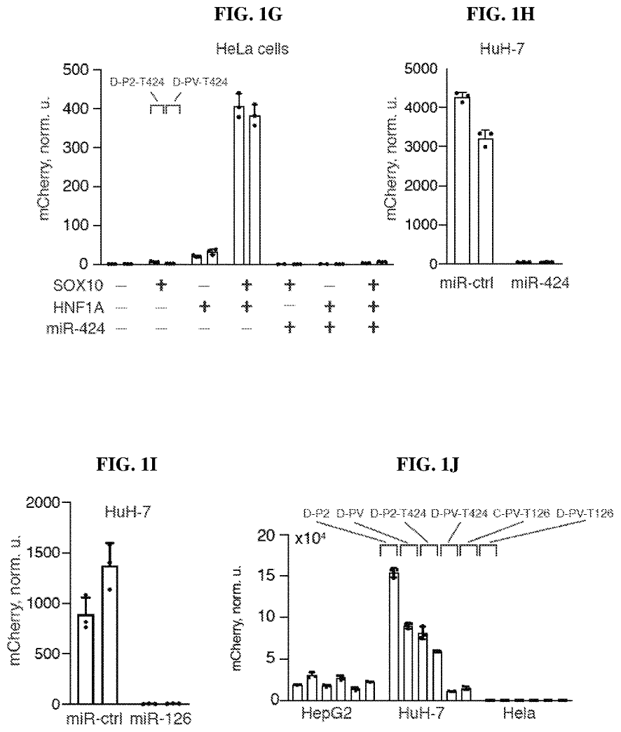

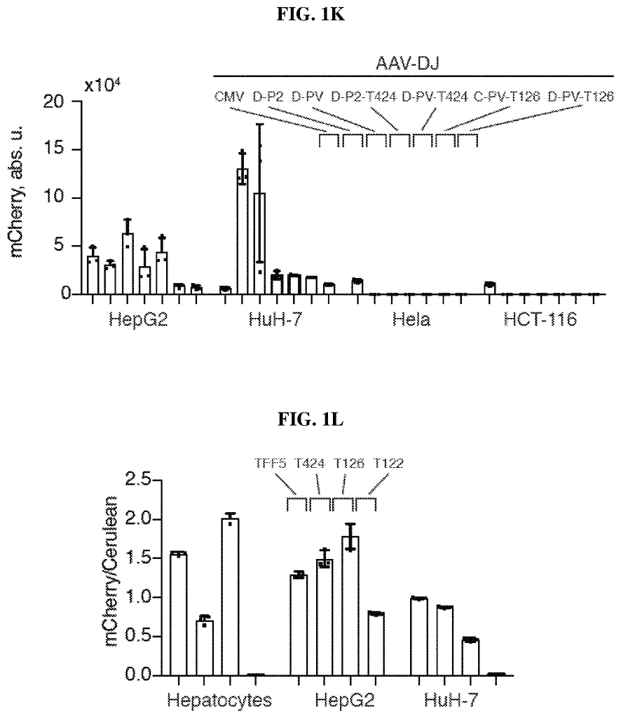

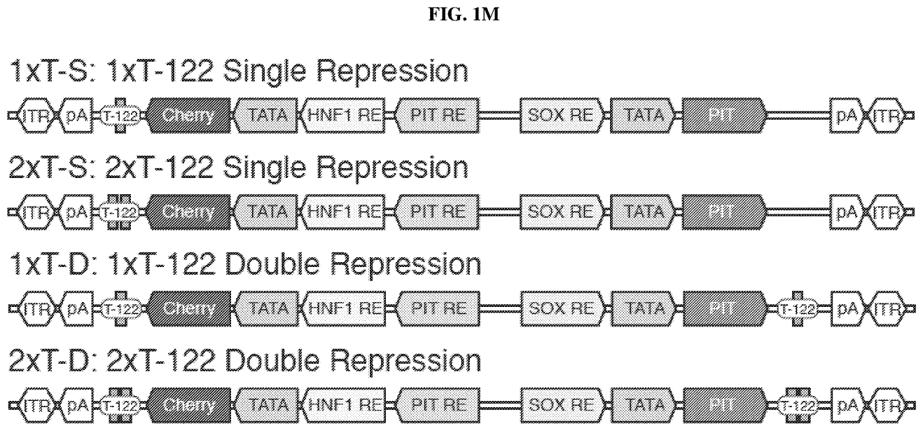

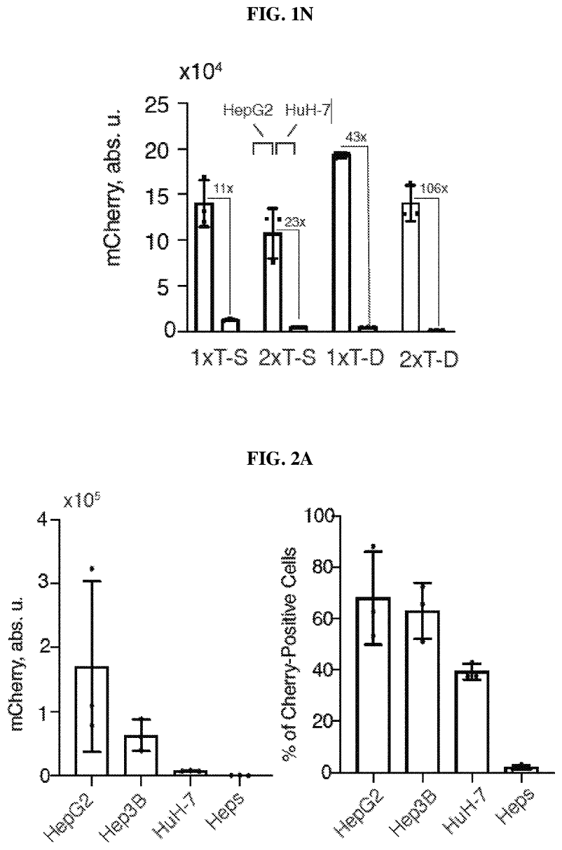

In some embodiments, a contiguous polynucleic acid molecule comprises two or more cassettes and at least two cassettes are in a divergent orientation. The term “divergent orientation,” as used herein, refers to a configuration in which: (i) transcription of a first cassette and a second cassette proceeds on different strands of the contiguous polynucleic acid molecule and (ii) transcription of the first cassette is directed away from the second cassette and transcription of the second cassette is directed away from the first cassette. FIG. 1A (upper schematic) provides examples of various divergent configurations.

In some embodiments, a contiguous polynucleic acid molecule comprises two or more cassettes and at least two cassettes are in a convergent orientation. As used herein, the term “convergent orientation” refers to a configuration in which: (i) transcription of a first cassette and a second cassette proceeds on different strands of the contiguous polynucleic acid molecule and (ii) transcription of the first cassette is directed toward the second cassette and transcription of the second cassette is directed toward the first cassette. In some embodiments, two convergent cassettes share a polyadenylation sequence. FIG. 1A (lower schematic) provides examples of various convergent configurations.

In some embodiments, a contiguous polynucleic acid molecule comprises two or more cassettes and at least two cassettes are in a head-to-tail orientation. As used herein, the term “head-to-tail” refers to a configuration in which: (i) transcription or translation of the first cassette and the second cassettes proceeds on the same strand of the contiguous polynucleic acid molecule and (ii) transcription or translation of the first cassette is directed toward the second cassette and transcription or translation of the second cassette is directed away from the first cassette (5′ . . . → . . . → . . . 3′).

In some embodiments, two cassettes are separated by one or more insulators. Insulators are nucleic acid sequences that, when bound by insulator-binding proteins, shield a regulatory component or a response component from the effects of other nearby regulatory elements. For example, flanking the cassettes of a contiguous polynucleic acid molecule can shield each cassette from the effects of regulatory elements of the other cassettes. Examples of insulators are known to those having skill in the art.

The gene circuits described herein utilize one or more mechanisms to regulate expression levels of an output molecule (i.e., a gene of interest). Therefore, each of the contiguous polynucleic acids described herein comprises a cassette encoding an RNA comprising the nucleic acid sequence of an output. Exemplary output molecules are provided below. The RNA comprising the nucleic acid sequence of the output is operably linked to a transactivator response element (and, optionally, one or more additional nucleic acid sequences that regulate expression of the RNA, such as a transcription factor response element, a minimal promoter, and/or a promoter element).

To regulate the expression levels of the output molecule (i.e., gene of interest), each of the contiguous polynucleic acids described herein further comprises: (i) a cassette encoding an RNA (e.g., mRNA) comprising the nucleic acid sequence of a transactivator; and (ii) a cassette encoding an RNA comprising a miRNA target site. Exemplary transactivators and miRNA target sites are provided below.

The cassette encoding the RNA (e.g., mRNA) comprising the nucleic acid sequence of the transactivator may be operably linked to a nucleic acid sequence that regulates expression of the RNA (e.g., a transactivator response element, a transcription factor response element, a minimal promoter, and/or a promoter and/or enhancer element). In some embodiments, the cassette encoding the RNA comprising the nucleic acid sequence of the transactivator is the same cassette encoding the RNA comprising the nucleic acid sequence of the output (i.e., a single RNA comprises the nucleic acid sequences of both the transactivator and the output).

The cassette encoding the RNA comprising the miRNA target site may be the same cassette encoding the RNA comprising the nucleic acid sequence of the output (i.e., the RNA comprising the nucleic acid sequence of the output further comprises a miRNA target site). Alternatively or in addition, the cassette encoding the RNA comprising the miRNA target site may be the same cassette encoding the RNA comprising the nucleic acid sequence of the transactivator (i.e., the nucleic acid sequence of the transactivator further comprises a miRNA target site).

In some embodiments, the nucleic acid sequence of an RNA encoded by a cassette further comprises a polyadenylation sequence. In some embodiments, the polyadenylation sequence is suitable for transcription termination and polyadenylation in mammalian cells.

(i) MiRNA Target Sites

Each of the contiguous polynucleic acids described herein comprise one or more cassettes encoding an RNA (e.g., the RNA comprising the nucleic sequence encoding the output and/or the RNA comprising the nucleic acid sequence of the transactivator) that comprises a miRNA target site. MiRNAs are a class of small non-coding RNAs that are typically 21-25 nucleotides in length that downregulate the levels of RNAs to which they bind in a variety of manners, including translational repression, mRNA cleavage, and deadenylation. The term “miRNA target site,” as used herein, refers to a sequence that complements and is regulated by a miRNA. A miRNA target site may have at least 25%, at least 30%, at least 40%, at least 50%, at least 60%, at least 70%, at least 80%, at least 85%, at least 90%, at least 95%, at least 96%, at least 97%, at least 98%, at least 99%, or 100% complementarity to the miRNA that binds and regulates the miRNA target site.

In some embodiments, an RNA encoded by a cassette described herein comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 miRNA target sites. In some embodiments, an RNA encoded by a cassette described herein comprises 1, 2, 3, 4, 5, 6, 7, 8, 9, 10, 11, 12, 13, 14, 15, 16, 17, 18, 19, or 20 miRNA target sites. In some embodiments, an RNA encoded by a cassette described herein comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 miRNA target sites.

In some embodiments, an RNA encoded by a cassette described herein comprises multiple miRNA target sites and each of the miRNA target sites have identical sequences or comprise a different nucleic acid sequence that is regulated by the same miRNA. In other embodiments, an RNA encoded by a cassette described herein comprises two or more miRNA target sites that are regulated by distinct miRNAs (i.e., distinct miRNA target sites); comprising for example, at least 1, at least 2, at least 3, at least 4, at least 5, at least 6, at least 7, at least 8, at least 9, or at least 10 distinct miRNA target sites. In some embodiments, an RNA encoded by a cassette described herein comprises 1, 2, 3, 4, 5, 6, 7, 8, 9, or 10 distinct miRNA target sites. In some embodiments, an RNA encoded by a cassette described herein comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 distinct miRNA target sites.

A miRNA target site of an RNA encoded by a cassette described herein may be located anywhere within the sequence of the RNA. For example, in some embodiments an RNA encoded by a cassette described herein comprises a 3′ UTR, and the 3′ UTR comprises a miRNA target site. In some embodiments, an RNA encoded by a cassette described herein comprises a intron, and the intron comprises a miRNA target site. In some embodiments, an RNA encoded by a cassette described herein comprises a 5′ UTR, and the 5′ UTR comprises a miRNA target site.

Exemplary miRNAs and miRNA target sites are listed in TABLE 1. In some embodiments, an RNA encoded by a cassette described herein comprises a miRNA target site for a miRNA listed in TABLE 1. In some embodiments, an RNA encoded by a cassette described herein comprises multiple miRNA target sites corresponding to a miRNA listed in TABLE 1 (e.g., a combination including a let-7c target site and a miR-122 target site).

In some embodiments, an RNA encoded by a cassette described herein comprises a miRNA target site having at least at least 70%, at least 80%, at least 85%, at least 90%, at least 95%, at least 96%, at least 97%, at least 98%, at least 99%, or 100% identity to a miRNA target site listed in TABLE 1. In some embodiments, an RNA encoded by a cassette described herein comprises multiple miRNA target sites having at least 70%, at least 80%, at least 85%, at least 90%, at least 95%, at least 96%, at least 97%, at least 98%, at least 99%, or 100% identity to a miRNA target site listed in TABLE 1.

In some embodiments, an RNA encoded by a cassette described herein comprises a let-7a target site, a let-7b target site, a let-7c target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof (e.g., a combination of a let7c target site and a miR-122 target site).

In some embodiments, an RNA encoded by a cassette described herein comprises a let-7c target site (i.e., a nucleic acid sequence that complements and is regulated by hsa-let-7c). In some embodiments a let-7c target site consists of the nucleic acid sequence AACCATACAACCTACTACCTCA (SEQ ID NO: 42).

In some embodiments, an RNA encoded by a cassette described herein comprises a miR-22 target site (i.e., a nucleic acid sequence that complements and is regulated by miR-22). In some embodiments a miR-22 target site consists of the nucleic acid sequence ACAGTTCTTCAACTGGCAGCTT (SEQ ID NO: 43).

In some embodiments, an RNA encoded by a cassette described herein comprises a miR-26b target site (i.e., a nucleic acid sequence that complements and is regulated by miR-26b). In some embodiments a miR-26b target site consists of the nucleic acid sequence

(SEQ ID NO: 44)
ACCTATCCTGAATTACTTGAA.

In some embodiments, an RNA encoded by a cassette described herein comprises a miR-126-5p target site (i.e., a nucleic acid sequence that complements and is regulated by miR-126-5p). In some embodiments a miR-126-5p target site consists of the nucleic acid sequence CGTGTTCACAGCGGACCTTGAT (SEQ ID NO: 45).

In some embodiments, an RNA encoded by a cassette described herein comprises a miR-424 target site (i.e., a nucleic acid sequence that complements and is regulated by miR-424). In some embodiments a miR-424 target site consists of the nucleic acid sequence GTCCAAAACATGAATTGCTGCT (SEQ ID NO: 48).

In some embodiments, an RNA encoded by a cassette described herein comprises a miR-122 target site (i.e., a nucleic acid sequence that complements and is regulated by miR-122). In some embodiments a miR-122 target site consists of the nucleic acid sequence CAAACACCATTGTCACACTCCA (SEQ ID NO: 46).

TABLE 1
Exemplary miRNAs and exemplary miRNA target sites.

| SEQ | | | | SEQ | |
|---|---|---|---|---|---|
| ID | | | | ID | |
| NO: | miRNA | Accession | miRNA SEQUENCE | NO: | TARGET SEQUENCE |
| 1 | miR-let7c- | MIMAT0000064 | UGAGGUAGUAGG | 42 | AACCATACAACCTACT |
| | 5p | | UUGUAUGGUU | | ACCTCA |
| 2 | miR-22-3p | MIMAT0000077 | AAGCUGCCAGUUG | 43 | ACAGTTCTTCAACTGGC |
| | | | AAGAACUGU | | AGCTT |
| 3 | hsa-miR-26b | MIMAT0000083 | UUCAAGUAAUUC | 44 | ACCTATCCTGAATTACT |
| | | | AGGAUAGGU | | TGAA |
| 4 | hsa-miR- | MIMAT0000444 | CAUUAUUACUUU | 45 | CGCGTACCAAAAGTAA |
| | 126-5p | | UGGUACGCG | | TAATG |
| 5 | hsa-miR- | MIMAT0000421 | UGGAGUGUGACA | 46 | CAAACACCATTGTCAC |
| | 122-5p | | AUGGUGUUUG | | ACTCCA |
| 6 | Mmu-miR- | MIMAT0000548 | CAGCAGCAAUUCA | 47 | GTCCAAAACATGAATT |
| | 322-5p | | UGUUUUGGA | | GCTGCT |
| 7 | hsa-miR- | MIMAT0001341 | CAGCAGCAAUUCA | 48 | GTCCAAAACATGAATT |
| | 424-5p | | UGUUUUGAA | | GCTGCT |
| 8 | hsa-miR- | MIMAT0000241 | AUAAGACGAGCA | 49 | ACAAGCTTTTTGCTCGT |
| | 208a-3p | | AAAAGCUUGU | | CTTAT |
| 9 | hsa-miR- | MIMAT0004960 | AUAAGACGAACA | 50 | ACAAACCTTTTGTTCGT |
| | 208b-3p | | AAAGGUUUGU | | CTTAT |
| 10 | hsa-miR- | MIMAT0000273 | UAAUCUCAGCUGG | 51 | TCACAGTTGCCAGCTG |
| | 216a-5p | | CAACUGUGA | | AGATTA |
| 11 | mmu-miR- | MIMAT0000679 | UACUGCAUCAGGA | 52 | TCCAGTCAGTTCCTGAT |
| | 217-5p | | ACUGACUGGA | | GCAGTA |
| 12 | hsa-miR- | MIMAT0000274 | UACUGCAUCAGGA | 53 | TCCAATCAGTTCCTGAT |
| | 217-5p | | ACUGAUUGGA | | GCAGTA |
| 13 | hsa-miR- | MIMAT0000728 | UUUGUUCGUUCG | 54 | TCACGCGAGCCGAACG |
| | 375-3p | | GCUCGCGUGA | | AACAAA |
| 14 | hsa-miR- | MIMAT0000422 | UAAGGCACGCGGU | 55 | TTGGCATTCACCGCGTG |
| | 124-3p | | GAAUGCCAA | | CCTTA |
| 15 | hsa-miR-1- | MIMAT0000416 | UGGAAUGUAAAG | 56 | ATACATACTTCTTTACA |
| | 3p | | AAGUAUGUAU | | TTCCA |
| 16 | hsa-miR- | MIMAT0000427 | UUUGGUCCCCUUC | 57 | CAGCTGGTTGAAGGGG |
| | 133a-3p | | AACCAGCUG | | ACCAAA |
| 17 | hsa-miR- | MIMAT0000770 | UUUGGUCCCCUUC | 58 | TAGCTGGTTGAAGGGG |
| | 133b | | AACCAGCUA | | ACCAAA |
| 18 | hsa-miR-9- | MIMAT0000441 | UCUUUGGUUAUC | 59 | TCATACAGCTAGATAA |
| | 5p | | UAGCUGUAUGA | | CCAAAGA |
| 19 | hsa-miR- | MIMAT0000763 | UCCAGCAUCAGUG | 60 | TCCAGCATCAGTGATTT |
| | 338-3p | | AUUUUGUUG | | TGTTG |
| 20 | hsa-miR- | MIMAT0000276 | UGAUUGUCCAAAC | 61 | TGATTGTCCAAACGCA |
| | 219a-5p | | GCAAUUCU | | ATTCT |
| 21 | hsa-miR507 | MIMAT0002879 | UUUUGCACCUUUU | 62 | TTCACTCCAAAAGGTG |
| | | | GGAGUGAA | | CAAAA |
| 22 | hsa-miR- | MIMAT0002883 | AUUGACACUUCUG | 63 | ATTGACACTTCTGTGAG |
| | 514a-3p | | UGAGUAGA | | TAGA |
| 23 | hsa-miR- | MIMAT0004779 | UACUGCAGACAGU | 64 | TACTGCAGACAGTGGC |
| | 509-5p | | GGCAAUCA | | AATCA |
| 24 | hsa-miR-7- | MIMAT0000252 | UGGAAGACUAGU | 65 | AACAACAAAATCACTA |
| | 5p | | GAUUUUGUUGUU | | GTCTTCCA |
| 25 | hsa-miR- | MIMAT0000266 | UCCUUCAUUCCAC | 66 | CAGACTCCGGTGGAAT |
| | 205-5p | | CGGAGUCUG | | GAAGGA |
| 26 | hsa-miR- | MIMAT0000434 | UGUAGUGUUUCC | 67 | TCCATAAAGTAGGAAA |
| | 142-3p | | UACUUUAUGGA | | CACTACA |
| 27 | hsa-miR- | MIMAT0000232 | ACAGUAGUCUGCA | 68 | TAACCAATGTGCAGAC |
| | 199a-3p | | CAUUGGUUA | | TACTGT |
| 28 | hsa-miR- | MIMAT0000682 | UAACACUGUCUGG | 69 | ACATCGTTACCAGACA |
| | 200a-3p | | UAACGAUGU | | GTGTTA |
| 29 | hsa-miR- | MIMAT0000318 | UAAUACUGCCUGG | 70 | TCATCATTACCAGGCA |
| | 200b-3p | | UAAUGAUGA | | GTATTA |
| 30 | hsa-miR- | MIMAT0000222 | CUGACCUAUGAAU | 71 | GGCTGTCAATTCATAG |
| | 192-5p | | UGACAGCC | | GTCAG |
| 31 | has-miR- | MIMAT0000460 | UGUAACAGCAACU | 72 | TCCACATGGAGTTGCTG |
| | 194-5p | | CCAUGUGGA | | TTACA |
| 32 | hsa-miR- | MIMAT0001541 | UGGCAGUGUAUU | 73 | ACCAGCTAACAATACA |
| | 449a | | GUUAGCUGGU | | CTGCCA |
| 33 | hsa-let-7a- | MIMAT0000062 | UGAGGUAGUAGG | 74 | AACTATACAACCTACT |
| | 5p | | UUGUAUAGUU | | ACCTCA |
| 34 | hsa-let-7b- | MIMAT0000063 | UGAGGUAGUAGG | 75 | AACCACACAACCTACT |
| | 5p | | UUGUGUGGUU | | ACCTCA |
| 35 | hsa-let-7d- | MIMAT0000065 | AGAGGUAGUAGG | 76 | AACTATGCAACCTACT |
| | 5p | | UUGCAUAGUU | | ACCTCT |
| 36 | hsa-let-7e- | MIMAT0000066 | UGAGGUAGGAGG | 77 | AACTATACAACCTCCTA |
| | 5p | | UUGUAUAGUU | | CCTCA |
| 37 | hsa-let- | MIMAT0000067 | UGAGGUAGUAGA | 78 | AACTATACAATCTACTA |
| | 7f-5p | | UUGUAUAGUU | | CCTCA |
| 38 | hsa-let-7g- | MIMAT0000414 | UGAGGUAGUAGU | 79 | AACTGTACAAACTACT |
| | 5p | | UUGUACAGUU | | ACCTCA |
| 39 | hsa-let- | MIMAT0000415 | UGAGGUAGUAGU | 80 | AACAGCACAAACTACT |
| | 7i-5p | | UUGUGCUGUU | | ACCTCA |
| 40 | hsa-miR- | MIMAT000043 | UGAGAUGAAGCA | 81 | GAGCTACAGTGCTTCAT |
| | 143 | 5 | CUGUAGCUC | | CTCAT |
| 41 | hsa-miR- | MIMAT000024 | UCAGUGCACUAC | 82 | ACAAAGTTCTGTAGTG |
| | 148a-3p | 3 | AGAACUUUGU | | CACTGA |

In some embodiments, a contiguous polynucleic acid described herein consists of a single cassette, wherein the single cassette encodes an RNA comprising a miRNA target site (in addition to comprising the nucleic acid sequence of the output and the nucleic acid sequence of the trans activator).

In other embodiments, the contiguous polynucleic acid comprises two or more cassettes, at least one of which encodes an RNA comprising a miRNA target site.

In some embodiments, multiple cassettes of a contiguous polynucleic acid molecule comprise at least one miRNA target site. In some embodiments, each miRNA target site of a contiguous polynucleic acid is unique (i.e., the contiguous polynucleic acid includes only one copy of the miRNA target). In some embodiments, a contiguous polynucleic acid molecule comprises at least two cassettes that each comprise at least one miRNA target site that is the same nucleic acid sequence. In some embodiments, a contiguous polynucleic acid molecule comprises at least two cassettes that each comprise at least one miRNA target site, wherein at least one miRNA target site of each cassette comprises a different nucleic acid sequence that is regulated by the same miRNA. For example, a first cassette may comprise miRNA target site X and a second cassette may comprise miRNA target site Y and miRNA Z regulates target site X and target site Y.

In some embodiments, a miRNA (i.e., at least one miRNA) that regulates a miRNA target site of a contiguous polynucleic acid described herein is highly expressed and/or active in at least one cell type (e.g., of a multicellular organism, such as a mammal) in which the output expression must be low. A miRNA is highly expressed and/or active, as described herein, when output expression is decreased by at least 50% relative to the level of output expression of a reference contiguous polynucleic acid (i.e., lacking the miRNA target site(s) regulated by the miRNA, but otherwise containing the identical nucleic acid sequence) in said tissue cell type. In some embodiments, output is decreased, relative to the reference contiguous polynucleic acid, by at least 55%, at least 60%, at least 65%, at least 70%, at least 75%, at least 80%, at least 85%, at least 90%, at least 95%, at least 96%, at least 97%, at least 98%, at least 99%, or at least 99.9%.

In some embodiments, a miRNA (i.e., at least one miRNA) that regulates a miRNA target site of a contiguous polynucleic acid described herein is highly expressed and/or active in at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 150, at least 200, at least 500, at least 1000 cell types (e.g., of a multicellular organism, such as a mammal) in which the output expression must be low.

In some embodiments, a miRNA (i.e., at least one miRNA) that regulates a miRNA target of a contiguous polynucleic acid described herein has low expression and/or is inactive in at least one target cell type (e.g., of a multicellular organism, such as a mammal) in which output expression must be high. A miRNA has low expression and/or is inactive as described herein when output expression is decreased by less than 40% relative to the level of output expression of a reference contiguous polynucleic acid (i.e., lacking the miRNA target site(s) regulated by the miRNA, but otherwise containing the identical nucleic acid sequence) in said target cell type. In some embodiments, output is decreased, relative to the reference contiguous polynucleic acid, by less than 35%, less than 30%, less than 25%, less than 20%, less than 15%, less than 10%, less than 5%, less than 4%, less than 3%, less than 2%, or less than 1%. In some embodiments, there is no statistical difference between level of output expression from the contiguous polynucleic acid comprising the miRNA target and the reference continuous polynucleic acid molecule.

In some embodiments, a miRNA (i.e., at least one miRNA) that regulates a miRNA target site of a contiguous polynucleic acid described herein is expressed at low levels and/or inactive in at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 150, at least 200, at least 500, at least 1000 target cell types (e.g., of a multicellular organism, such as a mammal) in which the output expression must be high.

(ii) Exemplary Transactivators

Each of the contiguous polynucleic acids described herein comprises a cassette encoding an RNA (e.g., mRNA) comprising the nucleic acid sequence of a transactivator. In some embodiments, a contiguous polynucleic acid comprises the nucleic acid sequence of a single transactivator. In other embodiments, a contiguous polynucleic acid comprises the nucleic acid sequences of multiple transactivators (e.g., 2, 3, 4, 5, 6, 7, 8, 9, or 10 transactivators).

The terms “transactivator” or “transactivator protein,” as used herein, refer to a protein encoded on the contiguous polynucleic acid molecule that transactivates expression of an output (i.e., gene of interest) and that binds to a transactivator response element that is operably linked to the nucleic acid encoding an output (i.e., gene of interest). In some embodiments, the transactivator binds and transactivates the transactivator response element independently (i.e., in the absence of any additional factor). In other embodiments, the transactivator binds and transactivates the transactivator response element only in the presence of a transcription factor bound to the transcription factor response element.

In some embodiments, a transactivator protein comprises a DNA-binding domain. In some embodiments, the DNA-binding domain is engineered (i.e., not naturally-occurring) to bind a DNA sequence that is distinct from naturally-occurring sequences. Examples of DNA-binding domains are known to those having skill in the art and include, but are not limited to, DNA-binding domains derived using zinc-finger technology or TALEN technology or from mutant response regulators of two-component signaling pathways from bacteria.

In some embodiments, a DNA-binding domain is derived from a mammalian protein. In other embodiments a DNA binding domain is derived from a non-mammalian protein. For example, in some embodiments, a DNA-binding domain is derived from a protein originating in bacteria, yeast, or plants. In some embodiments, the DNA-binding domain requires an additional component (e.g., a protein or RNA) to target the transactivator response element. For example, in some embodiments, the DNA-binding domain is that of a CRISPR/Cas protein (e.g., Cas1, Cas2, Cas3, Cas5, Cas4, Cas6, Cas7, Cas8a, Cas8b, Cas8c, Cas9, Cas10, Cas10d, Cse1, Cse2, Csy1, Csy2, Csy3, Csm2, Cmr5, Csx10, Csx11, Csf1, Cpf1, C2c1, C2c2, C2c3) which requires the additional component of a guide RNA to target the transactivator response element.

In some embodiments, the transactivator protein is derived from a naturally-occurring transcription factor, wherein the DNA-binding domain of the naturally-occurring transcription factor has been mutated, resulting in an altered DNA binding specificity relative to the wild-type transcription factor. In some embodiments, the transactivator is a naturally-occurring transcription factor.

In some embodiments, a transactivator protein further comprises a transactivating domain (i.e., a fusion protein comprising a DNA binding domain and a transactivating domain). As used herein, the term “transactivating domain” refers to a protein domain that functions to recruit transcriptional machinery to a minimal promoter. In some embodiments, the transactivating domain does not trigger gene activation independently. In some embodiments, a transactivating domain is naturally-occurring. In other embodiments, a transactivating domain is engineered. Examples of transactivating domains are known to those having skill in the art and include, but are not limited to RelA transactivating domain, VP16, VP48, and VP64.

Exemplary transactivators are listed in TABLE 2. In some embodiments, the transactivator of at least one cassette is a transactivator listed in TABLE 2 or a transactivator having a least 70%, at least 80%, at least 85%, at least 90%, at least 95%, at least 96%, at least 97%, at least 98%, at least 99% identity of its amino acid sequence with one or more transactivator listed in TABLE 2. In some embodiments, a contiguous polynucleic acid molecule described herein encodes for a combination of transactivators listed in TABLE 2 or a combination of transactivators having a least 70%, at least 80%, at least 85%, at least 90%, at least 95%, at least 96%, at least 97%, at least 98%, at least 99% identity of its amino acid sequence with one or more transactivators listed in TABLE 2.

In some embodiments, the transactivator of at least one cassette is tTA, rtTA, PIT-RelA, PIT-VP16, ET-VP16, ET-RelA, NarLc-VP16, or NarLc-RelA. See e.g., Angelici B. et al., Cell Rep. 2016 Aug. 30; 16(9): 2525-2537.

TABLE 2
Exemplary transactivators. The DNA sequences are jusl examples (hat are
capable of encoding the protein sequences depicted; due to degenerate codons, very large sets
of DNA sequences can encode the same protein sequence. The transactivator domains such as
RclA and VP16 arc only examples of possible transactivator domains (TAD). “VP16 TAD”
stands for a transactivator domain derived from a VP 16 gene of a Herpes Simplex Virus;
multiple domains and their combinations and their mutants can serve as transactivalor
domains when fused to DNA binding domains. The DNA binding domains (DBD) of
transactivators, when derived from full-length proteins, arc merely examples of such
domains; they may be further decreased or increased to include more amino acids from their
full-length protein progenitor. The DBD derived from the response regulators of prokaryotic
two component signaling systems arc shown based on their protein sequence in E. coli.
however, the orthologs of these genes from other prokaryotic strains and species could he
used just as well. In addition. DNA binding domains of response regulators from two-
component signaling pathways that do not have orthologs in E. coli, can also be used for the
same purpose. M (underlined) represents a start codon introduced in front of various DBDs to
enable their translation. “::” represents a point of fusion between the DBD and TAD.

| | | Type of | |
|---|---|---|---|
| Seq ID | Name | sequence | Sequence DNA/Protein |
| 83 | RelA-TAD-1 | DNA | ATGATGAGTTTCCCACCATGGTGTTTCCTTCTGGGCAGATCA |
| | | sequence | GCCAGGCCTCGGCCTTGGCCCCGGCCCCTCCCCAAGTCCTGC |
| | | (example) | CCCAGGCTCCAGCCCCTGCCCCTGCTCCAGCCATGGTATCAG |
| | | | CTCTGGCCCAGGCCCCAGCCCCTGTCCCAGTCCTAGCCCCAG |
| | | | GCCCTCCTCAGGCTGTGGCCCCACCTGCCCCCAAGCCCACCC |
| | | | AGGCTGGGGAAGGAACGCTGTCAGAGGCCCTGCTGCAGCTG |
| | | | CAGTTTGATGATGAAGACCTGGGGGCCTTGCTTGGCAACAG |
| | | | CACAGACCCAGCTGTGTTCACAGACCTGGCATCCGTCGACA |
| | | | ACTCCGAGTTTCAGCAGCTGCTGAACCAGGGCATACCTGTGG |
| | | | CCCCCCACACAACTGAGCCCATGCTGATGGAGTACCCTGAG |
| | | | GCTATAACTCGCCTAGTGACAGGGGCCCAGAGGCCCCCCGA |
| | | | CCCAGCTCCTGCTCCACTGGGGGCCCCGGGGCTCCCCAATGG |
| | | | CCTCCTTTCAGGAGATGAAGACTTCTCCTCCATTGCGGACAT |
| | | | GGACTTCTCAGCCCTGCTGAGTCAGATCAGCTCCTAA |
| 84 | | Protein | HDEFPTMVFPSGQISQASALAPAPPQVLPQAPAPAPAPAMVSAL |
| | | sequence | AQAPAPVPVLAPGPPQAVAPPAPKPTQAGEGTLSEALLQLQFD |
| | | | DEDLGALLGNSTDPAVFTDLASVDNSEFQQLLNQGIPVAPHTTE |
| | | | PMLMEYPEAITRLVTGAQRPPDPAPAPLGAPGLPNGLLSGDEDF |
| | | | SSIADMDFSALLSQISS |
| 85 | RelA-TAD-2 | DNA | CAGGCTGGGGAAGGAACGCTGTCAGAGGCCCTGCTGCAGCT |
| | | sequence | GCAGTTTGATGATGAAGACCTGGGGGCCTTGCTTGGCAACA |
| | | (example) | GCACAGACCCAGCTGTGTTCACAGACCTGGCATCCGTCGAC |
| | | | AACTCCGAGTTTCAGCAGCTGCTGAACCAGGGCATACCTGTG |
| | | | GCCCCCCACACAACTGAGCCCATGCTGATGGAGTACCCTGA |
| | | | GGCTATAACTCGCCTAGTGACAGGCGCACAACGCCCACCTG |
| | | | ATCCGGCACCAGCACCCCTTGGAGCTCCCGGTCTCCCCAATG |
| | | | GCCTCCTTTCAGGAGATGAAGACTTCTCCTCCATTGCGGACA |
| | | | TGGACTTCTCAGCCCTGCTGAGTCAGATCAGCTCC |
| 86 | | Protein | QAGEGTLSEALLQLQFDDEDLGALLGNSTDPAVFTDLASVDNS |
| | | sequence | EFQQLINQGIPVAPHTTEPMLMEYPEAITRLVTGAQRPPDPAP |
| | | | APLGAPGLPNGLLSGDEDFSSIADMDFSALLSQISS |
| 87 | RelA-TAD-1 | DNA | CCCAAGCCAGCACCCCAGCCCTATCCCTTTACGTCATCCCTG |
| | | sequence | AGCACCATCAACTATGATGAGTTTCCCACCATGGTGTTTCCT |
| | | (example) | TCTGGGCAGATCAGCCAGGCCTCGGCCTTGGCCCCGGCCCCT |
| | | | CCCCAAGTCCTGCCCCAGGCTCCAGCCCCTGCCCCTGCTCCA |
| | | | GCCATGGTATCAGCTCTGGCCCAGGCCCCAGCCCCTGTCCCA |
| | | | GTCCTAGCCCCAGGCCCTCCTCAGGCTGTGGCCCCACCTGCC |
| | | | CCCAAGCCCACCCAGGCTGGGGAAGGAACGCTGTCAGAGGC |
| | | | CCTGCTGCAGCTGCAGTTTGATGATGAAGACCTGGGGGCCTT |
| | | | GCTTGGCAACAGCACAGACCCAGCTGTGTTCACAGACCTGG |
| | | | CATCCGTCGACAACTCCGAGTTTCAGCAGCTGCTGAACCAGG |
| | | | GCATACCTGTGGCCCCCCACACAACTGAGCCCATGCTGATGG |
| | | | AGTACCCTGAGGCTATAACTCGCCTAGTGACAGGGGCCCAG |
| | | | AGGCCCCCCGACCCAGCTCCTGCTCCACTGGGGGCCCCGGG |
| | | | GCTCCCCAATGGCCTCCTTTCAGGAGATGAAGACTTCTCCTC |
| | | | CATTGCGGACATGGACTTCTCAGCCCTGCTGAGTCAGATCAG |
| | | | CTCC |
| 88 | | Protein | PKPAPQPYPFTSSLSTINYDEFPTMVFPSGQISQASALAPAPPQ |
| | | sequence | VLPQAPAPAPAPAMVSALAQAPAPVPVLAPGPPQAVAPPAPKPT |
| | | | QAGEGTLSEALLQLQFDDEDLGALLGNSTDPAVFTDLASVDNSE |
| | | | FQQLLNQGIPVAPHTTEPMLMEYPEAITRLVTGAQRPPDPAPAP |
| | | | LGAPGLPNGLLSGDEDFSSIADMDFSALLSQISS |
| 89 | VP16-TAD-1 | DNA | GCCCCCCCGACCGATGTCAGCCTGGGGGACGAGCTCCACTT |
| | | sequence | AGACGGCGAGGACGTGGCGATGGCGCATGCCGACGCGCTAG |
| | | (example) | ACGATTTCGATCTGGACATGTTGGGGGACGGGGATTCCCCG |
| | | | GGTCCGGGATTTACCCCCCACGACTCCGCCCCCTACGGCGCT |
| | | | CTGGATATGGCCGACTTCGAGTTTGAGCAGATGTTTACCGAT |
| | | | GCCCTTGGAATTGACGAGTACGGTGGG |
| 90 | | Protein | APPTDVSLGDELHLDGEDVAMAHADALDDFDLDMLGDGDSPG |
| | | sequence | PGFTPHDSAPYGALDMADFEFEQMFTDALGIDEYGG |
| 91 | VP16-TAD-2 | DNA | CCGGCAGATGCCCTTGATGACTTCGATTTGGACATGCTCCCA |
| | | sequence | GCGGATGCCTTGGACGATTTTGATCTCGATATGCTTCCCGCC |
| | | (example) | GACGCACTCGATGATTTCGATCTGGATATGCTCCCGGGT |
| 92 | | Protein | PADALDDFDLDMLPADALDDFDLDMLPADALDDFDLDMLPG |
| | | sequence | |
| 93 | VP48-TAD-1 | DNA | GGTCCGGCAGATGCCCTTGATGACTTCGATTTGGACATGCTC |
| | | sequence | CCAGCGGATGCCTTGGACGATTTTGATCTCGATATGCTTCCC |
| | | (example) | GCCGACGCACTCGATGATTTCGATCTGGATATGCTCCCGGGT |
| 94 | | Protein | GPADALDDFDLDMLPADALDDFDLDMLPADALDDFDLDMLPG |
| | | sequence | |
| 95 | PT1::RelA TAD-1 | DNA | APGAGICGAGGAGAGGTGCGCATGGCGAAGGCAGGGCGGG |
| | | sequence | AGGGGCCGCGGGACAGCGTGTGGCTGTCGGGGGAGGGGCG |
| | | (example) | GCGCGGCGGTCGCCGTGGGGGGCAGCCGTCCGGGCTCGACC |
| | | | GGGACCGGATCACCGGGGTCACCGTCCGGCTGCTGGACACG |
| | | | GAGGGCCTGACGGGGTTCTCGATGCGCCGCCTGGCCGCCGA |
| | | | GCTGAACGTCACCGCGATGTCCGTGTACTGGTACGTCGACAC |
| | | | CAAGGACCAGTTGCTCGAGCTCGCCCTGGACGCCGTCTTCGG |
| | | | CGAGCTGCGCCACCCGGACCCGGACGCCGGGCTCGACTGGC |
| | | | GCGAGGAACTGCGGGCCCTGGCCCGGGAGAACCGGGCGCTG |
| | | | CTGGTGCGCCACCCCTGGTCGTCCCGGCTGGTCGGCACCTAC |
| | | | CTCAACATCGGCCCGCACTCGCTGGCCTTCTCCCGCGCGGTG |
| | | | CAGAACGTCGTGCGCCGCAGCGGGCTGCCCGCGCACCGCCT |
| | | | GACCGGCGCCATCTCGGCCGTCTTCCAGTTCGTCTACGGCTA |
| | | | CGGCACCATCGAGGGCCGCTTCCTCGCCCGGGTGGCGGACA |
| | | | CCGGGCTGAGTCCGGAGGAGTACTTCCAGGACTCGATGACC |
| | | | GCGGTGACCGAGGTGCCGGACACCGCGGGCGTCATCGAGGA |
| | | | CGCGCAGGACATCATGGCGGCCCGGGGCGGCGACACCGTGG |
| | | | CGGAGATGCTGGACCGGGACTTCGAGTTCGCCCTCGACCTGC |
| | | | TCGTCGCGGGCATCGACGCGATGGTCGAACAGGCCTCCGCG |
| | | | TACAGCCGCGCGC::ATGATGAGTTTCCCACCATGGTGTTTCC |
| | | | TTCTGGGCAGATCAGCCAGGCCTCGGCCTTGGCCCCGGCCCC |
| | | | TCCCCAAGTCCTGCCCCAGGCTCCAGCCCCTGCCCCTGCTCC |
| | | | AGCCATGGTATCAGCTCTGGCCCAGGCCCCAGCCCCTGTCCC |
| | | | AGTCCTAGCCCCAGGCCCTCCTCAGGCTGTGGCCCCACCTGC |
| | | | CCCCAAGCCCACCCAGGCTGGGGAAGGAACGCTGTCAGAGG |
| | | | CCCTGCTGCAGCTGCAGTTTGATGATGAAGACCTGGGGGCCT |
| | | | TGCTTGGCAACAGCACAGACCCAGCTGTGTTCACAGACCTG |
| | | | GCATCCGTCGACAACTCCGAGTTTCAGCAGCTGCTGAACCAG |
| | | | GGCATACCTGTGGCCCCCCACACAACTGAGCCCATGCTGATG |
| | | | GAGTACCCTGAGGCTATAACTCGCCTAGTGACAGGGGCCCA |
| | | | GAGGCCCCCCGACCCAGCTCCTGCTCCACTGGGGGCCCCGG |
| | | | GGCTCCCCAATGGCCTCCTTTCAGGAGATGAAGACTTCTCCT |
| | | | CCATTGCGGACATGGACTTCTCAGCCCTGCTGAGTCAGATCA |
| | | | GCTCCTAA |
| 96 | | Protein | MSRGEVRMAKAGREGPRDSVWLSGEGRRGGRRGGQPSGLDR |
| | | sequence | DRITGVTVRLLDTEGLTGFSMRRLAAELNVTAMSVYWYVDTK |
| | | | DQLLELALDAVFGELRHPDPDAGLDWREELRALARENRALLV |
| | | | RHPWSSRLVGTYLNIGPHSLAFSRAVQNVVRRSGLPAHRLTG |
| | | | AISAVFQFVYGYGTIEGRFLARVADTGLSPEEYFQDSMTAVT |
| | | | EVPDTAGVIEDAQDIMAARGGDTVAEMLDRDFEFALDLLVAG |
| | | | IDAMVEQASAYSRA::HDEFPTMVFPSGQISQASALAPAPPQ |
| | | | VLPQAPAPAPAPAMVSALAQAPAPVPVLAPGPPQAVAPPAPK |
| | | | PTQAGEGTLSEALLQLQFDDEDLGALLGNSTDPAVFTDLASV |
| | | | DNSEFQQLLNQGIPVAPHTTEPMLMEYPEAITRLVTGAQRPP |
| | | | DPAPAPLGAPGLPNGLLSGDEDFSSIADMDFSALLSQISS |
| 97 | PT1::VP16 TAD-2 | DNA | APGAGTCGAGGAGAGGTGCGCATGGCGAAGGCAGGGCGGG |
| | | sequence | AGGGGCCGCGGGACAGCGTGTGGCTGTCGGGGGAGGGGCG |
| | | (example) | GCGCGGCGGTCGCCGTGGGGGGCAGCCGTCCGGGCTCGACC |
| | | | GGGACCGGATCACCGGGGTCACCGTCCGGCTGCTGGACACG |
| | | | GAGGGCCfGACGGGGTTCTCGATGCGCCGCCTGGCCGCCGA |
| | | | GCTGAACGTCACCGCGATGTCCGTGTACTGGTACGTCGACAC |
| | | | CAAGGACCAGTTGCTCGAGCTCGCCCTGGACGCCGTCTTCGG |
| | | | CGAGCTGCGCCACCCGGACCCGGACGCCGGGCTCGACTGGC |
| | | | GCGAGGAACTGCGGGCCCTGGCCCGGGAGAACCGGGCGCTG |
| | | | CTGGTGCGCCACCCCTGGTCGTCCCGGCTGGTCGGCACCTAC |
| | | | CTCAACATCGGCCCGCACTCGCTGGCCTTCTCCCGCGCGGTG |
| | | | CAGAACGTCGTGCGCCGCAGCGGGCTGCCCGCGCACCGCCT |
| | | | GACCGGCGCCATCTCGGCCGTCTTCCAGTTCGTCTACGGCTA |
| | | | CGGCACCATCGAGGGCCGCTTCCTCGCCCGGGTGGCGGACA |
| | | | CCGGGCTGAGTCCGGAGGAGTACTTCCAGGACTCGATGACC |
| | | | GCGGTGACCGAGGTGCCGGACACCGCGGGCGTCATCGAGGA |
| | | | CGCGCAGGACATCATGGCGGCCCGGGGCGGCGACACCGTGG |
| | | | CGGAGATGCTGGACCGGGACTTCGAGTTCGCCCTCGACCTGC |
| | | | TCGTCGCGGGCATCGACGCGATGGTCGAACAGGCCTCCGCG |
| | | | TACAGCCGCGCGCGTACGAAAAACAATTACGGGTCTACCAT |
| | | | CGAGGGCCTGCTCGATCTCCCGGACGACGACGCCCCCGAAG |
| | | | AGGCGGGGCTGGCGGCTCCGCGCCTGTCCTTTCTCCCCGCGG |
| | | | GACACACGCGCAGACTGTCGACG::GCCCCCCCGACCGATGT |
| | | | CAGCCTGGGGGACGAGCTCCACTTAGACGGCGAGGACGTGG |
| | | | CGATGGCGCATGCCGACGCGCTAGACGATTTCGATCTGGAC |
| | | | ATGTTGGGGGACGGGGATTCCCCGGGTCCGGGATTTACCCCC |
| | | | CACGACTCCGCCCCCTACGGCGCTCTGGATATGGCCGACTTC |
| | | | GAGTTTGAGCAGATGTTTACCGATGCCCTTGGAATTGACGAG |
| | | | TACGGTGGG |
| 98 | | Protein | MSRGEVRMAKAGREGPRDSVWLSGEGRRGGRRGGQPSGLDR |
| | | sequence | DRITGVTVRLLDTEGLTGFSMRRLAAELNVTAMSVYWYVDTK |
| | | | DQLLELALDAVFGELRHPDPDAGLDWREELRALARENRALLV |
| | | | RHPWSSRLVGTYLNIGPHSLAFSRAVQNVVRRSGLPAHRITGAI |
| | | | SAVFQFVYGYGTIEGRFLARVADTGLSPEEYFQDSMTAVTEVP |
| | | | DTAGVIEDAQDIMAARGGDTVAEMLDRDFEFALDLLVAGIDA |
| | | | MVEQASAYSRARTKNNYGSTIEGLLDLPDDDAPEEAGLAAPRL |
| | | | SFLPAGHTRRLST::APPTDVSLGDELHLDGEDVAMAHADALDD |
| | | | FDLDMLGDGDSPGPGFTPHDSAPYGALDMADFEFEQMFTDAL |
| | | | GIDEYGG |
| 99 | ET::RelA TAD-1 | DNA | ATGCCCCGCCCCAAGCTCAAGTCCGATGACGAGGTACTCGA |
| | | sequence | GGCCGCCACCGTAGTGCTGAAGCGTTGCGGTCCCATAGAGTT |
| | | (example) | CACGCTCAGCGGAGTAGCAAAGGAGGTGGGGCTCTCCCGCG |
| | | | CAGCGlTAATCCAGCGCTTCACCAACCGCGATACGCTGCTGG |
| | | | TGAGGATGATGGAGCGCGGCGTCGAGCAGGTGCGGCATTAC |
| | | | CTGAATGCGATACCGATAGGCGCAGGGCCGCAAGGGCTCTG |
| | | | GGAATTTTTGCAGGTGCTCGTTCGGAGCATGAACACTCGCAA |
| | | | CGACTTCTCGGTGAACTATCTCATCTCCTGGTACGAGCTCCA |
| | | | GGTGCCGGAGCTACGCACGCTTGCGATCCAGCGGAACCGCG |
| | | | CGGTGGTGGAGGGGATCCGCAAGCGACTGCCCCCAGGTGCT |
| | | | CCTGCGGCAGCTGAGTTGCTCCTGCACTCGGTCATCGCTGGC |
| | | | GCGACGATGCAGTGGGCCGTCGATCCGGATGGTGAGCTAGC |
| | | | TGATCATGTGCTGGCTCAGATCGCTGCCATCCTGTGTTTAAT |
| | | | GTTTCCCGAACACGACGATTTCCAACTCCTCCAGGCACATGC |
| | | | GTCCGCGTACAGCCGCGCGC-ATGATGAGTTTCCCACCATGG |
| | | | TGTTTCCTTCTGGGCAGATCAGCCAGGCCTCGGCCTTGGCCC |
| | | | CGGCCCCTCCCCAAGTCCTGCCCCAGGCTCCAGCCCCTGCCC |
| | | | CTGCTCCAGCCATGGTATCAGCTCTGGCCCAGGCCCCAGCCC |
| | | | CTGTCCCAGTCCTAGCCCCAGGCCCTCCTCAGGCTGTGGCCC |
| | | | CACCTGCCCCCAAGCCCACCCAGGCTGGGGAAGGAACGCTG |
| | | | TCAGAGGCCCTGCTGCAGCTGCAGTTTGATGATGAAGACCTG |
| | | | GGGGCCTTGCTTGGCAACAGCACAGACCCAGCTGTGTTCAC |
| | | | AGACCTGGCATCCGTCGACAACTCCGAGTTTCAGCAGCTGCT |
| | | | GAACCAGGGCATACCTGTGGCCCCCCACACAACTGAGCCCA |
| | | | TGCTGATGGAGTACCCTGAGGCTATAACTCGCCTAGTGACAG |
| | | | GGGCCCAGAGGCCCCCCGACCCAGCTCCTGCTCCACTGGGG |
| | | | GCCCCGGGGCTCCCCAATGGCCTCCTTTCAGGAGATGAAGA |
| | | | CTTCTCCTCCATTGCGGACATGGACTTCTCAGCCCTGCTGAG |
| | | | TCAGATCAGCTCCTAA |
| 100 | | Protein | MPRPKLKSDDEVLEAATVVLKRCGPIEFTLSGVAKEVGLSRAA |
| | | sequence | LIQRFTNRDTILVRMMERGVEQVRHYLNAIPIGAGPQGLWEFL |
| | | | QVLVRSMNTRNDFSVNYLISWYELQVPELRTLAIQRNRAVVEG |
| | | | IRKRLPPGAPAAAELLLHSVIAGATMQWAVDPDGELADHVLAQ |
| | | | IAAILCLMFPEHDDFQLLQAHASAYSRA::HDEFPTMVFPSGQI |
| | | | SQASALAPAPPQVLPQAPAPAPAPAMVSALAQAPAPVPVLAPG |
| | | | PPQAVAPPAPKPTQAGEGTLSEALLQLQFDDEDLGALLGNSTDP |
| | | | AVFTDLASVDNSEFQQLLNQGIPVAPHTTEPMLMEYPEAITRLV |
| | | | TGAQRPPDPAPAPLGAPGLPNGLLSGDEDFSSIADMDFSALLS |
| | | | QISS |
| 101 | ET::VP16TAD-2 | DNA | ATGCCCCGCCCCAAGCTCAAGTCCGATGACGAGGTACTCGA |
| | | sequence | GGCCGCCACCGTAGTGCTGAAGCGTTGCGGTCCCATAGAGTT |
| | | (example) | CACGCTCAGCGGAGTAGCAAAGGAGGTGGGGCTCTCCCGCG |
| | | | CAGCGlTAATCCAGCGCTTCACCAACCGCGATACGCTGCTGG |
| | | | TGAGGATGATGGAGCGCGGCGTCGAGCAGGTGCGGCATTAC |
| | | | CTGAATGCGATACCGATAGGCGCAGGGCCGCAAGGGCTCTG |
| | | | GGAATTTTTGCAGGTGCTCGTTCGGAGCATGAACACTCGCAA |
| | | | CGACTTCTCGGTGAACTATCTCATCTCCTGGTACGAGCTCCA |
| | | | GGTGCCGGAGCTACGCACGCTTGCGATCCAGCGGAACCGCG |
| | | | CGGTGGTGGAGGGGATCCGCAAGCGACTGCCCCCAGGTGCT |
| | | | CCTGCGGCAGCTGAGTTGCTCCTGCACTCGGTCATCGCTGGC |
| | | | GCGACGATGCAGTGGGCCGTCGATCCGGATGGTGAGCTAGC |
| | | | TGATCATGTGCTGGCTCAGATCGCTGCCATCCTGTGTTTAAT |
| | | | GTTTCCCGAACACGACGATTTCCAACTCCTCCAGGCACATGC |
| | | | GTCCGCGTACAGCCGCGCGCGTACGAAAAACAATTACGGGT |
| | | | CTACCATCGAGGGCCTGCTCGATCTCCCGGACGACGACGCCC |
| | | | CCGAAGAGGCGGGGCTGGCGGCTCCGCGCCTGTCCTTTCTCC |
| | | | CCGCGGGACACACGCGCAGACTGTCGACG::GCCCCCCCGAC |
| | | | CGATGTCAGCCTGGGGGACGAGCTCCACTTAGACGGCGAGG |
| | | | ACGTGGCGATGGCGCATGCCGACGCGCTAGACGATTTCGAT |
| | | | CTGGACATGTTGGGGGACGGGGATTCCCCGGGTCCGGGATT |
| | | | TACCCCCCACGACTCCGCCCCCTACGGCGCTCTGGATATGGC |
| | | | CGACTTCGAGTTTGAGCAGATGTTTACCGATGCCCTTGGAAT |
| | | | TGACGAGTACGGTGGG |
| 102 | | Protein | MPRPKLKSDDEVLEAATVVLKRCGPIEFTLSGVAKEVGLSRAA |
| | | sequence | LIQRFTNRDTILVRMMERGVEQVRHYLNAIPIGAGPQGLWEFL |
| | | | QVLVRSMNTRNDFSVNYLISWYELQVPELRTLAIQRNRAVVEG |
| | | | IRKRLPPGAPAAAELLLHSVIAGATMQWAVDPDGELADHVLA |
| | | | QIAAILCIMFPEHDDFQLIQAHASAYSRARTKNNYGSTIEGLLD |
| | | | LPDDDAPEEAGLAAPRLSFLPAGHTRRISI::APPTDVSLGDELH |
| | | | LDGEDVAMAHADALDDIDLDMLGDGDSPGPGFTPHDSAPYGA |
| | | | LDMADFEFEQMFTDALGIDEYGG |
| 103 | LEX::VP16 TAD-2 | | ATGAAAGCGTTAACGGCCAGGCAACAAGAGGTGTTTGATCT |
| | | DNA | CATCCGTGATCACATCAGCCAGACAGGTATGCCGCCGACGC |
| | | sequence | GTGCGGAAATCGCGCAGCGTTTGGGGTTCCGTTCCCCAAACG |
| | | (example) | CGGNTGAAGAACATCTGAAGGCGCTGGCACGCAAAGGCGTT |
| | | | ATTGAAATTGTTTCCGGCGCATCACGCGGGATTCGTCTGTTG |
| | | | CAGGAAGAGGAAGAAGGGTTGCCGCTGGTAGGTCGTGTGGC |
| | | | TGCCGGTGAACCACTTCTGGCGCAACAGCATATTGAAGGTC |
| | | | ATTATCAGGTCGATCCTTCCTTATTCAAGCCGAATGCTGATT |
| | | | TCCTGCTGCGCGTCAGCGGGATGTCGATGAAAGATATCGGC |
| | | | ATTATGGATGGTGACTTGCTGGCAGTGCATAAAACTCAGGAT |
| | | | GTACGTAACGGTCAGGTCGTTGTCGCACGTATTGATGACGAA |
| | | | GTTACCGTTAAGCGCCTGAAAAAACAGGGCAATAAAGTCGA |
| | | | ACTGTTGCCAGAAAATAGCGAGTTTAAACCAATTGTCGTTGA |
| | | | CCTTCGTCAGCAGAGCTTCACCATTGAAGGTCTGGCGGTTGG |
| | | | GGTTATTCGCAACGGCGACTGGCTGTCTAGCTATCCTTATGA |
| | | | CGTGCCTGACTATGCCAGCCTGGGAGGATCTAGA::GCCCCCC |
| | | | CGACCGATGTCAGCCTGGGGGACGAGCTCCACTTAGACGGC |
| | | | GAGGACGTGGCGATGGCGCATGCCGACGCGCTAGACGATTT |
| | | | CGATCTGGACATGTTGGGGGACGGGGATTCCCCGGGTCCGG |
| | | | GATTTACCCCCCACGACTCCGCCCCCTACGGCGCTCTGGATA |
| | | | TGGCCGACTTCGAGTTTGAGCAGATGTTTACCGATGCCCTTG |
| | | | GAATTGACGAGTACGGTGGGTAGTG |
| 104 | | Protein | MKALTARQQEVFDLIRDHISQTGMPPTRAEIAQRLGFRSPNA7E |
| | | sequence | EHLKALARKGVIEIVSGASRGIRLLQEEEEGLPLVGRVAAGEPL |
| | | | LAQQHIEGHYQVDPSIFKPNADFILRVSGMSMKDIGIMDGDLL |
| | | | AVHKTQDVRNGQVVVARIDDEVTVKRIKKQGNKVELLPENSE |
| | | | FKPIVVDLRQQSFTIEGIAVGVIRNGDWLSSYPYDVPDYASLGG |
| | | | SR::APPTDVSIGDEIHLDGEDVAMAHADALDDFDLDMLGDGD |
| | | | SPGPGFTPHDSAPYGALDMADFEFEQMFTDALGIDEYGG |
| 105 | NarL DBD | DNA | ATGGCTACGACCGAGCGGGACGTAAACCAGCTTACTCCGAG |
| | [NARL_ECOLI | sequence | AGAGAGGGACATTTTGAAGCTGATTGCGCAGGGGCTTCCCA |
| | UniProtKB- | (example) | ATAAGATGATTGCCAGACGCCTTGATATCACGGAAAGCACT |
| | P0AF28I147 | | GTGAAAGTCCACGTGAAACACATGCTCAAAAAGATGAAACT |
| | -215]::VP16 | | CAAGTCCCGCGTGGAAGCTGCGGTCTGGGTACATCAGGAGC |
| | TAD-2 | | GAATCTTTGGT::CCGGCAGATGCCCTTGATGACTTCGATTTG |
| | | | GACATGCTCCCAGCGGATGCCTTGGACGATTTTGATCTCGAT |
| | | | ATGCTTCCCGCCGACGCACTCGATGATTTCGATCTGGATATG |
| | | | CTCCCGGGT |
| 106 | | Protein | MATTERDVNQLTPRERDILKLIAQGLPNKMIARRLDITESTVKV |
| | | sequence | HVKHMLKKMKLKSRVEAAVWVHQERIFG::PADALDDFDLDM |
| | | | LPADALDDFDLDMLPADALDDFDLDMLPG |
| 107 | NarL DBD | DNA | ATGGCTACGACCGAGCGGGACGTAAACCAGCTTACTCCGAG |
| | [NARL_ECOLI | sequence | AGAGAGGGACATTTTGAAGCTGATTGCGCAGGGGCTTCCCA |
| | UniProtKB- | (example) | ATAAGATGATTGCCAGACGCCTTGATATCACGGAAAGCACT |
| | POAF28I147 | | GTGAAAGTCCACGTGAAACACATGCTCAAAAAGATGAAACT |
| | -215]::VP16 | | CAAGTCCCGCGTGGAAGCTGCGGTCTGGGTACATCAGGAGC |
| | TAD-1 | | GAATCTTTGCCAGC::GCCCCCCCGACCGATGTCAGCCTGGGG |
| | | | GACGAGCTCCACTTAGACGGCGAGGACGTGGCGATGGCGCA |
| | | | TGCCGACGCGCTAGACGATTTCGATCTGGACATGTTGGGGG |
| | | | ACGGGGATTCCCCGGGTCCGGGATTTACCCCCCACGACTCCG |
| | | | CCCCCTACGGCGCTCTGGATATGGCCGACTTCGAGTTTGAGC |
| | | | AGATGTTTACCGATGCCCTTGGAATTGACGAGTACGGTGGGT |
| | | | GA |
| 108 | | Protein | MATTERDVNQLTPRERDILKLIAQGLPNKMIARRLDITESTVKV |
| | | sequence | HVKHMLKKMKLKSRVEAAVWVHQERIFAS::APPTDVSLGDEL |
| | | | HLDGEDVAMAHADALDDFDLDMLGDGDSPGPGFTPHDSAPYG |
| | | | ALDMADFEFEQMFTDALGIDEYGG |
| 109 | NarL DBD | DNA | ATGGCTACGACCGAGCGGGACGTAAACCAGCTTACTCCGAG |
| | [NARL_ECOLI | sequence | AGAGAGGGACATTTTGAAGCTGATTGCGCAGGGGCTTCCCA |
| | UniProtKB - | (example) | ATAAGATGATTGCCAGACGCCTTGATATCACGGAAAGCACT |
| | POAF28I147 | | GTGAAAGTCCACGTGAAACACATGCTCAAAAAGATGAAACT |
| | -215]::VP48 | | CAAGTCCCGCGTGGAAGCTGCGGTCTGGGTACATCAGGAGC |
| | TAD-1 | | GAATCTTTGCCAGC::GGTCCGGCAGATGCCCTTGATGACTTC |
| | | | GATTTGGACATGCTCCCAGCGGATGCCTTGGACGATTTTGAT |
| | | | CTCGATATGCTTCCCGCCGACGCACTCGATGATTTCGATCTG |
| | | | GATATGCTCCCGGGTTGA |
| 110 | | Protein | MATTERDVNQLTPRERDILKLIAQGLPNKMIARRLDITESTVKV |
| | | sequence | HVKHMLKKMKLKSRVEAAVWVHQERIFAS::GPADALDDFDL |
| | | | DMLPADALDDFDLDMLPADALDDFDLDMLPG |
| 111 | OMPR-D55E:: | DNA | ATGCAAGAAAACTACAAGATTCTCGTGGTGGATGATGACAT |
| | VP16 TAD-2 | sequence | GCGACTTCGCGCATTGCTCGAtAAGATATCTGACCGAGCAGG |
| | | (example) | GATTTCAAGTGCGCTCCGTGGCCAATGCCGAGCAGATGGAT |
| | | | AGGCTCTTGACGAGGGAGTCGTTCCATCTGATGGTGCTGGAA |
| | | | TTGATGCTTCCCGGTGAGGACGGATTGTCCATTTGCCGGAGA |
| | | | CTTAGGTCGCAGTCAAACCCCATGCCGATCATCATGGTCACA |
| | | | GCGAAGGGAGAGGAGGTCGATAGAATTGTAGGTCTTGAGAT |
| | | | TGGGGCAGACGACTACATCCCCAAGCCGTTCAATCCCCGGG |
| | | | AACTTCTTGCGCGAATCCGAGCCGTGCTCAGGCGACAGGCC |
| | | | AACGAGCTGCCCGGAGCTCCATCGCAAGAGGAAGCGGTCAT |
| | | | CGCGTTCGGGAAGTTCAAGTTGAACCTCGGCACGAGAGAGA |
| | | | TGTTTCGGGAAGATGAACCTATGCCGCTCACATCGGGGGAG |
| | | | TTTGCGGTCTTGAAAGCACTTGTCTCACACCCGAGAGAACCT |
| | | | CTGTCGCGGGATAAACTCATGAATCTGGCGAGAGGCAGAGA |
| | | | GTATAGCGCGATGGAAAGGTCCATCGATGTCCAGATTAGCC |
| | | | GCCTCCGCCGCATGGTGGAGGAAGATCCAGCCCACCCTCGG |
| | | | TACATCCAGACTGTATGGGGATTGGGGTATGTGTTCGTACCG |
| | | | GATGGGTCAAAAGCAGGA::CCGGCGGACGCACTGGATGACT |
| | | | TTGACTTGGATATGCTCCCAGCGGATGCGTTGGACGATTTTG |
| | | | ACCTTGACATGTTGCCTGCCGACGCGCTTGACGACTTCGACT |
| | | | TGGACATGCTGCCCGGT |
| 112 | | Protein | MQENYKILVVDDDMRLRALIERYLTIQGFQVRSVANAEQMD |
| | | sequence | RLLTRESFHLMVLELMLPGEDGLSICRRLRSQSNPMPIIMVTAK |
| | | | GEEVDRIVGIEIGADDYIPKPFNPREILARIRAVLRRQANELPGA |
| | | | PSQEEAVIAFGKFKLNLGTREMFREDEPMPLTSGEFAVLKALVS |
| | | | HPREPLSRDKLMNLARGREYSAMERSIDVQISRLRRMVEEDPA |
| | | | HPRYIQTVWGLGYVFVPDGSKAG::PADALDDFDLDMLPADAL |
| | | | DDFDLDMLPADALDDFDIDMLPG |
| 113 | ArcA DBD | Protein | MVESYKFNGWELDINSRSLIGPDGEQYKLPRSEFRAMLHFCENP |
| | [UniProtKB- | sequence | GKIQSRAELLKKMTGRELKPHDRTVDVTIRRIRKHFESTPDTPEI |
| | P0A9QI1134 | | IATiHGEGYRFCG::PADALDDFDLDMLPADALDDFDLDMLPAD |
| | -234]::VP16 | | ALDDFDLDML |
| | TAD-2 | | |
| 114 | AtoC DBD | Protein | MQLQSMKKEIRHLHQAISTSWQWGHIITNSPAMMDICKDTAK |
| | [UniProtKB- | sequence | IALSQASVLISGESGTGKELIARAIHYNSRRAKGPFIKVNCAALP |
| | Q060651121- | | ESLLESELFGHEKGAFTGAQTLRQGLFERANEGTLLLDEIGEMP |
| | 461]::VP16 | | LVLQAKLLRILQEREFERIGGHQTIKVDIRIIAATNRDLQAMVKE |
| | TAD-2 | | GTFREDLFYRLNVIHLILPPLRDRREDISLLANHFLQKFSSENQR |
| | | | DIIDIDPMAMSLLTAWSWPGNIRELSNVIERAVVMNSGPIIFSED |
| | | | LPPQIRQPVCNAGEVKTAPVGERNLKEEIKRVEKRIIMEVLEQQ |
| | | | EGNRTRTALMLGISRRALMYKLQEYGIDPADV::PADALDDFDL |
| | | | DMLPADALDDFDLDMLPADALDDFDLDML |
| 115 | BaeR DBD | Protein | MQRELQQQDAESPLiIDEGRFQASWRGKMLDLTPAEFRLLKTL |
| | [UniProtKB- | sequence | SHEPGKVFSREQLLNHLYDDYRVVTDRTIDSHIKNLRRKLESLD |
| | P69228I131- | | AEQSFIRAVYGVGYRWEA::PADALDDFDLDMLPADALDDFDL |
| | 234]::VP16 | | DMLPADALDDFDLDML |
| | TAD-2 | | |
| 116 | PhoB DBD | Protein | MEEVIEMQGLSLDPTSHRVMAGEEPLEMGPTEFKLLHFFMTHP |
| | [UniProtKB- | sequence | ERVYSREQLLNHVWGTNVYVEDRTVDVHIRRLRKALEPGGHD |
| | POAFJ5I129- | | RMVQTVRGTGYRFST::PADALDDFDLDMLPADALDDFDLDML |
| | 227]:: VP16 | | PADALDDFDLDML |
| | TAD-2 | | |
| 117 | EvgA DBD | Protein | MNGYCYFPFSLNRFVGSLTSDQQKLDSLSKQEISVMRYILDGK |
| | [UniProtKB- | sequence | DNNDIAEKMFISNKTVSTYKSRLMEKLECKSLMDLYTFAQRNK |
| | P0ACZ4I118 | | IG::PADALIJDIDLDMLPADALDDFDLIMLPADALDDFDLDML |
| | -204]::VP16 | | |
| | TAD-2 | | |
| 118 | NtrC DBD | Protein | MSHYQEQQQPRNVQLNGPTTDIIGEAPAMQDVFRIIGRLSRSSIS |
| | [UniProtKB- | sequence | VLINGESGTGKELVAHALHRHSPRAKAPFIALNMAAIPKDLIES |
| | P0AFB81120 | | ELFGHEKGAFTGANTIRQGRFEQADGGTLFLDEIGDMPLDVQT |
| | -469]::VP16 | | RLLRVLADGQFYRVGGYAPVKVDVRIIAATHQNLEQRVQEGK |
| | TAD-2 | | FREDLFHRLNVIRVHLPPLRERREDIPRLARHFLQVAARELGVE |
| | | | AKLLHPETEAALTRLAWPGNVRQLENTCRWLTVMAAGQEVLI |
| | | | QDLPGELFESTVAESTSQMQPDSWATLLAQWADRALRSGHQN |
| | | | LLSEAQPELERTLLTTALRHTQGHKQEAARLLGWGRNTLTRKL |
| | | | KELGME::PADALDDFDLDMLPADALDDFDLDMLPADALDDFD |
| | | | LDML |
| 119 | NarP DBD | Protein | MGSKVFSERVNQYLREREMFGAEEDPFSVLTERELDVLHELAQ |
| | [UniProtKB- | sequence | GLSNKQIASVLNISEQTVKVHIRNLLRKLNVRSRVAATILFLQQ |
| | P31802I125- | | RGAQ::PADALDDFDLDMLPADALDDFDLDMLPADALDDFDLD |
| | 2i5]::VP16 | | ML |
| | TAD-2 | | |
| 120 | BasR DBD | Protein | MRRHNNQGESELIVGNLTLNMGRRQVWMGGEELILTPKEYAL |
| | [UniProtKB- | sequence | LSRLMLKAGSPVHREILYNDIYNWDNEPSTNTLEVHIHNLRDK |
| | P30843I117- | | VGKARIRTVRGFGYMLVANEEN::PADALDDFDLDMLPADALD |
| | 222]::VP16 | | DIDLDMLPADALDDFLLIML |
| | TAD-2 | | |
| 121 | BtsR DBD | Protein | MQERSKQDVSLLPENQQALKFIPCTGHSRIYLLQMKDVAFVSS |
| | [UniProtKB | sequence | RMSGVYVTSHEGKEGFTELTLRTLESRTPLLRCHRQYLVNLAH |
| | POAFT5I117 | | LQEIRLEDNGQAELILRNGLTVPVSRRYLKSLKEAIGL::PADAL |
| | -239]::VP16 | | DDFDLDMLPADALDDFDLDMLPADALDDFDLDML |
| | TAD-2 | | |
| 122 | CpxR DBD | Protein | MRRSHWSEQQQNNDNGSPTLEVDALVLNPGRQEASFDGQTLE |
| | [UniProtKB | sequence | LTGTEFTLLYLLAQHLGQVVSREHLSQEVLGKRLTPFDRAIDM |
| | POAE88I116 | | HISNLRRKLPDRKDGHPWIKTLRGRGYIMVSAS::PADALDDFD |
| | -232]::VP16 | | LDMLPADALDDFDLDMLPADALDDFDLDML |
| | TAD-2 | | |
| 123 | CreB DBD | Protein | MRRVKKFSTPSPVIRIGHFELNEPAAQISWFDTPLALTRYEFLLL |
| | [UniProtKB- | sequence | KTLLKSPGRVWSRQQLMDSVWEDAQDTYDRTVDTHIKTLRAK |
| | P08368I116- | | LRAINPDLSPINTHRGMGYSLRGL:PADALDDFDLDMLPADAL |
| | 232]::VP16 | | DDFDLDMLPADALDDFDLDML |
| | TAD-2 | | |
| 124 | CusR DBD | Protein | MRRGAAVIIESQFQVADLMVDLVSRKVTRSGTRITLTSKEFTLL |
| | [UniProtKB- | sequence | EFFLRHQGEVLPRSLIASQVWDMNFDSDTNAIDVAVKRLRGKI |
| | POACZ8I117 | | DNDFEPKLTQTVRGVGYMLEVPDGQ::PADALDDFDLDMLPAD |
| | -227]::VP16 | | ALDDFDLDMLPADALDDFDLDML |
| | TAD-2 | | |
| 125 | DcuR DBD | Protein | MQKKMALEKHQYYDQAELDQLIHGSSSNEQDPRRLPKGLTPQ |
| | [UniProtKB- | sequence | TLRTLCQWIDAHQDYEFSTDELANEVNISRVSCRKYIIWIVNC |
| | POAD01I122 | | HILFTSIHYGVTGRPVYRYRIQMHYSILKQYCQ::PADALDDFD |
| | -239]::VP16 | | LDMLPADALDDFDLDMLPADALDDFDLDML |
| | TAD-2 | | |
| 126 | DpiA DBD | Protein | MQRKHMLESIDSASQKQIDEMFNAYARGEPKDELPTGIDPLTL |
| | [UniProtKB- | sequence | NAVRKLFKEPGVQHTA |
| | POAEF4I123 | | ETVAQALTISRTTARRYLEYCASRHLIIAEIVHGKVGRPQR1YHS |
| | -226]::VP16 | | G::PADALDDFDLDMLPADALDDFDLDMLPADALDDFDLDML |
| | TAD-2 | | |
| 127 | GlrR DBD | Protein | QQSAPATDERWREAIVTRSPLMLRLLEQARLVAQSDVSVLING |
| | [UniProtKB | sequence | QSGTGKEIFAQAIHNA |
| | POAFU4I122 | | SPRNSKPFIAINCGALPEQLLESELFGHARGAFTGAVSNREGLFQ |
| | -444]::VP16 | | AAEGGTLFLDEIGDMPAPLQVKLLRVLQERKVRPLGSNRDIDIN |
| | TAD-2 | | VRIISATHRDLPKAMARGEFREDLYYRLNVVSLKIPALAERTED |
| | | | IPLLANHLLRQAAERHKPFVRAFSTDAMKRLMTASWPGNVRQ |
| | | | LVNVIEQCVALTSSPVISDALVEQALEGENTALPTFVEARNQFE |
| | | | LNYLRKLLQITKGNVTHAARMAGRNRTEFYKLLSRHELDAND |
| | | | FKE::PADALDDFDLDMLPADALDDFDLDMLPADALDDFDLDM |
| | | | L |
| 128 | HprR DBD | Protein | MQHHALNSTLEISGLRMDSVSHSVSRDNISITLTRKEFQLLWLL |
| | [UniProtKB- | sequence | ASRAGEIIPRTVIASEIWGINFDSDTNTVDVAIRRLRAKVDDPFP |
| | P76340I116- | | EKLIATIRGMGSIVAVKK::PADALDDFDLDMLPADALDDFDL |
| | 223]::VP16 | | DMLPADALDDFDLDML |
| | TAD-2 | | |
| 129 | PhoP DBD | Protein | MRRNSGLASQVISLPPFQVDLSRRELSINDEVIKLTAFEYTIMET |
| | [UniProtKB- | sequence | LIRNNGKVVSKDSLMLQLYPDAELRESHT1DVLMGRLRKKIQA |
| | P23836I117- | | QYPQEVITTVRGQGYLFELR::PADALDDFDLDMLPADALDDFD |
| | 223]::VP16 | | LDMLPADALDDFDLDML |
| | TAD-2 | | |
| 130 | QseB DBD | Protein | MRTNGQASNELRHGNVMLDPGKRIATLAGEPLTLKPKEFALLE |
| | [UniProtKB- | sequence | LLMRNAGRVLSRKLIEEKLYTWDEEVTSNAVEVHVHHLRRKL |
| | P52076I117- | | GSDFIRTVHGIGYTLGEK::PADALDDFDLDMLPADALDDFDLD |
| | 219]::VP16 | | MLPADALDDFDLDML |
| | TAD-2 | | |
| 131 | RcsB | Protein | MGKKFTPESVSRLLEKISAGGYGDKRLSPKESEVLRLFAEGFLV |
| | UniProtKB- | sequence | TEIAKKLNRSIK |
| | PODMC7 | | TISSQKKSAMMKLGVENDIALLNYLSSVTLSPADKD::PADALD |
| | (RCSB_EC | | DFDLDMLPADALDDFDLDMLPADALDDFDLDML |
| | OLI) | | |
| | DBD::VP16 | | |
| | TAD-2 | | |
| 132 | RstA DBD | Protein | MRQNEQATLTKGLQETSLTPYKALHFGTLTIDPINRVVTLANTE |
| | [UniProtKB- | sequence | ISLSTADFELLWELATHAGQIMDRDALLKNLRGVSYDGLDRSV |
| | P52108I125- | | DVAISRLRKKLLDNAAEPYRIKTVRNKGYLFAPHAWE::PADAL |
| | 216]::VP16 | | DDFDLDMLPADALDDFDLDMLPADALDDFDLDML |
| | TAD-2 | | |
| 133 | UhpA DBD | Protein | MTGGCYLTPDIAIKLASGRQDPLTKRERQVAEKLAQGMAVKEI |
| | [UniProtKB- | sequence | AAELGLSPKTVHVHRANLMEKIGVSNDVEIARRMFDGW::PA |
| | POAGA6I11 | | DALDDFDLDMIPADALDDFDLDMLPADALDDFDIDML |
| | 7- | | |
| | 196]::VP16 | | |
| | TAD-2 | | |
| 134 | YpdB DBD | Protein | MAAWQQQQTSSTPAATVTRENDTLNLVKDERIIVTPINDIYYAE |
| | [UniProtKB- | sequence | AHEKMTFVYTRRESYVMPMNTTEFCSKLPPSHFFRCHRSFCVNL |
| | POAE39I117 | | NK1REIEPWFNNTYILRLKDLDFEVPVSRSKVKEFRQLMHL::PA |
| | -244]::VP16 | | DALDDFDLDMLPADALDDFDLDMLPADALDDFDLDML |
| | TAD-2 | | |
| 135 | ZraR DBD | Protein | MHTHSIDAETPAVTASQFGMVGKSPAMQHLLSEIALVAPSEAT |
| | [UniProtKB- | sequence | VLIHGDSGTGKELVARAIHASSARSEKPLVTLNCAALNESLLES |
| | P14375I122- | | ELFGHEKGAFTGADKRREGRFVEADGGTLFLDEIGDISPMMQV |
| | 441]::VP16 | | RLLRAIQEREVQRVGSNQIISVDVRLIAATHRDLAAEVNAGRFR |
| | TAD-2 | | QDLYYRLNVVAIEVPSLRQRREDIPLLAGHFLQRFAERNRKAV |
| | | | KGFTPQAMDLLIHYDWPGNIRELENAVERAVVLLTGEYISEREL |
| | | | PLAIASTPIPLGQSQDIQPLVEVEKEVILAALEKTGGNKTEAARQ |
| | | | LGITRKTLLAKLSR::PADALDDFDLDMLPADALDDFDLDMLPA |
| | | | DALDDFDLDML |
| 136 | HSFY1 | Protein | MAHVSSETQDVSPKDELTASEASTRSPLCEHTFPGDSDLRSMIE |
| | UniProtKB- | sequence | EHAFQVLSQGSLLESPSYTVCVSEPDKDDDFLSLNFPRKLWKIV |
| | Q96LI6(HS | | ESDQFKSISWDENGTCIVINEELFKKEILETKAPYRIFQTDAIKSF |
| | FY1_HUMAN) | | VRQLNLYGFSKIQQNFQRSAFLATFLSEEKESSVLSKLKFYYNP |
| | | | NFKRGYPQLLVRVKRRIGVKNASPISTLFNEDFNKKHFRAGAN |
| | | | MENHNSAlAAEASEESLFSASKNINMPLTRESSVRQIIANSSVPI |
| | | | RSGFPPPSPSTSVGPSEQIATDQHAILNQLTTIHMHSHSTYMQAR |
| | | | GHIVNFITTTTSQYHIISPLQNGYFGLTVEPSAVPTRYPLVSVNE |
| | | | APYRNMLPAGNPWLQMPIIADRSAAPHSRLALQPSPLDKYHPN |
| | | | YN |
| 137 | OLIG3 | Protein | MNSDSSSVSSRASSPDMDEMYLRDHHHRHHHHQESRLNSVSST |
| | UniProtKB- | sequence | QGDMMQKMPGESLSRAGAKAAGESSKYKIKKQLSEQDLQQLR |
| | Q7RTU3 | | LKINGRERKRMHDLNLAMDGLREVMPYAHGPSVRKLSKIATL |
| | (OLIG3_ | | LLARNYILMLTSSLEEMKRLVGEIYGGHHSAFHCGTVGHSAGH |
| | HUMAN) | | PAHAANSVHPVHPILGGALSSGNASSPLSAASLPAIGTIRPPHSL |
| | | | LKAPSTPPALQLGSGFQHWAGLPCPCTICQMPPPPHLSALSTAN |
| | | | MARLSAESKDLLK |
| 138 | MSGN1 | | MDNLRETFLSLEDGLGSSDSPGLLSSWDWKDRAGPFELNQASP |
| | UniProtKB- | | SQSLSPAPSLESYSSSPCPAVAGLPCEHGGASSGGSEGCSVGGAS |
| | A6NI15 | | GLVEVDYNMLAFQPTHLQGGGGPKAQKGTKVRMSVQRRRKA |
| | (MSGN1_ | | SEREKLRMRTLADALHTLRNYLPPVYSQRGQPLTKIQTLKYTIK |
| | HUMAN) | | YIGELTDLLNRGREPRAQSA |

(iii) Exemplary Output Molecules

Each of the contiguous polynucleic acids described herein comprises a cassette encoding an RNA (e.g., mRNA) comprising the nucleic acid sequence of an output (i.e., a gene of interest). In some embodiments, a contiguous polynucleic acid comprises the nucleic acid sequence of a single output. In other embodiments, a contiguous polynucleic acid comprises the nucleic acid sequences of multiple outputs (e.g., 2, 3, 4, 5, 6, 7, 8, 9, or 10 outputs).

In some embodiments, the output is an RNA molecule. In some embodiments, the RNA molecule is an mRNA encoding for a protein. In some embodiments, the output is a non-coding RNA molecule. Examples of non-coding RNA molecules are known to those having skill in the art and include, but are not limited to, include transfer RNAs (tRNAs), ribosomal RNAs (rRNAs), miRNAs, siRNAs, piRNAs, snoRNAs, snRNAs, exRNAs, scaRNAs, and long ncRNAs.

In some embodiments, the output is a therapeutic molecule (i.e., related to the treatment of disease), such as a therapeutic protein or RNA molecule. Examples of therapeutic molecules include, but are not limited to, antibodies (e.g., monoclonal or polyclonal; chimeric; humanized; including antibody fragments and antibody derivatives (bispecific, trispecific, scFv, and Fab)), enzymes, hormones, inflammatory molecules, anti-inflammatory molecules, immunomodulatory molecules, anti-cancer molecules, short-hairpin RNAs, short interfering RNAs and miRNAs. Specific examples of the foregoing classes of therapeutic molecules are known in the art, any of which may be used in accordance with the present disclosure.

In some embodiments, the output encodes for an antigen protein, protein domain, or peptide derived from a pathogen and known to elicit an immune response when produced in the body.

In some embodiments, the output is a detectable protein, such as a fluorescent protein.

In some embodiments, the output is a cytotoxin. As used herein, the term “cytotoxin” refers to a substance that is toxic to a cell. For example, in some embodiments, the output is a cytoxic protein. Examples of cytotoxic proteins are known to those having skill in the art and include, but are not limited to, granulysin, perforin/granzyme B, and the Fas/Fas ligand.

In some embodiments, the output is an enzyme that catalyzes activation of a prodrug. Examples of enzymes that catalyze prodrug activation are known to those having skill in the art, and include, but are not limited to carboxylesterases, acetylcholinesterases, butyrlylcholinesterases, paraxonases, matrix metalloproteinases, alkaline phosphatases, β-glucuronidases, valacyclovirases, prostate-specific antigens, purine-nucleoside phosphorylases, carboxypeptidases, amidases, β-lactamases, β-galactosidases, and cytosine deaminases. See e.g., Yang Y. et al., Enzyme-mediated hydrolytic activation of prodrugs. Acta. Pharmaceutica. Sinica B. 2011 October; 1(3): 143-159. Likewise, various prodrugs are known to those having skill in the art and include, but are not limited to, acyclovir, allopurinol, azidothymidine, bambuterol, bacampicillin, capecitabine, captopril, carbamazepine, carisoprodol, cyclophosphamide, diethylstilbestrol diphosphate, dipivefrin, enalapril, famciclovir, fludarabine triphosphate, fluorouracil, fosamprenavir, fosphentoin, fursultiamine, gabapentin encarbil, ganciclovir, gemcitabine, hydrazide MAO inhibitors, leflunomide, levodopa, methanamine, mercaptopurine, mitomycin, molsidomine, nabumetone, olsalazine, omeprazole, paliperidone, phenacetin, pivampicillin, primidone, proguanil, psilocybin, ramipril, S-methyldopa, simvastatin, sulfasalazine, sulindac, tegafur, terfenadine, valacyclovir, valganciclovir, and zidovudine.

In some embodiments, the output is HSV-TK, a thymidine kinase from Human alphaherpesvirus 1 (HHV-1), UniProtKB—Q9QNF7 (KITH_HHV1).

In some embodiments, the output is an immunomodulatory protein and/or RNA. As used herein, the term “immunomodulatory protein” (or immunomodulatory RNA) refers to a protein (or RNA) that modulates (stimulates (i.e., an immunostimulatory protein or RNA) or inhibits, (i.e., an immunoinhibitory protein or RNA)) the immune system by inducing activation and/or increasing activity of immune system components. Various immunomodulatory proteins are known to those having skill in the art. See e.g., Shahbazi S. and Bolhassani A. Immunostimulants: Types and Funtions. J. Med. Microbiol. Infec. Dis. 2016; 4(3-4): 45-51. In some embodiments, the immunomodulatory protein is a cytokine, chemokine (e.g., IL-2, IL-5, IL-6, IL-10, IL-12, IL-13, IL-15, IL-18, CCR3, CXCR3, CXCR4, and CCR10) or a colony stimulating factor.

In some embodiments, the output is a DNA-modifying factor. As used herein the term “DNA-modifying factor” refers to a factor that alters the structure of DNA and/or alters the sequence of DNA (e.g., by inducing recombination or introduction of mutations). In some embodiments, the DNA-modifying factor is a gene encoding a protein intended to correct a genetic defect, a DNA-modifying enzyme, and/or a component of a DNA-modifying system. In some embodiments, the DNA-modifying enzyme is a site-specific recombinase, homing endonuclease, or a protein component of a CRISPR/Cas DNA modification system.

In some embodiments, the output is a cell-surface receptor. In some embodiments, the output is a kinase.

In some embodiments, the output is a gene expression-regulating factor. The term “gene expression-regulating factor,” as used herein, refers to any factor that, when present, increases or decreases transcription of at least one gene. In some embodiments, the gene expression-regulating factor is a protein. In some embodiments, the gene expression-regulating factor is an RNA. In some embodiments, the gene expression-regulating factor is a component of a multi-component system capable of regulating gene expression.

In some embodiments, the output is an epigenetic modifier. The term “epigenetic modifier,” as used herein, refers to a factor (e.g., protein or RNA) that increases, decreases, or alters an epigenetic modification. Examples of epigenetic modifications are known to those of skill in the art and include, but are not limited to, DNA methylation and histone modifications.

In some embodiments, the output is a factor necessary for vector replication. Examples of factors necessary for vector replication are known to those having skill in the art.

(iv) Regulatory Component

A cassette encoding an RNA (e.g., comprising the nucleic acid sequence of an output and/or a transactivator) may further comprise a regulatory component. As described herein, a regulatory component is a nucleic acid sequence that controls expression of (i.e., stimulates increased or decreased expression of) the RNA. For example, in some embodiments, a cassette described herein may encode an RNA that is operably linked to a transactivator response element, a transcription factor response element, a minimal promoter, and/or a promoter element. A regulatory component is “operably linked” to a nucleic acid encoding an RNA when it is in a correct functional location and orientation in relation to the nucleic acid sequence such that it regulates (or drives) transcriptional initiation and/or expression of that sequence.

In some embodiments, the regulatory component comprises a transactivator response element. The “transactivator response element” can comprise a minimal DNA sequence that is bound and recognized by a transactivator protein. In some embodiments the transactivator response elements comprises more than one copy (i.e., repeats) of a minimal DNA sequence that is bound and recognized by a transactivator protein. In some embodiments, a transactivator response element comprises at least 2, at least 3, at least 4, at least 5, at least 6, at least 7, at least 8, at least 9, or at least 10 repeats of a minimal DNA sequence that is bound and recognized by a transactivator protein. In some embodiments the repeats are tandem repeats. In some embodiments, the transactivator response element comprises a combination of minimal DNA sequences. In some embodiments, minimal DNA sequences are interspersed with spacer sequences. In some embodiments, a spacer sequence is 1, 2, 3, 4, 5, 6, 7, 8, 9, 10, 11, 12, 13, 14, 15, 16, 17, 18, 19, 20, or greater than 20 nucleotides in length.

In some embodiments, the transactivator response element comprises deviations from the minimal DNA sequence, or is flanked by additional DNA sequence, while still being able to bind a transactivator protein. In some embodiments, different transactivator response elements can be placed next to each other, while all being able to bind to the same transactivator protein.

Exemplary transactivator response elements are listed in TABLE 3. In some embodiments, a transactivator response element consists of a nucleic acid sequence listed in TABLE 3 or a nucleic acid sequence having at least 70%, at least 80%, at least 85%, at least 90%, at least 95%, at least 96%, at least 97%, at least 98%, at least 99%, or 100% identity to a nucleic acid sequence listed in TABLE 3.

TABLE 3
Exemplary transactivator response elements. ″::″ represents fusion point
between the transactivator domain (TAD) and the DNA binding domain (DBD). Shorthand
notation of sequences of TADs and DBDs correspond to TABLE 2. DNA sequences use the
following nomenclature: W = A or T; S = C or G; K = A or C; M = G or T; Y = A or G; R = C
or T; V = C,G, or T; H = A, G or T; D = A, C or T; B = A, C, or G; N = A,C,G, or T. Capital
letter represent strong conservation; low-case symbol represents weaker conservation.

| | Examples | Examples of transactivators |
|---|---|---|
| SeqID | of Transactivator response element | capable of binding the sequence |
| 139 | GAAATAGCGCTGTACAGCGTATGGGAATCTCT | PIT::RELA TAD-1, PIT::RELA |
| | TGTACGGTGTACGAGTATCTTCCCGTACACCGT | TAD-2, PIT::RELA TAD-2, |
| | AC | PIT::RELA TAD-3, PIT::VP16 |
| | | TAD-1, PIT::VP16 TAD-2 |
| 140 | CATGTGATTGAATATAACCGACGTGACTGTTA | ET::RELA TAD-1, ET::RELA |
| | CATTTAGOGG | TAD-2, ET::RELA TAD-3, |
| | | ET::VP16 TAD-1, ET::VP16 TAD- |
| | | 2 |
| 141 | TACTGTATATATATACAGTATACTGTATATATA | Lex::RELA TAD-1, Lex::RELA |
| | TACAGTA | TAD-2, Lex::RELA TAD-3, |
| | | Lex::VP16 TAD-1, Lex::VP16 |
| | | TAD-2 |
| 142 | TACCCCTATAGGGGTATAGCGCCGGCTACCCC | NarL DBD::RELA TAD-1, NarL |
| | TATAGGGGTAT | DBD::RELA TAD-2, NarL |
| 143 | TACCCCTATAGGGGTATAGCGCCGGCTACCCC | DBD::RELA TAD-3, NarL |
| | TATAGGGGTATTACCCCTATAOGGGTATAGCG | DBD::VP16 TAD-1, NarL |
| | CCGGCTACCCCTATAGGGGTA | DBD::VP16 TAD-2 |
| 144 | wakrrkTA | |
| 145 | ATTTACATTTTGAAACATCTA | OMPR-D55E::RELA TAD-1, |
| 146 | wAhaTGwWACmAArwdTww | OMPR-D55E::RELA TAD-2, |
| | | OMPR-D55E::RELA TAD-3, |
| | | OMPR-D55E::VP16 TAD-1, |
| | | OMPR-D55E::VP16 TAD-2 |
| 147 | ATGTTAATAA | ArcA DBD::RELA TAD-1, ArcA |
| 148 | ATGTTAATAATATGTGGCATAAGCGTTAAATG | DBD::RELA TAD-2, ArcA |
| 149 | wamawwTwrTTAAma | DBD::RELA TAD-3, ArcA |
| | | DBD::VP16 TAD-1, ArcA |
| | | DBD::VP16 TAD-2 |
| 150 | GCTATGCAGAAATTTGCACA | AtoC DBD::RELA TAD-1, AtoC |
| | | DBD::RELA TAD-2, AtoC |
| | | DBD::RELA TAD-3, AtoC |
| | | DBD::VP16 TAD-1, AtoC |
| | | DBD::VP16 TAD-2 |
| 151 | TTCTYCMYdATYKSYkS | BaeR DBD::RELA TAD-1, BaeR |
| | | DBD::RELA TAD-2, BaeR |
| | | DBD::RELA TAD-3, BaeR |
| | | DBD::VP16 TAD-1, BaeR |
| | | DBD::VP16 TAD-2 |
| 152 | TGTCATAAAACTGTCATATTCCTTACATATAAC | PhoB DBD::RELA TAD-1, PhoB |
| | TGTCA | DBD::RELA TAD-2, PhoB |
| 153 | cTgwcAyAAAwcTgwm | DBD::RELA TAD-3, PhoB |
| | | DBD::VP16 TAD-1, PhoB |
| | | DBD::VP16 TAD-2 |
| 154 | TTCTTACGCCTGTAGGATTAGTAAGAA | EvgA DBD::RELA TAD-1, EvgA |
| 155 | TkCYTACAmCTGTARGA | DBD::RELA TAD-2, EvgA |
| | | DBD::RELA TAD-3, EvgA |
| | | DBD::VP16 TAD-1, EvgA |
| | | DBD::VP16 TAD-2 |
| 156 | TGCACCAWWWTGGTGCA | NtrC DBD::RELA TAD-1, NtrC |
| 157 | tGCmCyAaaATsGtGCA | DBD::RELA TAD-2, NtrC |
| | | DBD::RELA TAD-3, NtrC |
| | | DBD::VP16 TAD-1, NtrC |
| | | DBD::VP16 TAD-2 |
| 158 | NTACCCCTA | 1. NarPDBD::RELA TAD- |
| 159 | mTACyycT | 1, NarP DBD::RELA TAD-2, NarP |
| | | DBD::RELA TAD-3, NarP |
| | | DBD::VP16 TAD-1, NarP |
| | | DBD::VP16 TAD-2 |
| 160 | CTTAAGGTTNNCTTAAGGTT | 2. BasRDBD::RELA TAD- |
| | | 1, BasR DBD::RELA TAD-2, BasR |
| | | DBD::RELA TAD-3, BasR |
| | | DBD::VP16 TAD-1, BasR |
| | | DBD::VP16 TAD-2 |
| 161 | ANCNCTAAANT | BtsR DBD::RELA TAD-1, BtsR |
| | | DBD::RELA TAD-2, BtsR |
| | | DBD::RELA TAD-3, BtsR |
| | | DBD::VP16 TAD-1, BtsR |
| | | DBD::VP16 TAD-2 |
| 162 | GTAAANNNNNGTAAA | CpxR DBD::RELA TAD-1, CpxR |
| 163 | GTAAArmwrygwaAr | DBD::RELA TAD-2, CpxR |
| | | DBD::RELA TAD-3, CpxR |
| | | DBD::VP16 TAD-1, CpxR |
| | | DBD::VP16 TAD-2 |
| 164 | TTCACNNNNNNTTCAC | CreB DBD::RELA TAD-1, CreB |
| | | DBD::RELA TAD-2, CreB |
| | | DBD::RELA TAD-3, CreB |
| | | DBD::VP16 TAD-1, CreB |
| | | DBD::VP16 TAD-2 |
| 165 | AAAATGACAANNTTGTCATTTTT | CusR DBD::RELA TAD-1, CusR |
| | | DBD::RELA TAD-2, CusR |
| | | DBD::RELA TAD-3, CusR |
| | | DBD::VP16 TAD-1, CusR |
| | | DBD::VP16 TAD-2 |
| 166 | TGATTACAAAACTTTAAAAAGTGCTG | DcuR DBD::RELA TAD-1, DcuR |
| 167 | TGATTACAAAACTTTAAAAAGTGCTGCATAGC | DBD::RELA TAD-2, DcuR |
| | GCCGGCCGCGCCTGATTACAAAACTTTAAAAA | DBD::RELA TAD-3, DcuR |
| | GTGCTG | DBD::VP16 TAD-1, DcuR |
| 168 | TGATTACAAAACTTTAAAAAGTGCTGTAGCGC | DBD::VP16 TAD-2 |
| | CGGCTGATTACAAAACTTTAAAAAGTGCTG | |
| 169 | TkwwTTwAaTTwykwwA | |
| 170 | GATCTATTCTTTT | DpiA DBD::RELA TAD-1, DpiA |
| 171 | TATCTTTTTTTAT | DBD::RELA TAD-2, DpiA |
| | | DBD::RELA TAD-3, DpiA |
| | | DBD::VP16 TAD-1, DpiA |
| | | DBD::VP16 TAD-2 |
| 172 | TGTCN₁₋₁₀GACA | GlrR DBD::RELA TAD-1, GlrR |
| | | DBD::RELA TAD-2, GlrR |
| | | DBD::RELA TAD-3, GlrR |
| | | DBD::VP16 TAD-1, GlrR |
| | | DBD::VP16 TAD-2 |
| 173 | CATTACAANTTGTAATG | HprR DBD::RELA TAD-1, HprR |
| | | DBD::RELA TAD-2, HprR |
| | | DBD::RELA TAD-3, HprR |
| | | DBD::VP16 TAD-1, HprR |
| | | DBD::VP16 TAD-2 |
| 174 | CATGAANNNNNTGTTTA | PhoP DBD::RELA TAD-1, PhoP |
| 175 | WrTTTAkswwyyGTTtA | DBD::RELA TAD-2, PhoP |
| | | DBD::RELA TAD-3, PhoP |
| | | DBD::VP16 TAD-1, PhoP |
| | | DBD::VP16 TAD-2 |
| 176 | rTTAAmNNNNNrTTAAm | QseB DBD::RELA TAD-1, QseB |
| | | DBD::RELA TAD-2, QseB |
| | | DBD::RELA TAD-3, QseB |
| | | DBD::VP16 TAD-1, QseB |
| | | DBD::VP16 TAD-2 |
| 177 | TAAGAATATTCCTA | RcsB DBD::RELA TAD-1, RcsB |
| 178 | AwYmGAyKWwTYT | DBD::RELA TAD-2, RcsB |
| | | DBD::RELA TAD-3, RcsB |
| | | DBD::VP16 TAD-1, RcsB |
| | | DBD::VP16 TAD-2 |
| 179 | NNTACANNNNNNTACTNN | RstA DBD::RELA TAD-1, RstA |
| 180 | KWCWTWTvGTTACA | DBD::RELA TAD-2, RstA |
| | | DBD::RELA TAD-3, RstA |
| | | DBD::VP16 TAD-1, RstA |
| | | DBD::VP16 TAD-2 |
| 181 | GGCAAAACTAAGAAATTTTCCAGGTTTTGCC | UhpA DBD::RELA TAD-1, UhpA |
| | | DBD::RELA TAD-2, UhpA |
| | | DBD::RELA TAD-3, UhpA |
| | | DBD::VP16 TAD-1, UhpA |
| | | DBD::VP16 TAD-2 |
| 182 | GGCATTTCAT | YpdB DBD::RELA TAD-1, YpdB |
| | | DBD::RELA TAD-2, YpdB |
| | | DBD::RELA TAD-3, YpdB |
| | | DBD::VP16 TAD-1, YpdB |
| | | DBD::VP16 TAD-2 |
| 183 | GCGAGTCAAAAAAACTCA | ZraR DBD::RELA TAD-1, ZraR |
| | | DBD::RELA TAD-2, ZraR |
| | | DBD::RELA TAD-3, ZraR |
| | | DBD::VP16 TAD-1, ZraR |
| | | DBD::VP16 TAD-2 |
| 184 | TTCGAANNNTTCGAA | HSFY1 UniProtKB-Q96LI6 |
| 185 | rCrTTCGAAaCRTTCgAww | (HSFY1_HUMAN) |
| 186 | rTTCGAAhsdTTCGAAy | |
| 187 | rCATTCyAAACATTCyAhw | |
| 188 | rTTCGAAysdTTCGAAy | |
| 189 | ACCATATGTT | OLIG3 UniProtKB-Q7RTU3 |
| 190 | rcCATATGkr | (OLIG3_HUMAN) |
| 191 | AvCAkmTGTT | |
| 192 | rcCATATGkT | |
| 193 | acCATATGkt | |
| 194 | amCAkmTGTt | |
| 195 | ACCATATGKT | |
| 196 | AmCATATGby | |
| 197 | srCCAwwTGkys | MSGN1 UniProtKB-A6NI15 |
| 198 | brcCAwwTGkyv | (MSGN1_HUMAN) |

In some embodiments, the regulatory component comprises a transcription factor response element. The term “transcription factor response element” refers to a DNA sequence that is bound and recognized by a transcription factor. As used herein, the term “transcription factor” refers to a protein that is not encoded on the contiguous polynucleic acid that modulates gene transcription. In some embodiments, a transcription factor is a transcription activator (i.e., increases transcription). In other embodiments, a transcription factor is a transcription inhibitor (i.e., inhibits transcription). In some embodiments, a transcription factor is an endogenous transcription factor of a cell.

In some embodiments, the transcription factor response element is engineered to bind to directly, or be affected indirectly, by one or more of the following transcription factors: ABL1, CEBPA, ERCC3, HIST1H2BE, MDM4, PAX7, SMARCA4, TFPT, AFF1, CHD1, ERCC6, HIST1H2BG, MED12, PAX8, SMARCB1, THRAP3, AFF3, CHD2, ERF, HLF, MEF2B, PBX1, SMARCD1, TLX1, AFF4, CHD4, ERG, HMGA1, MEF2C, PEG3, SMARCE1, TLX3, APC, CHD5, ESPL1, HMGA2, MEN1, PER1, SMURF2, TNFAIP3, AR, CHD7, ESR1, HOXA11, MITF, PHF3, SOX2, SOX4, TP53, ARID1A, CIC, ETS1, HOXA13, MKL1, PHF6, SOX5, TRIM24, ARID1B, CIITA, ETV1, HOXA7, MLLT1, PHOX2B, SOX9, TRIM33, ARID3B, CNOT3, ETV4, HOXA9, MLLT10, PLAG1, SRCAP, TRIP11, ARID5B, CREB1, ETV5, HOXC11, MLLT3, PML, SS18L1, TRPS1, ARNT, CREB3L1, ETV6, HOXC13, MLLT6, PMS1, SSB, TRRAP, ARNT2, CREBBP, EWSR1, HOXD11, MYB, PNN, SSX1, TSC22D1, ASB15, CRTC1, EYA4, HOXD13, MYBL1, MYBL2, POU2AF1, SSX2, TSHZ3, ASXL1, CSDE1, EZH2, ID3, MYC, POU2F2, SSX4, VHL, ATF1, CTCF, FEV, IRF2, MYCN, POU5F1, STAT3, WHSC1, ATF7IP, CTNNB1, FLI1, IRF4, MYOD1, PPARG, STAT4, WHSC1L1, ATM, DACH1, FOXA1, IRF6, NCOA1, PRDM1, STAT5B, WT1, ATRX, DACH2, FOXE1, IRF8, NCOA2, PRDM16, STAT6, WWP1, BAZ2B, DAXX, FOXL2, IRX6, NCOA4, PRDM9, SUFU, WWTR1, BCL11A, DDB2, FOXP1, JUN, NCOR1, PRRX1, SUZ12, XBP1, BCL11B, DDIT3, FOXQ1, KHDRBS2, NCOR2, PSIP1, TAF1, XPC, BCL3, DDX5, FUBP1, KHSRP, NEUROG2, RARA, TAF15, ZBTB16, BCL6, DEK, FUS, KLF2, NFE2L2, RB1, TAL1, ZBTB20, BCLAF1, DIP2C, FXR1, KLF4, NFE2L3, RBM15, TAL2, ZFP36L1, BCOR, DNMT1, GATA1, KLF5, NFIB, RBMX, TBX18, ZFX, BRCA1, DNMT3A, GATA2, KLF6, NFKB2, REL, TBX22, ZHX2, BRCA2, DOT1L, GATA3, LDB1, NFKBIA, RUNX1, TBX3, ZIC3, BRD7, EED, GLI3, LMO1, NONO, RUNX1T1, TCEA1, ZIM2, BRD8, EGR2, GTF2I, LMO2, NOTCH2, RXRA, TCEB1, ZNF208, BRIP1, ELAVL2, HDAC9, LMX1A, NOTCH3, SALL3, TCERG1, ZNF226, BRPF3, ELF3, HEY1, LYL1, NPM1, SATB2, TCF12, ZNF331, BTG1, ELF4, HIST1H1B, LZTR1, NR3C2, SETBP1, TCF3, ZNF384, BTG2, ELK4, HIST1H1C, MAF, NR4A3, SFPQ, TCF7L2, ZNF469, CBFA2T3, ELL, HIST1H1D, MAFA, NSD1, SIN3A, TFAP2D, ZNF595, CBFB, EP300, HIST1H1E, MAFB, OLIG2, SMAD2, TFDP1, ZNF638, CDX2, EPC1, HIST1H2BC, MAML1, PAX3, SMAD4, TFE3, CDX4, ERCC2, HIST1H2BD, MAX, PAX5, SMARCA1, and TFEB.

The “transcription factor response element” can comprise a minimal DNA sequence that is bound and recognized by a transcription factor. In some embodiments the transcription factor response element comprises more than one copy (i.e., repeats) of a minimal DNA sequence that is bound and recognized by a transcription factor. In some embodiments, a transcription factor response element comprises at least 2, at least 3, at least 4, at least 5, at least 6, at least 7, at least 8, at least 9, or at least 10 repeats of a minimal DNA sequence that is bound and recognized by a transcription factor. In some embodiments the repeats are tandem repeats. In some embodiments, the transcription factor response element comprises a combination of minimal DNA sequences. In some embodiments, minimal DNA sequences are interspersed with spacer sequences. In some embodiments, a spacer sequence is 1, 2, 3, 4, 5, 6, 7, 8, 9, 10, 11, 12, 13, 14, 15, 16, 17, 18, 19, 20, or greater than 20 nucleotides in length. In some embodiments, the transactivator response element comprises deviations from the minimal DNA sequence, or is flanked by additional DNA sequence, while still being able to bind a transactivator protein. In some embodiments, different transactivator response elements can be placed next to each other, while all being able to bind to the same transactivator protein.

In some embodiments, the transcription factor response element is unique (i.e., the contiguous polynucleic acid includes only one copy of the transcription factor response element). In other embodiments, the transcription factor response element is not unique. In some embodiments, a transcription factor that binds to the transcription factor response element activates expression of the RNA to which it is operably linked. In other embodiments, a transcription factor that binds to the transcription factor response element inhibits expression of the RNA to which it is operably linked.

In some embodiments, the regulatory component comprises at least 2, at least 3, at least 4, at least 5, at least 6, at least 7, at least 8, at least 9, or at least 10 different transcription factor response elements, each bound by a different transcription factor. In some embodiments, the regulatory component comprises 2, 3, 4, 5, 6, 7, 8, 9, or 10 different transcription factor response elements, each bound by a different transcription factor.

Exemplary transcription factor response elements are listed in TABLE 4. In some embodiments, a transcription factor response element consists of a nucleic acid sequence listed in TABLE 4 or a nucleic acid sequence having at least 70%, at least 80%, at least 85%, at least 90%, at least 95%, at least 96%, at least 97%, at least 98%, at least 99%, or 100% identity to a nucleic acid sequence listed in TABLE 4.

TABLE 4
Exemplary transcription factor response elements.

| | | | Input |
|---|---|---|---|
| Name | Sensor | Response Element Sequence | TFs/Pathways |
| 199 | TCF/LEF 1x | AGATCAAAGGGGGTA | ICF/LEF, Beta |
| | | | Catenin, WNT |
| | | | Pathway Activation |
| 200 | TCF/LEF 3x | AGATCAAAGGGGGTAAGATCAAAG | TCF/LEF, Beta |
| | | GGGGTAAGATCAAAGGGGGTA | Catenin, WNT |
| | | | Pathway Activation |
| 201 | TCF/LEF 6x | AGATCAAAGGGGGTAAGATCAAAG | TCF/LEF, Beta |
| | | GGGGTAAGATCAAAGGGGGTAAGA | Catenin, WNT |
| | | TCAAAGGGGGTAAGATCAAAGGGG | Pathway Activation |
| | | GTAAGATCAAAGGGGGTA | |
| 202 | Myc 1x | CGCGCCGACCACGTGGTCCA | Myc |
| 203 | Myc 2x | CGCGCCGACCACGTGGTCGACCAC | Myc |
| | | GTGGTCCA | |
| 204 | Myc 3x | CGCGCCGACCACGTGGTCGACCAC | Myc |
| | | GTGGTCGACCACGTGGTCCA | |
| 205 | HIF-1A 1x | GACCTTGAGTACGTGCGTCTCTGCA | HIF-1 Alpha, |
| | | CGTATG | Hypoxia Response |
| 206 | HIF-1A2x | GACCTTGAGTACGTGCGTCTCTGCA | HIF-1 Alpha, |
| | | CGTATGGACCTTGAGTACGTGCGTC | Hypoxia Response |
| | | TCTGCACGTATG | |
| 207 | HIF-1A3x | GACCTTGAGTACGTGCGTCTCTGCA | HIF-1 Alpha, |
| | | CGTATGGACCTTGAGTACGTGCGTC | Hypoxia Response |
| | | TCTGCACGTATGGACCTTGAGTACG | |
| | | TGCGTCTCTGCACGTATG | |
| 208 | 3x FOXM1 | TGTTTATTGTTTATTGTTTAT | FOXM1 |
| | Vitro | | |
| 209 | 6x FOXM1 | TGTTTATTGTTTATTGTTTATTGTTT | FOXM1 |
| | Vitro | ATTGTTTATTGTTTAT | |
| 210 | 3x FOXM1 | GCAAAGCAAACAGCAAAGCAAACA | FOXM1 |
| | ChipSeq Fwd | GCAAAGCAAACA | |
| 211 | 6x FOXM1 | GCAAAGCAAACAGCAAAGCAAACA | FOXM1 |
| | ChipSeq Fwd | GCAAAGCAAACAGCAAAGCAAACA | |
| | | GCAAAGCAAACAGCAAAGCAAACA | |
| 212 | 3x FOXM1 | TGTTTGCTTTGCTGTTTGCTTTGCTG | FOXM1 |
| | ChipSeq Rev | TTTGCTTTGC | |
| 213 | 6x FOXM1 | TGTTTGCTTTGCTGTTTGCTTTGCTG | FOXM1 |
| | ChipSeq Rev | TTTGCTTTGCTGTTTGCTTTGCTGTT | |
| | | TGCTTTGCTGTTTGCTTTGC | |
| 214 | 8x Gli2 (3,4) | GAACACCCAGAACACCCAGAACAC | Gli2, Gli1, SHH |
| | | CCAGAACACCCAGAACACCCAGAA | Pathway Activation |
| | | CACCCAGAACACCCAGAACACCCA | |
| 215 | 6x Gli2 (3,4) | GAACACCCAGAACACCCAGAACAC | Gli2, Gli1, SHH |
| | | CCAGAACACCCAGAACACCCAGAA | Pathway Activation |
| | | CACCCA | |
| 216 | HNF1 1x | AGTTAATAATTTAAC | HNF1A, HNF1B |
| 217 | HNF1 2x | AGTTAATAATTTAACAGTTAATAAT | HNF1A, HNF1B |
| | | TTAAC | |
| 218 | HNF1 3x | AGTTAATAATTTAACAGTTAATAAT | HNF1A, HNF1B |
| | | TTAAC AGTTAATAATTTAAC | |
| 219 | HNF1 4x | AGTTAATAATTTAACAGTTAATAAT | HNF1A, HNF1B |
| | | TTAACAGTTAATAATTTAACAGTTA | |
| | | ATAATTTAAC | |
| 220 | 2x SOX9/10 | CTACACAAAGCCCTCTGTGTAAGAC | SOX9., SOX10, |
| | C-C’ | TACACAAAGCCCTCTGTGTAAGA | SOX6, SOX8 Low |
| | | | affinity: SOX4, |
| | | | SOX2, SOX21 |
| | | | (Noon cooperative) |
| 221 | 3x SOX9/10 | CTACACAAAGCCCTCTGTGTAAGAC | SOX9., SOX10, |
| | C-C’ | TACACAAAGCCCTCTGTGTAAGACT | SOX6, SOX8 |
| | | ACACAAAGCCCTCTGTGTAAGA | Low affinity: SOX4, |
| | | | SOX2, SOX21 |
| | | | (Noon cooperative) |
| 222 | 2x SOX9/10 | CTACACAAAGCCCTCTTTGTGAGAC | SOX9., SOX10, |
| | C-C | TACACAAAGCCCTCTTTGTGAGA | SOX6, SOX8 |
| | | | SOX4, SOX2, |
| | | | SOX21 |
| 223 | 3x SOX9/10 | CTACACAAAGCCCTCTTTGTGAGAC | SOX9., SOX10, |
| | C-C | TACACAAAGCCCTCTTTGTGAGACT | SOX6, SOX8 |
| | | ACACAAAGCCCTCTTTGTGAGA | SOX4, SOX2, |
| | | | SOX21 |
| 224 | 3X Sox 4/9 | CCATTGTTCT CCATTGTTCT | SOX4 SOX9 |
| | | CCATTGTTCT | |
| 225 | 6X Sox 4/9 | CCATTGTTCTCCATTGTTCTCCATTG | SOX4 SOX9 |
| | | TTCTCCATTGTTCTCCATTGTTCTCC | |
| | | ATTGTTCT | |
| 226 | 6X Sox 4/11 | AACAAAGAACAAAGAACAAAGAAC | SOXC Family |
| | | AAAG | |
| 227 | 3x MYBL2 | AACCGTTAAACGGTTAACCGTTAAA | MYBL2 |
| | | CGGTTAACCGTTAAACGGTT | |
| 228 | MYBL2- | AGAGATATTTAGTGAATCAGCAAGT | MYBL2 MuvB |
| | CCNB1 | GGAACCAAAAAGACTTGAGGACTG | FoxM1 |
| | | ATTGGATGAGGAGAGGTTAG | |
| 229 | 2x MYBL2- | AGAGATATTTAGTGAATCAGCAAGT | MYBL2 MuvB |
| | CCNB1 | GGAACCAAAAAGACTTGAGGACTG | FoxM1 |
| | | ATTGGATGAGGAGAGGTTAGAGAG | |
| | | ATATTTAGTGAATCAGCAAGTGGAA | |
| | | CCAAAAAGACTTGAGGACTGATTG | |
| | | GATGAGGAGAGGTTAG | |
| 230 | MYBL2-Plk1 | ACTGGTGCCCTCCTCAACTCCCACC | MYBL2 MuvB |
| | | TGCATCTGGGGCCCATACTGGTTGG | FoxM1 |
| | | CTCCCGCGGTGCCATGTCTGCAGTG | |
| | | TGCCCCCCAGCCCCGG | |
| 231 | 2x MYBL2- | ACTGGTGCCCTCCTCAACTCCCACC | MYBL2 MuvB |
| | Plkl | TGCATCTGGGGCCCATACTGGTTGG | FoxM1 |
| | | CTCCCGCGGTGCCATGTCTGCAGTG | |
| | | TGCCCCCCAGCCCCGGACTGGTGCC | |
| | | CTCCTCAACTCCCACCTGCATCTGG | |
| | | GGCCCATACTGGTTGGCTCCCGCGG | |
| | | TGCCATGTCTGCAGTGTGCCCCCCA | |
| | | GCCCCGG | |
| 232 | Myc 8x | CGCGCCGACCACGTGGTCGACCAC | Myc |
| | | GTGGTCCACGCGCCGACCACGTGGT | |
| | | CGACCACGTGGTCCACGCGCCGACC | |
| | | ACGTGGTCGACCACGTGGTCCACGC | |
| | | GCCGACCACGTGGTCGACCACGTG | |
| | | GTCCA | |
| 233 | Myc/USF1 4x | GTCACGTGGCTCAGTCACGTGGCTC | Myc USF1 |
| | | AGTCACGTGGCTCAGTCACGTGGC | |
| 234 | Myc/USF1 8x | GTCACGTGGCTCAGTCACGTGGCTC | Myc USF1 |
| | | AGTCACGTGGCTCAGTCACGTGGCG | |
| | | TCACGTGGCTCAGTCACGTGGCTCA | |
| | | GTCACGTGGCTCAGTCACGTGGC | |
| 235 | EBOX Myc | GACCACGTGGTCGACCACGTGGTCG | Myc |
| | 4x | ACCACGTGGTCGACCACGTGGTC | |
| 236 | EBOX Myc | GACCACGTGGTCGACCACGTGGTCG | Myc |
| | 8x | ACCACGTGGTCGACCACGTGGTCGA | |
| | | CCACGTGGTCGACCACGTGGTCGAC | |
| | | CACGTGGTCGACCACGTGGTC | |
| 237 | 8x TCF/LEF | CCTCTACCCCCTTTGATCTTACCCCC | TCF/LEF, Beta |
| | (Beta Catenin) | TTTGATCTTACCCCCTTTGATCTTAC | Catenin, WNT |
| | | CCCCTTTGATCTTACCCCCTTTGATC | Pathway Activation |
| | | TTACCCCCTTTGATCTTACCCCCTTT | |
| | | GATCTTACCCCCTTTGATCT | |

In some embodiments, a regulatory component comprises a promoter element (or a promoter fragment). Exemplary promoter elements are listed in TABLE 5. In some embodiments, a promoter element consists of a nucleic acid sequence listed in TABLE 5 or a nucleic acid sequence having at least 70%, at least 80%, at least 85%, at least 90%, at least 95%, at least 96%, at least 97%, at least 98%, at least 99%, or 100% identity to a nucleic acid sequence listed in TABLE 5.

TABLE 5
Exemplary promoter elements.

| Seq | | |
|---|---|---|
| ID | Name | SEQUENCE |
| 238 | AFP 0.5 | GGCCTGAAATAACCTCTGAAAGAGGAACTTGGTTAGGTACCTTCTGAGGCT |
| | Core | GAAAGAACCAGCTGTGGAATGTGTGTCAGTTAGGGTGTGGAAAGTCCCCA |
| | | GGCTCCCCAGCAGGCAGAAGTATGCAAAGCATGCATCTCAATTAGTCAGCA |
| | | ACCAGGTGTGGAAAGTCCCCAGGCTCCCCAGCAGGCAGAAGTATGCAAAG |
| | | CATGCATCTCAATTAGTCAGCAACCATAGTCCCACTGCAGTTTGAGGAGAA |
| | | TATTTGTTATATTTGCAAAATAAAATAAGTTTGCAAGTTTTTTTTTTCTGCCC |
| | | CAAAGAGCTCTGTGTCCTTGAACATAAAATACAAATAACCGCTATGCTGTT |
| | | AATTATTGGCAAATGTCCCATTTTCAACCTAAGGAAATACCATAAAGTAAC |
| | | AGATATACCAACAAAAGGTTACTAGTTAACAGGCATTGCCTGAAAAGAGT |
| | | ATAAAAGAATTTCAGCATGATTTTCCATATTGTGCTTCCACCACTGCCAATA |
| | | ACAC |
| 239 | AFP 0.2 | CTGTGTCCTTGAACATAAAATACAAATAACCGCTATGCTGTTAATTATTGGC |
| | Core | AAATGTCCCATTTTCAACCTAAGGAAATACCATAAAGTAACAGATATACCA |
| | | ACAAAAGGTTACTAGTTAACAGGCATTGCCTGAAAAGAGTATAAAAGAAT |
| | | TTCAGCATGATTTTCCATATTGTGCTTCCACCACTGCCAATAACAC |
| 240 | AFP | AAATTAGTTTTGAATCTTTCTAATACCAAAGTTCAGTTTACTGTTCCATGTT |
| | Enhancer | GCTTCTGAGTGGCTTCACAGACTTATGAAAAAGTAAACGGAATCAGAATTA |
| | +0.2 Core | CATCAATGCAAAAGCATTGCTGTGAACTCTGTACTTAGGACTAAACTTTGA |
| | | GCAATAACACATATAGATTGAGGATTGTTTGCTGTTAGTATACAAACTCTG |
| | | GTTCAAAGCTCCTCTTTATTGCTTGTCTTGGAAAATTTGCTGTTCTTCATGGT |
| | | TTCTCTTTTCACTGCTATCTATTTTTCTCAACCACTCACATGGCTACAATAAC |
| | | TGTCTGCAAGCTTATGATTCCCAAATATCTATCTCTAGCCTCAATCTTGTTC |
| | | CAGAAGATAAAAAGTAGTATTCAAATGCACATCAACGTCTCCACTTGGAGG |
| | | GCTTAAAGACGTTTCAACATACAAACCGGGGAGTTTTGCCTGGAATGTTTC |
| | | CTAAAATGTGTCCTGTAGCACATAGGGTCCTCTTGTTCCTTAAAATCTAATT |
| | | ACTTTTAGCCCAGTGCTCATCCCACCTATGGGGAGATGAGAGTGAAAAGGG |
| | | AGCCTGATTAATAATTACACTAAGTCAATAGGCATAGAGCCAGGACTGTTT |
| | | GGGTAAACTGGTCACTTTATCTTAAACTAAATATATCCAAAACTGAACATG |
| | | TACTTAGTTACTAAGTCTTTGACTTTATCTCATTCATACCACTCAGCTTTATC |
| | | CAGGCCACTTATTTGACAGTATTATTGCGAAAACTTCCTAACTGGTCTCCTT |
| | | ATCATAGTCTTATCCCCTTTTGAAACAAAAGAGACAGTTTCAAAATACAAA |
| | | TATGATTTTTATTAGCTCCCTTTTGTTGTCTATAATAGTCCCAGAAGGAGTT |
| | | ATAAACTCCATTTAAAAAGTCTTTGAGATGTGGCCCTTGCCAACTTTGCCAG |
| | | GCTGTGTCCTTGAACATAAAATACAAATAACCGCTATGCTGTTAATTATTG |
| | | GCAAATGTCCCATTTTCAACCTAAGGAAATACCATAAAGTAACAGATATAC |
| | | CAACAAAAGGTTACTAGTTAACAGGCATTGCCTGAAAAGAGTATAAAAGA |
| | | ATTTCAGCATGATTTTCCCAAGTTTGCTTATTTATGAAAAGTTATCGATAAT |
| | | TTCTTTAGTTTTGTAT |
| 241 | Midkine | TCCCTGCCCACCCGCGGAAACCGCCCCAGGTGGGCCGCGCCCCCTCCCCAG |
| | 600 | CAGCCAGCAGGGCGCCAGGGCTGAGCCGGCCGTGGAGGGGAGCGGGTCCC |
| | | GCGGGTTATACAGGCGCCGGGGCTCCGCGGCAGGCAAGAGAAGCTGAGGC |
| | | CTGAGAACGGCCCGGGCCTTGGCGTACGGCAGGGGACGACCTGGGATGGG |
| | | GGCAGCGGGCGGCGGCGCAGGGAGTGGGCCGGGGGCCGGTGTGCGCGGGC |
| | | GGGACGGGGCCCGGGGTCGGGAGACCACCGCTCGGAAGATGGGGCCGGGA |
| | | GAGGCCGCCGTCGCAGCGCAGAGGGCACCGGCGGGGAGACGCGAGGACGC |
| | | GGGGCCGGGAACACGGACGCCGGAGTAGAAGCGCGGGGGGCGCGGGCTG |
| | | GAGCGGGGGCGGGGACGCCGGGGTCGGGGGCGGTGCGGGTTTGAGGGGAG |
| | | GGGGCGGGGCGGGTCCTTCCCTGGGGGGGTGGGGAGAGGGGGCGGGGGCC |
| | | CATGTGACCGGCTCAGACCGGTTCTGGAGACAAAAGGGGCCGCGGCGGCC |
| | | GGAGCGGGACGGGCCCGGCGCGGGAGGGAGCGAAGCAGCGCGGGCAGCG |
| | | AGCGAGTGAG |
| 242 | Midkine | ACCACCGCTCGGAAGATGGGGCCGGGAGAGGCCGCCGTCGCAGCGCAGAG |
| | 300 | GGCACCGGCGGGGAGACGCGAGGACGCGGGGCCGGGAACACGGACGCCG |
| | | GAGTAGAAGCGCGGGGGGCGCGGGCTGGAGCGGGGGCGGGGACGCCGGG |
| | | GTCGGGGGCGGTGCGGGTTTGAGGGGAGGGGGCGGGGCGGGTCCTTCCCT |
| | | GGGGGGGTGGGGAGAGGGGGCGGGGGCCCATGTGACCGGCTCAGACCGGT |
| | | TCTGGAGACAAAAGGGGCCGCGGCGGCCGGAGCGGGACGGGCCCGGCGCG |
| | | GGAGGGAGCGAAGCAGCGCGG |
| 243 | Midkine | CCGCGGCGGCCGGAGCGGGACGGGCCCGGCGCGGGAGGGAGCGAAGCAG |
| | 70 | CGCGGGCAGCGAGCGAGTGAG |
| 244 | Glypican-3 | GGAGTCTCACTCTGTCGCCCAAGCTGGAGTGCAGTAGTGCGATCTCAGCTC |
| | 1.5 | ACTGCAACCTCTGCCCTCTGAGTTCAAGTGATTCTCCTGCCTCAGCCTCCCG |
| | | AGTAGCTGGGATTACAGGCGCCTGCCACCGCGCCCAGCTAATTTTTTGTATT |
| | | TTTGGTAGAGACGGGGTTTCACCATCTTGGCCAGGCTGGTCTTGAACTCCTG |
| | | ACCTCATGATCCACCCGCCTCGGCTTCCCAAAGTGCTGGGATTACAGGCGT |
| | | GAGCCACCGTGCCTGGCCTAAAGAACTGGATTTCTAATGGTGAAATCTAAG |
| | | CAGGAGAGGTGGGATTTGGGTGTAGGATACCTTTCAAATAGCCTTCTACTC |
| | | CATCTATGAAATAGGCTAGCTTTGGCTCAGTAAATTTGCTGTGTAATGATTT |
| | | TCTAATGAGTTAGGCTGGCTTTAAGCCCCTGGTTATTTCGTTGTAACCAGTT |
| | | AGGCTTTGCCTCTTGAAGGGCCACCTGGGACrGTCGTGCAGTAGATTTTCTT |
| | | TTAACGCCCCAGAATCAGGTGCTTTCTCTGACTTTGTGTGGCTCTACTGAAT |
| | | CAAATCTAGCAAGCCACAGAGGCTTTCAGACTTTTAAGATACAATATTCAA |
| | | AGGTGAGGCAGGCTGTGAAAAGCCCAGCGGTCCCTGGCTGTCCCTGAACGC |
| | | GACTATTTGCAGGTTGGCTTTGAGAACCCGGTCAGAGCTGCGTAGGAAAA |
| | | CGGTTCCCGGGAAGCTCCTCAGAGAGTAGAATGAGGAGGTGGATTTTGTGT |
| | | GAAGGAACACCTTGTGTGGCTCTGGTGGCCAGGAAAGAGCTGGCACAAGC |
| | | TGAAAGAAGGCCTGTGGCGAAGCGGAGGGGGACCTAAGTCAGGGACCCCC |
| | | ACCTGCCCCCAGGAAGGATGAAAAGGAGACAAAAATCCTAAAGGGAAAAG |
| | | CCCTCCAGGCTGTAGGCCAATGAGCGGCGGGAAGGAGGAGTGAGGCTGGG |
| | | GAACTTCTCCCAGAGCCAGTCAGAGCGGACGGCTGCTGGGAAGCCAATCA |
| | | GCGCGCTCGAGCCTGCAGCCCCTCTGCAGTAGTTATGCCAGAGCGCCCTGT |
| | | GTAGAGCGGCTGCGAGCGGGCAGCTGGGCTCGGCTGCCGGGAGCCACCGC |
| | | GCGGGCTCCGCACCCTCCTCTCGCACTGCCTTCGCCCGGTCCCCGCGCCGCG |
| | | GTGCCCCAGTGGCCCCCGCCGCGCTCCACGCCGCGCCCCCGCACCCCGCCG |
| | | GCTACCGGCCGCACAACCGCCACCGCCCCCTGGCCGCGCGGCTCGCCTCGC |
| | | CCCGCCCCGTCCCTCCTCGCCCCGCCCCACCCCAGTCAGCCCCGCCCTGCCC |
| | | CGCGCCGCCAAGCGGTTCCCGCCCTCGCCCAGCGCCCAGGTAGCTGCGAGG |
| | | AAACTTTTGCAGCGGCTGGGTAGCAGCACGTCTCTTGCTCCTCAGGGCCAC |
| | | TGCCAGGCTTGCCGAGTCCTGGGACTGCTCTCGCTCCGGCTGCCACTCTCCC |
| | | GCGCTCTCCTAGCTCCCTGCGAAGCAGG |
| 245 | Glypican-3 | GGAGAGGTGGGATTTGGGTGTAGGATACCTTTCAAATAGCCTTCTACTCCA |
| | 1.2 | TCTATGAAATAGGCTAGCTTTGGCTCAGTAAATTTGCTGTGTAATGATTTTC |
| | | TAATGAGTTAGGCTGGCTTTAAGCCCCTGGTTATTTCGTTGTAACCAGTTAG |
| | | GCTTTGCCTCTTGAAGGGCCACCTGGGACTGTCGTGCAGTAGATTTTCTTTT |
| | | AACGCCCCAGAATCAGGTGCTTTCTCTGACTTTGTGTGGCTCTACTGAATCA |
| | | AATCTAGCAAGCCACAGAGGCTTTCAGACTTTTAAGATACAATATTCAAAG |
| | | GTGAGGCAGGCTGTGAAAAGCCCAGCGGTCCCTGGCTGTCCCTGAACGCGA |
| | | CTATTTGCAGGTTGGCTTTGAGAACCCGGTCAGAGCTGCGTTAGGAAAACG |
| | | GTTCCCGGGAAGCTCCTCAGAGAGTAGAATGAGGAGGTGGATTTTGTGTGA |
| | | AGGAACACCTTGTGTGGCTCTGGTGGCCAGGAAAGAGCTGGCACAAGCTG |
| | | AAAGAAGGCCTGTGGCGAAGCGGAGGGGGACCTAAGTCAGGGACCCCCAC |
| | | CTGCCCCCAGGAAGGATGAAAAGGAGACAAAAATCCTAAAGGGAAAAGCC |
| | | CTCCAGGCTGTAGGCCAATGAGCGGCGGGAAGGAGGAGTGAGGCTGGGGA |
| | | ACTTCTCCCAGAGCCAGTCAGAGCGGACGGCTGCTGGGAAGCCAATCAGC |
| | | GCGCTCGAGCCTGCAGCCCCTCTGCAGTAGTTATGCCAGAGCGCCCTGTGT |
| | | AGAGCGGCTGCGAGCGGGCAGCTGGGCTCGGCTGCCGGGAGCCACCGCGC |
| | | GGGCTCCGCACCCTCCTCTCGCACTGCCTTCGCCCGGTCCCCGCGCCGCGGT |
| | | GCCCCAGTGGCCCCCGCCGCGCTCCACGCCGCGCCCCCGCACCCCGCCGGC |
| | | TACCGGCCGCACAACCGCCACCGCCCCCTGGCCGCGCGGCTCGCCTCGCCC |
| | | CGCCCCGTCCCTCCTCGCCCCGCCCCACCCCAGTCAGCCCCGCCCTGCCCCG |
| | | CGCCGCCAAGCGGTTCCCGCCCTCGCCCAGCGCCCAGGTAGCTGCGAGGAA |
| | | ACTTTTGCAGCGGCTGGGTAGCAGCACGTCTCTTGCTCCTCAGGGCCACTG |
| | | CCAGGCTTGCCGAGTCCTGGGACTGCTCTCGCTCCGGCTGCCACTCTCCCGC |
| | | GCTCTCCTAGCTCCCTGCGAAGCAGG |
| 246 | Glypican-3 | AAAGGGAAAAGCCCTCCAGGCTGTAGGCCAATGAGCGGCGGGAAGGAGGA |
| | 0.6 | GTGAGGCTGGGGAACTTCTCCCAGAGCCAGTCAGAGCGGACGGCTGCTGG |
| | | GAAGCCAATCAGCGCGCTCGAGCCTGCAGCCCCTCTGCAGTAGTTATGCCA |
| | | GAGCGCCCTGTGTAGAGCGGCTGCGAGCGGGCAGCTGGGCTCGGCTGCCG |
| | | GGAGCCACCGCGCGGGCTCCGCACCCTCCTCTCGCACTGCCTTCGCCCGGT |
| | | CCCCGCGCCGCGGTGCCCCAGTGGCCCCCGCCGCGCTCCACGCCGCGCCCC |
| | | CGCACCCCGCCGGCTACCGGCCGCACAACCGCCACCGCCCCCTGGCCGCGC |
| | | GGCTCGCCTCGCCCCGCCCCGTCCCTCCTCGCCCCGCCCCACCCCAGTCAGC |
| | | CCCGCCCTGCCCCGCGCCGCCAAGCGGTTCCCGCCCTCGCCCAGCGCCCAG |
| | | GTAGCTGCGAGGAAACTTTTGCAGCGGCTGGGTAGCAGCACGTCTCTTGCT |
| | | CCTCAGGGCCACTGCCAGGCTTGCCGAGTCCTGGGACTGCTCTCGCTCCGG |
| | | CTGCCACTCTCCCGCGCTCTCCTAGCTCCCTGCGAAGCAGG |
| 247 | Glypican-3 | CCCCGCACCCCGCCGGCTACCGGCCGCACAACCGCCACCGCCCCCTGGCCG |
| | 0.3 | CGCGGCTCGCCTCGCCCCGCCCCGTCCCTCCTCGCCCCGCCCCACCCCAGTC |
| | | AGCCCCGCCCTGCCCCGCGCCGCCAAGCGGTTCCCGCCCTCGCCCAGCGCC |
| | | CAGGTAGCTGCGAGGAAACTTTTGCAGCGGCTGGGTAGCAGCACGTCTCTT |
| | | GCTCCTCAGGGCCACTGCCAGGCTTGCCGAGTCCTGGGACTGCTCTCGCTC |
| | | CGGCTGCCACTCTCCCGCGCTCTCCTAGCTCCCTGCGAAGCAGG |
| 248 | Glypican-3 | GTCAGCCCCGCCCTGCCCCGCGCCGCCAAGCGGTTCCCGCCCTCGCCCAGC |
| | 0.2 | GCCCAGGTAGCTGCGAGGAAACTTTTGCAGCGGCTGGGTAGCAGCACGTCT |
| | | CTTGCTCCTCAGGGCCACTGCCAGGCTTGCCGAGTCCTGGGACTGCTCTCGC |
| | | TCCGGCTGCCACTCTCCCGCGCTCTCCTAGCTCCCTGCGAAGCAGG |
| 249 | Glypican-3 | CGCCCAGGTAGCTGCGAGGAAACTTTTGCAGCGGCTGGGTAGCAGCACGTC |
| | 150 bp | TCTTGCTCCTCAGGGCCACTGCCAGGCTTGCCGAGTCCTGGGACTGCTCTCG |
| | | CTCCGGCTGCCACTCTCCCGCGCTCTCCTAGCTCCCTGCGAAGCAGG |
| 250 | hTERT | TGGCCCCTCCCTCGGGTTACCCCACAGCCTAGGCCGATTCGACCTCTCTCCG |
| | 455 | CTGGGGCCCTCGCTGGCGTCCCTGCACCCTGGGAGCGCGAGCGGCGCGCGG |
| | | GCGGGGAAGCGCGGCCCAGACCCCCGGGTCCGCCCGGAGCAGCTGCGCTG |
| | | TCGGGGCCAGGCCGGGCTCCCAGTGGATTCGCGGGCACAGACGCCCAGGA |
| | | CCGCGCTTCCCACGTGGCGGAGGGACTGGGGACCCGGGCACCCGTCCTGCC |
| | | CCTTCACCTTCCAGCTCCGCCTCCTCCGCGCGGACCCCGCCCCGTCCCGACC |
| | | CCTCCCGGGTCCCCGGCCCAGCCCCCTCCGGGCCCTCCCAGCCCCTCCCCTT |
| | | CCTTTCCGCGGCCCCGCCCTCTCCTCGCGGCGCGAGTTTCAGGCAGCGCTGC |
| | | GTCCTGCTGCGCACGTGGGAAGCCCTGGCCCCGGCCACCCCCGCG |
| 251 | hTERT | CCAGGACCGCGCTTCCCACGTGGCGGAGGGACTGGGGACCCGGGCACCCG |
| | 258 | TCCTGCCCCTTCACCTTCCAGCTCCGCCTCCTCCGCGCGGACCCCGCCCCGT |
| | | CCCGACCCCTCCCGGGTCCCCGGCCCAGCCCCCTCCGGGCCCTCCCAGCCC |
| | | CTCCCCTTCCTTTCCGCGGCCCCGCCCTCTCCTCGCGGCGCGAGTTTCAGGC |
| | | AGCGCTGCGTCCTGCTGCGCACGTGGGAAGCCCTGGCCCCGGCCACCCCCG |
| | | CG |
| 252 | hTERT | CGTCCCGACCCCTCCCGGGTCCCCGGCCCAGCCCCCTCCGGGCCCTCCCAG |
| | 159 | CCCCTCCCCTTCCTTrCCGCGGCCCCGCCCrCTCCTCGCGGCGCGAGTTTCA |
| | | GGCAGCGCTGCGTCCTGCTGCGCACGTGGGAAGCCCTGGCCCCGGCCACCC |
| | | CCGCG |
| 253 | hTERT | CCCCTCCCCTTCCTTTCCGCGGCCCCGCCCTCTCCTCGCGGCGCGAGTTTCA |
| | 108 | GGCAGCGCTGCGTCCTGCTGCGCACGTGGGAAGCCCTGGCCCCGGCCACCC |
| | | CCGCG |
| 254 | hTERT 83 | CCCGGGTCCCCGGCCCAGCCCCCTCCGGGCCCTCCCAGCCCCTCCCCTTCCI |
| | | TTCCGCGGCCCCGCCCTCTCCTCGCGGCGCG |
| 255 | Survivin | CCATAGAACCAGAGAAGTGAGTGGATGTGATGCCCAGCTCCAGAAGTGAC |
| | 976 | TCCAGAACACCCTGTTCCAAAGCAGAGGACACACTGATTTTTTTTTTAATAG |
| | (BIRC5) | GCTGCAGGACTTACTGTTGGTGGGACGCCCTGCTTTGCGAAGGGAAAGGAG |
| | | GAGTTTGCCCTGAGCACAGGCCCCCACCCTCCACTGGGCTTTCCCCAGCTCC |
| | | CTTGTCTTCTTATCACGGTAGTGGCCCAGTCCCTGGCCCCTGACTCCAOAAG |
| | | GTGGCCCTCCTGGAAACCCAGGTCGTGCAGTCAACGATGTACTCGCCGGGA |
| | | CAGCGATGTCTGCTGCACTCCATCCCTCCCCTGTTCATTTGTCCTTCATGCC |
| | | CGTCTGGAGTAGATGCTTTTTGCAGAGGTGGCACCCTGTAAAGCTCTCCTGT |
| | | CTGACTTTTTTTTTTTTTTTAGACTGAGTTTTGCTCTTGTTGCCTAGGCTGGA |
| | | GTGCAATGGCACAATCTCAGCTCACTGCACCCTCTGCCTCCCGGGTTCAAG |
| | | CGATTCTCCTGCCTCAGCCTCCCGAGTAGTTGGGATTACAGGCATGCACCA |
| | | CCACGCCCAGCTAATTTTTGTATTTTTAGTAGAGACAAGGTTTCACCGTGAT |
| | | GGCCAGGCTGGTCTTGAACTCCAGGACTCAAGTGATGCTCCTGCCTAGGCC |
| | | TCTCAAAGTGTTGGGATTACAGGCGTGAGCCACTGCACCCGGCCTGCACGC |
| | | GTTCTTTGAAAGCAGTCGAGGGGGCGCTAGGTGTGGGCAGGGACGAGCTG |
| | | GCGCGGCGTCGCTGGGTGCACCGCGACCACGGGCAGAGCCACGCGGCGGG |
| | | AGGACTACAACTCCCGGCACACCCCGCGCCGCCCCGCCTCTACTCCCAGAA |
| | | GGCCGCGGGGGGTGGACCGCCTAAGAGGGCGTGCGCTCCCGACATGCCCC |
| | | GCGGCGCGCCATTAACCGCCAGATTTGAATCGCGGGACCCGTTGGCAGAGG |
| | | TGG |
| 256 | Survivin | CAATCTCAGCTCACTGCACCCTCTGCCTCCCGGGTTCAAGCGATTCTCCTGC |
| | 500 | CTCAGCCTCCCGAGTAGTTGGGATTACAGGCATGCACCACCACGCCCAGCT |
| | | AATTTTTGTATTTTTAGTAGAGACAAGGTTTCACCGTGATGGCCAGGCTGGT |
| | | CTTGAACTCCAGGACTCAAGTGATGCTCCTGCCTAGGCCTCTCAAAGTGTT |
| | | GGGATTACAGGCGTGAGCCACTGCACCCGGCCTGCACGCGTTCTTTGAAAG |
| | | CAGTCGAGGGGGCGCTAGGTGTGGGCAGGGACGAGCTGGCGCGGCGTCGC |
| | | TGGGTGCACCGCGACCACGGGCAGAGCCACGCGGCGGGAGGACTACAACT |
| | | CCCGGCACACCCCGCGCCGCCCCGCCTCTACTCCCAGAAGGCCGCGGGGGG |
| | | TGGACCGCCTAAGAGGGCGTGCGCTCCCGACATGCCCCGCGGCGCGCCATT |
| | | AACCGCCAGATTTGAATCGCGGGACCCGTTGGCAGAGGTGG |
| 257 | Survivin | TTGAAAGCAGTCGAGGGGGCGCTAGGTGTGGGCAGGGACGAGCTGGCGCG |
| | 250 | GCGTCGCTGGGTGCACCGCGACCACGGGCAGAGCCACGCGGCGGGAGGAC |
| | | TACAACTCCCGGCACACCCCGCGCCGCCCCGCCTCTACTCCCAGAAGGCCG |
| | | CGGGGGGTGGACCGCCTAAGAGGGCGTGCGCTCCCGACATGCCCCGCGGC |
| | | GCGCCATTAACCGCCAGATTTGAATCGCGGGACCCGTTGGCAGAGGTGG |
| 258 | Survivin | TACAACTCCCGGCACACCCCGCGCCGCCCCGCCTCTACTCCCAGAAGGCCG |
| | 150 | CGGGGGGTGGACCGCCTAAGAGGGCGTGCGCTCCCGACATGCCCCGCGGC |
| | | GCGCCATTAACCGCCAGATTTGAATCGCGGGACCCGTTGGCAGAGGTGG |
| 259 | Survivin | CCTAAGAGGGCGTGCGCTCCCGACATGCCCCGCGGCGCGCCATTAACCGCC |
| | 85 | AGATTTGAATCGCGGGACCCGTTGGCAGAGGTGG |
| 260 | ANGPTL-3 | AATTCTAGTTTGGTCCTAGATGACCACATATCCATTGTTCCTTCAACGAGCA |
| | -784 67 | CATGGTAAAGAGCCTAGAACACAGAGACACAGAACACAGTGGAGAAAAG |
| | | GGAGTGAAATGTCTTTAATGACACTTACTATATATGGGATTTTGTGACAAT |
| | | ATACAAGGATGGTTAAGACATATAAGGTGATGCAAAAAAACATATTAACA |
| | | ATTATAGTGACAAAAAATGAGGAGCATATAATTATACATTGATTTATACAG |
| | | AGTACCAGAGGAACACAGCATTGAGAGCCGTAACACCACCTGAGGGAGTG |
| | | GAGAAAGGCTTCAGAGAGAAAGTGTTTTTTGGAATGGATCACTGTTTCCAA |
| | | AAGAACTAAAGTACAGTTTGAGAAATGCATACTTAATTCATTACTTTTTTCC |
| | | CCTCAACTTTAATAATAAATTTACCCAACAAAAAAGTTTATTTTTGACTTGT |
| | | AAATCTCTTAAAATCATAAAAAAGTAAAATTAGCTTTTAAAAACAGGTAGT |
| | | CACCATAGCATTGAATGTGTAGTTTATAATACAGCAAAGTTAAATACAATT |
| | | TCAAATTACCTATTAAGTTAGTTGCTCATTTCTTTGATTTCATTTAGCATTGA |
| | | TCTAACTCAATGTGGAAGAAGGTTACATTCGTGCAAGTTAACACGGCTTAA |
| | | TGATTAACTATGTTCACCTACCAACCTTACCTTTTCTGGGCAAATATTGGTA |
| | | TATATAGAGTTAAGAAGTCTAGGTCTGCTTCCAGAAGAAAACAGTTCCACG |
| | | TTGCTTGAAATTGAAAATCAAGATAAAAATGTTCACAATTAAGCTCCTTCTT |
| | | TTTATTGTTCCTCTAGTTATTTCCTCCAGAATTGATCAAGA |
| 261 | ANGPTL-3 | ATAGCATTGAATGTGTAGTTTATAATACAGCAAAGTTAAATACAATTTCAA |
| | -282 67 | ATTACCTATTAAGTTAGTTGCTCATTTCTTTGATTTCATTTAGCATTGATCTA |
| | | ACTCAATGTGGAAGAAGGTTACATTCGTGCAAGTTAACACGGCTTAATGAT |
| | | TAACTATGTTCACCTACCAACCTTACCTTTTCTGGGCAAATATTGGTATATA |
| | | TAGAGTTAAGAAGTCTAGGTCTGCTTCCAGAAGAAAACAGTTCCACGTTGC |
| | | TTGAAATTGAAAATCAAGATAAAAATGTTCACAATTAAGCTCCTTCTTTTTA |
| | | TTGTTCCTCTAGTTATTTCCTCCAGAATTGATCAAGA |
| 262 | ANGPTL-3 | TCAATGTGGAAGAAGGTTACATTCGTGCAAGTTAACACGGCTTAATGATTA |
| | -175 67 | ACTATGTTCACCTACCAACCTTACCTTTTCTGGGCAAATATTGGTATATATA |
| | | GAGTTAAGAAGTCTAGGTCTGCTTCCAGAAGAAAACAGTTCCACGTTGCTT |
| | | GAAATTGAAAATCAAGATAAAAATGTTCACAATTAAGCTCCTTCTTTTTATT |
| | | GTTCCTCTAGTTATTTCCTCCAGAATTGATCAAGA |
| 263 | AFP | TGGGATGTTTCGAGCAGTCCTGCTGAAGTCCTTTTATATCCTGTTTAAGGGA |
| | Proximal | TGCCTGTTAACTAGTAACCTTCAGTGAGCAAACATATGACTCTATTTCCTTA |
| | Compact | CGTTGAAGTTAGGCAATTTGCCAATAATTAACAGAGCAGGGGTCACTTGTA |
| | | TCCTATGTTCAAGGACAAAGACCACTTCAGAGTGGAAAAAAAATCTAAACT |
| | | GTTCAAATAGATTATTTCCCCTGAAGAATAATTCATTCATCTCAACATAAGA |
| | | CATAGATATAGCCATAAAGAAAAGGTAGCAGACTTACTATGTAACTCCAAA |
| | | TACAAGTTCAGGCTATTCATTAGTGGATATATTTCTTGATTATCCAGTTATA |
| | | GTATATTTTATTTTATTTAGTGTATCGCATCTGGTTTAACATA |
| 264 | AFP | ATGAGGGAAGCGGGTGTGATCCACTTgAaaaCTGCTGGTTCCTTCACCGCAG |
| | Proximal | GCAGTGCTGGAAGTGGGATGTTTCGAGCAGTCCTGCTGAAGTCCTTTTATA |
| | Compact | TCCTGTTTAAGGGATGCCTGTTAACTAGTAACCTTCAGTGAGCAAACATAT |
| | 1^{st} exon | GACTCTATTTCCTTACGTTGAAGTTAGGCAATTTGCCAATAATTAACAGAGC |
| | | AGGGGTCACTTGTATCCTATGTTCAAGGACAAAGACCACTTCAGAGTGGAA |
| | | AAAAAATCTAAACTGTTCAAATAGATTATTTCCCCTGAAGAATAATTCATT |
| | | CATCTCAACATAAGACATAGATATAGCCATAAAGAAAAGGTAGCAGACTT |
| | | ACTATGTAACTCCAAATACAAGTTCAGGCTATTCATTAGTGGATATATTTCT |
| | | TGATTATCCAGTTATAGTATATTTTATTTTATTTAGTGTATCGCATCTGGTTT |
| | | AACATAG |
| 265 | AFP Long | ATGAGGGAAGCGGGTGTGATCCACTTgAaaaCTGCTGGTTCCTTCACCGCAG |
| | TATA 1^{st} | GCAGTGCTGGAAGTGGGATGTTTCGAGCAGTCCTGCTGAAGTCCTTTTATA |
| | exon | TCCTGTTTAAGGGATGCCTGTTAACTAGTAACCTTCAGTGAGCAAACATAT |
| | | GACTCTATTTCCTTACGTTGAAGTTAGGCAATTTGCCAATAATTAACAGAGC |
| | | AGGGGTCACTTGTATCCTATGTTCAAGGACAAAGACCACTTCAGAGTGGAA |
| | | AAAAAATCTAAACTGTTCAAATAGATTATTTCCCCTGAAGAATAATTCATT |
| | | CATCTCAACATAAGACATAGATATAGCCATAAAGAAAAGGTAGCAGACTT |
| | | ACTATGTAACTCCAAATACAAGTTCAGGCTATTCATTAGTGGATATATTTCT |
| | | TGATTATCCAGTTATAGTATATTTTATTTTATTTAGTGTATCGCATCTGGTTT |
| | | AACATAGAAAACTTACAGCACAAAACCTGATGAGCCAGCTCCCATTCTAAT |
| | | TTTATGTGCCAAAGAATAATTCCATATGTATGTCACAGGTGCATGGGTCAG |
| | | CTGCAACATCCTCTCAAGCCCTAAGATGATGATGCTAACAGCAACAAATGG |
| | | GCACTGATAGTTTCCATTTCTCTACACATTAGAGTTGATGGAAAACTTTTAA |
| | | AACTTCCCAGTGCGTATCGAAACTAGAACTCAGACGTTGGCGTGTCAGAGT |
| | | CTGTGTGTCTAGAGGTCCAGACATGTTTGCTAAGGCTTCATATGTAGTTGAG |
| | | TTTATTTTTTATTTTTTTAAATTCATGGC |
| 266 | AFP Long | ATGAGGGAAGCGGGTGTGATCCACTTgAaaaCTGCTGGTTCCTTCACCGCAG |
| | MRE | GCAGTGCTGGAAGTGGGATGTTTCGAGCAGTCCTGCTGAAGTCCTTTTATA |
| | TATA 1^{st} | TCCTGTTTAAGGGATGCCTGTTAACTAGTAACCTTCAGTGAGCAAACATAT |
| | exon | GACTCTATTTCCTTACGTTGAAGTTAGGCAATTTGCCAATAATTAACAGAGC |
| | | AGGGGTCACTTGTATCCTATGTTCAAGGACAAAGACCACTTCAGAGTGGAA |
| | | AAAAAATCTAAACTGTTCAAATAGATTATTTCCCCTGAAGAATAATTCATT |
| | | CATCTCAACATAAGACATAGATATAGCCATAAAGAAAAGGTAGCAGACTT |
| | | ACTATGTAACTCCAAATACAAGTTCAGGCTATTCATTAGTGGATATATTTCT |
| | | TGATTATCCAGTTATAGTATATTTTATTTTATTTAGTGTATCGCATCTGGTTT |
| | | AACATAGAAAACTTACAGCACAAAACCTGATGAGCCAGCTCCCATTCTAAT |
| | | TTTATGTGCCAAAGAATAATTCCATATGTATGTCACAGGTGCATGGGTCAG |
| | | CTGCAACATCCTCTCAAGCCCTAAGATGATGATGCTAACAGCAACAAATGG |
| | | GCACTGATAGTTTCCATTTCTCTACACATTAGAGTTGATGGAAAACTTTTAA |
| | | AACTTCCCAGTGCGTATCGAAACTAGAACTCAGACGTTGGCGTGTCAGAGT |
| | | CTGTGTGTCTAGAGGTCCAGACATGTTTGCTAAGGCTTCATATGTAGTTGAG |
| | | TTTATTTTTTATTTTTTTAAATTCAGGCGACTGGGTTTGAATTTTGCCCTCTC |
| | | CGTTATCTGCCACATGACTTTGTGTGAGGTtTCTAATACCAACTGCAAACAA |
| | | CCCTAAGCCCACGTGTGCTGTTGCTCAAAGCTTTGTCGCAAATACTGAGCTC |
| | | ACACCACATACCTCTCATAGCTCTATGTCTGGTTCTGTTTGTCACTTCCTGA |
| | | GCCCATGAAACCTCTCAGAAGCAATATGGTTAAACAAACTGGACTTTAGTC |
| | | TATGAAAGGCTCTACCCTTGACTATTCAAACTGTCAGCCAGATGACAAAAA |
| | | CTCAAACCAGCTTTATTCTGGC |
| 267 | AFP Long | ATGAGGGAAGCGGGTGTGATCCACTTgAaaaCTGCTGGTTCCTTCACCGCAG |
| | No | GCAGTGCTGGAAGTGGGATGTTTCGAGCAGTCCTGCTGAAGTCCTTTTATA |
| | Deletions | TCCTGTTTAAGGGATGCCTGTTAACTAGTAACCTTCAGTGAGCAAACATAT |
| | | GACTCTATTTCCTTACGTTGAAGTTAGGCAATTTGCCAATAATTAACAGAGC |
| | | AGGGGTCACTTGTATCCTATGTTCAAGGACAAAGACCACTTCAGAGTGGAA |
| | | AAAAATCTTGCAAATGCTGCAAATGTTCTTCACCATCTAAACTGTTCAAAT |
| | | AGATTATTTCCCCTGAAGAATAATTCATTCATCTCAACATAAGACATAGAT |
| | | ATAGCCATAAAGAAAAGGTAGCAGACTTACTATGTAACTCCAAATACATTC |
| | | TTTTTGAAAGAAATAATAAAATGCACACCATATGCTAGGCACTGAACAAAT |
| | | TGTTTCAGTAGTTCAGGCTATTCATTAGTGGATATATTTCTTGATTATCCAG |
| | | TTATTATTTCGCTCAAAACCATCGGTCAAGTATATTTTATTTTATTTAGTGTA |
| | | TCGCATCTGGTTTAACATAGAAAACTTACAGCACAAAACCTGATGAGCCAG |
| | | CTCCCATTCTAATTTTATGTGCCAAAGAATAATTCCATATGTATGTCACAGG |
| | | TGCATGGGTCAGCTGCAACATCCTCTCAAGCCCTAAGATGATGATGCTAAC |
| | | AGCAACAAATGGGCACTGACATACTTCTGACCCTAAGAGTGCTTCACTCAT |
| | | ACCTTCACCCTCAATGCCGTAGAGTCTATGATAGTTTCCATTTCTCTACACA |
| | | TTAGAGTTGATGGAAAACTTTTAAAACTTCCCAGTGCGTATCGAAACTAGA |
| | | ACTCAGACGTTGGCGTGTCAGAGTCTGTGTGTCTAGAGGTCCAGACATGTT |
| | | TGCTAAGGCTTCATATG |
| 268 | GPC3 ikb | tAGCCCGACAGAGCAAGAGAGGAGCCGCTACCCAGCCGCCGCAAAAGTTTC |
| | | CTCGCAGCTACCTGGGCGCTGGGCGAGGGCGGGAACAGCTTGGCGGTGCG |
| | | GGGCGGCCCGGGGCGGAGCCTTGTGGGCGTGGCGAGGAGGGACGGGGCGG |
| | | GGCGAGGCAAGGCGAGCCGCGCTGCCTGGAGGACGGCGTGGGGTCGTGTA |
| | | GCTGCTGGCCTGCGGGATGCGGGGCGTGGCAAGGAGCTTAGCTGGGAGAT |
| | | TGGGTTTACCAAGGTGGCGGGCAAGCCTTGGTGGGAGAGGCGCGGGAAGA |
| | | GGATAAGGAGCGTGTGCGGTGGCTCCCGGCAATCCTGCCCTGACACTCGCT |
| | | CGCCGCTGCTCTACACTGGGCGCTCTGGCATAACTACTGCAGAGGGGCTGC |
| | | AGGCTCAGGCACGCTGATTGGCTTCCCAGCAGCAGTCCCCTCTGACTGGCT |
| | | CTGGGAGAAGTTCCCCAGCCTCACTCCTCCTTTCCGCCTCCCTTTGGCCTAC |
| | | AGCCGGGAGGGCTTTTCCTTTTCAGCCTTTGCAAGCTCTCCATCTTCCTTGG |
| | | AGTGGAGTGGAGGTCTGCGGTTTAGGTACCCGACTCGACCCTAGGCCTTCT |
| | | CCCACCCAGATCTGGCTCCTTCTGGCCACCAGAGCCCACACAAGGTTTCCT |
| | | AAGCACAAAATCCCTCTCCTTGCTGTTTTCTGAGAAAGGTTTCTTGGGAACC |
| | | CTTTCCCAATGCAGCTGTGGCCAAGCCCTCAAAGCCTACCCACAAATAGTC |
| | | ACGTTCCAGAGCGCTGGGGACCTCTGGATTTCACAGCCTGGCTCATCTTTGT |
| | | ACCTAAAAGGTCTGGAAGCCCGTGTAGCTTGCTGGGTTTCATTCAATAGAA |
| | | CCACACAAAGTAAATGTGTGCAAATTTAGGCACTTGATCCTGATTCCTAGG |
| | | TGAATCATATCATCTACAGGATAATCACGGGCGACCCTCATAAAGCAAAGT |
| | | GTAGCTGGTGAGAGTAACTCATTCAGGAAATCATTTTACAGATGAAATTCA |
| | | TTAAGTCATGGTTAGTCTGTTTCATACCTGGAGTAGAGCCCTATTTAGAAGA |
| | | TTTCCTGGATGTCAATCCACGTTTCT |

In some embodiments, the promoter element comprises a transcription factor response element and a minimal promoter. In some embodiments, the promoter element comprises a mammalian promoter or promoter fragment. In some embodiments, the mammalian promoter or promoter fragment is unique (i.e., the contiguous polynucleic acid includes only one copy of the mammalian promoter or promoter fragment). In other embodiments, the mammalian promoter or promoter fragment is not unique.

In some embodiments, a regulatory component comprises a minimal promoter. As used herein, the term “minimal promoter” refers to a nucleic acid sequence that is necessary but not sufficient to initiate expression of an output. In some embodiments, a minimal promoter is naturally occurring. In other embodiments, a minimal promoter is engineered, such as by altering and/or shortening a natural occurring sequence, combining natural occurring sequences, or combining naturally occurring sequences with non-naturally occurring sequences; in each case an engineered minimal promoter is a non-naturally occurring sequence. In some embodiments, the minimal promoter is engineered from a viral or non-viral source. Examples of minimal promoters are known to those having skill in the art.

In some embodiments, a regulatory component comprises a transactivator response element, a transcription factor response element, and a minimal promoter. One having skill in the art will appreciate that these elements may be oriented in various configurations. For example, a transactivator response element may be 5′ or 3′ to a promoter element and/or transcription factor response element; a transcription factor response element may be 5′ or 3′ to a promoter element and/or transactivator response element; a promoter element may be 5′ or 3′ to a transcription factor response element and/or a transactivator response element.

In some embodiments, the regulatory component of a cassette comprises, from 5′ to 3′: a transactivator response element, a transcription factor response element, and a minimal promoter. In some embodiments, a regulatory component comprises from 5′ to 3′: a transcription factor response element, a transactivator response element, and a minimal promoter.

In some embodiments, the regulatory component of a cassette comprises a transactivator response element and a promoter element. In some embodiments, the regulatory component of a cassette comprises, from 5′ to 3′: a transactivator response element and a promoter element. In some embodiments, the regulatory component of a cassette comprises a transactivator response element, a promoter element and a minimal promoter. In some embodiments, the regulatory component of a cassette comprises, from 5′ to 3′: a transactivator response element, a promoter element and a minimal promoter. In some embodiments, the regulatory component of a cassette comprises, from 5′ to 3′: a promoter element and a transactivator response element. In some embodiments, the regulatory component of a cassette comprises, from 5′ to 3′: a promoter element, a transactivator response element and a minimal promoter. In some embodiments, the promoter element is a mammalian promoter. In some embodiments, the promoter element is a promoter fragment.

(v) Exemplary Contiguous Polynucleic Acids

In some embodiments, a contiguous polynucleic acid molecule comprises a gene circuit having a single cassette. For example, in some embodiments, a contiguous polynucleic acid molecule comprises a cassette encoding an RNA whose expression is operably linked to a transactivator response element, wherein the RNA comprises: (i) a nucleic acid sequence of an output; (ii) a nucleic acid sequence of a transactivator; and (iii) a miRNA target site (e.g., a let-7c target site, a miR-22 target site, a miR-26b target site, or a combination thereof); wherein the transactivator, when expressed as a protein, binds and transactivates the transactivator response element.

In some embodiments, the mRNA further comprises a nucleic acid sequence of a polycistronic expression element. The term “polycistronic response element,” as used herein, refers to a nucleic acid sequence that facilitates the generation of two or more proteins from a single mRNA. A polycistronic response element may comprise a polynucleic acid encoding an internal recognition sequence (IRES) or a 2A peptide. See e.g., Liu et al., Systematic comparison of 2A peptides for cloning multi-genes in a polycistronic vector. Sci. Rep. 2017 May 19; 7(1): 2193. In some embodiments, the polycistronic expression element separates the nucleic acid sequences of the output and the transactivator.

In some embodiments, the mRNA comprises a 3′ UTR, wherein the 3′ UTR comprises a miRNA target site (e.g., a let-7c target site, a miR-22 target site, a miR-26b target site, or a combination thereof). In some embodiments, the mRNA comprises a 5′ UTR, wherein the 5′ UTR comprises a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising the transactivator response element and the transcription factor response element; (ii) the nucleic acid sequence encoding the output and the transactivator; and (iii) a downstream component comprising a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising the transcription factor response element and the transactivator response element; (ii) the nucleic acid sequence encoding the output and the transactivator; and (iii) a downstream component comprising a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising the transactivator response element and the transcription factor response element; (ii) the nucleic acid sequence encoding the transactivator and the output; and (iii) a downstream component comprising a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising the transcription factor response element and the transactivator response element; (ii) the nucleic acid sequence encoding the transactivator and the output; and (iii) a downstream component comprising a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising a promoter element and the transactivator response element; (ii) the nucleic acid sequence encoding the transactivator and the output; and (iii) a downstream component comprising a miRNA target site (e.g., a let-7c target site, a miR-22 target site, a miR-26b target site, or a combination thereof).

In some embodiments, the contiguous polynucleic acid molecules comprise, from 5′ to 3′: (i) an upstream regulatory component comprising the transactivator response element and a promoter element; (ii) the nucleic acid sequence encoding the transactivator and the output; and (iii) a downstream component comprising a miRNA target site (e.g., a let-7c target site, a miR-22 target site, a miR-26b target site, or a combination thereof).

In some embodiments, the promoter element comprises a mammalian promoter or promoter fragment.

In some embodiments, a contiguous polynucleic acid molecule comprises a gene circuit having multiple cassettes. For example, in some embodiments, a contiguous polynucleic acid molecule comprising: a) a first cassette encoding a first RNA whose expression is operably linked to a transactivator response element, wherein the first RNA comprises: (i) a nucleic acid sequence of an output; and (ii) a miRNA target site (e.g., a let-7c target site, a miR-22 target site, a miR-26b target site, or a combination thereof); and b) a second cassette encoding a second RNA, wherein the second RNA comprises a nucleic acid sequence of a transactivator; wherein the transactivator of the second cassette, when expressed as a protein, binds and transactivates the transactivator response element of the first cassette.

In some embodiments, the first RNA comprises a 3′ UTR, and the 3′ UTR comprises a miRNA target site (e.g., a let-7c target site, a miR-22 target site, a miR-26b target site, or a combination thereof). In some embodiments, the first RNA comprises a 5′ UTR, and the 5′ UTR comprises a miRNA target site (e.g., a let-7c target site, a miR-22 target site, a miR-26b target site, or a combination thereof).

In some embodiments, the second RNA comprises a miRNA target site (e.g., a let-7c target site, a miR-22 target site, a miR-26b target site, or a combination thereof). In some embodiments, the second RNA comprises a 3′ UTR, and the 3′ UTR comprises a miRNA target site (e.g., a let-7c target site, a miR-22 target site, a miR-26b target site, or a combination thereof). In some embodiments, the second RNA comprises a 5′ UTR, and the 5′ UTR comprises a miRNA target site (e.g., a let-7c target site, a miR-22 target site, a miR-26b target site, or a combination thereof). In some embodiments, at least one miRNA target site of the first cassette and at least one miRNA target site of the second cassette are the same nucleic acid sequence or are different sequences regulated by the same miRNA.

In some embodiments, the first RNA is operably linked to a transcription factor response element. In some embodiments, the second RNA is operably linked to a transcription factor response element. In some embodiments, the transcription factor response element of the first cassette and the transcription factor response element of the second cassette consist of identical nucleic acid sequences. In some embodiments, the transcription factor response element of the first cassette and the transcription factor response element of the second cassette consist of different nucleic acid sequences. In some embodiments, either the first cassette or the second cassette or both, comprise at least two, at least three . . . types of transcription factor response elements.

In some embodiments, the first cassette comprises, from 5′ to 3′: (i) an upstream regulatory component comprising the transactivator response element and the transcription factor response element; (ii) the nucleic acid sequence encoding the output; and (iii) a downstream component comprising a let-7c target site; and the second cassette comprises, from 5′ to 3′: (i) an upstream regulatory component comprising the transcription factor response element; (ii) the nucleic acid sequence encoding the transactivator; and (iii) a downstream component comprising a let-7c target site.

In some embodiments, the first cassette comprises, from 5′ to 3′: (i) an upstream regulatory component comprising the transcription factor response element and the transactivator response element; (ii) the nucleic acid sequence encoding the output; and (iii) a downstream component comprising a let-7c target site; and the second cassette comprises, from 5′ to 3′: (i) an upstream regulatory component comprising the transcription factor response element; (ii) the nucleic acid sequence encoding the transactivator; and (iii) a downstream component comprising a let-7c target site.

In some embodiments, the first cassette comprises, from 5′ to 3′: (i) an upstream regulatory component comprising the transactivator response element and the transcription factor response element; (ii) the nucleic acid sequence encoding the output; and (iii) a downstream component comprising a let-7c target site; and the second cassette comprises, from 5′ to 3′: (i) an upstream regulatory component comprising a promoter element; (ii) the nucleic acid sequence encoding the transactivator; and (iii) a downstream component comprising a let-7c target site.

In some embodiments, the first cassette comprises, from 5′ to 3′: (i) an upstream regulatory component comprising the transcription factor response element and the transactivator response element; (ii) the nucleic acid sequence encoding the output; and (iii) a downstream component comprising a let-7c target site; and the second cassette comprises, from 5′ to 3′: (i) an upstream regulatory component comprising promoter element; (ii) the nucleic acid sequence encoding the transactivator; and (iii) a downstream component comprising a let-7c target site.

In some embodiments, the upstream regulatory component of the first cassette comprises a promoter element in addition to the transcription factor response element. In some embodiments, a promoter element replaces the transcription factor response element. In some embodiments, the promoter element comprises a mammalian promoter or promoter fragment.

In some embodiments, the first cassette and the second cassette are in a convergent orientation. In some embodiments, the first cassette and the second cassette are in a divergent orientation. In some embodiments, the first cassette and the second cassette are in a head-to-tail orientation.

The first and/or second cassette may be flanked by one or more insulators (e.g., 1, 2, 3, 4, 5, 6, 7, 8, 9, or 10 insulators). For example, in some embodiments, the first cassette or the second cassette is flanked by an insulator. In some embodiments, both the first cassette and the second cassette are flanked by an insulator. In some embodiments, the first cassette or the second cassette is flanked on both sides by an insulator.

Exemplary contiguous polynucleic acids are listed in TABLE 6. In some embodiments, a contiguous polynucleic acid comprises a nucleic acid sequence listed in TABLE 6 or a nucleic acid sequence having at least 70%, at least 80%, at least 85%, at least 90%, at least 95%, at least 96%, at least 97%, at least 98%, at least 99%, or 100% identity to a nucleic acid sequence listed in TABLE 6.

TABLE 6
Exemplary contiguous polynucleic acids.

| Seq ID | Name | SEQUENCE |
|---|---|---|
| 269 | ITR.C.CMV. | CCTGCAGGCAGCTGCGCGCTCGCTCGCTCACTGAGGCCGCCCGGGCAAAGCCCGGGCGTCG |
| | Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTC |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCAAGCACTCTGATTTGACAATTAAAGCACTCTGATTTGACAATTAAAGCACT |
| | | CTGATTTGACAATTAAAGCACTCTGATTTGACAATTAGTCGACCTCGAGAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTTCTGATTTTGTAGGTAACCACGTGCG |
| | | GACCGAGCGGCCGCAGGAACCCCTAGTGATGGAGTGGCCACTCCCTCTCTGCGCGCTCGCT |
| | | CGCTCACTGAGGCCGGGCGACCAAAGGTCGCCCGACGCCCGGGCTTTGCCCGGGCGGCCTC |
| | | AGTGAGCGAGCGAGCGCGCAGCTGCCTGCAGG |
| 270 | ITR.c.Let7c. | CCTGCAGGCAGCTGCGCGCTCGCTCGCTCACTGAGGCCGCCCGGGCAAAGCCCGGGCGTCG |
| | Mcherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTC |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTGATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCAACCATACAACCTACTACCTCAAACCATACAACCTACTACCTCAAACCATA |
| | | CAACCTACTACCTCAAACCATACAACCTACTACCTCAGTCGACCTCGAGAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTTCTGATTTTGTAGGTAACCACGTGCG |
| | | GACCGAGCGGCCGCAGGAACCCCTAGTGATGGAGTGGCCACTCCCTCTCTGCGCGCTCGCT |
| | | CGCTCACTGAGGCCGGGCGACCAAAGGTCGCCCGACGCCCGGGCTTTGCCCGGGCGGCCTC |
| | | AGTGAGCGAGCGAGCGCGCAGCTGCCTGCAGG |
| 271 | ITR.C.TF- | CCTGCAGGCAGCTGCGCGCTCGCTCGCTCACTGAGGCCGCCCGGGCAAAGCCCGGGCGTCG |
| | AND.Mcherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGCTTCGAATCGATGAATTCGA |
| | | AGCTTCTACCCACCGTACTCGTCAATTCCAAGGGCATCGGTAAACATCTGCTCAAACTCGAA |
| | | GTCGGCCATATCCAGAGCGCCGTAGGGGGCGGAGTCGTGGGGGGTAAATCCCGGACCCGGG |
| | | GAATCCCCGTCCCCCAACATGTCCAGATCGAAATCGTCTAGCGCGTCGGCATGCGCCATCGC |
| | | CACGTCCTCGCCGTCTAAGTGGAGCTCGTCCCCCAGGCTGACATCGGTCGGGGGGGCCGTCG |
| | | ACAGTCTGCGCGTGTGTCCCGCGGGGAGAAAGGACAGGCGCGGAGCCGCCAGCCCCGCCTC |
| | | TTCGGGGGCGTCGTCGTCCGGGAGATCGAGCAGGCCCTCGATGGTAGACCCGTAATTGTTTT |
| | | TCGTACGCGCGCGGCTGTACGCGGAGGCCTGTTCGACCATCGCGTCGATGCCCGCGACGAG |
| | | CAGGTCGAGGGCGAACTCGAAGTCCCGGTCCAGCATCTCCGCCACGGTGTCGCCGCCCCGG |
| | | GCCGCCATGATGTCCTGCGCGTCCTCGATGACGCCCGCGGTGTCCGGCACCTCGGTCACCGC |
| | | GGTCATCGAGTCCTGGAAGTACTCCTCCGGACTCAGCCCGGTGTCCGCCACCCGGGCGAGG |
| | | AAGCGGCCCTCGATGGTGCCGTAGCCGTAGACGAACTGGAAGACGGCCGAGATGGCGCCGG |
| | | TCAGGCGGTGCGCGGGCAGCCCGCTGCGGCGCACGACGTTCTGCACCGCGCGGGAGAAGGC |
| | | CAGCGAGTGCGGGCCGATGTTGAGGTAGGTGCCGACCAGCCGGGACGACCAGGGGTGGCG |
| | | CACCAGCAGCGCCCGGTTCTCCCGGGCCAGGGCCCGCAGTTCCTCGCGCCAGTCGAGCCCG |
| | | GCGTCCGGGTCCGGGTGGCGCAGCTCGCCGAAGACGGCGTCCAGGGCGAGCTCGAGCAACT |
| | | GGTCCTTGGTGTCGACGTACCAGTACACGGACATCGCGGTGACGTTCAGCTCGGCGGCCAG |
| | | GCGGCGCATCGAGAACCCCGTCAGGCCCTCCGTGTCCAGCAGCCGGACGGTGACCCCGGTG |
| | | ATCCGGTCCCGGTCGAGCCCGGACGGCTGCCCCCCACGGCGACCGCCGCGCCGCCCCTCCCC |
| | | CGACAGCCACACGCTGTCCCGCGGCCCCTCCCGCCCTGCCTTCGCCATGCGCACCTCTCCTC |
| | | GACTCATACCGGTAGCGCTAGCGATGAGCTCTGGTAGTAGACTAGTGGCCCCCATTATATAC |
| | | CCTCTAGAGCATATGTCTCACAAAGAGGGCTTTGTGTAGTCTCACAAAGAGGGCTTTGTGTA |
| | | GTCTCACAAAGAGGGCTTTGTGTAGGGCGCGCCCCCGTAGCTTGGCGTAATCACATGTCCGT |
| | | CGTTTTACAACGTCGTGACTGGGAAAACCCTGGCCTGCAAGGCGATTAAGTTGGGTAACGC |
| | | CAGGGTTTTCCCAGTCACGACGTTGTAAAACGACGGACATGTGAAATAGCGCTGTACAGCG |
| | | TATGGGAATCTCTTGTACGGTGTACGAGTATCTTCCCGTACACCGTACGGCGCGCCAGTTAA |
| | | TAATTAACTAGTTAATAATTAACTAGTTAATAATTAACTCATATGCTCTAGAGGGTATATAA |
| | | TGGGGGCCACTAGTCTACTACCAGAGCTCATCGCTAGCGCTGGATCCGCCACCATGGTGAGC |
| | | AAGGGCGAGGAGGATAACATGGCCATCATCAAGGAGTTCATGCGCTTCAAGGTGCACATGG |
| | | AGGGCTCCGTGAACGGCCACGAGTTCGAGATCGAGGGCGAGGGCGAGGGCCGCCCCTACG |
| | | AGGGCACCCAGACCGCCAAGCTGAAGGTGACCAAGGGTGGCCCCCTGCCCTTCGCCTGGGA |
| | | CATCCTGTCCCCTCAGTTCATGTACGGCTCCAAGGCCTACGTGAAGCACCCCGCCGACATCC |
| | | CCGACTACTTGAAGCTGTCCTTCCCCGAGGGCTTCAAGTGGGAGCGCGTGATGAACTTCGAG |
| | | GACGGCGGCGTGGTGACCGTGACCCAGGACTCCTCCCTCCAGGACGGCGAGTTCATCTACA |
| | | AGGTGAAGCTGCGCGGCACCAACTTCCCCTCCGACGGCCCCGTAATGCAGAAGAAGACCAT |
| | | GGGCTGGGAGGCCTCCTCCGAGCGGATGTACCCCGAGGACGGCGCCCTGAAGGGCGAGATC |
| | | AAGCAGCGGCTGAAGCTGAAGGACGGCGGCCACTACGACGCTGAGGTCAAGACCACCTAC |
| | | AAGGCCAAGAAGCCCGTGCAGCTGCCCGGCGCCTACAACGTCAACATCAAGTTGGACATCA |
| | | CCTCCCACAACGAGGACTACACCATCGTGGAACAGTACGAACGCGCCGAGGGCCGCCACTG |
| | | CACCGGCGGCATGGACGAGCTGTACAAGTAGGGTACCGTCGACCTCGAGAGATCTACGGGT |
| | | GGCATCCCTGTGACCCCTCCCCAGTGCCTCTCCTGGCCCTGGAAGTTGCCACTCCAGTGCCC |
| | | ACCAGCCTTGTCCTAATAAAATTAAGTTGCATCATTTTGTCTGACTAGGTGTCCTTCTATAAT |
| | | ATTATGGGGTGGAGGGGGGTGGTATGGAGCAAGGGGCAAGTTGGGAAGACAACCTGTAGG |
| | | GCCTGCGGGGTCTATTGGGAACCAAGCTGGAGTGCAGTGGCACAATCTTGGCTCACTGCAA |
| | | TCTCCGCCTCCTGGGTTCAAGCGATTCTCCTGCCTCAGCCTCCCGAGTTGTTGGGATTCCAGG |
| | | CATGCATGACCAGGCTCAGCTAATTTTTGTTTTTTTGGTAGAGACGGGGTTTCACCATATTGG |
| | | CCAGGCTGGTCTCCAACTCCTAATCTCAGGTGATCTACCCACCTTGGCCTCCCAAATTGCTG |
| | | GGATTACAGGCGTGAACCACTGCTCCCTTCCCTGTCCTTCTGATTTTGTAGGTAACCACGTGC |
| | | GGACCGAGCGGCCGCAGGAACCCCTAGTGATGGAGTTGGCCACTCCCTCTCTGCGCGCTCG |
| | | CTCGCTCACTGAGGCCGGGCGACCAAAGGTCGCCCGACGCCCGGGCTTTGCCCGGGCGGCC |
| | | TCAGTGAGCGAGCGAGCGCGCAGCTGCCTGCAGG |
| 272 | ITR.HCC.V2. | CCTGCAGGCAGCTGCGCGCTCGCTCGCTCACTGAGGCCGCCCGGGCAAAGCCCGGGCGTCG |
| | Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGTGAGGTAGTAGGTTGTATGG |
| | | TTTGAGGTAGTAGGTTGTATGGTTTGAGGTAGTAGGTTGTATGGTTTGAGGTAGTAGGTTGT |
| | | ATGGTTATCGATGAATTCGAAGCTTCTACCCACCGTACTCGTCAATTCCAAGGGCATCGGTA |
| | | AACATCTGCTCAAACTCGAAGTCGGCCATATCCAGAGCGCCGTAGGGGGCGGAGTCGTGGG |
| | | GGGTAAATCCCGGACCCGGGGAATCCCCGTCCCCCAACATGTCCAGATCGAAATCGTCTAG |
| | | CGCGTCGGCATGCGCCATCGCCACGTCCTCGCCGTCTAAGTGGAGCTCGTCCCCCAGGCTGA |
| | | CATCGGTCGGGGGGGCCGTCGACAGTCTGCGCGTGTGTCCCGCGGGGAGAAAGGACAGGCG |
| | | CGGAGCCGCCAGCCCCGCCTCTTCGGGGGCGTCGTCGTCCGGGAGATCGAGCAGGCCCTCG |
| | | ATGGTAGACCCGTAATTGTTTTTCGTACGCGCGCGGCTGTACGCGGAGGCCTGTTCGACCAT |
| | | CGCGTCGATGCCCGCGACGAGCAGGTCGAGGGCGAACTCGAAGTCCCGGTCCAGCATCTCC |
| | | GCCACGGTGTCGCCGCCCCGGGCCGCCATGATGTCCTGCGCGTCCTCGATGACGCCCGCGGT |
| | | GTCCGGCACCTCGGTCACCGCGGTCATCGAGTCCTGGAAGTACTCCTCCGGACTCAGCCCGG |
| | | TGTCCGCCACCCGGGCGAGGAAGCGGCCCTCGATGGTGCCGTAGCCGTAGACGAACTGGAA |
| | | GACGGCCGAGATGGCGCCGGTCAGGCGGTGCGCGGGCAGCCCGCTGCGGCGCACGACGTTC |
| | | TGCACCGCGCGGGAGAAGGCCAGCGAGTGCGGGCCGATGTTGAGGTAGGTGCCGACCAGCC |
| | | GGGACGACCAGGGGTGGCGCACCAGCAGCGCCCGGTTCTCCCGGGCCAGGGCCCGCAGTTC |
| | | CTCGCGCCAGTCGAGCCCGGCGTCCGGGTCCGGGTGGCGCAGCTCGCCGAAGACGGCGTCC |
| | | AGGGCGAGCTCGAGCAACTGGTCCTTGGTGTCGACGTACCAGTACACGGACATCGCGGTGA |
| | | CGTTCAGCTCGGCGGCCAGGCGGCGCATCGAGAACCCCGTCAGGCCCTCCGTGTCCAGCAG |
| | | CCGGACGGTGACCCCGGTGATCCGGTCCCGGTCGAGCCCGGACGGCTGCCCCCCACGGCGA |
| | | CCGCCGCGCCGCCCCTCCCCCGACAGCCACACGCTGTCCCGCGGCCCCTCCCGCCCTGCCTT |
| | | CGCCATGCGCACCTCTCCTCGACTCATACCGGTAGCGCTAGCGATGAGCTCTGGTAGTAGAC |
| | | TAGTGGCCCCCATTATATACCCTCTAGAGCATATGTCTCACAAAGAGGGCTTTGTGTAGTCT |
| | | CACAAAGAGGGCTTTGTGTAGTCTCACAAAGAGGGCTTTGTGTAGGGCGCGCCCCCGTAGC |
| | | TTGGCGTAATCACATGTCCGTCGTTTTACAACGTCGTGACTGGGAAAACCCTGGCCTGCAAG |
| | | GCGATTAAGTTGGGTAACGCCAGGGTTTTCCCAGTCACGACGTTGTAAAACGACGGACATG |
| | | TGAAATAGCGCTGTACAGCGTATGGGAATCTCTTGTACGGTGTACGAGTATCTTCCCGTACA |
| | | CCGTACGGCGCGCCAGTTAATAATTAACTAGTTAATAATTAACTAGTTAATAATTAACTCAT |
| | | ATGCTCTAGAGGGTATATAATGGGGGCCACTAGTCTACTACCAGAGCTCATCGCTAGCGCTG |
| | | GATCCGCCACCATGGTGAGCAAGGGCGAGGAGGATAACATGGCCATCATCAAGGAGTTCAT |
| | | GCGCTTCAAGGTGCACATGGAGGGCTCCGTGAACGGCCACGAGTTCGAGATCGAGGGCGAG |
| | | GGCGAGGGCCGCCCCTACGAGGGCACCCAGACCGCCAAGCTGAAGGTGACCAAGGGTGGC |
| | | CCCCTGCCCTTCGCCTGGGACATCCTGTCCCCTCAGTTCATGTACGGCTCCAAGGCCTACGT |
| | | GAAGCACCCCGCCGACATCCCCGACTACTTGAAGCTGTCCTTCCCCGAGGGCTTCAAGTGGG |
| | | AGCGCGTGATGAACTTCGAGGACGGCGGCGTGGTGACCGTGACCCAGGACTCCTCCCTCCA |
| | | GGACGGCGAGTTCATCTACAAGGTGAAGCTGCGCGGCACCAACTTCCCCTCCGACGGCCCC |
| | | GTAATGCAGAAGAAGACCATGGGCTGGGAGGCCTCCTCCGAGCGGATGTACCCCGAGGACG |
| | | GCGCCCTGAAGGGCGAGATCAAGCAGCGGCTGAAGCTGAAGGACGGCGGCCACTACGACG |
| | | CTGAGGTCAAGACCACCTACAAGGCCAAGAAGCCCGTGCAGCTGCCCGGCGCCTACAACGT |
| | | CAACATCAAGTTGGACATCACCTCCCACAACGAGGACTACACCATCGTGGAACAGTACGAA |
| | | CGCGCCGAGGGCCGCCACTCCACCGGCGGCATGGACGAGCTGTACAAGTAGGGTACCAACC |
| | | ATACAACCTACTACCTCAAACCATACAACCTACTACCTCAAACCATACAACCTACTACCTCA |
| | | AACCATACAACCTACTACCTCAAGATCTACGGGTGGCATCCCTGTGACCCCTCCCCAGTGCC |
| | | TCTCCTGGCCCTGGAAGTTGCCACTCCAGTGCCCACCAGCCTTGTCCTAATAAAATTAAGTT |
| | | GCATCATTTTGTCTGACTAGGTGTCCTTCTATAATATTATGGGGTGGAGGGGGGTGGTATGG |
| | | AGCAAGGGGCAAGTTGGGAAGACAACCTGTAGGGCCTGCGGGGTCTATTGGGAACCAAGCT |
| | | GGAGTGCAGTGGCACAATCTTGGCTCACTGCAATCTCCGCCTCCTGGGTTCAAGCGATTCTC |
| | | CTGCCTCAGCCTCCCGAGTTGTTGGGATTCCAGGCATGCATGACCAGGCTCAGCTAATTTTT |
| | | GTTTTTTTGGTAGAGACGGGGTTTCACCATATTGGCCAGGCTGGTCTCCAACTCCTAATCTCA |
| | | GGTGATCTACCCACCTTGGCCTCCCAAATTGCTGGGATTACAGGCGTGAACCACTGCTCCCT |
| | | TCCCTGTCCTTCTGATTTTGTAGGTAACCACGTGCGGACCGAGCGGCCGCAGGAACCCCTAG |
| | | TGATGGAGTTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCGCAGCTGCC |
| | | TGCAGG |
| 273 | ITR.C.HNF1- | CCTGCAGGCAGCTGCGCGCTCGCTCGCTCACTGAGGCCGCCCGGGCAAAGCCCGGGCGTCG |
| | FB.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGGGGTCCACTTGCTCCTGGGC |
| | | CCACACAGTCCTGCAGTATTGTGTATATAAGGCCAGGGCAAAGAGGAGCAGGTTTTAAAGT |
| | | GAAAGGCAGGCAGGTGTTGGGGAGGCAGTTACCGGGGCAACGGGAACAGGGCGTTTCGGA |
| | | GGTGGTTGCCATGGGGACCTGGATGCTGTTCCATTCGCCATTCAGGCTGCGCAACTGTTGGG |
| | | AAGGGCGATCGGTGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGTAAAACGACGGAA |
| | | TTCGAAGCTTACGACGGACATGTGAAATAGCGCTGTACAGCGTATGGGAATCTCTTGTACGG |
| | | TGTACGAGTATCTTCCCGTACACCGTACGGCGCGCCAGTTAATAATTAACTAGTTAATAATT |
| | | AACTAGTTAATAATTAACTCATATGCTCTAGAGGGTATATAATGGGGGCCACTAGTCTACTA |
| | | CCAGAGCTCATCGCTAGCGCTGGATCCGCCACCATGGTGAGCAAGGGCGAGGAGGATAACA |
| | | TGGCCATCATCAAGGAGTTCATGCGCTTCAAGGTGCACATGGAGGGCTCCGTGAACGGCCA |
| | | CGAGTTCGAGATCGAGGGCGAGGGCGAGGGCCGCCCCTACGAGGGCACCCAGACCGCCAA |
| | | GCTGAAGGTGACCAAGGGTGGCCCCCTGCCCTTCGCCTGGGACATCCTGTCCCCTCAGTTCA |
| | | TGTACGGCTCCAAGGCCTACGTGAAGCACCCCGCCGACATCCCCGACTACTTGAAGCTGTCC |
| | | TTCCCCGAGGGCTTCAAGTGGGAGCGCGTGATGAACTTCGAGGACGGCGGCGTGGTGACCG |
| | | TGACCCAGGACTCCTCCCTCCAGGACGGCGAGTTCATCTACAAGGTGAAGCTGCGCGGCAC |
| | | CAACTTCCCCTCCGACGGCCCCGTAATGCAGAAGAAGACCATGGGCTGGGAGGCCTCCTCC |
| | | GAGCGGATGTACCCCGAGGACGGCGCCCTGAAGGGCGAGATCAAGCAGCGGCTGAAGCTG |
| | | AAGGACGGCGGCCACTACGACGCTGAGGTCAAGACCACCTACAAGGCCAAGAAGCCCGTG |
| | | CAGCTGCCCGGCGCCTACAACGTCAACATCAAGTTGGACATCACCTCCCACAACGAGGACT |
| | | ACACCATCGTGGAACAGTACGAACGCGCCGAGGGCCGCCACTCCACCGGCGGCATGGACGA |
| | | GCTGTACAAGTCCGGAAGAGCCGAGGGCAGGGGAAGTCTTCTAACATGCGGGGACGTGGA |
| | | GGAAAATCCCGGGCCCAGATCTATGAGTCGAGGAGAGGTGCGCATGGCGAAGGCAGGGCG |
| | | GGAGGGGCCGCGGGACAGCGTGTGGCTGTCGGGGGAGGGGCGGCGCGGCGGTCGCCGTGG |
| | | GGGGCAGCCGTCCGGGCTCGACCGGGACCGGATCACCGGGGTCACCGTCCGGCTGCTGGAC |
| | | ACGGAGGGCCTGACGGGGTTCTCGATGCGCCGCCTGGCCGCCGAGCTGAACGTCACCGCGA |
| | | TGTCCGTGTACTGGTACGTCGACACCAAGGACCAGTTGCTCGAGCTCGCCCTGGACGCCGTC |
| | | TTCGGCGAGCTGCGCCACCCGGACCCGGACGCCGGGCTCGACTGGCGCGAGGAACTGCGGG |
| | | CCCTGGCCCGGGAGAACCGGGCGCTGCTGGTGCGCCACCCCTGGTCGTCCCGGCTGGTCGG |
| | | CACCTACCTCAACATCGGCCCGCACTCGCTGGCCTTCTCCCGCGCGGTGCAGAACGTCGTGC |
| | | GCCGCAGCGGGCTGCCCGCGCACCGCCTGACCGGCGCCATCTCGGCCGTCTTCCAGTTCGTC |
| | | TACGGCTACGGCACCATCGAGGGCCGCTTCCTCGCCCGGGTGGCGGACACCGGGCTGAGTC |
| | | CGGAGGAGTACTTCCAGGACTCGATGACCGCGGTGACCGAGGTGCCGGACACCGCGGGCGT |
| | | CATCGAGGACGCGCAGGACATCATGGCGGCCCGGGGCGGCGACACCGTGGCGGAGATGCT |
| | | GGACCGGGACTTCGAGTTCGCCCTCGACCTGCTCGTCGCGGGCATCGACGCGATGGTCGAA |
| | | CAGGCCTCCGCGTACAGCCGCGCGCATGATGAGTTTCCCACCATGGTGTTTCCTTCTGGGCA |
| | | GATCAGCCAGGCCTCGGCCTTGGCCCCGGCCCCTCCCCAAGTCCTGCCCCAGGCTCCAGCCC |
| | | CTGCCCCTGCTCCAGCCATGGTATCAGCTCTGGCCCAGGCCCCAGCCCCTGTCCCAGTCCTA |
| | | GCCCCAGGCCCTCCTCAGGCTGTGGCCCCACCTGCCCCCAAGCCCACCCAGGCTGGGGAAG |
| | | GAACGCTGTCAGAGGCCCTGCTGCAGCTGCAGTTTGATGATGAAGACCTGGGGGCCTTGCTT |
| | | GGCAACAGCACAGACCCAGCTGTGTTCACAGACCTGGCATCCGTCGACAACTCCGAGTTTG |
| | | AGCAGCTGCTGAACCAGGGCATACCTGTGGCCCCCCACACAACTGAGCCCATGCTGATGGA |
| | | GTACCCTGAGGCTATAACTCGCCTAGTGACAGGGGCCCAGAGGCCCCCCGACCCAGCTCCT |
| | | GCTCCACTGGGGGCCCCGGGGCTCCCCAATGGCCTCCTTTCAGGAGATGAAGACTTCTCCTC |
| | | CATTGCGGACATGGACTTCTCAGCCCTGCTGAGTCAGATCAGCTCCTAAGGAAGCTTGGTAC |
| | | CGTCGACCTCGAGAGATCTACGGGTGGCATCCCTGTGACCCCTCCCCAGTGCCTCTCCTGGC |
| | | CCTGGAAGTTGCCACTCCAGTGCCCACCAGCCTTGTCCTAATAAAATTAAGTTGCATCATTT |
| | | TGTCTGACTAGGTGTCCTTCTATAATATTATGGGGTGGAGGGGGGTGGTATGGAGCAAGGG |
| | | GCAAGTTGGGAAGACAACCTGTAGGGCCTGCGGGGTCTATTGGGAACCAAGCTGGAGTGCA |
| | | GTGGCACAATCTTGGCTCACTGCAATCTCCGCCTCCTGGGTTCAAGCGATTCTCCTGCCTCA |
| | | GCCTCCCGAGTTGTTGGGATTCCAGGCATGCATGACCAGGCTCAGCTAATTTTTGTTTTTTTG |
| | | GTAGAGACGGGGTTTCACCATATTGGCCAGGCTGGTCTCCAACTCCTAATCTCAGGTGATCT |
| | | ACCCACCTTGGCCTCCCAAATTGCTGGGATTACAGGCGTGAACCACTGCTCCCTTCCCTGTC |
| | | CTTCTGATTTTGTAGGTAACCACGTGCGGACCGAGCGGCCGCAGGAACCCCTAGTGATGGA |
| | | GTTGGCCACTCCCTCTCTGCGCGCTCGCTCGCTCACTGAGGCCGGGCGACCAAAGGTCGCCC |
| | | GACGCCCGGGCTTTGCCCGGGCGGCCTCAGTGAGCGAGCGAGCGCGCAGCTGCCTGCAGG |
| 274 | ITR.C.SOX- | CCTGCAGGCAGCTGCGCGCTCGCTCGCTCACTGAGGCCGCCCGGGCAAAGCCCGGGCGTCG |
| | FB.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGGGGTCCACTTGCTCCTGGGC |
| | | CCACACAGTCCTGCAGTATTGTGTATATAAGGCCAGGGCAAAGAGGAGCAGGTTTTAAAGT |
| | | GAAAGGCAGGCAGGTGTTGGGGAGGCAGTTACCGGGGCAACGGGAACAGGGCGTTTCGGA |
| | | GGTGGTTGCCATGGGGACCTGGATGCTGTTCCATTCGCCATTCAGGCTGCGCAACTGTTGGG |
| | | AAGGGCGATCGGTGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGTAAAACGACGGAA |
| | | TTCGAAGCTTACGACGGACATGTGAAATAGCGCTGTACAGCGTATGGGAATCTCTTGTACGG |
| | | TGTACGAGTATCTTCCCGTACACCGTACGGCGCGCCCTACACAAAGCCCTCTTTGTGAGACT |
| | | ACACAAAGCCCTCTTTGTGAGACTACACAAAGCCCTCTTTGTGAGACATATGCTCTAGAGGG |
| | | TATATAATGGGGGCCACTAGTCTACTACCAGAGCTCATCGCTAGCGCTGGATCCGCCACCAT |
| | | GGTGAGCAAGGGCGAGGAGGATAACATGGCCATCATCAAGGAGTTCATGCGCTTCAAGGTG |
| | | CACATGGAGGGCTCCGTGAACGGCCACGAGTTCGAGATCGAGGGCGAGGGCGAGGGCCGC |
| | | CCCTACGAGGGCACCCAGACCGCCAAGCTGAAGGTGACCAAGGGTGGCCCCCTGCCCTTCG |
| | | CCTGGGACATCCTGTCCCCTCAGTTCATGTACGGCTCCAAGGCCTACGTGAAGCACCCCGCC |
| | | GACATCCCCGACTACTTGAAGCTGTCCTTCCCCGAGGGCTTCAAGTGGGAGCGCGTGATGAA |
| | | CTTCGAGGACGGCGGCGTGGTGACCGTGACCCAGGACTCCTCCCTCCAGGACGGCGAGTTC |
| | | ATCTACAAGGTGAAGCTGCGCGGCACCAACTTCCCCTCCGACGGCCCCGTAATGCAGAAGA |
| | | AGACCATGGGCTGGGAGGCCTCCTCCGAGCGGATGTACCCCGAGGACGGCGCCCTGAAGGG |
| | | CGAGATCAAGCAGCGGCTGAAGCTGAAGGACGGCGGCCACTACGACGCTGAGGTCAAGAC |
| | | CACCTACAAGGCCAAGAAGCCCGTGCAGCTGCCCGGCGCCTACAACGTCAACATCAAGTTG |
| | | GACATCACCTCCCACAACGAGGACTACACCATCGTGGAACAGTACGAACGCGCCGAGGGCC |
| | | GCCACTCCACCGGCGGCATGGACGAGCTGTACAAGTCCGGAAGAGCCGAGGGCAGGGGAA |
| | | GTCTTCTAACATGCGGGGACGTGGAGGAAAATCCCGGGCCCAGATCTATGAGTCGAGGAGA |
| | | GGTGCGCATGGCGAAGGCAGGGCGGGAGGGGCCGCGGGACAGCGTGTGGCTGTCGGGGGA |
| | | GGGGCGGCGCGGCGGTCGCCGTGGGGGGCAGCCGTCCGGGCTCGACCGGGACCGGATCACC |
| | | GGGGTCACCGTCCGGCTGCTGGACACGGAGGGCCTGACGGGGTTCTCGATGCGCCGCCTGG |
| | | CCGCCGAGCTGAACGTCACCGCGATGTCCGTGTACTGGTACGTCGACACCAAGGACCAGTT |
| | | GCTCGAGCTCGCCCTGGACGCCGTCTTCGGCGAGCTGCGCCACCCGGACCCGGACGCCGGG |
| | | CTCGACTGGCGCGAGGAACTGCGGGCCCTGGCCCGGGAGAACCGGGCGCTGCTGGTGCGCC |
| | | ACCCCTGGTCGTCCCGGCTGGTCGGCACCTACCTCAACATCGGCCCGCACTCGCTGGCCTTC |
| | | TCCCGCGCGGTGCAGAACGTCGTGCGCCGCAGCGGGCTGCCCGCGCACCGCCTGACCGGCG |
| | | CCATCTCGGCCGTCTTCCAGTTCGTCTACGGCTACGGCACCATCGAGGGCCGCTTCCTCGCC |
| | | CGGGTGGCGGACACCGGGCTGAGTCCGGAGGAGTACTTCCAGGACTCGATGACCGCGGTGA |
| | | CCGAGGTGCCGGACACCGCGGGCGTCATCGAGGACGCGCAGGACATCATGGCGGCCCGGG |
| | | GCGGCGACACCGTGGCGGAGATGCTGGACCGGGACTTCGAGTTCGCCCTCGACCTGCTCGT |
| | | CGCGGGCATCGACGCGATGGTCGAACAGGCCTCCGCGTACAGCCGCGCGCATGATGAGTTT |
| | | CCCACCATGGTGTTTCCTTCTGGGCAGATCAGCCAGGCCTCGGCCTTGGCCCCGGCCCCTCC |
| | | CCAAGTCCTGCCCCAGGCTCCAGCCCCTGCCCCTGCTCCAGCCATGGTATCAGCTCTGGCCC |
| | | AGGCCCCAGCCCCTGTCCCAGTCCTAGCCCCAGGCCCTCCTCAGGCTGTGGCCCCACCTGCC |
| | | CCCAAGCCCACCCAGGCTGGGGAAGGAACGCTGTCAGAGGCCCTGCTGCAGCTGCAGTTTG |
| | | ATGATGAAGACCTGGGGGCCTTGCTTGGCAACAGCACAGACCCAGCTGTGTTCACAGACCT |
| | | GGCATCCGTCGACAACTCCGAGTTTCAGCAGCTGCTGAACCAGGGCATACCTGTGGCCCCCC |
| | | ACACAACTGAGCCCATGCTGATGGAGTACCCTGAGGCTATAACTCGCCTAGTGACAGGGGC |
| | | CCAGAGGCCCCCCGACCCAGCTCCTGCTCCACTGGGGGCCCCGGGGCTCCCCAATGGCCTCC |
| | | TTTCAGGAGATGAAGACTTCTCCTCCATTGCGGACATGGACTTCTCAGCCCTGCTGAGTCAG |
| | | ATCAGCTCCTAAGGAAGCTTGGTACCGTCGACCTCGAGAGATCTACGGGTGGCATCCCTGTG |
| | | ACCCCTCCCCAGTGCCTCTCCTGGCCCTGGAAGTTGCCACTCCAGTGCCCACCAGCCTTGTC |
| | | CTAATAAAATTAAGTTGCATCATTTTGTCTGACTAGGTGTCCTTCTATAATATTATGGGGTGG |
| | | AGGGGGGTGGTATGGAGCAAGGGGCAAGTTGGGAAGACAACCTGTAGGGCCTGCGGGGTC |
| | | TATTGGGAACCAAGCTGGAGTGCAGTGGCACAATCTTGGCTCACTGCAATCTCCGCCTCCTG |
| | | GGTTCAAGCGATTCTCCTGCCTCAGCCTCCCGAGTTGTTGGGATTCCAGGCATGCATGACCA |
| | | GGCTCAGCTAATTTTTGTTTTTTTGGTAGAGACGGGGTTTCACCATATTGGCCAGGCTGGTCT |
| | | CCAACTCCTAATCTCAGGTGATCTACCCACCTTGGCCTCCCAAATTGCTGGGATTACAGGCG |
| | | TGAACCACTGCTCCCTTCCCTGTCCTTCTGATTTTGTAGGTAACCACGTGCGGACCGAGCGG |
| | | CCGCAGGAACCCCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCCTCAGTGAGCGAG |
| | | CGAGCGCGCAGCTGCCTGCAGG |
| 275 | ITR.Reporter | CCTGCAGGCAGCTGCGCGCTCGCTCGCTCACTGAGGCCGCCCGGGCAAAGCCCGGGCGTCG |
| | 26B.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCACCTATCCTGAATTACTTGAAACCTATCCTGAATTACTTGAAACCTATCCT |
| | | GAATTACTTGAAACCTATCCTGAATTACTTGAAGTCGACCTCGAGAGATCTACGGGTGGCAT |
| | | CCCTGTGACCCCTCCCCAGTGCCTCTCCTGGCCCTGGAAGTTGCCACTCCAGTGCCCACCAG |
| | | CCTTGTCCTAATAAAATTAAGTTGCATCATTTTGTCTGACTAGGTGTCCTTCTATAATATTAT |
| | | GGGGTGGAGGGGGGTGGTATGGAGCAAGGGGCAAGTTGGGAAGACAACCTGTAGGGCCTG |
| | | CGGGGTCTATTGGGAACCAAGCTGGAGTGCAGTGGCACAATCTTGGCTCACTGCAATCTCCG |
| | | CCTCCTGGGTTCAAGCGATTCTCCTGCCTCAGCCTCCCGAGTTGTTGGGATTCCAGGCATGC |
| | | ATGACCAGGCTCAGCTAATTTTTGTTTTTTTGGTAGAGACGGGGTTTCACCATATTGGCCAG |
| | | GCTGGTCTCCAACTCCTAATCTCAGGTGATCTACCCACCTTGGCCTCCCAAATTGCTGGGATT |
| | | ACAGGCGTGAACCACTGCTCCCTTCCCTGTCCTTCTGATTTTGTAGGTAACCACGTGCGGAC |
| | | CGAGCGGCCGCAGGAACCCCTAGTGATGGAGTTGGCCACTCCCTCTCTGCGCGCTCGCTCGC |
| | | TCACTGAGGCCGGGCGACCAAAGGTCGCCCGACGCCCGGGCTTTGCCCGGGCGGCCTCAGT |
| | | GAGCGAGCGAGCGCGCAGCTGCCTGCAGG |
| 276 | ITR.Reporter | CCTGCAGGCAGCTGCGCGCTCGCTCGCTCACTGAGGCCGCCCGGGCAAAGCCCGGGCGTCG |
| | 22.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCACAGTTCTTCAACTGGCAGCTTACAGTTCTTCAACTGGCAGCTTACAGTTC |
| | | TTCAACTGGCAGCTTACAGTTCTTCAACTGGCAGCTTGTCGACCTCGAGAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTTCTGATTTTGTAGGTAACCACGTGCG |
| | | GACCGAGCGGCCGCAGGAACCCCTAGTGATGGAGTGGCCACTCCCTCTCTGCGCGCTCGCT |
| | | CGCTCACTGAGGCCGGGCGACCAAAGGTCGCCCGACGCCCGGGCTTTGCCCGGGCGGCCTC |
| | | AGTGAGCGAGCGAGCGCGCAGCTGCCTGCAGG |
| 277 | ITR.Reporter | CCTGCAGGCAGCTGCGCGCTCGCTCGCTCACTGAGGCCGCCCGGGCAAAGCCCGGGCGTCG |
| | 126.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCCGTGTTCACAGCGGACCTTGATCGTGTTCACAGCGGACCTTGATCGTGTTC |
| | | ACAGCGGACCTTGATCGTGTTCACAGCGGACCTTGATGTCGACCTCGAGAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTTCTGATTTTGTAGGTAACCACGTGCG |
| | | GACCGAGCGGCCGCAGGAACCCCTAGTGATGGAGTGGCCACTCCCTCTCTGCGCGCTCGCT |
| | | CGCTCACTGAGGCCGGGCGACCAAAGGTCGCCCGACGCCCGGGCTTTGCCCGGGCGGCCTC |
| | | AGTGAGCGAGCGAGCGCGCAGCTGCCTGCAGG |
| 278 | ITR.Reporter | CCTGCAGGCAGCTGCGCGCTCGCTCGCTCACTGAGGCCGCCCGGGCAAAGCCCGGGCGTCG |
| | 424.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCTCCAAAACATGAATTGCTGCTGTCCAAAACATGAATTGCTGCTGTCCAAAAC |
| | | ATGAATTGCTGCTGTCCAAAACATGAATTGCTGCTGGTCGACCTCGAGAGATCTACGGGTGG |
| | | CATCCCTGTGACCCCTCCCCAGTGCCTCTCCTGGCCCTGGAAGTTGCCACTCCAGTGCCCAC |
| | | CAGCCTTGTCCTAATAAAATTAAGTTGCATCATTTTGTCTGACTAGGTGTCCTTCTATAATAT |
| | | TATGGGGTGGAGGGGGGTGGTATGGAGCAAGGGGCAAGTTGGGAAGACAACCTGTAGGGC |
| | | CTGCGGGGTCTATTGGGAACCAAGCTGGAGTGCAGTGGCACAATCTTGGCTCACTGCAATCT |
| | | CCGCCTCCTGGGTTCAAGCGATTCTCCTGCCTCAGCCTCCCGAGTTGTTGGGATTCCAGGCA |
| | | TGCATGACCAGGCTCAGCTAATTTTTGTTTTTTTGGTAGAGACGGGGTTTCACCATATTGGCC |
| | | AGGCTGGTCTCCAACTCCTAATCTCAGGTGATCTACCCACCTTGGCCTCCCAAATTGCTGGG |
| | | ATTACAGGCGTGAACCACTGCTCCCTTCCCTGTCCTTCTGATTTTGTAGGTAACCACGTGCGG |
| | | ACCGAGCGGCCGCAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCCCGGGCGGCCTCA |
| | | GTGAGCGAGCGAGCGCGCAGCTGCCTGCAGG |
| 279 | ITR.Reporter | CCTGCAGGCAGCTGCGCGCTCGCTCGCTCACTGAGGCCGCCCGGGCAAAGCCCGGGCGTCG |
| | 122.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCCAAACACCATTGTCACACTCCACAAACACCATTGTCACACTCCACAAACA |
| | | CCATTGTCACACTCCACAAACACCATTGTCACACTCCAGTCGACCTCGAGAGATCTACGGGT |
| | | GGCATCCCTGTGACCCCTCCCCAGTGCCTCTCCTGGCCCTGGAAGTTGCCACTCCAGTGCCC |
| | | ACCAGCCTTGTCCTAATAAAATTAAGTTGCATCATTTTGTCTGACTAGGTGTCCTTCTATAAT |
| | | ATTATGGGGTGGAGGGGGGTGGTATGGAGCAAGGGGCAAGTTGGGAAGACAACCTGTAGG |
| | | GCCTGCGGGGTCTATTGGGAACCAAGCTGGAGTGCAGTGGCACAATCTTGGCTCACTGCAA |
| | | TCTCCGCCTCCTGGGTTCAAGCGATTCTCCTGCCTCAGCCTCCCGAGTTGTTGGGATTCCAGG |
| | | CATGCATGACCAGGCTCAGCTAATTTTTGTTTTTTTGGTAGAGACGGGGTTTCACCATATTGG |
| | | CCAGGCTGGTCTCCAACTCCTAATCTCAGGTGATCTACCCACCTTGGCCTCCCAAATTGCTG |
| | | GGATTACAGGCGTGAACCACTGCTCCCTTCCCTGTCCTTCTGATTTTGTAGGTAACCACGTGC |
| | | GGACCGAGCGGCCGCAGGAACCCCTAGTGATGGAGTTGGCCACTCCCTCTCTGCGCGCTCG |
| | | CTCGCTCACTGAGGCCGGGCGACCAAAGGTCGCCCGACGCCCGGGCTTTGCCCGGGCGGCC |
| | | TCAGTGAGCGAGCGAGCGCGCAGCTGCCTGCAGG |
| 280 | ITR.Reporter | CCTGCAGGCAGCTGCGCGCTCGCTCGCTCACTGAGGCCGCCCGGGCAAAGCCCGGGCGTCG |
| | 217.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTC |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCTCCAGTCAGTTCCTGATGCAGTATCCAGTCAGTTCCTGATGCAGTATCCAG |
| | | TCAGTTCCTGATGCAGTATCCAGTCAGTTCCTGATGCAGTAGTCGACCTCGAGAGATCTACG |
| | | GGTGGCATCCCTGTGACCCCTCCCCAGTGCCTCTCCTGGCCCTGGAAGTTGCCACTCCAGTG |
| | | CCCACCAGCCTTGTCCTAATAAAATTAAGTTGCATCATTTTGTCTGACTAGGTGTCCTTCTAT |
| | | AATATTATGGGGTGGAGGGGGGTGGTATGGAGCAAGGGGCAAGTTGGGAAGACAACCTGT |
| | | AGGGCCTGCGGGGTCTATTGGGAACCAAGCTGGAGTGCAGTGGCACAATCTTGGCTCACTG |
| | | CAATCTCCGCCTCCTGGGTTCAAGCGATTCTCCTGCCTCAGCCTCCCGAGTTGTTGGGATTCC |
| | | AGGCATGCATGACCAGGCTCAGCTAATTTTTGTTTTTTTGGTAGAGACGGGGTTTCACCATA |
| | | TTGGCCAGGCTGGTCTCCAACTCCTAATCTCAGGTGATCTACCCACCTTGGCCTCCCAAATT |
| | | GCTGGGATTACAGGCGTGAACCACTGCTCCCTTCCCTGTCCTTCTGATTTTGTAGGTAACCAC |
| | | GTGCGGACCGAGCGGCCGCAGGAACCCCTAGTGATGGAGTTGGCCACTCCCTCTCTGCGCG |
| | | CTCGCTCGCTCACTGAGGCCGGGCGACCAAAGGTCGCCCGACGCCCGGGCTTTGCCCGGGC |
| | | GGCCTCAGTGAGCGAGCGAGCGCGCAGCTGCCTGCAGG |
| 281 | ITR.Reporter | CCTGCAGGCAGCTGCGCGCTCGCTCGCTCACTGAGGCCGCCCGGGCAAAGCCCGGGCGTCG |
| | 216A.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTG |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCTCACAGTTGCCAGCTGAGATTATCACAGTTGCCAGCTGAGATTATCACAGT |
| | | TGCCAGCTGAGATTATCACAGTTGCCAGCTGAGATTAGTCGACCTCGAGAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTTCTGATTTTGTAGGTAACCACGTGCG |
| | | GACCGAGCGGCCGCAGGAACCCCTAGTGATGGAGTGGCCACTCCCTCTCTGCGCGCTCGCT |
| | | CGCTCACTGAGGCCGGGCGACCAAAGGTCGCCCGACGCCCGGGCTTTGCCCGGGCGGCCTC |
| | | AGTGAGCGAGCGAGCGCGCAGCTGCCTGCAGG |
| 282 | ITR.Reporter | CCTGCAGGCAGCTGCGCGCTCGCTCGCTCACTGAGGCCGCCCGGGCAAAGCCCGGGCGTCG |
| | 208A.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTC |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCACAAGCTTTTTGCTCGTCTTATACAAGCTTTTTGCTCGTCTTATACAAGCTT |
| | | TTTGCTCGTCTTATACAAGCTTTTTGCTCGTCTTATGTCGACCTCGAGAGATCTACGGGTGGC |
| | | ATCCCTGTGACCCCTCCCCAGTGCCTCTCCTGGCCCTGGAAGTTGCCACTCCAGTGCCCACC |
| | | AGCCTTGTCCTAATAAAATTAAGTTGCATCATTTTGTCTGACTAGGTGTCCTTCTATAATATT |
| | | ATGGGGTGGAGGGGGGTGGTATGGAGCAAGGGGCAAGTTGGGAAGACAACCTGTAGGGCC |
| | | TGCGGGGTCTATTGGGAACCAAGCTGGAGTGCAGTGGCACAATCTTGGCTCACTGCAATCTC |
| | | CGCCTCCTGGGTTCAAGCGATTCTCCTGCCTCAGCCTCCCGAGTTGTTGGGATTCCAGGCAT |
| | | GCATGACCAGGCTCAGCTAATTTTTGTTTTTTTGGTAGAGACGGGGTTTCACCATATTGGCC |
| | | AGGCTGGTCTCCAACTCCTAATCTCAGGTGATCTACCCACCTTGGCCTCCCAAATTGCTGGG |
| | | ATTACAGGCGTGAACCACTGCTCCCTTCCCTGTCCTTCTGATTTTGTAGGTAACCACGTGCGG |
| | | ACCGAGCGGCCGCAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCCCGGGCGGCCTCA |
| | | GTGAGCGAGCGAGCGCGCAGCTGCCTGCAGG |
| 283 | ITR.Reporter | CCTGCAGGCAGCTGCGCGCTCGCTCGCTCACTGAGGCCGCCCGGGCAAAGCCCGGGCGTCG |
| | 208B.Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCTAGACTGCAGCCTCAGGAGATCTGG |
| | | GCCCCCGCGGCATATGTTACTTGTACAGCTCGTCCATGCCGAGAGTGATCCCGGCGGCGGTC |
| | | ACGAACTCCAGCAGGACCATGTGATCGCGCTTCTCGTTGGGGTCTTTGCTCAGCTTGGACTG |
| | | GGTGCTCAGGTAGTGGTTGTCGGGCAGCAGCACGGGGCCGTCGCCGATGGGGGTGTTCTGC |
| | | TGGTAGTGGTCGGCGAGCTGCACGCTGCCGTCCTCGATGTTGTGGCGGATCTTGAAGTTGGC |
| | | CTTGATGCCGTTCTTCTGCTTGTCGGCGGTGATATAGACGTTGTCGCTGATGGCGTTGTACTC |
| | | CAGCTTGTGCCCCAGGATGTTGCCGTCCTCCTTGAAGTCGATGCCCTTCAGCTCGATGCGGT |
| | | TCACCAGGGTGTCGCCCTCGAACTTCACCTCGGCGCGGGTCTTGTAGTTGCCGTCGTCCTTG |
| | | AAGAAGATGGTGCGCTCCTGGACGTAGCCTTCGGGCATGGCGGACTTGAAGAAGTCGTGCT |
| | | GCTTCATGTGGTCGGGGTAGCGGGCGAAGCACTGCACGCCCCAGGTCAGGGTGGTCACGAG |
| | | GGTGGGCCAGGGCACGGGCAGCTTGCCGGTGGTGCAGATGAACTTCAGGGTCAGCTTGCCG |
| | | TAGGTGGCATCGCCCTCGCCCTCGCCGGACACGCTGAACTTGTGGCCGTTTACGTCGCCGTC |
| | | CAGCTCGACCAGGATGGGCACCACCCCGGTGAACAGCTCCTCGCCCTTGCTCACCATGGTGG |
| | | CGAATTCGCGGATCTGACGGTTCACTAAACCAGCTCTGCTTATATAGACCTCCCACCGTACA |
| | | CGCCTACCGCCCATTTGCGTCAATGGGGCGGAGTTGTTACGACATTTTGGAAAGTCCCGTTG |
| | | ATTTTGGTGCCAAAACAAACTCCCATTGACGTCAATGGGGTGGAGACTTGGAAATCCCCGTG |
| | | AGTCAAACCGCTATCCACGCCCATTGATGTACTGCCAAAACCGCATCACACTAGTTATTAAT |
| | | AGTAATCAATTACGGGGTCATTAGTTCATAGCCCATATATGGAGTTCCGCGTTACATAACTT |
| | | ACGGTAAATGGCCCGCCTGGCTGACCGCCCAACGACCCCCGCCCATTGACGTCAATAATGA |
| | | CGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTGATGCGGTTTTGGCAGT |
| | | ACATCAATGGGCGTGGATAGCGGTTTGACTCACGGGGATTTCCAAGTCTCCACCCCATTGAC |
| | | GTCAATGGGAGTTTGTTTTGGCACCAAAATCAACGGGACTTTCCAAAATGTCGTAACAACTC |
| | | CGCCCCATTGACGCAAATGGGCGGTAGGCGTGTACGGTGGGAGGTCTATATAAGCAGAGCT |
| | | CGTTTCGTACGTTCGAAGCCACCATGGTGAGCAAGGGCGAGGAGGATAACATGGCCATCAT |
| | | CAAGGAGTTCATGCGCTTCAAGGTGCACATGGAGGGCTCCGTGAACGGCCACGAGTTCGAG |
| | | ATCGAGGGCGAGGGCGAGGGCCGCCCCTACGAGGGCACCCAGACCGCCAAGCTGAAGGTG |
| | | ACCAAGGGTGGCCCCCTGCCCTTCGCCTGGGACATCCTGTCCCCTCAGTTCATGTACGGCTC |
| | | CAAGGCCTACGTGAAGCACCCCGCCGACATCCCCGACTACTTGAAGCTGTCCTTCCCCGAGG |
| | | GCTTCAAGTGGGAGCGCGTGATGAACTTCGAGGACGGCGGCGTGGTGACCGTGACCCAGGA |
| | | CTCCTCCCTCCAGGACGGCGAGTTCATCTACAAGGTGAAGCTGCGCGGCACCAACTTCCCCT |
| | | CCGACGGCCCCGTAATGCAGAAGAAGACCATGGGCTGGGAGGCCTCCTCCGAGCGGATGTA |
| | | CCCCGAGGACGGCGCCCTGAAGGGCGAGATCAAGCAGCGGCTGAAGCTGAAGGACGGCGG |
| | | CCACTACGACGCTGAGGTCAAGACCACCTACAAGGCCAAGAAGCCCGTGCAGCTGCCCGGC |
| | | GCCTACAACGTCAACATCAAGTTGGACATCACCTCCCACAACGAGGACTACACCATCGTGG |
| | | AACAGTACGAACGCGCCGAGGGCCGCCACTCCACCGGCGGCATGGACGAGCTGTACAAGTA |
| | | GACGCGGATCCACAAACCTTTTGTTCGTCTTATACAAACCTTTTGTTCGTCTTATACAAACCT |
| | | TTTGTTCGTCTTATACAAACCTTTTGTTCGTCTTATGTCGACCTCGAGAGATCTACGGGTGGC |
| | | ATCCCTGTGACCCCTCCCCAGTGCCTCTCCTGGCCCTGGAAGTTGCCACTCCAGTGCCCACC |
| | | AGCCTTGTCCTAATAAAATTAAGTTGCATCATTTTGTCTGACTAGGTGTCCTTCTATAATATT |
| | | ATGGGGTGGAGGGGGGTGGTATGGAGCAAGGGGCAAGTTGGGAAGACAACCTGTAGGGCC |
| | | TGCGGGGTCTATTGGGAACCAAGCTGGAGTGCAGTGGCACAATCTTGGCTCACTGCAATCTC |
| | | CGCCTCCTGGGTTCAAGCGATTCTCCTGCCTCAGCCTCCCGAGTTGTTGGGATTCCAGGCAT |
| | | GCATGACCAGGCTCAGCTAATTTTTGTTTTTTTGGTAGAGACGGGGTTTCACCATATTGGCC |
| | | AGGCTGGTCTCCAACTCCTAATCTCAGGTGATCTACCCACCTTGGCCTCCCAAATTGCTGGG |
| | | ATTACAGGCGTGAACCACTGCTCCCTTCCCTGTCCTTCTGATTTTGTAGGTAACCACGTGCGG |
| | | ACCGAGCGGCCGCAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCCCGGGCGGCCTCA |
| | | GTGAGCGAGCGAGCGCGCAGCTGCCTGCAGG |
| 284 | ITR.HCC.V1. | CCTGCAGGCAGCTGCGCGCTCGCTCGCTCACTGAGGCCGCCCGGGCAAAGCCCGGGCGTCG |
| | Cherry | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGCTTCGAATCGATGAATTCGA |
| | | AGCTTCTACCCACCGTACTCGTCAATTCCAAGGGCATCGGTAAACATCTGCTCAAACTCGAA |
| | | GTCGGCCATATCCAGAGCGCCGTAGGGGGCGGAGTCGTGGGGGGTAAATCCCGGACCCGGG |
| | | GAATCCCCGTCCCCCAACATGTCCAGATCGAAATCGTCTAGCGCGTCGGCATGCGCCATCGC |
| | | CACGTCCTCGCCGTCTAAGTGGAGCTCGTCCCCCAGGCTGACATCGGTCGGGGGGGCCGTCG |
| | | ACAGTCTGCGCGTGTGTCCCGCGGGGAGAAAGGACAGGCGCGGAGCCGCCAGCCCCGCCTC |
| | | TTCGGGGGCGTCGTCGTCCGGGAGATCGAGCAGGCCCTCGATGGTAGACCCGTAATTGTTTT |
| | | TCGTACGCGCGCGGCTGTACGCGGAGGCCTGTTCGACCATCGCGTCGATGCCCGCGACGAG |
| | | CAGGTCGAGGGCGAACTCGAAGTCCCGGTCCAGCATCTCCGCCACGGTGTCGCCGCCCCGG |
| | | GCCGCCATGATGTCCTGCGCGTCCTCGATGACGCCCGCGGTGTCCGGCACCTCGGTCACCGC |
| | | GGTCATCGAGTCCTGGAAGTACTCCTCCGGACTCAGCCCGGTGTCCGCCACCCGGGCGAGG |
| | | AAGCGGCCCTCGATGGTGCCGTAGCCGTAGACGAACTGGAAGACGGCCGAGATGGCGCCGG |
| | | TCAGGCGGTGCGCGGGCAGCCCGCTGCGGCGCACGACGTTCTGCACCGCGCGGGAGAAGGC |
| | | CAGCGAGTGCGGGCCGATGTTGAGGTAGGTGCCGACCAGCCGGGACGACCAGGGGTGGCG |
| | | CACCAGCAGCGCCCGGTTCTCCCGGGCCAGGGCCCGCAGTTCCTCGCGCCAGTCGAGCCCG |
| | | GCGTCCGGGTCCGGGTGGCGCAGCTCGCCGAAGACGGCGTCCAGGGCGAGCTCGAGCAACT |
| | | GGTCCTTGGTGTCGACGTACCAGTACACGGACATCGCGGTGACGTTCAGCTCGGCGGCCAG |
| | | GCGGCGCATCGAGAACCCCGTCAGGCCCTCCGTGTCCAGCAGCCGGACGGTGACCCCGGTG |
| | | ATCCGGTCCCGGTCGAGCCCGGACGGCTGCCCCCCACGGCGACCGCCGCGCCGCCCCTCCCC |
| | | CGACAGCCACACGCTGTCCCGCGGCCCCTCCCGCCCTGCCTTCGCCATGCGCACCTCTCCTC |
| | | GACTCATACCGGTAGCGCTAGCGATGAGCTCTGGTAGTAGACTAGTGGCCCCCATTATATAC |
| | | CCTCTAGAGCATATGTCTCACAAAGAGGGCTTTGTGTAGTCTCACAAAGAGGGCTTTGTGTA |
| | | GTCTCACAAAGAGGGCTTTGTGTAGGGCGCGCCCCCGTAGCTTGGCGTAATCACATGTCCGT |
| | | CGTTTTACAACGTCGTGACTGGGAAAACCCTGGCCTGCAAGGCGATTAAGTTGGGTAACGC |
| | | CAGGGTTTTCCCAGTCACGACGTTGTAAAACGACGGACATGTGAAATAGCGCTGTACAGCG |
| | | TATGGGAATCTCTTGTACGGTGTACGAGTATCTTCCCGTACACCGTACGGCGCGCCAGTTAA |
| | | TAATTAACTAGTTAATAATTAACTAGTTAATAATTAACTCATATGCTCTAGAGGGTATATAA |
| | | TGGGGGCCACTAGTCTACTACCAGAGCTCATCGCTAGCGCTGGATCCGCCACCATGGTGAGC |
| | | AAGGGCGAGGAGGATAACATGGCCATCATCAAGGAGTTCATGCGCTTCAAGGTGCACATGG |
| | | AGGGCTCCGTGAACGGCCACGAGTTCGAGATCGAGGGCGAGGGCGAGGGCCGCCCCTACG |
| | | AGGGCACCCAGACCGCCAAGCTGAAGGTGACCAAGGGTGGCCCCCTGCCCTTCGCCTGGGA |
| | | CATCCTGTCCCCTCAGTTCATGTACGGCTCCAAGGCCTACGTGAAGCACCCCGCCGACATCC |
| | | CCGACTACTTGAAGCTGTCCTTCCCCGAGGGCTTCAAGTGGGAGCGCGTGATGAACTTCGAG |
| | | GACGGCGGCGTGGTGACCGTGACCCAGGACTCCTCCCTCCAGGACGGCGAGTTCATCTACA |
| | | AGGTGAAGCTGCGCGGCACCAACTTCCCCTCCGACGGCCCCGTAATGCAGAAGAAGACCAT |
| | | GGGCTGGGAGGCCTCCTCCGAGCGGATGTACCCCGAGGACGGCGCCCTGAAGGGCGAGATC |
| | | AAGCAGCGGCTGAAGCTGAAGGACGGCGGCCACTACGACGCTGAGGTCAAGACCACCTAC |
| | | AAGGCCAAGAAGCCCGTGCAGCTGCCCGGCGCCTACAACGTCAACATCAAGTTGGACATCA |
| | | CCTCCCACAACGAGGACTACACCATCGTGGAACAGTACGAACGCGCCGAGGGCCGCCACTG |
| | | CACCGGCGGCATGGACGAGCTGTACAAGTAGGGTACCCAAACACCATTGTCACACTCCAAG |
| | | ATCTACGGGTGGCATCCCTGTGACCCCTCCCCAGTGCCTCTCCTGGCCCTGGAAGTTGCCAC |
| | | TCCAGTGCCCACCAGCCTTGTCCTAATAAAATTAAGTTGCATCATTTTGTCTGACTAGGTGTC |
| | | CTTCTATAATATTATGGGGTGGAGGGGGGTGGTATGGAGCAAGGGGCAAGTTGGGAAGACA |
| | | ACCTGTAGGGCCTGCGGGGTCTATTGGGAACCAAGCTGGAGTGCAGTGGCACAATCTTGGC |
| | | TCACTGCAATCTCCGCCTCCTGGGTTCAAGCGATTCTCCTGCCTCAGCCTCCCGAGTTGTTGG |
| | | GATTCCAGGCATGCATGACCAGGCTCAGCTAATTTTTGTTTTTTTGGTAGAGACGGGGTTTC |
| | | ACCATATTGGCCAGGCTGGTCTCCAACTCCTAATCTCAGGTGATCTACCCACCTTGGCCTCC |
| | | CAAATTGCTGGGATTACAGGCGTGAACCACTGCTCCCTTCCCTGTCCTTCTGATTTTGTAGGT |
| | | AACCACGTGCGGACCGAGCGGCCGCAGGAACCCCTAGTGATGGAGTTGGCCACTCCCTCTC |
| | | TGCGCGCTCGCTCGCTCACTGAGGCCGGGCGACCAAAGGTCGCCCGACGCCCGGGCTTTGC |
| | | CCGGGCGGCCTCAGTGAGCGAGCGAGCGCGCAGCTGCCTGCAGG |
| 285 | ITR.HCC.V1. | CCTGCAGGCAGCTGCGCGCTCGCTCGCTCACTGAGGCCGCCCGGGCAAAGCCCGGGCGTCG |
| | HSVTK | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGCTTCGAATCGATGAATTCGA |
| | | AGCTTCTACCCACCGTACTCGTCAATTCCAAGGGCATCGGTAAACATCTGCTCAAACTCGAA |
| | | GTCGGCCATATCCAGAGCGCCGTAGGGGGCGGAGTCGTGGGGGGTAAATCCCGGACCCGGG |
| | | GAATCCCCGTCCCCCAACATGTCCAGATCGAAATCGTCTAGCGCGTCGGCATGCGCCATCGC |
| | | CACGTCCTCGCCGTCTAAGTGGAGCTCGTCCCCCAGGCTGACATCGGTCGGGGGGGCCGTCG |
| | | ACAGTCTGCGCGTGTGTCCCGCGGGGAGAAAGGACAGGCGCGGAGCCGCCAGCCCCGCCTC |
| | | TTCGGGGGCGTCGTCGTCCGGGAGATCGAGCAGGCCCTCGATGGTAGACCCGTAATTGTTTT |
| | | TCGTACGCGCGCGGCTGTACGCGGAGGCCTGTTCGACCATCGCGTCGATGCCCGCGACGAG |
| | | CAGGTCGAGGGCGAACTCGAAGTCCCGGTCCAGCATCTCCGCCACGGTGTCGCCGCCCCGG |
| | | GCCGCCATGATGTCCTGCGCGTCCTCGATGACGCCCGCGGTGTCCGGCACCTCGGTCACCGC |
| | | GGTCATCGAGTCCTGGAAGTACTCCTCCGGACTCAGCCCGGTGTCCGCCACCCGGGCGAGG |
| | | AAGCGGCCCTCGATGGTGCCGTAGCCGTAGACGAACTGGAAGACGGCCGAGATGGCGCCGG |
| | | TCAGGCGGTGCGCGGGCAGCCCGCTGCGGCGCACGACGTTCTGCACCGCGCGGGAGAAGGC |
| | | CAGCGAGTGCGGGCCGATGTTGAGGTAGGTGCCGACCAGCCGGGACGACCAGGGGTGGCG |
| | | CACCAGCAGCGCCCGGTTCTCCCGGGCCAGGGCCCGCAGTTCCTCGCGCCAGTCGAGCCCG |
| | | GCGTCCGGGTCCGGGTGGCGCAGCTCGCCGAAGACGGCGTCCAGGGCGAGCTCGAGCAACT |
| | | GGTCCTTGGTGTCGACGTACCAGTACACGGACATCGCGGTGACGTTCAGCTCGGCGGCCAG |
| | | GCGGCGCATCGAGAACCCCGTCAGGCCCTCCGTGTCCAGCAGCCGGACGGTGACCCCGGTG |
| | | ATCCGGTCCCGGTCGAGCCCGGACGGCTGCCCCCCACGGCGACCGCCGCGCCGCCCCTCCCC |
| | | CGACAGCCACACGCTGTCCCGCGGCCCCTCCCGCCCTGCCTTCGCCATGCGCACCTCTCCTC |
| | | GACTCATACCGGTAGCGCTAGCGATGAGCTCTGGTAGTAGACTAGTGGCCCCCATTATATAC |
| | | CCTCTAGAGCATATGTCTCACAAAGAGGGCTTTGTGTAGTCTCACAAAGAGGGCTTTGTGTA |
| | | GTCTCACAAAGAGGGCTTTGTGTAGGGCGCGCCCCCGTAGCTTGGCGTAATCACATGTCCGT |
| | | CGTTTTACAACGTCGTGACTGGGAAAACCCTGGCCTGCAAGGCGATTAAGTTGGGTAACGC |
| | | CAGGGTTTTCCCAGTCACGACGTTGTAAAACGACGGACATGTGAAATAGCGCTGTACAGCG |
| | | TATGGGAATCTCTTGTACGGTGTACGAGTATCTTCCCGTACACCGTACGGCGCGCCAGTTAA |
| | | TAATTAACTAGTTAATAATTAACTAGTTAATAATTAACTCATATGCTCTAGAGGGTATATAA |
| | | TGGGGGCCACTAGTCTACTACCAGAGCTCATCGCTAGCGCTGGATCCCGCCACCATGGCTTC |
| | | GTACCCCTGCCATCAACACGCGTCTGCGTTCGACCAGGCTGCGCGTTCTCGCGGCCATAGCA |
| | | ACCGACGTACGGCGTTGCGCCCTCGCCGGCAGCAAGAAGCCACGGAAGTCCGCCTGGAGCA |
| | | GAAAATGCCCACGCTACTGCGGGTTTATATAGACGGTCCTCACGGGATGGGGAAAACCACC |
| | | ACCACGCAACTGCTGGTGGCCCTGGGTTCGCGCGACGATATCGTCTACGTACCCGAGCCGAT |
| | | GACTTACTGGCAGGTGCTGGGGGCTTCCGAGACAATCGCGAACATCTACACCACACAACAC |
| | | CGCCTCGACCAGGGTGAGATATCGGCCGGGGACGCGGCGGTGGTAATGACAAGCGCCCAGA |
| | | TAACAATGGGCATGCCTTATGCCGTGACCGACGCCGTTCTGGCTCCTCATATCGGGGGGGAG |
| | | GCTGGGAGCTCACATGCCCCGCCCCCGGCCCTCACCCTCATCTTCGACCGCCATCCCATCGC |
| | | CGCCCTCCTGTGCTACCCGGCCGCGCGATACCTTATGGGCAGCATGACCCCCCAGGCCGTGC |
| | | TGGCGTTCGTGGCCCTCATCCCGCCGACCTTGCCCGGCACAAACATCGTGTTGGGGGCCCTT |
| | | CCGGAGGACAGACACATCGACCGCCTGGCCAAACGCCAGCGCCCCGGCGAGCGGCTTGACC |
| | | TGGCTATGCTGGCCGCGATTCGCCGCGTTTACGGGCTGCTTGCCAATACGGTGCGGTATCTG |
| | | CAGGGCGGCGGGTCGTGGCGGGAGGATTGGGGACAGCTTTCGGGGACGGCCGTGCCGCCCC |
| | | AGGGTGCCGAGCCCCAGAGCAACGCGGGCCCACGACCCCATATCGGGGACACGTTATTTAC |
| | | CCTGTTTCGGGCCCCCGAGTTGCTGGCCCCCAACGGCGACCTGTACAACGTGTTTGCCTGGG |
| | | CCTTGGACGTCTTGGCCAAACGCCTCCGTCCCATGCACGTCTTTATCCTGGATTACGACCAA |
| | | TCGCCCGCCGGCTGCCGGGACGCCCTGCTGCAACTTACCTCCGGGATGGTCCAGACCCACGT |
| | | CACCACCCCCGGCTCCATACCGACGATCTGCGACCTGGCGCGCACGTTTGCCCGGGAGATG |
| | | GGGGAGGCTAACTGAGGTACCCAAACACCATTGTCACACTCCAAGATCTACGGGTGGCATC |
| | | CCTGTGACCCCTCCCCAGTGCCTCTCCTGGCCCTGGAAGTTGCCACTCCAGTGCCCACCAGC |
| | | CTTGTCCTAATAAAATTAAGTTGCATCATTTTGTCTGACTAGGTGTCCTTCTATAATATTATG |
| | | GGGTGGAGGGGGGTGGTATGGAGCAAGGGGCAAGTTGGGAAGACAACCTGTAGGGCCTGC |
| | | GGGGTCTATTGGGAACCAAGCTGGAGTGCAGTGGCACAATCTTGGCTCACTGCAATCTCCGC |
| | | CTCCTGGGTTCAAGCGATTCTCCTGCCTCAGCCTCCCGAGTTGTTGGGATTCCAGGCATGCA |
| | | TGACCAGGCTCAGCTAATTTTTGTTTTTTTGGTAGAGACGGGGTTTCACCATATTGGCCAGG |
| | | CTGGTCTCCAACTCCTAATCTCAGGTGATCTACCCACCTTGGCCTCCCAAATTGCTGGGATTA |
| | | CAGGCGTGAACCACTGCTCCCTTCCCTGTCCTTCTGATTTTGTAGGTAACCACGTGCGGACC |
| | | GAGCGGCCGCAGGAACCCCTAGTGATGGAGTTGGCCACTCCCTCTCTGCGCGCTCGCTCGCT |
| | | CACTGAGGCCGGGCGACCAAAGGTCGCCCGACGCCCGGGCTTTGCCCGGGCGGCCTCAGTG |
| | | AGCGAGCGAGCGCGCAGCTGCCTGCAGG |
| 286 | ITR.HCC.V2. | CCTGCAGGCAGCTGCGCGCTCGCTCGCTCACTGAGGCCGCCCGGGCAAAGCCCGGGCGTCG |
| | HSVTK | GGCGACCTTTGGTCGCCCGGCCTCAGTGAGCGAGCGAGCGCGCAGAGAGGGAGTGGCCAAC |
| | | TCCATCACTAGGGGTTCCTGCGGCCGCACGCGTAACTTGTGGACTAAGTTTGTTCACATCCC |
| | | CTTCTCCAACCCCCTCAGTACATCACCCTGGGGGAACAGGGTCCACTTGCTCCTGGGCCCAC |
| | | ACAGTCCTGCAGTATTGTGTATATAAGGCCAGGGCAAAGAGGAGCAGGTTTTAAAGTGAAA |
| | | GGCAGGCAGGTGTTGGGGAGGCAGTTACCGGGGCAACGGGAACAGGGCGTTTCGGAGGTG |
| | | GTTGCCATGGGGACCTGGATGCTGACGAAGGCTCGATTATTGAAGCATTTATCAGGGTTATT |
| | | GTCTCATGAGCGGATACATATTTGAATGTATTTAGAAAAATAAACAAATAGGGGTTCCGCG |
| | | CACATTTCCCCGAAAAGTGCCACCTGACGTCGGCAGTGAAAAAAATGCTTTATTTGTGAAAT |
| | | TTGTGATGCTATTGCTTTATTTGTAACCATTATAAGCTGCAATAAACAAGTTAACAACAACA |
| | | ATTGCATTCATTTTATGTTTCAGGTTCAGGGGGAGGTGTGGGAGGTTTTTTAAAGCAAGTAA |
| | | AACCTCTACAAATGTGGTATGGCTGATTATGATCCTCCTAGGTGAGGTAGTAGGTTGTATGG |
| | | TTTGAGGTAGTAGGTTGTATGGTTTGAGGTAGTAGGTTGTATGGTTTGAGGTAGTAGGTTGT |
| | | ATGGTTATCGATGAATTCGAAGCTTCTACCCACCGTACTCGTCAATTCCAAGGGCATCGGTA |
| | | AACATCTGCTCAAACTCGAAGTCGGCCATATCCAGAGCGCCGTAGGGGGCGGAGTCGTGGG |
| | | GGGTAAATCCCGGACCCGGGGAATCCCCGTCCCCCAACATGTCCAGATCGAAATCGTCTAG |
| | | CGCGTCGGCATGCGCCATCGCCACGTCCTCGCCGTCTAAGTGGAGCTCGTCCCCCAGGCTGA |
| | | CATCGGTCGGGGGGGCCGTCGACAGTCTGCGCGTGTGTCCCGCGGGGAGAAAGGACAGGCG |
| | | CGGAGCCGCCAGCCCCGCCTCTTCGGGGGCGTCGTCGTCCGGGAGATCGAGCAGGCCCTCG |
| | | ATGGTAGACCCGTAATTGTTTTTCGTACGCGCGCGGCTGTACGCGGAGGCCTGTTCGACCAT |
| | | CGCGTCGATGCCCGCGACGAGCAGGTCGAGGGCGAACTCGAAGTCCCGGTCCAGCATCTCC |
| | | GCCACGGTGTCGCCGCCCCGGGCCGCCATGATGTCCTGCGCGTCCTCGATGACGCCCGCGGT |
| | | GTCCGGCACCTCGGTCACCGCGGTCATCGAGTCCTGGAAGTACTCCTCCGGACTCAGCCCGG |
| | | TGTCCGCCACCCGGGCGAGGAAGCGGCCCTCGATGGTGCCGTAGCCGTAGACGAACTGGAA |
| | | GACGGCCGAGATGGCGCCGGTCAGGCGGTGCGCGGGCAGCCCGCTGCGGCGCACGACGTTC |
| | | TGCACCGCGCGGGAGAAGGCCAGCGAGTGCGGGCCGATGTTGAGGTAGGTGCCGACCAGCC |
| | | GGGACGACCAGGGGTGGCGCACCAGCAGCGCCCGGTTCTCCCGGGCCAGGGCCCGCAGTTC |
| | | CTCGCGCCAGTCGAGCCCGGCGTCCGGGTCCGGGTGGCGCAGCTCGCCGAAGACGGCGTCC |
| | | AGGGCGAGCTCGAGCAACTGGTCCTTGGTGTCGACGTACCAGTACACGGACATCGCGGTGA |
| | | CGTTCAGCTCGGCGGCCAGGCGGCGCATCGAGAACCCCGTCAGGCCCTCCGTGTCCAGCAG |
| | | CCGGACGGTGACCCCGGTGATCCGGTCCCGGTCGAGCCCGGACGGCTGCCCCCCACGGCGA |
| | | CCGCCGCGCCGCCCCTCCCCCGACAGCCACACGCTGTCCCGCGGCCCCTCCCGCCCTGCCTT |
| | | CGCCATGCGCACCTCTCCTCGACTCATACCGGTAGCGCTAGCGATGAGCTCTGGTAGTAGAC |
| | | TAGTGGCCCCCATTATATACCCTCTAGAGCATATGTCTCACAAAGAGGGCTTTGTGTAGTCT |
| | | CACAAAGAGGGCTTTGTGTAGTCTCACAAAGAGGGCTTTGTGTAGGGCGCGCCCCCGTAGC |
| | | TTGGCGTAATCACATGTCCGTCGTTTTACAACGTCGTGACTGGGAAAACCCTGGCCTGCAAG |
| | | GCGATTAAGTTGGGTAACGCCAGGGTTTTCCCAGTCACGACGTTGTAAAACGACGGACATG |
| | | TGAAATAGCGCTGTACAGCGTATGGGAATCTCTTGTACGGTGTACGAGTATCTTCCCGTACA |
| | | CCGTACGGCGCGCCAGTTAATAATTAACTAGTTAATAATTAACTAGTTAATAATTAACTCAT |
| | | ATGCTCTAGAGGGTATATAATGGGGGCCACTAGTCTACTACCAGAGCTCATCGCTAGCGCTG |
| | | GATCCCGCCACCATGGCTTCGTACCCCTGCCATCAACACGCGTCTGCGTTCGACCAGGCTGC |
| | | GCGTTCTCGCGGCCATAGCAACCGACGTACGGCGTTGCGCCCTCGCCGGCAGCAAGAAGCC |
| | | ACGGAAGTCCGCCTGGAGCAGAAAATGCCCACGCTACTGCGGGTTTATATAGACGGTCCTC |
| | | ACGGGATGGGGAAAACCACCACCACGCAACTGCTGGTGGCCCTGGGTTCGCGCGACGATAT |
| | | CGTCTACGTACCCGAGCCGATGACTTACTGGCAGGTGCTGGGGGCTTCCGAGACAATCGCG |
| | | AACATCTACACCACACAACACCGCCTCGACCAGGGTGAGATATCGGCCGGGGACGCGGCGG |
| | | TGGTAATGACAAGCGCCCAGATAACAATGGGCATGCCTTATGCCGTGACCGACGCCGTTCT |
| | | GGCTCCTCATATCGGGGGGGAGGCTGGGAGCTCACATGCCCCGCCCCCGGCCCTCACCCTCA |
| | | TCTTCGACCGCCATCCCATCGCCGCCCTCCTGTGCTACCCGGCCGCGCGATACCTTATGGGC |
| | | AGCATGACCCCCCAGGCCGTGCTGGCGTTCGTGGCCCTCATCCCGCCGACCTTGCCCGGCAC |
| | | AAACATCGTGTTGGGGGCCCTTCCGGAGGACAGACACATCGACCGCCTGGCCAAACGCCAG |
| | | CGCCCCGGCGAGCGGCTTGACCTGGCTATGCTGGCCGCGATTCGCCGCGTTTACGGGCTGCT |
| | | TGCCAATACGGTGCGGTATCTGCAGGGCGGCGGGTCGTGGCGGGAGGATTGGGGACAGCTT |
| | | TCGGGGACGGCCGTGCCGCCCCAGGGTGCCGAGCCCCAGAGCAACGCGGGCCCACGACCCC |
| | | ATATCGGGGACACGTTATTTACCCTGTTTCGGGCCCCCGAGTTGCTGGCCCCCAACGGCGAC |
| | | CTGTACAACGTGTTTGCCTGGGCCTTGGACGTCTTGGCCAAACGCCTCCGTCCCATGCACGT |
| | | CTTTATCCTGGATTACGACCAATCGCCCGCCGGCTGCCGGGACGCCCTGCTGCAACTTACCT |
| | | CCGGGATGGTCCAGACCCACGTCACCACCCCCGGCTCCATACCGACGATCTGCGACCTGGC |
| | | GCGCACGTTTGCCCGGGAGATGGGGGAGGCTAACTGAGGTACCAACCATACAACCTACTAC |
| | | CTCAAACCATACAACCTACTACCTCAAACCATACAACCTACTACCTCAAACCATACAACCTA |
| | | CTACCTCAAGATCTACGGGTGGCATCCCTGTGACCCCTCCCCAGTGCCTCTCCTGGCCCTGG |
| | | AAGTTGCCACTCCAGTGCCCACCAGCCTTGTCCTAATAAAATTAAGTTGCATCATTTTGTCTG |
| | | ACTAGGTGTCCTTCTATAATATTATGGGGTGGAGGGGGGTGGTATGGAGCAAGGGGCAAGT |
| | | TGGGAAGACAACCTGTAGGGCCTGCGGGGTCTATTGGGAACCAAGCTGGAGTGCAGTGGCA |
| | | CAATCTTGGCTCACTGCAATCTCCGCCTCCTGGGTTCAAGCGATTCTCCTGCCTCAGCCTCCC |
| | | GAGTTGTTGGGATTCCAGGCATGCATGACCAGGCTCAGCTAATTTTTGTTTTTTTGGTAGAG |
| | | ACGGGGTTTCACCATATTGGCCAGGCTGGTCTCCAACTCCTAATCTCAGGTGATCTACCCAC |
| | | CTTGGCCTCCCAAATTGCTGGGATTACAGGCGTGAACCACTGCTCCCTTCCCTGTCCTTCTGA |
| | | TTTTGTAGGTAACCACGTGCGGACCGAGCGGCCGCAGGAACCCCTAGTGATGGAGTTGGCC |
| | | ACTCCCTCTCTGCGCGCTCGCTCGCTCACTGAGGCCGGGCGACCAAAGGTCGCCCGACGCCC |
| | | GGGCTTTGCCCGGGCGGCCTCAGTGAGCGAGCGAGCGCGCAGCTGCCTGCAGG |
| 287 | C.CMV.Cherry | CAGTATTGTGTATATAAGGCCAGGGCAAAGAGGAGCAGGTTTTAAAGTGAAAGGCAGGCAG |
| | | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCAAGCACTCTGATTTGACAATTAAAGCACTCTGATTTGACAATTAAAGCACTCTGATTTGA |
| | | CAATTAAAGCACTCTGATTTGACAATTAGTCGACCTCGAGAGATCTACGGGTGGCATCCCTG |
| | | TGACCCCTCCCCAGTGCCTCTCCTGGCCCTGGAAGTTGCCACTCCAGTGCCCACCAGCCTTG |
| | | TCCTAATAAAATTAAGTTGCATCATTTTGTCTGACTAGGTGTCCTTCTATAATATTATGGGGT |
| | | GGAGGGGGGTGGTATGGAGCAAGGGGCAAGTTGGGAAGACAACCTGTAGGGCCTGCGGGG |
| | | TCTATTGGGAACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCATATTGGCCAGGCTGGT |
| | | CTCCAACTCCTAATCTCAGGTGATCTACCCACCTTGGCCTCCCAAATTGCTGGGATTACAGG |
| | | CGTGAACCACTGCTCCCTTCCCTGTCCTT |
| 288 | c.Letc.Cherry | CAGTATTGTGTATATAAGGCCAGGGCAAAGAGGAGCAGGTTTTAAAGTGAAAGGCAGGCAG |
| | | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCAACCATACAACCTACTACCTCAAACCATACAACCTACTACCTCAAACCATACAACCTACT |
| | | ACCTCAAACCATACAACCTACTACCTCAGTCGACCTCGAGAGATCTACGGGTGGCATCCCTG |
| | | TGACCCCTCCCCAGTGCCTCTCCTGGCCCTGGAAGTTGCCACTCCAGTGCCCACCAGCCTTG |
| | | TCCTAATAAAATTAAGTTGCATCATTTTGTCTGACTAGGTGTCCTTCTATAATATTATGGGGT |
| | | GGAGGGGGGTGGTATGGAGCAAGGGGCAAGTTGGGAAGACAACCTGTAGGGCCTGCGGGG |
| | | TCTATTGGGAACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCATATTGGCCAGGCTGGT |
| | | CTCCAACTCCTAATCTCAGGTGATCTACCCACCTTGGCCTCCCAAATTGCTGGGATTACAGG |
| | | CGTGAACCACTGCTCCCTTCCCTGTCCTT |
| 289 | C.TF-AND. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGCTTCGAATCGATGAATTCGAAGCTTCTACCC |
| | | ACCGTACTCGTCAATTCCAAGGGCATCGGTAAACATCTGCTCAAACTCGAAGTCGGCCATAT |
| | | CCAGAGCGCCGTAGGGGGCGGAGTCGTGGGGGGTAAATCCCGGACCCGGGGAATCCCCGTC |
| | | CCCCAACATGTCCAGATCGAAATCGTCTAGCGCGTCGGCATGCGCCATCGCCACGTCCTCGC |
| | | CGTCTAAGTGGAGCTCGTCCCCCAGGCTGACATCGGTCGGGGGGGCCGTCGACAGTCTGCG |
| | | CGTGTGTCCCGCGGGGAGAAAGGACAGGCGCGGAGCCGCCAGCCCCGCCTCTTCGGGGGCG |
| | | TCGTCGTCCGGGAGATCGAGCAGGCCCTCGATGGTAGACCCGTAATTGTTTTTCGTACGCGC |
| | | GCGGCTGTACGCGGAGGCCTGTTCGACCATCGCGTCGATGCCCGCGACGAGCAGGTCGAGG |
| | | GCGAACTCGAAGTCCCGGTCCAGCATCTCCGCCACGGTGTCGCCGCCCCGGGCCGCCATGAT |
| | | GTCCTGCGCGTCCTCGATGACGCCCGCGGTGTCCGGCACCTCGGTCACCGCGGTCATCGAGT |
| | | CCTGGAAGTACTCCTCCGGACTCAGCCCGGTGTCCGCCACCCGGGCGAGGAAGCGGCCCTC |
| | | GATGGTGCCGTAGCCGTAGACGAACTGGAAGACGGCCGAGATGGCGCCGGTCAGGCGGTGC |
| | | GCGGGCAGCCCGCTGCGGCGCACGACGTTCTGCACCGCGCGGGAGAAGGCCAGCGAGTGCG |
| | | GGCCGATGTTGAGGTAGGTGCCGACCAGCCGGGACGACCAGGGGTGGCGCACCAGCAGCG |
| | | CCCGGTTCTCCCGGGCCAGGGCCCGCAGTTCCTCGCGCCAGTCGAGCCCGGCGTCCGGGTCC |
| | | GGGTGGCGCAGCTCGCCGAAGACGGCGTCCAGGGCGAGCTCGAGCAACTGGTCCTTGGTGT |
| | | CGACGTACCAGTACACGGACATCGCGGTGACGTTCAGCTCGGCGGCCAGGCGGCGCATCGA |
| | | GAACCCCGTCAGGCCCTCCGTGTCCAGCAGCCGGACGGTGACCCCGGTGATCCGGTCCCGG |
| | | TCGAGCCCGGACGGCTGCCCCCCACGGCGACCGCCGCGCCGCCCCTCCCCCGACAGCCACA |
| | | CGCTGTCCCGCGGCCCCTCCCGCCCTGCCTTCGCCATGCGCACCTCTCCTCGACTCATACCGG |
| | | TAGCGCTAGCGATGAGCTCTGGTAGTAGACTAGTGGCCCCCATTATATACCCTCTAGAGCAT |
| | | ATGTCTCACAAAGAGGGCTTTGTGTAGTCTCACAAAGAGGGCTTTGTGTAGTCTCACAAAGA |
| | | GGGCTTTGTGTAGGGCGCGCCCCCGTAGCTTGGCGTAATCACATGTCCGTCGTTTTACAACG |
| | | TCGTGACTGGGAAAACCCTGGCCTGCAAGGCGATTAAGTTGGGTAACGCCAGGGTTTTCCC |
| | | AGTCACGACGTTGTAAAACGACGGACATGTGAAATAGCGCTGTACAGCGTATGGGAATCTC |
| | | TTGTACGGTGTACGAGTATCTTCCCGTACACCGTACGGCGCGCCAGTTAATAATTAACTAGT |
| | | TAATAATTAACTAGTTAATAATTAACTCATATGCTCTAGAGGGTATATAATGGGGGCCACTA |
| | | GTCTACTACCAGAGCTCATCGCTAGCGCTGGATCCGCCACCATGGTGAGCAAGGGCGAGGA |
| | | GGATAACATGGCCATCATCAAGGAGTTCATGCGCTTCAAGGTGCACATGGAGGGCTCCGTG |
| | | AACGGCCACGAGTTCGAGATCGAGGGCGAGGGCGAGGGCCGCCCCTACGAGGGCACCCAG |
| | | ACCGCCAAGCTGAAGGTGACCAAGGGTGGCCCCCTGCCCTTCGCCTGGGACATCCTGTCCCC |
| | | TCAGTTCATGTACGGCTCCAAGGCCTACGTGAAGCACCCCGCCGACATCCCCGACTACTTGA |
| | | AGCTGTCCTTCCCCGAGGGCTTCAAGTGGGAGCGCGTGATGAACTTCGAGGACGGCGGCGT |
| | | GGTGACCGTGACCCAGGACTCCTCCCTCCAGGACGGCGAGTTCATCTACAAGGTGAAGCTG |
| | | CGCGGCACCAACTTCCCCTCCGACGGCCCCGTAATGCAGAAGAAGACCATGGGCTGGGAGG |
| | | CCTCCTCCGAGCGGATGTACCCCGAGGACGGCGCCCTGAAGGGCGAGATCAAGCAGCGGCT |
| | | GAAGCTGAAGGACGGCGGCCACTACGACGCTGAGGTCAAGACCACCTACAAGGCCAAGAA |
| | | GCCCGTGCAGCTGCCCGGCGCCTACAACGTCAACATCAAGTTGGACATCACCTCCCACAAC |
| | | GAGGACTACACCATCGTGGAACAGTACGAACGCGCCGAGGGCCGCCACTCCACCGGCGGCA |
| | | TGGACGAGCTGTACAAGTAGGGTACCGTCGACCTCGAGAGATCTACGGGTGGCATCCCTGT |
| | | GACCCCTCCCCAGTGCCTCTCCTGGCCCTGGAAGTTGCCACTCCAGTGCCCACCAGCCTTGT |
| | | CCTAATAAAATTAAGTTGCATCATTTTGTCTGACTAGGTGTCCTTCTATAATATTATGGGGTG |
| | | GAGGGGGGTGGTATGGAGCAAGGGGCAAGTTGGGAAGACAACCTGTAGGGCCTGCGGGGT |
| | | CTATTGGGAACCAAGCTGGAGTGCAGTGGCACAATCTTGGCTCACTGCAATCTCCGCCTCCT |
| | | GGGTTCAAGCGATTCTCCTGCCTCAGCCTCCCGAGTTGTTGGGATTCCAGGCATGCATGACC |
| | | AGGCTCAGCTAATTTTTGTTTTTTTGGTAGAGACGGGGTTTCACCATATTGGCCAGGCTGGTC |
| | | TCCAACTCCTAATCTCAGGTGATCTACCCACCTTGGCCTCCCAAATTGCTGGGATTACAGGC |
| | | GTGAACCACTGCTCCCTTCCCTGTCCTT |
| 290 | HCC.V2.Cherry | CAGTATTGTGTATATAAGGCCAGGGCAAAGAGGAGCAGGTTTTAAAGTGAAAGGCAGGCAG |
| | | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGTGAGGTAGTAGGTTGTATGGTTTGAGGTAGT |
| | | AGGTTGTATGGTTTGAGGTAGTAGGTTGTATGGTTTGAGGTAGTAGGTTGTATGGTTATCGA |
| | | TGAATTCGAAGCTTCTACCCACCGTACTCGTCAATTCCAAGGGCATCGGTAAACATCTGCTC |
| | | AAACTCGAAGTCGGCCATATCCAGAGCGCCGTAGGGGGCGGAGTCGTGGGGGGTAAATCCC |
| | | GGACCCGGGGAATCCCCGTCCCCCAACATGTCCAGATCGAAATCGTCTAGCGCGTCGGCAT |
| | | GCGCCATCGCCACGTCCTCGCCGTCTAAGTGGAGCTCGTCCCCCAGGCTGACATCGGTCGGG |
| | | GGGGCCGTCGACAGTCTGCGCGTGTGTCCCGCGGGGAGAAAGGACAGGCGCGGAGCCGCC |
| | | AGCCCCGCCTCTTCGGGGGCGTCGTCGTCCGGGAGATCGAGCAGGCCCTCGATGGTAGACC |
| | | CGTAATTGTTTTTCGTACGCGCGCGGCTGTACGCGGAGGCCTGTTCGACCATCGCGTCGATG |
| | | CCCGCGACGAGCAGGTCGAGGGCGAACTCGAAGTCCCGGTCCAGCATCTCCGCCACGGTGT |
| | | CGCCGCCCCGGGCCGCCATGATGTCCTGCGCGTCCTCGATGACGCCCGCGGTGTCCGGCACC |
| | | TCGGTCACCGCGGTCATCGAGTCCTGGAAGTACTCCTCCGGACTCAGCCCGGTGTCCGCCAC |
| | | CCGGGCGAGGAAGCGGCCCTCGATGGTGCCGTAGCCGTAGACGAACTGGAAGACGGCCGA |
| | | GATGGCGCCGGTCAGGCGGTGCGCGGGCAGCCCGCTGCGGCGCACGACGTTCTGCACCGCG |
| | | CGGGAGAAGGCCAGCGAGTGCGGGCCGATGTTGAGGTAGGTGCCGACCAGCCGGGACGAC |
| | | CAGGGGTGGCGCACCAGCAGCGCCCGGTTCTCCCGGGCCAGGGCCCGCAGTTCCTCGCGCC |
| | | AGTCGAGCCCGGCGTCCGGGTCCGGGTGGCGCAGCTCGCCGAAGACGGCGTCCAGGGCGAG |
| | | CTCGAGCAACTGGTCCTTGGTGTCGACGTACCAGTACACGGACATCGCGGTGACGTTCAGCT |
| | | CGGCGGCCAGGCGGCGCATCGAGAACCCCGTCAGGCCCTCCGTGTCCAGCAGCCGGACGGT |
| | | GACCCCGGTGATCCGGTCCCGGTCGAGCCCGGACGGCTGCCCCCCACGGCGACCGCCGCGC |
| | | CGCCCCTCCCCCGACAGCCACACGCTGTCCCGCGGCCCCTCCCGCCCTGCCTTCGCCATGCG |
| | | CACCTCTCCTCGACTCATACCGGTAGCGCTAGCGATGAGCTCTGGTAGTAGACTAGTGGCCC |
| | | CCATTATATACCCTCTAGAGCATATGTCTCACAAAGAGGGCTTTGTGTAGTCTCACAAAGAG |
| | | GGCTTTGTGTAGTCTCACAAAGAGGGCTTTGTGTAGGGCGCGCCCCCGTAGCTTGGCGTAAT |
| | | CACATGTCCGTCGTTTTACAACGTCGTGACTGGGAAAACCCTGGCCTGCAAGGCGATTAAGT |
| | | TGGGTAACGCCAGGGTTTTCCCAGTCACGACGTTGTAAAACGACGGACATGTGAAATAGCG |
| | | CTGTACAGCGTATGGGAATCTCTTGTACGGTGTACGAGTATCTTCCCGTACACCGTACGGCG |
| | | CGCCAGTTAATAATTAACTAGTTAATAATTAACTAGTTAATAATTAACTCATATGCTCTAGA |
| | | GGGTATATAATGGGGGCCACTAGTCTACTACCAGAGCTCATCGCTAGCGCTGGATCCGCCAC |
| | | CATGGTGAGCAAGGGCGAGGAGGATAACATGGCCATCATCAAGGAGTTCATGCGCTTCAAG |
| | | GTGCACATGGAGGGCTCCGTGAACGGCCACGAGTTCGAGATCGAGGGCGAGGGCGAGGGC |
| | | CGCCCCTACGAGGGCACCCAGACCGCCAAGCTGAAGGTGACCAAGGGTGGCCCCCTGCCCT |
| | | TCGCCTGGGACATCCTGTCCCCTCAGTTCATGTACGGCTCCAAGGCCTACGTGAAGCACCCC |
| | | GCCGACATCCCCGACTACTTGAAGCTGTCCTTCCCCGAGGGCTTCAAGTGGGAGCGCGTGAT |
| | | GAACTTCGAGGACGGCGGCGTGGTGACCGTGACCCAGGACTCCTCCCTCCAGGACGGCGAG |
| | | TTCATCTACAAGGTGAAGCTGCGCGGCACCAACTTCCCCTCCGACGGCCCCGTAATGCAGAA |
| | | GAAGACCATGGGCTGGGAGGCCTCCTCCGAGCGGATGTACCCCGAGGACGGCGCCCTGAAG |
| | | GGCGAGATCAAGCAGCGGCTGAAGCTGAAGGACGGCGGCCACTACGACGCTGAGGTCAAG |
| | | ACCACCTACAAGGCCAAGAAGCCCGTGCAGCTGCCCGGCGCCTACAACGTCAACATCAAGT |
| | | TGGACATCACCTCCCACAACGAGGACTACACCATCGTGGAACAGTACGAACGCGCCGAGGG |
| | | CCGCCACTCCACCGGCGGCATGGACGAGCTGTACAAGTAGGGTACCAACCATACAACCTAC |
| | | TACCTCAAACCATACAACCTACTACCTCAAACCATACAACCTACTACCTCAAACCATACAAC |
| | | CTACTACCTCAAGATCTACGGGTGGCATCCCTGTGACCCCTCCCCAGTGCCTCT'CCTGGCCCT |
| | | GGAAGTTGCCACTCCAGTGCCCACCAGCCTTGTCCTAATAAAATTAAGTTGCATCATTTTGT |
| | | CTGACTAGGTGTCCTTCTATAATATTATGGGGTGGAGGGGGGTGGTATGGAGCAAGGGGCA |
| | | AGTTGGGAAGACAACCTGTAGGGCCTGCGGGGTCTATTGGGAACCAAGCTGGAGTGCAGTG |
| | | GCACAATCTTGGCTCACTGCAATCTCCGCCTCCTGGGTTCAAGCGATTCTCCTGCCTCAGCCT |
| | | CCCGAGTTGTTGGGATTCCAGGCATGCATGACCAGGCTCAGCTAATTTTTGTTTTTTTGGTAG |
| | | AGACGGGGTTTCACCATATTGGCCAGGCTGGTCTCCAACTCCTAATCTCAGGTGATCTACCC |
| | | ACCTTGGCCTCCCAAATTGCTGGGATTACAGGCGTGAACCACTGCTCCCTTCCCTGTCCTT |
| 291 | C.HNF1-FB. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGGGGTCCACTTGCTCCTGGGCCCACACAGTCC |
| | | TGCAGTATTGTGTATATAAGGCCAGGGCAAAGAGGAGCAGGTTTTAAAGTGAAAGGCAGGC |
| | | AGGTGTTGGGGAGGCAGTTACCGGGGCAACGGGAACAGGGCGTTTCGGAGGTGGTTGCCAT |
| | | GGGGACCTGGATGCTGTTCCATTCGCCATTCAGGCTGCGCAACTGTTGGGAAGGGCGATCG |
| | | GTGCGGGCCTCTTCGCTATTACGCCAGCTGGCGAAAGGGGGATGTGCTGCAAGGCGATTAA |
| | | GTTGGGTAACGCCAGGGTTTTCCCAGTCACGACGTTGTAAAACGACGGAATTCGAAGCTTAC |
| | | GACGGACATGTGAAATAGCGCTGTACAGCGTATGGGAATCTCTTGTACGGTGTACGAGTAT |
| | | CTTCCCGTACACCGTACGGCGCGCCAGTTAATAATTAACTAGTTAATAATTAACTAGTTAAT |
| | | AATTAACTCATATGCTCTAGAGGGTATATAATGGGGGCCACTAGTCTACTACCAGAGCTCAT |
| | | CGCTAGCGCTGGATCCGCCACCATGGTGAGCAAGGGCGAGGAGGATAACATGGCCATCATC |
| | | AAGGAGTTCATGCGCTTCAAGGTGCACATGGAGGGCTCCGTGAACGGCCACGAGTTCGAGA |
| | | TCGAGGGCGAGGGCGAGGGCCGCCCCTACGAGGGCACCCAGACCGCCAAGCTGAAGGTGA |
| | | CCAAGGGTGGCCCCCTGCCCTTCGCCTGGGACATCCTGTCCCCTCAGTTCATGTACGGCTCC |
| | | AAGGCCTACGTGAAGCACCCCGCCGACATCCCCGACTACTTGAAGCTGTCCTTCCCCGAGGG |
| | | CTTCAAGTGGGAGCGCGTGATGAACTTCGAGGACGGCGGCGTGGTGACCGTGACCCAGGAC |
| | | TCCTCCCTCCAGGACGGCGAGTTCATCTACAAGGTGAAGCTGCGCGGCACCAACTTCCCCTC |
| | | CGACGGCCCCGTAATGCAGAAGAAGACCATGGGCTGGGAGGCCTCCTCCGAGCGGATGTAC |
| | | CCCGAGGACGGCGCCCTGAAGGGCGAGATCAAGCAGCGGCTGAAGCTGAAGGACGGCGGC |
| | | CACTACGACGCTGAGGTCAAGACCACCTACAAGGCCAAGAAGCCCGTGCAGCTGCCCGGCG |
| | | CCTACAACGTCAACATCAAGTTGGACATCACCTCCCACAACGAGGACTACACCATCGTGGA |
| | | ACAGTACGAACGCGCCGAGGGCCGCCACTCCACCGGCGGCATGGACGAGCTGTACAAGTCC |
| | | GGAAGAGCCGAGGGCAGGGGAAGTCTTCTAACATGCGGGGACGTGGAGGAAAATCCCGGG |
| | | CCCAGATCTATGAGTCGAGGAGAGGTGCGCATGGCGAAGGCAGGGCGGGAGGGGCCGCGG |
| | | GACAGCGTGTGGCTGTCGGGGGAGGGGCGGCGCGGCGGTCGCCGTGGGGGGCAGCCGTCC |
| | | GGGCTCGACCGGGACCGGATCACCGGGGTCACCGTCCGGCTGCTGGACACGGAGGGCCTGA |
| | | CGGGGTTCTCGATGCGCCGCCTGGCCGCCGAGCTGAACGTCACCGCGATGTCCGTGTACTGG |
| | | TACGTCGACACCAAGGACCAGTTGCTCGAGCTCGCCCTGGACGCCGTCTTCGGCGAGCTGCG |
| | | CCACCCGGACCCGGACGCCGGGCTCGACTGGCGCGAGGAACTGCGGGCCCTGGCCCGGGAG |
| | | AACCGGGCGCTGCTGGTGCGCCACCCCTGGTCGTCCCGGCTGGTCGGCACCTACCTCAACAT |
| | | CGGCCCGCACTCGCTGGCCTTCTCCCGCGCGGTGCAGAACGTCGTGCGCCGCAGCGGGCTGC |
| | | CCGCGCACCGCCTGACCGGCGCCATCTCGGCCGTCTTCCAGTTCGTCTACGGCTACGGCACC |
| | | ATCGAGGGCCGCTTCCTCGCCCGGGTGGCGGACACCGGGCTGAGTCCGGAGGAGTACTTCC |
| | | AGGACTCGATGACCGCGGTGACCGAGGTGCCGGACACCGCGGGCGTCATCGAGGACGCGCA |
| | | GGACATCATGGCGGCCCGGGGCGGCGACACCGTGGCGGAGATGCTGGACCGGGACTTCGAG |
| | | TTCGCCCTCGACCTGCTCGTCGCGGGCATCGACGCGATGGTCGAACAGGCCTCCGCGTACAG |
| | | CCGCGCGCATGATGAGTTTCCCACCATGGTGTTTCCTTCTGGGCAGATCAGCCAGGCCTCGG |
| | | CCTTGGCCCCGGCCCCTCCCCAAGTCCTGCCCCAGGCTCCAGCCCCTGCCCCTGCTCCAGCC |
| | | ATGGTATCAGCTCTGGCCCAGGCCCCAGCCCCTGTCCCAGTCCTAGCCCCAGGCCCTCCTCA |
| | | GGCTGTGGCCCCACCTGCCCCCAAGCCCACCCAGGCTGGGGAAGGAACGCTGTCAGAGGCC |
| | | CTGCTGCAGCTGCAGTTTGATGATGAAGACCTGGGGGCCTTGCTTGGCAACAGCACAGACC |
| | | CAGCTGTGTTCACAGACCTGGCATCCGTCGACAACTCCGAGTTTCAGCAGCTGCTGAACCAG |
| | | GGCATACCTGTGGCCCCCCACACAACTGAGCCCATGCTGATGGAGTACCCTGAGGCTATAA |
| | | CTCGCCTAGTGACAGGGGCCCAGAGGCCCCCCGACCCAGCTCCTGCTCCACTGGGGGCCCC |
| | | GGGGCTCCCCAATGGCCTCCTTTCAGGAGATGAAGACTTCTCCTCCATTGCGGACATGGACT |
| | | TCTCAGCCCTGCTGAGTCAGATCAGCTCCTAAGGAAGCTTGGTACCGTCGACCTCGAGAGAT |
| | | CTACGGGTGGCATCCCTGTGACCCCTCCCCAGTGCCTCTCCTGGCCCTGGAAGTTGCCACTC |
| | | CAGTGCCCACCAGCCTTGTCCTAATAAAATTAAGTTGCATCATTTTGTCTGACTAGGTGTCCT |
| | | TCTATAATATTATGGGGTGGAGGGGGGTGGTATGGAGCAAGGGGCAAGTTGGGAAGACAAC |
| | | CTGTAGGGCCTGCGGGGTCTATTGGGAACCAAGCTGGAGTGCAGTGGCACAATCTTGGCTC |
| | | ACTGCAATCTCCGCCTCCTGGGTTCAAGCGATTCTCCTGCCTCAGCCTCCCGAGTTGTTGGG |
| | | ATTCCAGGCATGCATGACCAGGCTCAGCTAATTTTTGTTTTTTTGGTAGAGACGGGGTTTCA |
| | | CCATATTGGCCAGGCTGGTCTCCAACTCCTAATCTCAGGTGATCTACCCACCTTGGCCTCCC |
| | | AAATTGCTGGGATTACAGGCGTGAACCACTGCTCCCTTCCCTGTCCTT |
| 292 | C.SOX-FB. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGGGGTCCACTTGCTCCTGGGCCCACACAGTCC |
| | | TGCAGTATTGTGTATATAAGGCCAGGGCAAAGAGGAGCAGGTTTTAAAGTGAAAGGCAGGC |
| | | AGGTGTTGGGGAGGCAGTTACCGGGGCAACGGGAACAGGGCGTTTCGGAGGTGGTTGCCAT |
| | | GGGGACCTGGATGCTGTTCCATTCGCCATTCAGGCTGCGCAACTGTTGGGAAGGGCGATCG |
| | | GTGCGGGCCTCTTCGCTATTACGCCAGCTGGCGAAAGGGGGATGTGCTGCAAGGCGATTAA |
| | | GTTGGGTAACGCCAGGGTTTTCCCAGTCACGACGTTGTAAAACGACGGAATTCGAAGCTTAC |
| | | GACGGACATGTGAAATAGCGCTGTACAGCGTATGGGAATCTCTTGTACGGTGTACGAGTAT |
| | | CTTCCCGTACACCGTACGGCGCGCCCTACACAAAGCCCTCTTTGTGAGACTACACAAAGCCC |
| | | TCTTTGTGAGACTACACAAAGCCCTCTTTGTGAGACATATGCTCTAGAGGGTATATAATGGG |
| | | GGCCACTAGTCTACTACCAGAGCTCATCGCTAGCGCTGGATCCGCCACCATGGTGAGCAAG |
| | | GGCGAGGAGGATAACATGGCCATCATCAAGGAGTTCATGCGCTTCAAGGTGCACATGGAGG |
| | | GCTCCGTGAACGGCCACGAGTTCGAGATCGAGGGCGAGGGCGAGGGCCGCCCCTACGAGG |
| | | GCACCCAGACCGCCAAGCTGAAGGTGACCAAGGGTGGCCCCCTGCCCTTCGCCTGGGACAT |
| | | CCTGTCCCCTCAGTTCATGTACGGCTCCAAGGCCTACGTGAAGCACCCCGCCGACATCCCCG |
| | | ACTACTTGAAGCTGTCCTTCCCCGAGGGCTTCAAGTGGGAGCGCGTGATGAACTTCGAGGAC |
| | | GGCGGCGTGGTGACCGTGACCCAGGACTCCTCCCTCCAGGACGGCGAGTTCATCTACAAGG |
| | | TGAAGCTGCGCGGCACCAACTTCCCCTCCGACGGCCCCGTAATGCAGAAGAAGACCATGGG |
| | | CTGGGAGGCCTCCTCCGAGCGGATGTACCCCGAGGACGGCGCCCTGAAGGGCGAGATCAAG |
| | | CAGCGGCTGAAGCTGAAGGACGGCGGCCACTACGACGCTGAGGTCAAGACCACCTACAAG |
| | | GCCAAGAAGCCCGTGCAGCTGCCCGGCGCCTACAACGTCAACATCAAGTTGGACATCACCT |
| | | CCCACAACGAGGACTACACCATCGTGGAACAGTACGAACGCGCCGAGGGCCGCCACTCCAC |
| | | CGGCGGCATGGACGAGCTGTACAAGTCCGGAAGAGCCGAGGGCAGGGGAAGTCTTCTAAC |
| | | ATGCGGGGACGTGGAGGAAAATCCCGGGCCCAGATCTATGAGTCGAGGAGAGGTGCGCAT |
| | | GGCGAAGGCAGGGCGGGAGGGGCCGCGGGACAGCGTGTGGCTGTCGGGGGAGGGGCGGCG |
| | | CGGCGGTCGCCGTGGGGGGCAGCCGTCCGGGCTCGACCGGGACCGGATCACCGGGGTCACC |
| | | GTCCGGCTGCTGGACACGGAGGGCCTGACGGGGTTCTCGATGCGCCGCCTGGCCGCCGAGC |
| | | TGAACGTCACCGCGATGTCCGTGTACTGGTACGTCGACACCAAGGACCAGTTGCTCGAGCTC |
| | | GCCCTGGACGCCGTCTTCGGCGAGCTGCGCCACCCGGACCCGGACGCCGGGCTCGACTGGC |
| | | GCGAGGAACTGCGGGCCCTGGCCCGGGAGAACCGGGCGCTGCTGGTGCGCCACCCCTGGTC |
| | | GTCCCGGCTGGTCGGCACCTACCTCAACATCGGCCCGCACTCGCTGGCCTTCTCCCGCGCGG |
| | | TGCAGAACGTCGTGCGCCGCAGCGGGCTGCCCGCGCACCGCCTGACCGGCGCCATCTCGGC |
| | | CGTCTTCCAGTTCGTCTACGGCTACGGCACCATCGAGGGCCGCTTCCTCGCCCGGGTGGCGG |
| | | ACACCGGGCTGAGTCCGGAGGAGTACTTCCAGGACTCGATGACCGCGGTGACCGAGGTGCC |
| | | GGACACCGCGGGCGTCATCGAGGACGCGCAGGACATCATGGCGGCCCGGGGCGGCGACAC |
| | | CGTGGCGGAGATGCTGGACCGGGACTTCGAGTTCGCCCTCGACCTGCTCGTCGCGGGCATCG |
| | | ACGCGATGGTCGAACAGGCCTCCGCGTACAGCCGCGCGCATGATGAGTTTCCCACCATGGT |
| | | GTTTCCTTCTGGGCAGATCAGCCAGGCCTCGGCCTTGGCCCCGGCCCCTCCCCAAGTCCTGC |
| | | CCCAGGCTCCAGCCCCTGCCCCTGCTCCAGCCATGGTATCAGCTCTGGCCCAGGCCCCAGCC |
| | | CCTGTCCCAGTCCTAGCCCCAGGCCCTCCTCAGGCTGTGGCCCCACCTGCCCCCAAGCCCAC |
| | | CCAGGCTGGGGAAGGAACGCTGTCAGAGGCCCTGCTGCAGCTGCAGTTTGATGATGAAGAC |
| | | CTGGGGGCCTTGCTTGGCAACAGCACAGACCCAGCTGTGTTCACAGACCTGGCATCCGTCGA |
| | | CAACTCCGAGTTTCAGCAGCTGCTGAACCAGGGCATACCTGTGGCCCCCCACACAACTGAG |
| | | CCCATGCTGATGGAGTACCCTGAGGCTATAACTCGCCTAGTGACAGGGGCCCAGAGGCCCC |
| | | CCGACCCAGCTCCTGCTCCACTGGGGGCCCCGGGGCTCCCCAATGGCCTCCTTTCAGGAGAT |
| | | GAAGACTTCTCCTCCATTGCGGACATGGACTTCTCAGCCCTGCTGAGTCAGATCAGCTCCTA |
| | | AGGAAGCTTGGTACCGTCGACCTCGAGAGATCTACGGGTGGCATCCCTGTGACCCCTCCCCA |
| | | GTGCCTCTCCTGGCCCTGGAAGTTGCCACTCCAGTGCCCACCAGCCTTGTCCTAATAAAATT |
| | | AAGTTGCATCATTTTGTCTGACTAGGTGTCCTTCTATAATATTATGGGGTGGAGGGGGGTGG |
| | | TATGGAGCAAGGGGCAAGTTGGGAAGACAACCTGTAGGGCCTGCGGGGTCTATTGGGAACC |
| | | AAGCTGGAGTGCAGTGGCACAATCTTGGCTCACTGCAATCTCCGCCTCCTGGGTTCAAGCGA |
| | | TTCTCCTGCCTCAGCCTCCCGAGTTGTTGGGATTCCAGGCATGCATGACCAGGCTCAGCTAA |
| | | TTTTTGTTTTTTTGGTAGAGACGGGGTTTCACCATATTGGCCAGGCTGGTCTCCAACTCCTAA |
| | | TCTCAGGTGATCTACCCACCTTGGCCTCCCAAATTGCTGGGATTACAGGCGTGAACCACTGC |
| | | TCCCTTCCCTGTCCTT |
| 293 | Reporter 26. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCACCTATCCTGAATTACTTGAAACCTATCCTGAATTACTTGAAACCTATCCTGAATTACTTG |
| | | AAACCTATCCTGAATTACTTGAAGTCGACCTCGAGAGATCTACGGGTGGCATCCCTGTGACC |
| | | CCTCCCCAGTGCCTCTCCTGGCCCTGGAAGTTGCCACTCCAGTGCCCACCAGCCTTGTCCTA |
| | | ATAAAATTAAGTTGCATCATTTTGTCTGACTAGGTGTCCTTCTATAATATTATGGGGTGGAG |
| | | GGGGGTGGTATGGAGCAAGGGGCAAGTTGGGAAGACAACCTGTAGGGCCTGCGGGGTCTAT |
| | | TGGGAACCAAGCTGGAGTGCAGTGGCACAATCTTGGCTCACTGCAATCTCCGCCTCCTGGGT |
| | | TCAAGCGATTCTCCTGCCTCAGCCTCCCGAGTTGTTGGGATTCCAGGCATGCATGACCAGGC |
| | | TCAGCTAATTTTTGTTTTTTTGGTAGAGACGGGGTTTCACCATATTGGCCAGGCTGGTCTCCA |
| | | ACTCCTAATCTCAGGTGATCTACCCACCTTGGCCTCCCAAATTGCTGGGATTACAGGCGTGA |
| | | ACCACTGCTCCCTTCCCTGTCCTT |
| 294 | Repoter 22. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCACAGTTCTTCAACTGGCAGCTTACAGTTCTTCAACTGGCAGCTTACAGTTCTTCAACTGGC |
| | | AGCTTACAGTTCTTCAACTGGCAGCTTGTCGACCTCGAGAGATCTACGGGTGGCATCCCTGT |
| | | GACCCCTCCCCAGTGCCTCTCCTGGCCCTGGAAGTTGCCACTCCAGTGCCCACCAGCCTTGT |
| | | CCTAATAAAATTAAGTTGCATCATTTTGTCTGACTAGGTGTCCTTCTATAATATTATGGGGTG |
| | | GAGGGGGGTGGTATGGAGCAAGGGGCAAGTTGGGAAGACAACCTGTAGGGCCTGCGGGGT |
| | | CTATTGGGAACCAAGCTGGAGTGCAGTGGCACAATCTTGGCTCACTGCAATCTCCGCCTCCT |
| | | GGGTTCAAGCGATTCTCCTGCCTCAGCCTCCCGAGTTGTTGGGATTCCAGGCATGCATGACC |
| | | AGGCTCAGCTAATTTTTGTTTTTTTGGTAGAGACGGGGTTTCACCATATTGGCCAGGCTGGTC |
| | | TCCAACTCCTAATCTCAGGTGATCTACCCACCTTGGCCTCCCAAATTGCTGGGATTACAGGC |
| | | GTGAACCACTGCTCCCTTCCCTGTCCTT |
| 295 | Reporter 126. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCCGTGTTCACAGCGGACCTTGATCGTGTTCACAGCGGACCTTGATCGTGTTCACAGCGGAC |
| | | CTTGATCGTGTTCACAGCGGACCTTGATGTCGACCTCGAGAGATCTACGGGTGGCATCCCTG |
| | | TGACCCCTCCCCAGTGCCTCTCCTGGCCCTGGAAGTTGCCACTCCAGTGCCCACCAGCCTTG |
| | | TCCTAATAAAATTAAGTTGCATCATTTTGTCTGACTAGGTGTCCTTCTATAATATTATGGGGT |
| | | GGAGGGGGGTGGTATGGAGCAAGGGGCAAGTTGGGAAGACAACCTGTAGGGCCTGCGGGG |
| | | TCTATTGGGAACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCATATTGGCCAGGCTGGT |
| | | CTCCAACTCCTAATCTCAGGTGATCTACCCACCTTGGCCTCCCAAATTGCTGGGATTACAGG |
| | | CGTGAACCACTGCTCCCTTCCCTGTCCTT |
| 296 | Reporter 424. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CTCCAAAACATGAATTGCTGCTGTCCAAAACATGAATTGCTGCTGTCCAAAACATGAATTGC |
| | | TGCTGTCCAAAACATGAATTGCTGCTGGTCGACCTCGAGAGATCTACGGGTGGCATCCCTGT |
| | | GACCCCTCCCCAGTGCCTCTCCTGGCCCTGGAAGTTGCCACTCCAGTGCCCACCAGCCTTGT |
| | | CCTAATAAAATTAAGTTGCATCATTTTGTCTGACTAGGTGTCCTTCTATAATATTATGGGGTG |
| | | GAGGGGGGTGGTATGGAGCAAGGGGCAAGTTGGGAAGACAACCTGTAGGGCCTGCGGGGT |
| | | CTATTGGGAACCAAGCTGGAGTGCAGTGGCACAATCTTGGCTCACTGCAATCTCCGCCTCCT |
| | | GGGTTCAAGCGATTCTCCTGCCTCAGCCTCCCGAGTTGTTGGGATTCCAGGCATGCATGACC |
| | | AGGCTCAGCTAATTTTTGTTTTTTTGGTAGAGACGGGGTTTCACCATATTGGCCAGGCTGGTC |
| | | TCCAACTCCTAATCTCAGGTGATCTACCCACCTTGGCCTCCCAAATTGCTGGGATTACAGGC |
| | | GTGAACCACTGCTCCCTTCCCTGTCCTT |
| 297 | Reporter 122. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCCAAACACCATTGTCACACTCCACAAACACCATTGTCACACTCCACAAACACCATTGTCAC |
| | | ACTCCACAAACACCATTGTCACACTCCAGTCGACCTCGAGAGATCTACGGGTGGCATCCCTG |
| | | TGACCCCTCCCCAGTGCCTCTCCTGGCCCTGGAAGTTGCCACTCCAGTGCCCACCAGCCTTG |
| | | TCCTAATAAAATTAAGTTGCATCATTTTGTCTGACTAGGTGTCCTTCTATAATATTATGGGGT |
| | | GGAGGGGGGTGGTATGGAGCAAGGGGCAAGTTGGGAAGACAACCTGTAGGGCCTGCGGGG |
| | | TCTATTGGGAACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCATATTGGCCAGGCTGGT |
| | | CTCCAACTCCTAATCTCAGGTGATCTACCCACCTTGGCCTCCCAAATTGCTGGGATTACAGG |
| | | CGTGAACCACTGCTCCCTTCCCTGTCCTT |
| 298 | Reporter 217. | CAGTATTGTGTATATAAGGCCAGGGCAAAGAGGAGCAGGTTTTAAAGTGAAAGGCAGGCAG |
| | Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCTCCAGTCAGTTCCTGATGCAGTATCCAGTCAGTTCCTGATGCAGTATCCAGTCAGTTCCTG |
| | | ATGCAGTATCCAGTCAGTTCCTGATGCAGTAGTCGACCTCGAGAGATCTACGGGTGGCATCC |
| | | CTGTGACCCCTCCCCAGTGCCTCTCCTGGCCCTGGAAGTTGCCACTCCAGTGCCCACCAGCC |
| | | TTGTCCTAATAAAATTAAGTTGCATCATTTTGTCTGACTAGGTGTCCTTCTATAATATTATGG |
| | | GGTGGAGGGGGGTGGTATGGAGCAAGGGGCAAGTTGGGAAGACAACCTGTAGGGCCTGCG |
| | | GGGTCTATTGGGAACCAAGCTGGAGTGCAGTGGCACAATCTTGGCTCACTGCAATCTCCGCC |
| | | TCCTGGGTTCAAGCGATTCTCCTGCCTCAGCCTCCCGAGTTGTTGGGATTCCAGGCATGCAT |
| | | GACCAGGCTCAGCTAATTTTTGTTTTTTTGGTAGAGACGGGGTTTCACCATATTGGCCAGGC |
| | | TGGTCTCCAACTCCTAATCTCAGGTGATCTACCCACCTTGGCCTCCCAAATTGCTGGGATTAC |
| | | AGGCGTGAACCACTGCTCCCTTCCCTGTCCTT |
| 299 | Reporter | CAGTATTGTGTATATAAGGCCAGGGCAAAGAGGAGCAGGTTTTAAAGTGAAAGGCAGGCAG |
| | 216A.Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCTCACAGTTGCCAGCTGAGATTATCACAGTTGCCAGCTGAGATTATCACAGTTGCCAGCTG |
| | | AGATTATCACAGTTGCCAGCTGAGATTAGTCGACCTCGAGAGATCTACGGGTGGCATCCCTG |
| | | TGACCCCTCCCCAGTGCCTCTCCTGGCCCTGGAAGTTGCCACTCCAGTGCCCACCAGCCTTG |
| | | TCCTAATAAAATTAAGTTGCATCATTTTGTCTGACTAGGTGTCCTTCTATAATATTATGGGGT |
| | | GGAGGGGGGTGGTATGGAGCAAGGGGCAAGTTGGGAAGACAACCTGTAGGGCCTGCGGGG |
| | | TCTATTGGGAACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCATATTGGCCAGGCTGGT |
| | | CTCCAACTCCTAATCTCAGGTGATCTACCCACCTTGGCCTCCCAAATTGCTGGGATTACAGG |
| | | CGTGAACCACTGCTCCCTTCCCTGTCCTT |
| 300 | Reporter | CAGTATTGTGTATATAAGGCCAGGGCAAAGAGGAGCAGGTTTTAAAGTGAAAGGCAGGCAG |
| | 208A.Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCACAAGCTTTTTGCTCGTCTTATACAAGCTTTTTGCTCGTCTTATACAAGCTTTTTGCTCGTC |
| | | TTATACAAGCTTTTTGCTCGTCTTATGTCGACCTCGAGAGATCTACGGGTGGCATCCCTGTGA |
| | | CCCCTCCCCAGTGCCTCTCCTGGCCCTGGAAGTTGCCACTCCAGTGCCCACCAGCCTTGTCCT |
| | | AATAAAATTAAGTTGCATCATTTTGTCTGACTAGGTGTCCTTCTATAATATTATGGGGTGGA |
| | | GGGGGGTGGTATGGAGCAAGGGGCAAGTTGGGAAGACAACCTGTAGGGCCTGCGGGGTCT |
| | | ATTGGGAACCAAGCTGGAGTGCAGTGGCACAATCTTGGCTCACTGCAATCTCCGCCTCCTGG |
| | | GTTCAAGCGATTCTCCTGCCTCAGCCTCCCGAGTTGTTGGGATTCCAGGCATGCATGACCAG |
| | | GCTCAGCTAATTTTTGTTTTTTTGGTAGAGACGGGGTTTCACCATATTGGCCAGGCTGGTCTC |
| | | CAACTCCTAATCTCAGGTGATCTACCCACCTTGGCCTCCCAAATTGCTGGGATTACAGGCGT |
| | | GAACCACTGCTCCCTTCCCTGTCCTT |
| 301 | Reporter | CAGTATTGTGTATATAAGGCCAGGGCAAAGAGGAGCAGGTTTTAAAGTGAAAGGCAGGCAG |
| | 208B.Cherry | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCTAGACTGCAGCCTCAGGAGATCTGGGCCCCCGCGGC |
| | | ATATGTTACTTGTACAGCTCGTCCATGCCGAGAGTGATCCCGGCGGCGGTCACGAACTCCAG |
| | | CAGGACCATGTGATCGCGCTTCTCGTTGGGGTCTTTGCTCAGCTTGGACTGGGTGCTCAGGT |
| | | AGTGGTTGTCGGGCAGCAGCACGGGGCCGTCGCCGATGGGGGTGTTCTGCTGGTAGTGGTC |
| | | GGCGAGCTGCACGCTGCCGTCCTCGATGTTGTGGCGGATCTTGAAGTTGGCCTTGATGCCGT |
| | | TCTTCTGCTTGTCGGCGGTGATATAGACGTTGTCGCTGATGGCGTTGTACTCCAGCTTGTGCC |
| | | CCAGGATGTTGCCGTCCTCCTTGAAGTCGATGCCCTTCAGCTCGATGCGGTTCACCAGGGTG |
| | | TCGCCCTCGAACTTCACCTCGGCGCGGGTCTTGTAGTTGCCGTCGTCCTTGAAGAAGATGGT |
| | | GCGCTCCTGGACGTAGCCTTCGGGCATGGCGGACTTGAAGAAGTCGTGCTGCTTCATGTGGT |
| | | CGGGGTAGCGGGCGAAGCACTGCACGCCCCAGGTCAGGGTGGTCACGAGGGTGGGCCAGG |
| | | GCACGGGCAGCTTGCCGGTGGTGCAGATGAACTTCAGGGTCAGCTTGCCGTAGGTGGCATC |
| | | GCCCTCGCCCTCGCCGGACACGCTGAACTTGTGGCCGTTTACGTCGCCGTCCAGCTCGACCA |
| | | GGATGGGCACCACCCCGGTGAACAGCTCCTCGCCCTTGCTCACCATGGTGGCGAATTCGCGG |
| | | ATCTGACGGTTCACTAAACCAGCTCTGCTTATATAGACCTCCCACCGTACACGCCTACCGCC |
| | | CATTTGCGTCAATGGGGCGGAGTTGTTACGACATTTTGGAAAGTCCCGTTGATTTTGGTGCC |
| | | AAAACAAACTCCCATTGACGTCAATGGGGTGGAGACTTGGAAATCCCCGTGAGTCAAACCG |
| | | CTATCCACGCCCATTGATGTACTGCCAAAACCGCATCACACTAGTTATTAATAGTAATCAAT |
| | | TACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCC |
| | | ATAGTAACGCCAATAGGGACTTTCCATTGACGTCAATGGGTGGAGTATTTACGGTAAACTGC |
| | | CCACTTGGCAGTACATCAAGTGTATCATATGCCAAGTACGCCCCCTATTGACGTCAATGACG |
| | | GTAAATGGCCCGCCTGGCATTATGCCCAGTACATGACCTTATGGGACTTTCCTACTTGGCAG |
| | | TACATCTACGTATTAGTCATCGCTATTACCATGGTGATGCGGTTTTGGCAGTACATCAATGG |
| | | GCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCTCGTTTCGTAC |
| | | GTTCGAAGCCACCATGGTGAGCAAGGGCGAGGAGGATAACATGGCCATCATCAAGGAGTTC |
| | | ATGCGCTTCAAGGTGCACATGGAGGGCTCCGTGAACGGCCACGAGTTCGAGATCGAGGGCG |
| | | AGGGCGAGGGCCGCCCCTACGAGGGCACCCAGACCGCCAAGCTGAAGGTGACCAAGGGTG |
| | | GCCCCCTGCCCTTCGCCTGGGACATCCTGTCCCCTCAGTTCATGTACGGCTCCAAGGCCTAC |
| | | GTGAAGCACCCCGCCGACATCCCCGACTACTTGAAGCTGTCCTTCCCCGAGGGCTTCAAGTG |
| | | GGAGCGCGTGATGAACTTCGAGGACGGCGGCGTGGTGACCGTGACCCAGGACTCCTCCCTC |
| | | CAGGACGGCGAGTTCATCTACAAGGTGAAGCTGCGCGGCACCAACTTCCCCTCCGACGGCC |
| | | CCGTAATGCAGAAGAAGACCATGGGCTGGGAGGCCTCCTCCGAGCGGATGTACCCCGAGGA |
| | | CGGCGCCCTGAAGGGCGAGATCAAGCAGCGGCTGAAGCTGAAGGACGGCGGCCACTACGA |
| | | CGCTGAGGTCAAGACCACCTACAAGGCCAAGAAGCCCGTGCAGCTGCCCGGCGCCTACAAC |
| | | GTCAACATCAAGTTGGACATCACCTCCCACAACGAGGACTACACCATCGTGGAACAGTACG |
| | | AACGCGCCGAGGGCCGCCACTCCACCGGCGGCATGGACGAGCTGTACAAGTAGACGCGGAT |
| | | CCACAAACCTTTTGTTCGTCTTATACAAACCTTTTGTTCGTCTTATACAAACCTTTTGTTCGTC |
| | | TTATACAAACCTTTTGTTCGTCTTATGTCGACCTCGAGAGATCTACGGGTGGCATCCCTGTGA |
| | | CCCCTCCCCAGTGCCTCTCCTGGCCCTGGAAGTTGCCACTCCAGTGCCCACCAGCCTTGTCCT |
| | | AATAAAATTAAGTTGCATCATTTTGTCTGACTAGGTGTCCTTCTATAATATTATGGGGTGGA |
| | | GGGGGGTGGTATGGAGCAAGGGGCAAGTTGGGAAGACAACCTGTAGGGCCTGCGGGGTCT |
| | | ATTGGGAACCAAGCTGGAGTGCAGTGGCACAATCTTGGCTCACTGCAATCTCCGCCTCCTGG |
| | | GTTCAAGCGATTCTCCTGCCTCAGCCTCCCGAGTTGTTGGGATTCCAGGCATGCATGACCAG |
| | | GCTCAGCTAATTTTTGTTTTTTTGGTAGAGACGGGGTTTCACCATATTGGCCAGGCTGGTCTC |
| | | CAACTCCTAATCTCAGGTGATCTACCCACCTTGGCCTCCCAAATTGCTGGGATTACAGGCGT |
| | | GAACCACTGCTCCCTTCCCTGTCCTT |
| 302 | HCC.V1.Cherry | CAGTATTGTGTATATAAGGCCAGGGCAAAGAGGAGCAGGTTTTAAAGTGAAAGGCAGGCAG |
| | | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGCTTCGAATCGATGAATTCGAAGCTTCTACCC |
| | | ACCGTACTCGTCAATTCCAAGGGCATCGGTAAACATCTGCTCAAACTCGAAGTCGGCCATAT |
| | | CCAGAGCGCCGTAGGGGGCGGAGTCGTGGGGGGTAAATCCCGGACCCGGGGAATCCCCGTC |
| | | CCCCAACATGTCCAGATCGAAATCGTCTAGCGCGTCGGCATGCGCCATCGCCACGTCCTCGC |
| | | CGTCTAAGTGGAGCTCGTCCCCCAGGCTGACATCGGTCGGGGGGGCCGTCGACAGTCTGCG |
| | | CGTGTGTCCCGCGGGGAGAAAGGACAGGCGCGGAGCCGCCAGCCCCGCCTCTTCGGGGGCG |
| | | TCGTCGTCCGGGAGATCGAGCAGGCCCTCGATGGTAGACCCGTAATTGTTTTTCGTACGCGC |
| | | GCGGCTGTACGCGGAGGCCTGTTCGACCATCGCGTCGATGCCCGCGACGAGCAGGTCGAGG |
| | | GCGAACTCGAAGTCCCGGTCCAGCATCTCCGCCACGGTGTCGCCGCCCCGGGCCGCCATGAT |
| | | GTCCTGCGCGTCCTCGATGACGCCCGCGGTGTCCGGCACCTCGGTCACCGCGGTCATCGAGT |
| | | CCTGGAAGTACTCCTCCGGACTCAGCCCGGTGTCCGCCACCCGGGCGAGGAAGCGGCCCTC |
| | | GATGGTGCCGTAGCCGTAGACGAACTGGAAGACGGCCGAGATGGCGCCGGTCAGGCGGTGC |
| | | GCGGGCAGCCCGCTGCGGCGCACGACGTTCTGCACCGCGCGGGAGAAGGCCAGCGAGTGCG |
| | | GGCCGATGTTGAGGTAGGTGCCGACCAGCCGGGACGACCAGGGGTGGCGCACCAGCAGCG |
| | | CCCGGTTCTCCCGGGCCAGGGCCCGCAGTTCCTCGCGCCAGTCGAGCCCGGCGTCCGGGTCC |
| | | GGGTGGCGCAGCTCGCCGAAGACGGCGTCCAGGGCGAGCTCGAGCAACTGGTCCTTGGTGT |
| | | CGACGTACCAGTACACGGACATCGCGGTGACGTTCAGCTCGGCGGCCAGGCGGCGCATCGA |
| | | GAACCCCGTCAGGCCCTCCGTGTCCAGCAGCCGGACGGTGACCCCGGTGATCCGGTCCCGG |
| | | TCGAGCCCGGACGGCTGCCCCCCACGGCGACCGCCGCGCCGCCCCTCCCCCGACAGCCACA |
| | | CGCTGTCCCGCGGCCCCTCCCGCCCTGCCTTCGCCATGCGCACCTCTCCTCGACTCATACCGG |
| | | TAGCGCTAGCGATGAGCTCTGGTAGTAGACTAGTGGCCCCCATTATATACCCTCTAGAGCAT |
| | | ATGTCTCACAAAGAGGGCTTTGTGTAGTCTCACAAAGAGGGCTTTGTGTAGTCTCACAAAGA |
| | | GGGCTTTGTGTAGGGCGCGCCCCCGTAGCTTGGCGTAATCACATGTCCGTCGTTTTACAACG |
| | | TCGTGACTGGGAAAACCCTGGCCTGCAAGGCGATTAAGTTGGGTAACGCCAGGGTTTTCCC |
| | | AGTCACGACGTTGTAAAACGACGGACATGTGAAATAGCGCTGTACAGCGTATGGGAATCTC |
| | | TTGTACGGTGTACGAGTATCTTCCCGTACACCGTACGGCGCGCCAGTTAATAATTAACTAGT |
| | | TAATAATTAACTAGTTAATAATTAACTCATATGCTCTAGAGGGTATATAATGGGGGCCACTA |
| | | GTCTACTACCAGAGCTCATCGCTAGCGCTGGATCCGCCACCATGGTGAGCAAGGGCGAGGA |
| | | GGATAACATGGCCATCATCAAGGAGTTCATGCGCTTCAAGGTGCACATGGAGGGCTCCGTG |
| | | AACGGCCACGAGTTCGAGATCGAGGGCGAGGGCGAGGGCCGCCCCTACGAGGGCACCCAG |
| | | ACCGCCAAGCTGAAGGTGACCAAGGGTGGCCCCCTGCCCTTCGCCTGGGACATCCTGTCCCC |
| | | TCAGTTCATGTACGGCTCCAAGGCCTACGTGAAGCACCCCGCCGACATCCCCGACTACTTGA |
| | | AGCTGTCCTTCCCCGAGGGCTTCAAGTGGGAGCGCGTGATGAACTTCGAGGACGGCGGCGT |
| | | GGTGACCGTGACCCAGGACTCCTCCCTCCAGGACGGCGAGTTCATCTACAAGGTGAAGCTG |
| | | CGCGGCACCAACTTCCCCTCCGACGGCCCCGTAATGCAGAAGAAGACCATGGGCTGGGAGG |
| | | CCTCCTCCGAGCGGATGTACCCCGAGGACGGCGCCCTGAAGGGCGAGATCAAGCAGCGGCT |
| | | GAAGCTGAAGGACGGCGGCCACTACGACGCTGAGGTCAAGACCACCTACAAGGCCAAGAA |
| | | GCCCGTGCAGCTGCCCGGCGCCTACAACGTCAACATCAAGTTGGACATCACCTCCCACAAC |
| | | GAGGACTACACCATCGTGGAACAGTACGAACGCGCCGAGGGCCGCCACTCCACCGGCGGCA |
| | | TGGACGAGCTGTACAAGTAGGGTACCCAAACACCATTGTCACACTCCAAGATCTACGGGTG |
| | | GCATCCCTGTGACCCCTCCCCAGTGCCTCTCCTGGCCCTGGAAGTTGCCACTCCAGTGCCCA |
| | | CCAGCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAATC |
| | | TCCGCCTCCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACCATATTGGC |
| | | CAGGCTGGTCTCCAACTCCTAATCTCAGGTGATCTACCCACCTTGGCCTCCCAAATTGCTGG |
| | | GATTACAGGCGTGAACCACTGCTCCCTTCCCTGTCCTT |
| 303 | HCC.V1. | CAGTATTGTGTATATAAGGCCAGGGCAAAGAGGAGCAGGTTTTAAAGTGAAAGGCAGGCAG |
| | HSV-TK | GTGTTGGGGAGGCAGTTACCGGGGCAACGGGAACAGGGCGTTTCGGAGGTGGTTGCCATGG |
| | | GGACCTGGATGCTGACGAAGGCTCGATTATTGAAGCATTTATCAGGGTTATTGTCTCATGAG |
| | | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGCTTCGAATCGATGAATTCGAAGCTTCTACCC |
| | | ACCGTACTCGTCAATTCCAAGGGCATCGGTAAACATCTGCTCAAACTCGAAGTCGGCCATAT |
| | | CCAGAGCGCCGTAGGGGGCGGAGTCGTGGGGGGTAAATCCCGGACCCGGGGAATCCCCGTC |
| | | CCCCAACATGTCCAGATCGAAATCGTCTAGCGCGTCGGCATGCGCCATCGCCACGTCCTCGC |
| | | CGTCTAAGTGGAGCTCGTCCCCCAGGCTGACATCGGTCGGGGGGGCCGTCGACAGTCTGCG |
| | | CGTGTGTCCCGCGGGGAGAAAGGACAGGCGCGGAGCCGCCAGCCCCGCCTCTTCGGGGGCG |
| | | TCGTCGTCCGGGAGATCGAGCAGGCCCTCGATGGTAGACCCGTAATTGTTTTTCGTACGCGC |
| | | GCGGCTGTACGCGGAGGCCTGTTCGACCATCGCGTCGATGCCCGCGACGAGCAGGTCGAGG |
| | | GCGAACTCGAAGTCCCGGTCCAGCATCTCCGCCACGGTGTCGCCGCCCCGGGCCGCCATGAT |
| | | GTCCTGCGCGTCCTCGATGACGCCCGCGGTGTCCGGCACCTCGGTCACCGCGGTCATCGAGT |
| | | CCTGGAAGTACTCCTCCGGACTCAGCCCGGTGTCCGCCACCCGGGCGAGGAAGCGGCCCTC |
| | | GATGGTGCCGTAGCCGTAGACGAACTGGAAGACGGCCGAGATGGCGCCGGTCAGGCGGTGC |
| | | GCGGGCAGCCCGCTGCGGCGCACGACGTTCTGCACCGCGCGGGAGAAGGCCAGCGAGTGCG |
| | | GGCCGATGTTGAGGTAGGTGCCGACCAGCCGGGACGACCAGGGGTGGCGCACCAGCAGCG |
| | | CCCGGTTCTCCCGGGCCAGGGCCCGCAGTTCCTCGCGCCAGTCGAGCCCGGCGTCCGGGTCC |
| | | GGGTGGCGCAGCTCGCCGAAGACGGCGTCCAGGGCGAGCTCGAGCAACTGGTCCTTGGTGT |
| | | CGACGTACCAGTACACGGACATCGCGGTGACGTTCAGCTCGGCGGCCAGGCGGCGCATCGA |
| | | GAACCCCGTCAGGCCCTCCGTGTCCAGCAGCCGGACGGTGACCCCGGTGATCCGGTCCCGG |
| | | TCGAGCCCGGACGGCTGCCCCCCACGGCGACCGCCGCGCCGCCCCTCCCCCGACAGCCACA |
| | | CGCTGTCCCGCGGCCCCTCCCGCCCTGCCTTCGCCATGCGCACCTCTCCTCGACTCATACCGG |
| | | TAGCGCTAGCGATGAGCTCTGGTAGTAGACTAGTGGCCCCCATTATATACCCTCTAGAGCAT |
| | | ATGTCTCACAAAGAGGGCTTTGTGTAGTCTCACAAAGAGGGCTTTGTGTAGTCTCACAAAGA |
| | | GGGCTTTGTGTAGGGCGCGCCCCCGTAGCTTGGCGTAATCACATGTCCGTCGTTTTACAACG |
| | | TCGTGACTGGGAAAACCCTGGCCTGCAAGGCGATTAAGTTGGGTAACGCCAGGGTTTTCCC |
| | | AGTCACGACGTTGTAAAACGACGGACATGTGAAATAGCGCTGTACAGCGTATGGGAATCTC |
| | | TTGTACGGTGTACGAGTATCTTCCCGTACACCGTACGGCGCGCCAGTTAATAATTAACTAGT |
| | | TAATAATTAACTAGTTAATAATTAACTCATATGCTCTAGAGGGTATATAATGGGGGCCACTA |
| | | GTCTACTACCAGAGCTCATCGCTAGCGCTGGATCCCGCCACCATGGCTTCGTACCCCTGCCA |
| | | TCAACACGCGTCTGCGTTCGACCAGGCTGCGCGTTCTCGCGGCCATAGCAACCGACGTACGG |
| | | CGTTGCGCCCTCGCCGGCAGCAAGAAGCCACGGAAGTCCGCCTGGAGCAGAAAATGCCCAC |
| | | GCTACTGCGGGTTTATATAGACGGTCCTCACGGGATGGGGAAAACCACCACCACGCAACTG |
| | | CTGGTGGCCCTGGGTTCGCGCGACGATATCGTCTACGTACCCGAGCCGATGACTTACTGGCA |
| | | GGTGCTGGGGGCTTCCGAGACAATCGCGAACATCTACACCACACAACACCGCCTCGACCAG |
| | | GGTGAGATATCGGCCGGGGACGCGGCGGTGGTAATGACAAGCGCCCAGATAACAATGGGC |
| | | ATGCCTTATGCCGTGACCGACGCCGTTCTGGCTCCTCATATCGGGGGGGAGGCTGGGAGCTC |
| | | ACATGCCCCGCCCCCGGCCCTCACCCTCATCTTCGACCGCCATCCCATCGCCGCCCTCCTGTG |
| | | CTACCCGGCCGCGCGATACCTTATGGGCAGCATGACCCCCCAGGCCGTGCTGGCGTTCGTGG |
| | | CCCTCATCCCGCCGACCTTGCCCGGCACAAACATCGTGTTGGGGGCCCTTCCGGAGGACAGA |
| | | CACATCGACCGCCTGGCCAAACGCCAGCGCCCCGGCGAGCGGCTTGACCTGGCTATGCTGG |
| | | CCGCGATTCGCCGCGTTTACGGGCTGCTTGCCAATACGGTGCGGTATCTGCAGGGCGGCGGG |
| | | TCGTGGCGGGAGGATTGGGGACAGCTTTCGGGGACGGCCGTGCCGCCCCAGGGTGCCGAGC |
| | | CCCAGAGCAACGCGGGCCCACGACCCCATATCGGGGACACGTTATTTACCCTGTTTCGGGCC |
| | | CCCGAGTTGCTGGCCCCCAACGGCGACCTGTACAACGTGTTTGCCTGGGCCTTGGACGTCTT |
| | | GGCCAAACGCCTCCGTCCCATGCACGTCTTTATCCTGGATTACGACCAATCGCCCGCCGGCT |
| | | GCCGGGACGCCCTGCTGCAACTTACCTCCGGGATGGTCCAGACCCACGTCACCACCCCCGGC |
| | | TCCATACCGACGATCTGCGACCTGGCGCGCACGTTTGCCCGGGAGATGGGGGAGGCTAACT |
| | | GAGGTACCCAAACACCATTGTCACACTCCAAGATCTACGGGTGGCATCCCTGTGACCCCTCC |
| | | CCAGTGCCTCTCCTGGCCCTGGAAGTTGCCACTCCAGTGCCCACCAGCCTTGTCCTAATAAA |
| | | ATTAAGTTGCATCATTTTGTCTGACTAGGTGTCCTTCTATAATATTATGGGGTGGAGGGGGG |
| | | TGGTATGGAGCAAGGGGCAAGTTGGGAAGACAACCTGTAGGGCCTGCGGGGTCTATTGGGA |
| | | ACCAAGCTGGAGTGCAGTGGCACAATCTTGGCTCACTGCAATCTCCGCCTCCTGGGTTCAAG |
| | | CGATTCTCCTGCCTCAGCCTCCCGAGTTGTTGGGATTCCAGGCATGCATGACCAGGCTCAGC |
| | | TAATTTTTGTTTTTTTGGTAGAGACGGGGTTTCACCATATTGGCCAGGCTGGTCTCCAACTCC |
| | | TAATCTCAGGTGATCTACCCACCTTGGCCTCCCAAATTGCTGGGATTACAGGCGTGAACCAC |
| | | TGCTCCCTTCCCTGTCCTT |
| 304 | HCC.V2. | GGACCTGGATGCTGACGAAGGCTCGATTATTGAAGCATTTATCAGGGTTATTGTCTCATGAG |
| | HSV-TK | CGGATACATATTTGAATGTATTTAGAAAAATAAACAAATAGGGGTTCCGCGCACATTTCCCC |
| | | GAAAAGTGCCACCTGACGTCGGCAGTGAAAAAAATGCTTTATTTGTGAAATTTGTGATGCTA |
| | | TTGCTTTATTTGTAACCATTATAAGCTGCAATAAACAAGTTAACAACAACAATTGCATTCAT |
| | | TTTATGTTTCAGGTTCAGGGGGAGGTGTGGGAGGTTTTTTAAAGCAAGTAAAACCTCTACAA |
| | | ATGTGGTATGGCTGATTATGATCCTCCTAGGTGAGGTAGTAGGTTGTATGGTTTGAGGTAGT |
| | | AGGTTGTATGGTTTGAGGTAGTAGGTTGTATGGTTTGAGGTAGTAGGTTGTATGGTTATCGA |
| | | TGAATTCGAAGCTTCTACCCACCGTACTCGTCAATTCCAAGGGCATCGGTAAACATCTGCTC |
| | | AAACTCGAAGTCGGCCATATCCAGAGCGCCGTAGGGGGCGGAGTCGTGGGGGGTAAATCCC |
| | | GGACCCGGGGAATCCCCGTCCCCCAACATGTCCAGATCGAAATCGTCTAGCGCGTCGGCAT |
| | | GCGCCATCGCCACGTCCTCGCCGTCTAAGTGGAGCTCGTCCCCCAGGCTGACATCGGTCGGG |
| | | GGGGCCGTCGACAGTCTGCGCGTGTGTCCCGCGGGGAGAAAGGACAGGCGCGGAGCCGCC |
| | | AGCCCCGCCTCTTCGGGGGCGTCGTCGTCCGGGAGATCGAGCAGGCCCTCGATGGTAGACC |
| | | CGTAATTGTTTTTCGTACGCGCGCGGCTGTACGCGGAGGCCTGTTCGACCATCGCGTCGATG |
| | | CCCGCGACGAGCAGGTCGAGGGCGAACTCGAAGTCCCGGTCCAGCATCTCCGCCACGGTGT |
| | | CGCCGCCCCGGGCCGCCATGATGTCCTGCGCGTCCTCGATGACGCCCGCGGTGTCCGGCACC |
| | | TCGGTCACCGCGGTCATCGAGTCCTGGAAGTACTCCTCCGGACTCAGCCCGGTGTCCGCCAC |
| | | CCGGGCGAGGAAGCGGCCCTCGATGGTGCCGTAGCCGTAGACGAACTGGAAGACGGCCGA |
| | | GATGGCGCCGGTCAGGCGGTGCGCGGGCAGCCCGCTGCGGCGCACGACGTTCTGCACCGCG |
| | | CGGGAGAAGGCCAGCGAGTGCGGGCCGATGTTGAGGTAGGTGCCGACCAGCCGGGACGAC |
| | | CAGGGGTGGCGCACCAGCAGCGCCCGGTTCTCCCGGGCCAGGGCCCGCAGTTCCTCGCGCC |
| | | AGTCGAGCCCGGCGTCCGGGTCCGGGTGGCGCAGCTCGCCGAAGACGGCGTCCAGGGCGAG |
| | | CTCGAGCAACTGGTCCTTGGTGTCGACGTACCAGTACACGGACATCGCGGTGACGTTCAGCT |
| | | CGGCGGCCAGGCGGCGCATCGAGAACCCCGTCAGGCCCTCCGTGTCCAGCAGCCGGACGGT |
| | | GACCCCGGTGATCCGGTCCCGGTCGAGCCCGGACGGCTGCCCCCCACGGCGACCGCCGCGC |
| | | CGCCCCTCCCCCGACAGCCACACGCTGTCCCGCGGCCCCTCCCGCCCTGCCTTCGCCATGCG |
| | | CACCTCTCCTCGACTCATACCGGTAGCGCTAGCGATGAGCTCTGGTAGTAGACTAGTGGCCC |
| | | CCATTATATACCCTCTAGAGCATATGTCTCACAAAGAGGGCTTTGTGTAGTCTCACAAAGAG |
| | | GGCTTTGTGTAGTCTCACAAAGAGGGCTTTGTGTAGGGCGCGCCCCCGTAGCTTGGCGTAAT |
| | | CACATGTCCGTCGTTTTACAACGTCGTGACTGGGAAAACCCTGGCCTGCAAGGCGATTAAGT |
| | | TGGGTAACGCCAGGGTTTTCCCAGTCACGACGTTGTAAAACGACGGACATGTGAAATAGCG |
| | | CTGTACAGCGTATGGGAATCTCTTGTACGGTGTACGAGTATCTTCCCGTACACCGTACGGCG |
| | | CGCCAGTTAATAATTAACTAGTTAATAATTAACTAGTTAATAATTAACTCATATGCTCTAGA |
| | | GGGTATATAATGGGGGCCACTAGTCTACTACCAGAGCTCATCGCTAGCGCTGGATCCCGCCA |
| | | CCATGGCTTCGTACCCCTGCCATCAACACGCGTCTGCGTTCGACCAGGCTGCGCGTTCTCGC |
| | | GGCCATAGCAACCGACGTACGGCGTTGCGCCCTCGCCGGCAGCAAGAAGCCACGGAAGTCC |
| | | GCCTGGAGCAGAAAATGCCCACGCTACTGCGGGTTTATATAGACGGTCCTCACGGGATGGG |
| | | GAAAACCACCACCACGCAACTGCTGGTGGCCCTGGGTTCGCGCGACGATATCGTCTACGTA |
| | | CCCGAGCCGATGACTTACTGGCAGGTGCTGGGGGCTTCCGAGACAATCGCGAACATCTACA |
| | | CCACACAACACCGCCTCGACCAGGGTGAGATATCGGCCGGGGACGCGGCGGTGGTAATGAC |
| | | AAGCGCCCAGATAACAATGGGCATGCCTTATGCCGTGACCGACGCCGTTCTGGCTCCTCATA |
| | | TCGGGGGGGAGGCTGGGAGCTCACATGCCCCGCCCCCGGCCCTCACCCTCATCTTCGACCGC |
| | | CATCCCATCGCCGCCCTCCTGTGCTACCCGGCCGCGCGATACCTTATGGGCAGCATGACCCC |
| | | CCAGGCCGTGCTGGCGTTCGTGGCCCTCATCCCGCCGACCTTGCCCGGCACAAACATCGTGT |
| | | TGGGGGCCCTTCCGGAGGACAGACACATCGACCGCCTGGCCAAACGCCAGCGCCCCGGCGA |
| | | GCGGCTTGACCT'GGCTATGCTGGCCGCGATTCGCCGCGTTTACGGGCTGCTTGCCAATACGG |
| | | TGCGGTATCTGCAGGGCGGCGGGTCGTGGCGGGAGGATTGGGGACAGCTTTCGGGGACGGC |
| | | CGTGCCGCCCCAGGGTGCCGAGCCCCAGAGCAACGCGGGCCCACGACCCCATATCGGGGAC |
| | | ACGTTATTTACCCTGTTTCGGGCCCCCGAGTTGCTGGCCCCCAACGGCGACCTGTACAACGT |
| | | GTTTGCCTGGGCCTTGGACGTCTTGGCCAAACGCCTCCGTCCCATGCACGTCTTTATCCTGGA |
| | | TTACGACCAATCGCCCGCCGGCTGCCGGGACGCCCTGCTGCAACTTACCTCCGGGATGGTCC |
| | | AGACCCACGTCACCACCCCCGGCTCCATACCGACGATCTGCGACCTGGCGCGCACGTTTGCC |
| | | CGGGAGATGGGGGAGGCTAACTGAGGTACCAACCATACAACCTACTACCTCAAACCATACA |
| | | ACCTACTACCTCAAACCATACAACCTACTACCTCAAACCATACAACCTACTACCTCAAGATC |
| | | TACGGGTGGCATCCCTGTGACCCCTCCCCAGTGCCTCTCCTGGCCCTGGAAGTTGCCACTCC |
| | | AGTGCCCACCAGCCTTGTCCTAATAAAATTAAGTTGCATCATTTTGTCTGACTAGGTGTCCTT |
| | | CTATAATATTATGGGGTGGAGGGGGGTGGTATGGAGCAAGGGGCAAGTTGGGAAGACAAC |
| | | CTGTAGGGCCTGCGGGGTCTATTGGGAACCAAGCTGGAGTGCAGTGGCACAATCTTGGCTC |
| | | ACTGCAATCTCCGCCTCCTGGGTTCAAGCGATTCTCCTGCCTCAGCCTCCCGAGTTGTTGGG |
| | | ATTCCAGGCATGCATGACCAGGCTCAGCTAATTTTTGTTTTTTTGGTAGAGACGGGGTTTCA |
| | | CCATATTGGCCAGGCTGGTCTCCAACTCCTAATCTCAGGTGATCTACCCACCTTGGCCTCCC |
| | | AAATTGCTGGGATTACAGGCGTGAACCACTGCTCCCTTCCCTGTCCTT |

### II. Other Compositions

In other aspects, the disclosure relates to compositions of vectors. In some embodiments, a vector comprises a contiguous polynucleic acid molecule described above.

In other aspects, the disclosure relates to compositions of engineered viral genomes. In some embodiments, the viral genome comprises a contiguous polynucleic acid molecule described above. In some embodiments, the viral genome is an adeno-associated virus (AAV) genome, a lentivirus genome, an adenovirus genome, a herpes simplex virus (HSV) genome, a Vaccinia virus genome, a poxvirus genome, a Newcastle Disease virus (NDV) genome, a Coxsackievirus genome, a rheovirus genome, a measles virus genome, a Vesicular Stomatitis virus (VSV) genome, a Parvovirus genome, a Seneca valley viral genome, a Maraba virus genome, or a common cold virus genome.

In other aspects, the disclosure relates to compositions of virions. As used herein, the term “virion” refers to an infective form of a virus that is outside of a host cell (e.g., comprising a DNA/RNA genome and a capsid protein). In some embodiments, a virion comprises the engineered viral genome described above. In some embodiments, the virion comprises a AAV-DJ capsid protein. In some embodiments, the virion comprises a AAV-B1 capsid protein, an AAV8 capsid protein, or an AAV6 capsid protein.

In other aspects, the disclosure relates to compositions comprising a contiguous polynucleic acid molecule described above, a vector described above, an engineered viral genome described above, or a virion described above. In some embodiments, the composition is a therapeutic composition further comprising a pharmaceutically-acceptable excipient or buffer. Exemplary pharmaceutical excipients and buffers are known to those having ordinary skill in the art.

### III. Methods of Stimulating a Cell-Specific Event

In other aspects, the disclosure relates to methods of stimulating a cell-specific event in a population of cells. In some embodiments, the method of stimulating the cell-specific event comprises contacting a population of cells with a contiguous polynucleic acid molecule described above, a vector described above, an engineered viral genome described above, or a virion described above, wherein the cell-specific event is elicited via the level of output expressed in the cells of the population of cells.

In some embodiments, the population of cells comprises at least one target cell and at least one non-target cell. A target cell and a non-target cell type differ in levels of at least one endogenous transcription factor and/or the expression strength of at least one endogenous promoter or its fragment and/or at least one endogenous miRNA. In some embodiments, the expression levels of the output differs between target cells and non-target cells by at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1,000, or at least 10,000 fold.

In some embodiments, the method comprises contacting the population of cells with the contiguous polynucleic acid molecule or a composition comprising said contiguous polynucleic aid molecule, wherein: a) the population of cells comprises at least one target cell type and two or more non-target cell types, wherein the target cell type(s) and the non-target cell types differ in levels of one or more endogenous miRNAs (e.g., at least 1, at least 2, at least 3, at least 4, at least 5, at least 6, at least 7, at least 8, at least 9, at least 10, at least 11, at least 12, at least 13, at least 14, at least 15, at least 20 endogenous miRNAs), such that the levels of the one or more endogenous miRNAs are at least two times higher (e.g., at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 1000 times higher) in each of the two or more non-target cells relative to each of the target cells; and b) the contiguous polynucleic acid molecule comprises: (i) a first cassette encoding a RNA whose expression is operably linked to a transactivator response element, wherein the first RNA comprises: a nucleic acid sequence of an output; and one or more miRNA target sites corresponding to the one or more endogenous miRNAs; and (ii) a second cassette encoding a second RNA, wherein the second RNA comprises a nucleic acid sequence of a transactivator; wherein the transactivator of the second cassette, when expressed as a protein, binds and transactivates the transactivator response element of the first cassette.

In some embodiments, the method comprises contacting the population of cells with the contiguous polynucleic acid molecule or a composition comprising said contiguous polynucleic aid molecule, wherein: a) the population of cells comprises at least one target cell type and two or more non-target cell types, wherein the target cell type(s) and the non-target cell types differ in levels of one or more endogenous miRNAs (e.g., at least 1, at least 2, at least 3, at least 4, at least 5, at least 6, at least 7, at least 8, at least 9, at least 10, at least 11, at least 12, at least 13, at least 14, at least 15, at least 20 endogenous miRNAs), such that the levels of the one or more endogenous miRNAs are at least two times higher (e.g., at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 1000 times higher) in each of the two or more non-target cells relative to each of the target cells; and b) the contiguous polynucleic acid molecule comprises cassette encoding a mRNA whose expression is operably linked to a transactivator response element, wherein the RNA comprises: a nucleic acid sequence of an output; a nucleic acid sequence of a transactivator; and one or more miRNA target sites corresponding to the one or more endogenous miRNAs; and wherein the transactivator, when expressed as a protein, binds and transactivates the transactivator response element of the cassette.

In some embodiments, the target cell type(s) and the non-target cell types differ in levels of one or more endogenous transcription factors (e.g., at least 1, at least 2, at least 3, at least 4, at least 5, at least 6, at least 7, at least 8, at least 9, at least 10, at least 11, at least 12, at least 13, at least 14, at least 15, at least 20 endogenous transcription factors), wherein the contiguous nucleic acid molecule further comprises one or more transcription factor response element corresponding to the endogenous transcription factor(s).

In some embodiments, the contacting with the host cell with a contiguous polynucleic acid molecule described above or a vector described above occurs via a non-viral delivery method. Examples include, but are not limited to, transfection (e.g., DEAE dextran-mediated transfection, CaPO₄-mediated transfection, lipid-mediated uptake, PEI-mediated uptake, and laser transfection), transformation (e.g., calcium chloride, electroporation, and heat-shock), gene transfer, and particle bombardment.

In some embodiments, the population of cells is contacted ex vivo (i.e., a population of cells is isolated from an organism, and the population of cells is contacted outside of the organism). In some embodiments, the population of cells is contacted in vivo.

As used herein, the term “endogenous”—in the context of a cell—refers to a factor (e.g., protein or RNA) that is found in the cell in its natural state. In some embodiments, an endogenous transcription factor may bind and activate a promoter element of a regulatory component of at least one cassette (e.g., a transcription factor response element). In some embodiments, an endogenous miRNA may complement a miRNA target site of a regulatory component or response component of at least one cassette.

In some embodiments, a “transactivator” and corresponding “transactivator response element” will be selected such that the transactivator will specifically bind to the “transactivator response element” but bind as little as possible to response elements naturally present in the cell. In some embodiments, the DNA binding domain of a transactivator protein will not efficiently bind native regulatory sequences present in the cell and, therefore, will not trigger excessive side effects.

In some embodiments a target cell and a non-target cell are different cell types.

In some embodiments, a target cell is a cancerous cell and a non-target cell is a non-cancerous cell. In some embodiments, a target cell may be a cancerous hepatocellular carcinoma cell or a cholangiocarcinoma cell and a non-target cell may be a parenchymal and non-parenchymal liver cells, including hepatocytes, phagocytic Kupffer cells, stellate cells, sinusoidal endothelial cells.

In some embodiments, a target cell is a hepatocyte and a non-target cell is a non-hepatocyte (e.g., a myocyte). In other embodiments, a target cell and a non-target cell are the same cell-type (e.g., both are hepatocytes), but nonetheless, differ in levels of at least one endogenous transcription factor and/or at least one endogenous miRNA. For example, a target cell may be a senescent muscle cell and a non-target cell may be a non-senescent muscle cell.

In some embodiments, the target cells are tumor cells and the cell-specific event is cell death. In some embodiments, the target cells are senescent cells and the cell-specific event is cell death. In some embodiments, the cell death is mediated by immune targeting through the expression of activating receptor ligands, specific antigens, stimulating cytokines, or any combination thereof. In some embodiments, the method further comprises contacting the population of cells with a prodrug or a non-toxic precursor compound that is metabolized by the output into a therapeutic or a toxic compound.

In some embodiments, the target cells differentially express a factor relative to wild-type cells (e.g., healthy and/or non-diseased) of the same type and the cell-specific event is modulating expression levels of the factor.

In some embodiments, output expression ensures the survival of the target cell population while the non-target cells are eliminated due to lack of output expression and in the presence of a cell death-inducing agent. In other embodiments, the output ensures the survival of the non-target cell population while the target cells are eliminated due to output expression and in the presence of a cell death-inducing agent.

In some embodiments, the target cells comprise a particular phenotype of interest such that output expression is limited to the cells of this particular phenotype.

In some embodiments, the target cells are a cell type of choice and the cell-specific event is the encoding of a novel function, through the expression of a gene naturally absent or inactive in the cell type of choice.

In some embodiments, the population of cells comprises a multicellular organism. In some embodiments, the multicellular organism is an animal. In some embodiments, the animal is a human.

IV. Methods of Diagnosing and/or Treating a Disease or a Condition

In some aspects, the disclosure relates to methods of diagnosing a disease or a condition (e.g., cancer) in a subject exhibiting one or more signs or symptoms of the disease or condition. As used herein, the term “diagnose” refers to a process of identifying or determining the nature and/or cause of a disease or condition. In some embodiments, the method comprises administering a contiguous polynucleic acid molecule described above, a vector described above, an engineered viral genome described above, or a virion described above to a subject exhibiting one or more signs or symptoms associated with a disease or condition, wherein the levels of the output indicates the presence or absence of the disease or condition.

In some aspects, the disclosure relates to methods of treating a disease or condition (e.g., cancer). As used herein, the term “treat” refers to the act of preventing the worsening of one or more symptoms associated with a disease or condition and/or the act of mitigating one or more symptom associated with a disease or condition. In some embodiments, the method comprises administering a contiguous polynucleic acid molecule described above, a vector described above, an engineered viral genome described above, or a virion described above to a subject having the disease or condition.

In some embodiments related to treating the disease or condition, the method of administration comprises an intravenous delivery of the vectors described above. In some embodiments, the method of administration comprises more than one act of intravenous delivery of the vectors described above. In some embodiments, the method of administration comprises an intratumoral delivery of the vectors described above, in one or more dosing. In some embodiments, the method of administration comprises a transarterial delivery of the vectors described above, in one or more dosing. In some embodiments, the method of administration comprises an intramuscular delivery, an intranasal delivery, subretinal delivery, or oral delivery,

In some embodiments, the method of treating the disease further comprises the administration of a pro-drug in one or more dosings. In some embodiments, the delivery off the prodrug is intravenous, transarterial, or intraperitoneal. In some embodiments, the prodrug is ganciclovir.

In some embodiments, the method of treating the disease further comprises the administration of another therapy such as a small molecule, a biologic, a monoclonal antibody, another gene therapy product, or a cell-based therapeutic product.

In some embodiments, the diseases or condition is cancer. Exemplary cancers that can be treated by the methods described herein include, but are not limited to, .hepatocellular carcinoma (HCC), metastatic colorectal cancer (mCRC), any other cancer metastasized to the liver, lung cancer, breast cancer, retinoblastoma, and glioblastoma.

Exemplary cancers that can be treated by the methods described herein include, but are not limited to, hepatocellular carcinoma (HCC), metastatic colorectal cancer (mCRC), lung cancer, breast cancer, retinoblastoma, glioblastoma.

In some embodiments, the cancer is hepatocellular carcinoma (HCC)). Indeed, therapeutic options for HCC are limited (Llovet and Lencioni, 2020), creating an urgent need to explore novel modalities for breakthroughs. The methods described herein significantly advance current HCC treatment methodologies.

### EXAMPLES

### Example 1. Multiplex Diagnostic Circuits Translate to Gene Therapy Vectors

Experiments were designed to assess whether logic gates put together from multiple disjointed components (i.e., one gene per plasmid and characterized in transient transfection of cell lines) could be re-engineered to fit into a therapeutically relevant vector and studied as a therapeutic candidate in an animal disease model. It was previously shown that integration of sensors for transcription factors (TF) SOX9/10 and HNF1A/B by a multi-plasmid system implementing an AND logic between these sensor's activity elicited a strong response when transiently transfected into HuH-7 cells (Angelici et al., 2016). SOX9 is a prognostic marker associated with advanced HCC (Richtig et al., 2017). Interestingly, the SOX9 response element is likely to be bound by SOX4, another TF whose overexpression is associated with a malignant HCC phenotype (Liao et al., 2008; Uhlen et al., 2017). HNF1A and HNF1B are known liver housekeeping factors (Harries et al., 2009); although, they are also expressed in other organs of the GI tract.

Experiments were designed to gauge whether the previously described multi-plasmid system could be adapted to a contiguous DNA cassette and eventually packaged in a viral vector. To this end, circuit components shown to implement the logic “SOX9/10 AND HNF1A/B” in a multi-plasmid setting (Angelici et al., 2016), comprising a SOX9/10-driven PIT-based activator (PIT::RelA or PIT::VP16) (Fussenegger et al., 2000), as well as a fluorescent output protein synergistically driven by PIT and HNF1A/B, were cloned between ITRs in an adeno-associated viral (AAV) transfer vector either in a divergent or convergent orientation (FIG. 1A). The resulting plasmids were transiently transfected into HEK293 cells, and the TF inputs SOX10 and HNF1A were expressed ectopically from TRE-driven plasmids to generate all four logical input combinations to this gate. Interestingly, while the trend was preserved in all four cases, the different variants differ markedly in their absolute ON levels when both inputs are present (FIG. 1B). The same constructs were also transfected into HuH-7 and HeLa cells, where the endogenous expression of SOX9/10 and HNF1A/B is expected to induce the circuit in the former and not activate it in the latter. In this case, the differences were less pronounced, yet the divergent orientation generated somewhat higher output.

The AND gate strategy is a way to activate the output in the desired cell type, and the augmentation of this activation designed by incorporation of intentional “Off” switches, equivalent to NOT gates, which would comprise additional safety layer in the context of a therapy. To this end, microRNA targets were incorporated in the 3′-UTR of the output gene, as well as in the 3′-UTR of the PIT-derived component. The choice of specific inputs, including miR-424, miR-126 and miR-122, was made on the basis of previously-performed profiling (Dastor et al., 2018). The miR-424 target was initially introduced, and the four resulting constructs (FIG. 1D) were again tested for their response to ectopic TF combinations in HEK cells (FIG. 1E) and in the presence of endogenous inputs in HuH-7 and HeLa cells (FIG. 1F). Marked and consistent differences were observed in performance. The convergent constructs failed to respond to the ectopic inputs in HEK cells and responded with greatly reduced intensity in HuH-7 cells, compared to the divergent ones. This fact highlights the complexity of the transition from circuits carried on disparate plasmids and circuits integrated on a contiguous backbone compatible with a gene therapy delivery vector. Next, the two divergent cassettes underwent more extensive logic characterization including both the TF and the miR-424 mimic input. Both constructs responded as expected, implementing the logic “SOX10 AND HNF1A AND NOT(miR-424)” (FIG. 1G). To confirm that high miR-424 expression also overrides output activation with endogenous TF inputs, miR-424 mimic was transfected into HuH-7 cells and was found to turn off output expression to an almost background level (FIG. 1H). Next, the miR-424 targets were replaced with miR-126 targets. The new set of constructs was tested only in HuH-7 cells with respect to its response to exogenous miR-126, and the results were similar to miR-424 and consistent with expectation (FIG. 1I). To conclude this design stage, the divergent constructs without miRNA targets, with miR-424 or miR-126 targets were evaluated for their capacity to distinguish HCC cell lines HuH-7 and HepG2 from HeLa cells (FIG. 1J).

The next step is the incorporation of the cassettes into viral vectors and their evaluation with respect to logic performance prior to preclinical translation. It is known that AAV-delivered genomes form concatemers in human cells (Duan et al., 2003), and this would comprise additional layer of complexity compared to the DNA cassette encoding the AAV genome but not packaged and delivered with the help of an AAV capsid. To this end, ITR-flanked genomes were used, and small quantities of DJ-pseudotyped (Grimm et al., 2008) AAV vectors were manufactured. The vectors were used to transduce two HCC cell lines, HepG2 and HuH-7, and two non-HCC cell lines, HeLa and HCT-116. The results showed high expression in the target cells and very low expression in non-target cells (FIG. 1K). Some additional effects are apparent, for example the reduction of the output expression obtained with a vector bearing a T424 targets in HuH-7 cells, compared to the vector without miRNA targets, which is much stronger than the reduction observed with naked DNA cassettes.

In order to get preliminary information which of the two miRNA targets (T424 or T126) would fare better in vivo, experiments were designed to assess which of them would perform a key protecting function (i.e., enable discrimination between HCC cells and healthy hepatocytes). Primary mouse hepatocytes were isolated for in vitro culture. The primary hepatocytes and the HCC cell were transduced with AAV-DJ packaged genetic reporters (Dastor et al., 2018) for miR-424, miR-126 as well as miR-122, a known liver miRNA that was shown to turn off gene expression efficiently in the liver in vivo (Dastor et al., 2018; Della Peruta et al., 2015) and that is known to be downregulated in a subset of HCC tumors (Coulouarn et al., 2009). The results of this testing (FIG. 1L) show that surprisingly, high expression counts of miR-424 and miR-126 in the liver did not translate to high biological knock-down activity in hepatocytes. Only miR-122 was consistently active. miR-122 was inactive in HepG2 cell line, but it showed partial activity in HuH-7 cell line, suggesting that the inclusion of this miRNA target would be beneficial for a subset of HCC tumors but not for all of them. Despite this fact, the circuit was further investigated with miR-122 for its specificity and antitumor potential in a pilot experiment setting. The impact of different miRNA target arrangements was also tested to assess how their number affects the overall output suppression in the presence of the miRNA input. Four different cassettes were tested, and it was found that increasing the number of targets, and placing the targets both in the output and in the PIT 3′-UTR, increases the repression (FIGS. 1M-1N). This provides another knob that can be used in two ways: to increase the knockdown of the output in not-target cells, but also decrease the knockdown in target cells that express partial level of the miRNA input.

### Example 2. Initial Evaluation of the First HCC-Targeting Circuit Variant in the Translational Context

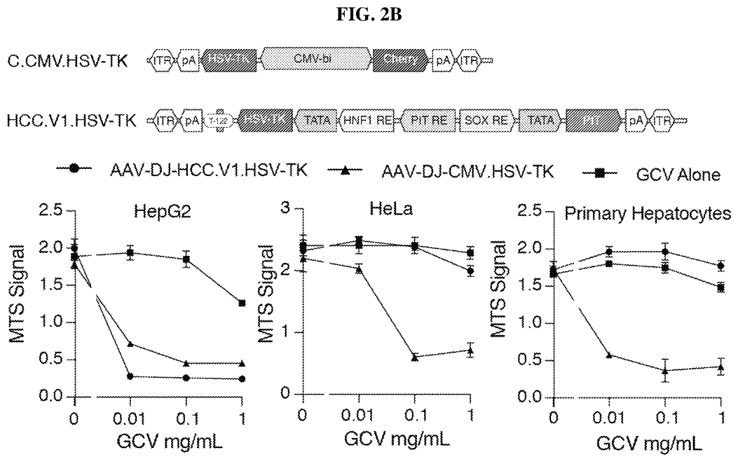

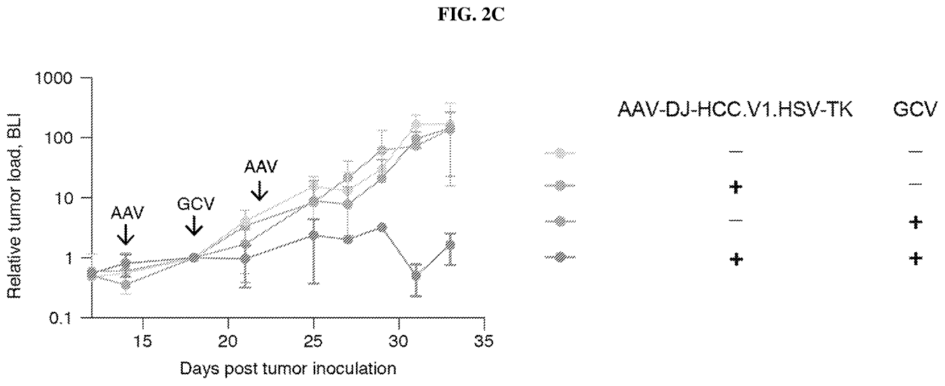

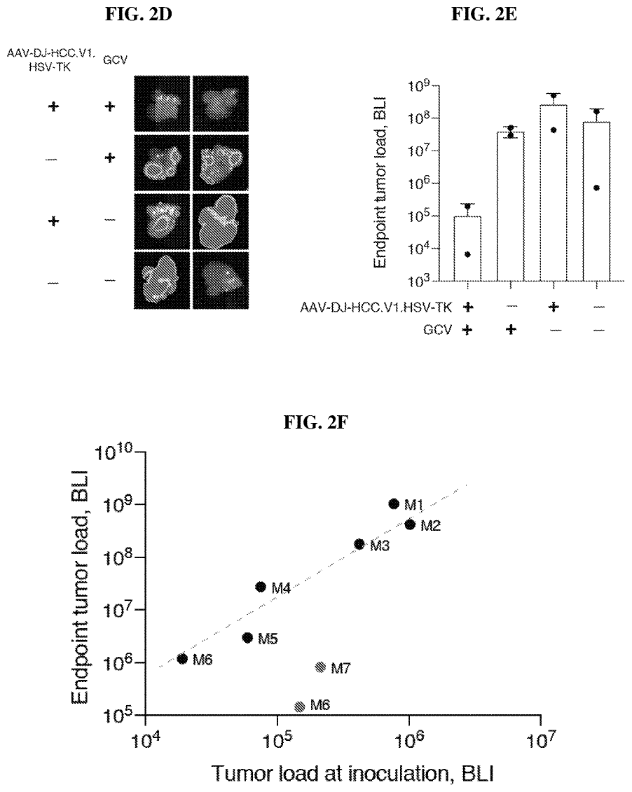

Based on the reporter investigation, a circuit variant was constructed bearing miR-122 targets. The PIT::VP16 activator variant was used due to its lower DNA payload and increased available footprint for the output gene. The circuit with mCherry output, dubbed HCC.V1-mCherry, was packaged into DJ-pseudotyped AAV vectors and re-tested in its ability to discriminate HCC cell lines from primary murine hepatocytes. The data highlight that the full circuit generates highly specific expression in HepG2 and Hep3B cell lines compared to primary hepatocytes, while in HuH-7 the circuit generates reduced output due to intermediate activity of miR-122 in these cell lines (FIG. 2A). Accordingly, this tumor-targeting program was evaluated in a pilot experiment in the context of orthotopic xenograft tumor model employing HepG2 cells in NSG mice. For the purpose of tumor establishment and tracking, HepG2 cells were stably modified with a lentiviral vector encoding an mCitrine fluorescent protein and firefly luciferase gene, and sorted for homogenous mCitrine expression. The tumors were established by splenic injection of 1M HepG2-LC cells and subsequent spleen dissection.

Prior to in vivo experiments, in vitro efficacy tests were performed comparing primary hepatocytes, HepG2 cells and HeLa cells as another negative control cell line. The vector, bearing HSV-TK output gene and dubbed AAV-DJ-HCC.V1-HSV-TK, requires GCV as a prodrug to elicit cytotoxicity with marked bystander effect (Freeman et al., 1993). The data (FIG. 2B) showed that indeed, HepG2 cells were selectively eliminated by the circuit as well as the control constitutive vector, while primary hepatocytes and HeLa cells were eliminated by the constitutive vector but were not affected by the circuit-bearing vector. Notably, the circuit eliminated HepG2 cells better than the constitutive control, highlighting the importance of high output expression driven by the tailored TF logic, compared to non-tailored constitutive vector.

To gauge antitumor efficacy in vivo, AAV-DJ-HCC.V1-HSV-TK was delivered to HepG2 tumor bearing mice in two consecutive injections, three days apart. The four experimental groups (n=2 in this pilot) included the AAV-DJ-HCC.V1-HSV-TK in combination with GCV regimen (treatment arm), the same vector alone without GCV, sham injection supplemented with GCV regimen, and a sham PBS injection and no GCV. Live imaging of tumor progression in the treated animals (FIG. 2C), and post-mortem analysis of the total tumor load in the liver with bioluminescence (FIGS. 2D-2E), clearly demonstrated that the gene therapy vector bearing the full circuit program in combination with the HSV-TK output and GCV regimen has strong antitumor activity, which is absent in any of the control arms. A low tumor load in one of the animals in the PBS control arm resulted from the initial poor tumor implantation (FIG. 2F), and in general all three control arms behaved the same, resulting in final tumor load proportional to the initial load, meaning that the tumor growth was governed by the same dynamics. The animals in the treatment arm of the pilot are obvious outliers, providing another evidence that the treatment was efficacious in reducing tumor load.

### Example 3. Engineering of a Tumor-Targeting Program with Higher Specificity and Broader Scope

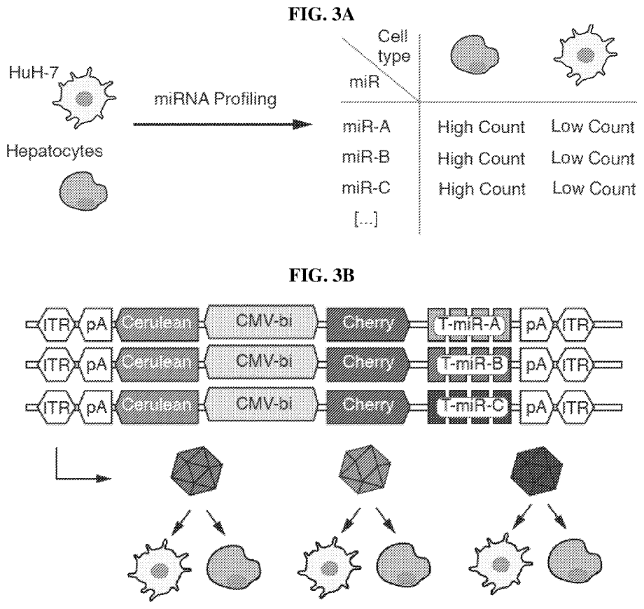

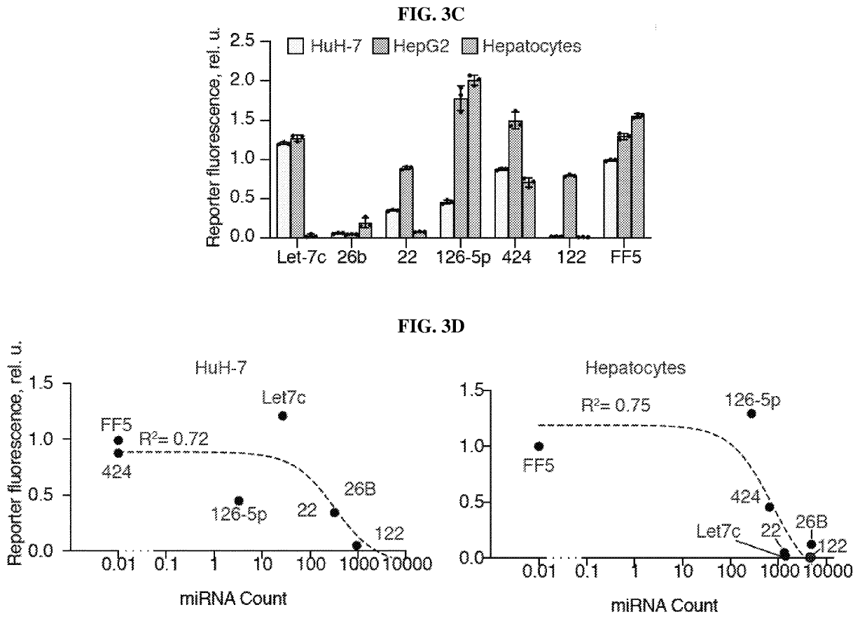

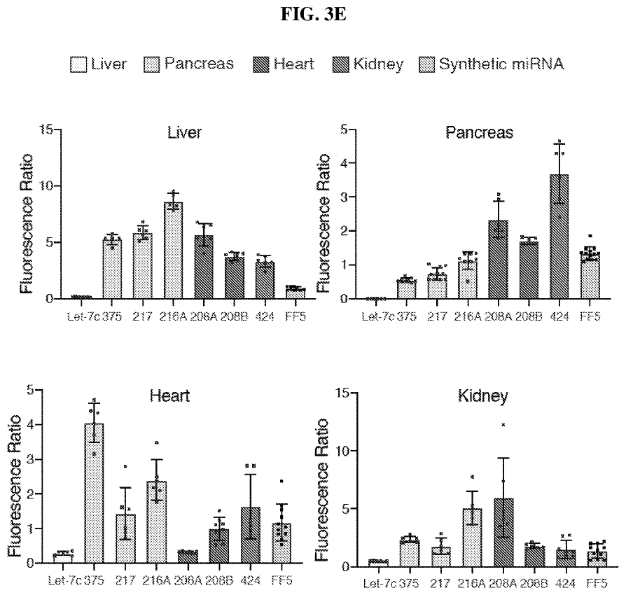

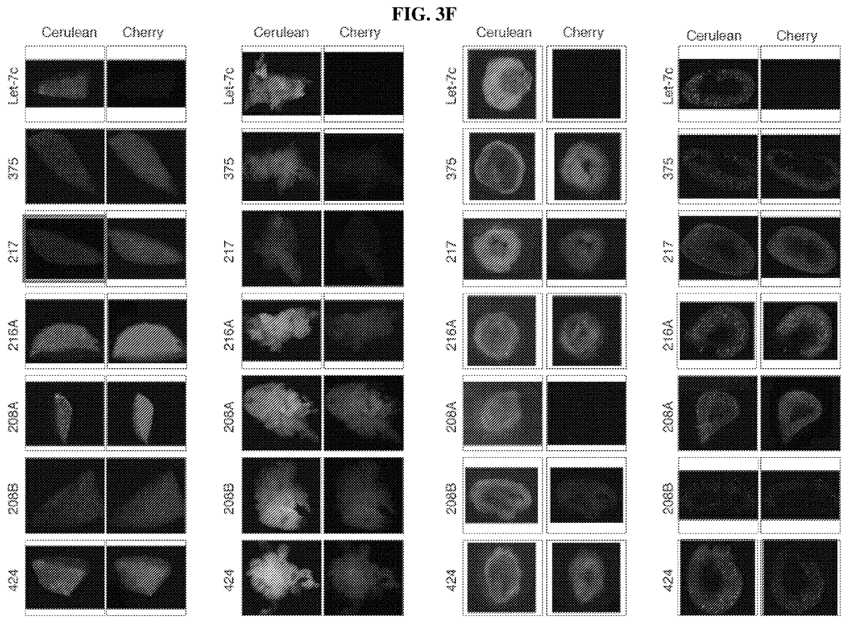

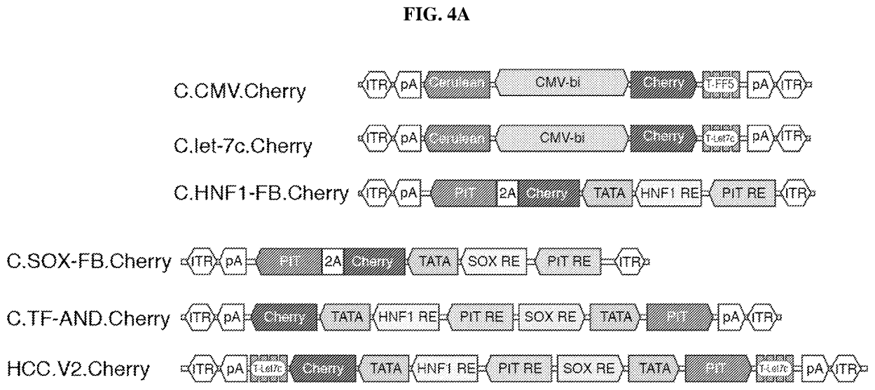

Encouraged by the outcome of the pilot experiment, it was sought to modify the tumor targeting program and in parallel to perform a more thorough evaluation of the circuit mechanism of action in vitro and in vivo. It was hypothesized that the combination of SOX9/10 and HNF1A/B inputs is a good starting point to restrict the expression to liver and liver tumors, however, previous data on miR-122 activity in vivo showed that its activity was restricted to liver (Dastor et al., 2018) and therefore one would have to rely on the TF-only component of the circuit for all other organs, which might become a problem if a vector capsid with broad organ specificity would be used. In addition, while miR-122 is a good classification marker to separate healthy hepatocytes from some HCC subtypes, it is not a universal HCC feature. Accordingly, the search was focused on miRNA inputs that might enable broader classification capacity of liver vs liver tumors, as well as protect additional organs. The point of origin for this search was 1) a miRNA profiling dataset obtained previously (Dastor et al., 2018) and 2) an extensive literature analysis for highly-expressed microRNAs in different organs. HuH-7 cells and healthy hepatocytes were profiled in the earlier experiments, and attempts were first made to identify a miRNA highly expressed in the hepatocytes but downregulated in HuH-7 cells (FIG. 3A). The miRNA set selected based on the count ratio in the NGS profiling dataset, included miR-122 (as a reference), miR-424, miR-126-5p, miR-22, miR-26b and let-7c. Bidirectional miRNA reporters (Dastor et al., 2018) were constructed and packaged into AAV-DJ vectors, to ensure high delivery efficiency to primary hepatocytes in vitro (FIG. 3B). Biological activity of the miRNA candidates was measured in HuH-7, HepG2, and primary isolated murine hepatocytes. Of the tested miRNAs, let-7c showed the highest differential activity; moreover, it was downregulated in both HuH-7 and HepG2 cells (FIG. 3C). Interestingly, retrospective analysis (FIG. 3D) comparing the NGS counts with the biological activity shows only a very superficial correlation, highlighting the importance of functional testing of candidate inputs.

Literature search and the examination of the profiling dataset for potential organ-protecting miRNA resulted in a set of miRNAs: miR-424 (kidney and other organs), miR-208a and miR-208 (heart), miR-216A, miR-217, and miR-375 (pancreas). Let-7c, a candidate for liver protection found based on the in vitro screening campaign, was added to this list. For each of these miRNAs, a bidirectional reporter was engineered and packaged in a B1-pseudotyped AAV vector (Choudhury et al., 2016), chosen due to its broad biodistribution. A control vector was made bearing a presumably neutral miRNA target (“TFF5”). (However, as the data revealed, this target was responding to miRNA inputs in at least some organs.) The vectors were injected systemically into healthy mice, and reporter expression was evaluated 3 weeks post-injection in the various organs. Strong biodistribution was found in liver, pancreas, heart and kidney, and the analysis was focused on these organs. Let-7c was the only miRNA from the set that showed potential as a healthy liver-specific input in vivo. In the pancreas in vivo, both miR-217 and miR-375 showed activity as expected from literature data; however, let-7c had the strongest response. In the heart, miR-208a and miR-208b showed activity consistent with prior data, yet again let-7c had the strongest response. Lastly, miR-424 was active in the kidney as expected, however, in this organ as well let-7c gave the strongest effect (FIG. 3EF).

In summary, the combination of in vitro and in vivo data showed that for the purpose of this study, let-7c could serve as a “universal” input, playing a role of a protective miRNA input for multiple organs at once and at the same time, being strongly downregulated in both HCC cell lines used in the tumor study. Accordingly, the next iteration of the circuit, dubbed HCC.V2, implements the program “SOX9/10 AND HNF1A/B AND NOT(let-7c)”.

### Example 4. Mechanism of Action In Vitro and In Vivo

Using AAV-DJ capsid as an efficient vehicle for cell transduction in vitro, and AAV-B1 as a capsid with broad biodistribution in vivo, an extensive mechanistic study of the AAV-packaged circuit was performed. Earlier in the study, the logic programs were analyzed and validated by transfecting circuit-carrying plasmid DNA into a background cell line that does not express any of the inputs; and then by systematic ectopic expression of all possible input combinations, comparing the results to the expectation. In the case of a viral vector, this strategy is now longer valid, because it is next to impossible to co-deliver individual ectopic inputs when the circuit itself is delivered via AAV transduction. Indeed, the more interesting question is how the vector responds to endogenously expressed inputs, because the cell classification in the context of a therapy has to rely on, and adequately respond to, endogenous inputs. A proof of mechanism thus comprises the question whether the output of the full circuit in a cell type is consistent with the activity of individual circuit inputs in these cells and the logic program of the circuit.

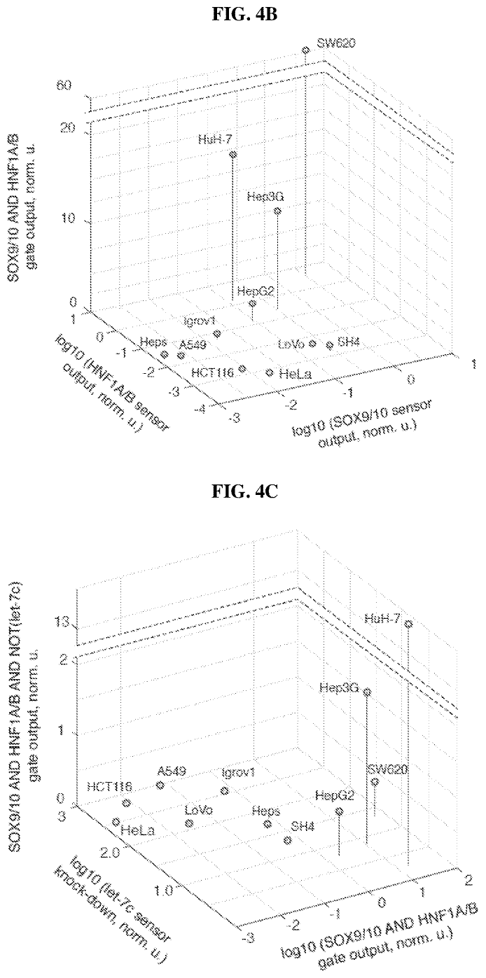

Accordingly, individual genetic sensors were created and packaged into AAV-DJ for every circuit input (AAV-DJ.C.SOX-FB.mCherry and AAV-DJ.C.HNF1-FB.mCherry for SOX9/10 and HNF1A/B feedback-amplified sensors, respectively); let-7c sensor (AAV-DJ.C.let-7c.mCherry); a partial circuit implementing AND gate only (AAV-DJ.C.TF-AND.mCherry); a full circuit (AAV-DJ.HCC.V2.mCherry); and a constitutive reporter serving as a reference (AAV-DJ.C.CMV.mCherry) (FIG. 4A). The outputs of these constructs were measured in 10 cell lines and primary hepatocytes. The results (FIGS. 4B-4C) show that the response of the multi input circuit is consistent with the expression of the individual inputs, confirming that the mechanism of action is preserved between the plasmid-based and viral vector-packaged system. Strong response of both individual sensors for SOX9/10 and HNF1A/B is needed to trigger high response of the TF-AND gate; and strong response of the TF-AND gate and the lack of response of the let-7c sensor is required to achieve high output of the complete program.

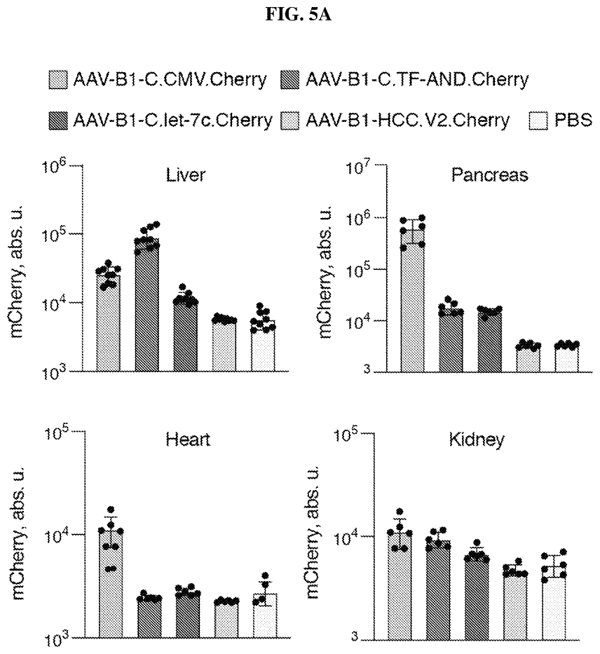

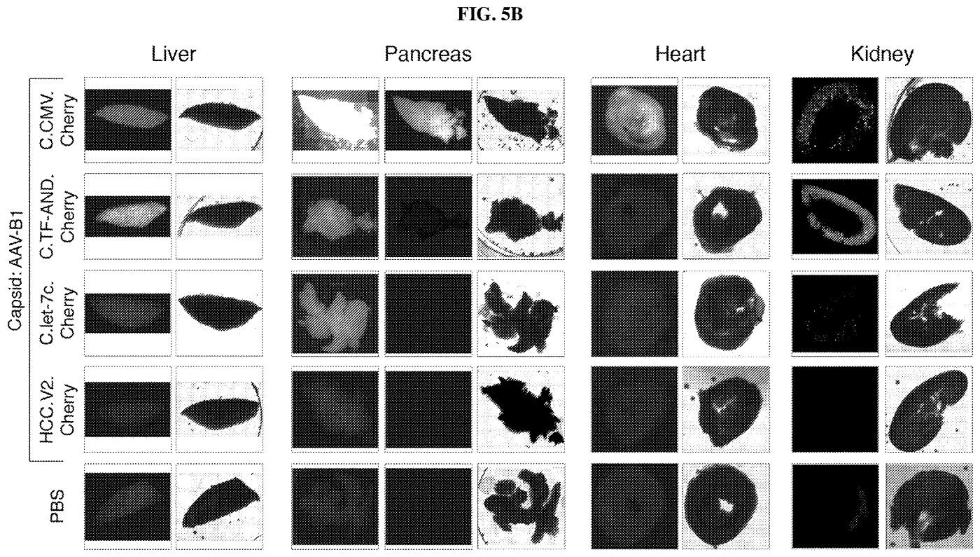

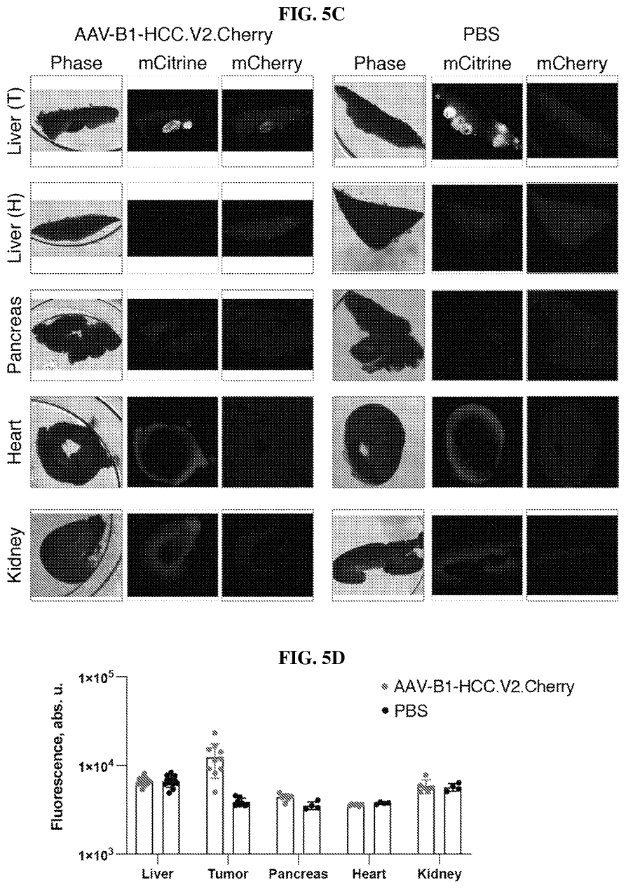

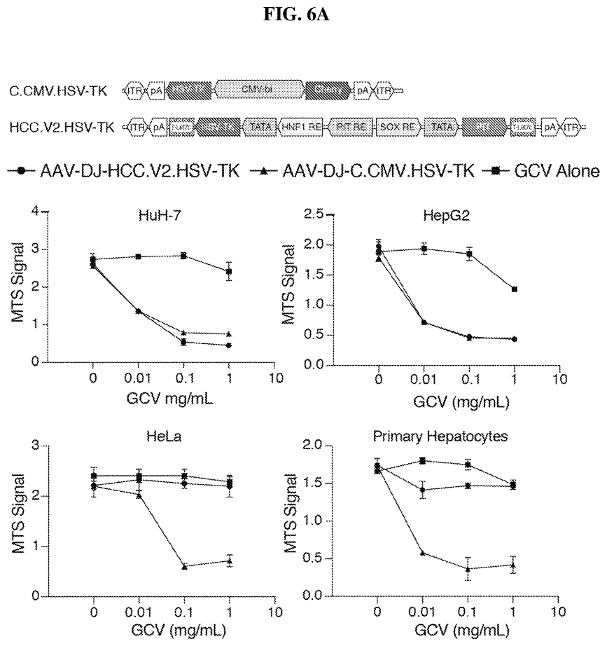

For in vivo characterization, B1-pseudotyped vectors packaging, respectively, a constitutive control AAV-B1.C.CMV.mCherry, a TF-only AND gate AAV-B1.C.TF-AND.mCherry, a let-7c reporter AAV-B1.C.let-7c.mCherry, and a full circuit AAV-B1.HCC.V2.mCherry, and expressing mCherry as the output, were systemically injected into mouse tail vein and the mCherry expression was evaluated 3 weeks post-injection in various organs. The expression was quantified in fresh organ slices by image processing. The results (FIGS. 5A-5B) highlight the complex synergistic action of the multiple inputs and their diverse role in different organs. In the liver, the AND-gate resulted in the reduction of the number of positive cells compared to the constitutive control, but in elevated expression on cells that exhibited positive expression. The let-7c reporter showed reduced expression compared to control, but the residual expression was clearly above background. The complete circuit resulted in expression virtually indistinguishable from background. In the pancreas, the AND gate-controlled expression and let-7c controlled expression resulted in large reduction in output expression, yet in each case the expression was above background. As in the liver, the complete targeting program did not generate any detectable expression above background. In the heart, either the AND gate or the let-7c rendered background-level expression on their own, and when combined in a complete circuit. In the kidney the situation is similar to pancreas, in that neither AND gate nor let-7c regulation bring down the expression to background, while the complete program does. In summary, the dataset strongly supports the hypothesis that a multi-input logic circuit is required to achieve highly efficient de-targeting from healthy organs in vivo; the synergistic effect of multiple inputs, as abstracted by the logic program “SOX9/10 AND HNF1A/B and NOT(let-7c)” is apparent in three out of four cases. Experiments were then designed to determine if the same program is able to efficiently target tumors in vivo, and injected a B1-typed AAV-B1.HCC.V2.mCherry circuit with mCherry output to tumor-bearing NSG mice. The data (FIG. 5C) show that indeed, the tumor is targeted specifically and efficiently in vivo while other organs do not express the output, consistent with data in FIGS. 5A-5B.

### Example 5. Antitumor Efficacy In Vitro and In Vivo

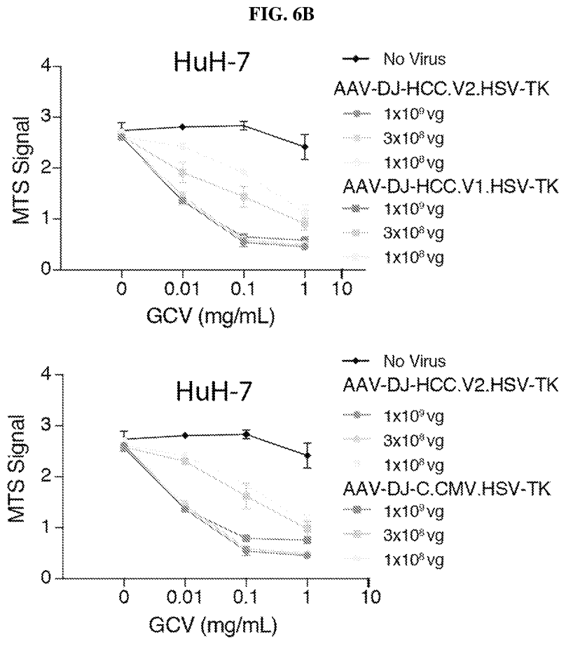

As the circuit program showed excellent tumor-specific expression and de-targeting from major organs in vivo, detailed evaluation of its antitumor activity was performed using HSV-TK enzyme in combination with the prodrug ganciclovir as a benchmark antitumor actuator. The circuit was dubbed HCC.V2-HSV-TK. The testing was done along the lines similar to the pilot experiment (FIG. 2) but with larger animal groups and extended number of experimental arms. DJ-pseudotyped vectors, including a constitutive control and a complete circuit were manufactured and their dose-response to ganciclovir evaluated in HuH-7, HepG2, and HeLa cell lines and in primary hepatocytes cultured in vitro. As expected, Huh-7 and HepG2 cells were targeted equally by the constitutive vector and the circuit AAV-DJ.HCC.V2-HSV-TK, while both HeLa negative control cells and primary hepatocytes were sensitive to the constitutive vectors but were not eliminated by the fully furnished circuit (FIG. 6A). In addition, AAV-DJ.HCC.V2-HSV-TK is more potent than AAV-DJ.HCC.V1-HSV-TK in HuH-7 cells, due to the use of let-7c sensor which is not downregulated in these cells. However, AAV-DJ.HCC.V1-HSV-TK was still active in HuH-7 cells due to incomplete shut-down by miR-122 (FIG. 6B).

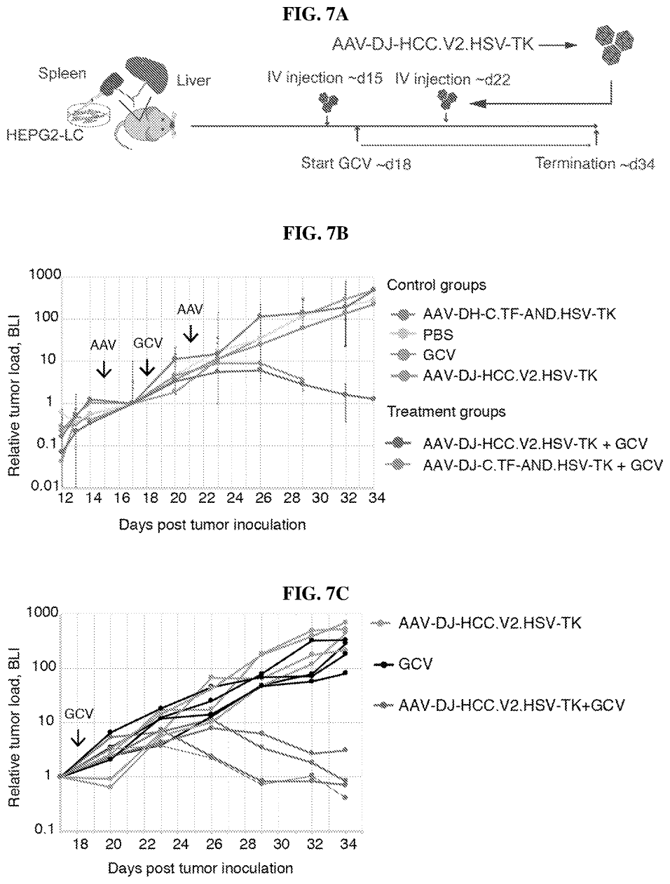

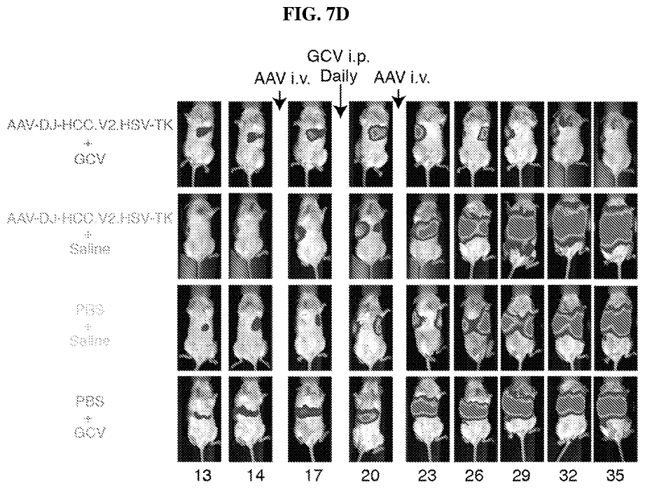

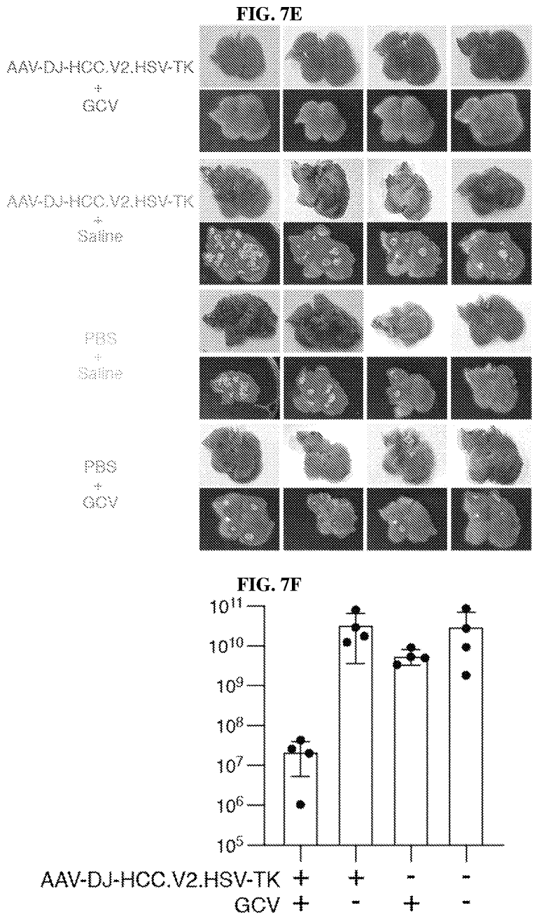

Next, DJ-pseudotyped AAV vectors harboring the circuit were delivered systemically to HepG2-LC tumor-bearing mice (FIG. 7A). The experimental arms without ganciclovir included the sham injection (saline); the vector AAV-DJ.C.TF-AND-HSV-TK encoding the TF-AND program; and the vector encoding the full circuit AAV-DJ.HCC.V2-HSV-TK. The arms with ganciclovir mirrored the arms above with respect to tail vein delivery of a vector or a sham, followed by a regimen of ganciclovir injections; namely: included sham injection+GCV; AND-gate circuit+GCV; and a complete circuit+GCV. The animals (n=4 per arm) were followed for their tumor load using in vivo bioluminescence, and for their well-being using score sheet criteria. The data (FIGS. 7B-7F) indicate that mice treated with the vector harboring the full HCC.V2-HSV-TK program furnished with HSV-TK output and supplemented with GCV regimen, show robust and reproducible containment and then regression of their tumor load, while the control groups without GCV, or the group that was only injected with GCV, show exponential tumor load increase over time. The vector encoding the AND gate with HSV-TK output, AAV-DJ-C.TF-AND-HSV-TK, exhibited similar antitumor effect compared to AAV-DJ.HCC.V2-HSV-TK, yet also triggered strong adverse effects, and therefore the animals in this arm had to be euthanized prior to scheduled completion. The arm treated with the complete AAV-DJ.HCC.V2-HSV-TK circuit, on the other hand, showed extended reduction in tumor load without obvious adverse effects. These results unequivocally illustrate the tight link between the targeting specificity in vivo (FIGS. 5A-5D) and the magnitude of adverse effects in vivo. Accordingly, in the future the presence of output expression outside of the tumor as gauged from a fluorescent output expression, will constitute a pre-screening stage that need not be evaluated for their toxicity with functional outputs.

### Example 6. In Vivo Comparison of AAV-B1 and AAV-DJ Pseudotypes Circuit Driven HCC Targeting

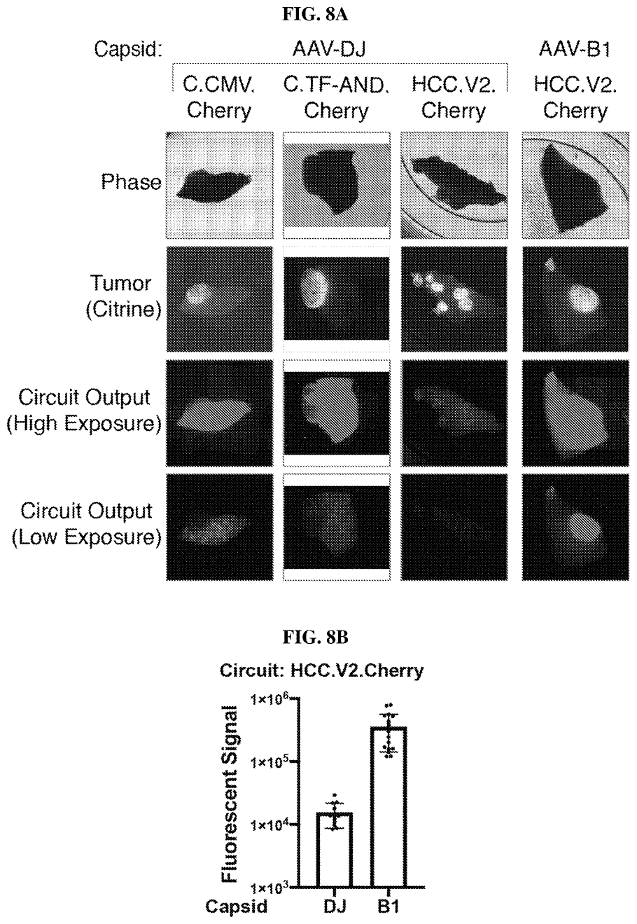

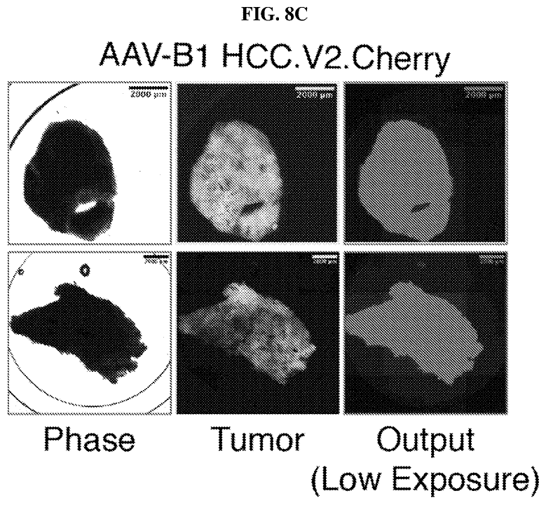

Given the broad tropism and strong in vivo transduction observed for the B1-typed AAV capsid and the extensive multi-organ detargeting accomplished placing gene expression under the control of the HCC.V2 program, it was reasoned that the resulting B1-typed AAV-B1.HCC.V2 circuit might yield high tumor transduction without compromising selectivity. To investigate this possibility, circuit output (mCherry) was compared when the AAV-B1.HCC.V2-mCherry full circuit output is delivered using a B1 capsid in place of the DJ capsid used in previous efficacy studies. The data (FIG. 8A) show that, when administered at the same dosage, the B1 typed circuit vastly outperforms the tumor expression levels of all DJ variants (AAV-DJ.HCC.V2.mCherry, TF-only AND gate AAV-DJ.C.TF-AND.mCherry or AAV-DJ.C.CMV.mCherry) while keeping its selectivity towards neighboring liver tissue. The intratumoral output expression was about 40 times higher (FIG. 8B) and resulted in intense fluorescence even in the core section of large tumor nodules. The strong selective expression combined with tumor penetration suggest circuit targeting, coupled to B1-typed capsid as promising candidates for HCC gene therapies.

### Example 7. Combination of miR-let-7c and miR-122

In vitro efficacy data show that while HCC.V1 fully protects hepatocytes even at high dosage (FIG. 2B), the same program shows only a partial reduction in HUH-7 cell killing efficiency when compared to HCC.V2 (FIG. 5B) and results in almost comparable performance for high viral dosage. This difference is in agreement with the tighter gene repression observed in Hepatocytes compared to HUH-7 cells (FIG. 2A).

As established herein, changes in the number and arrangement of miR-122 targets can be used to modulate the repression strength resulting in different expression levels in cell lines with different miR-122 levels (FIG. 1M). It was hypothesized that a reduction in miR-122 repression efficiency through changes in target number, arrangement, or via the use of imperfectly complementary targets could be used to increase circuit efficacy in HUH-7 (even at lower viral dosage), at the risk of a partial reduction of liver detargeting.

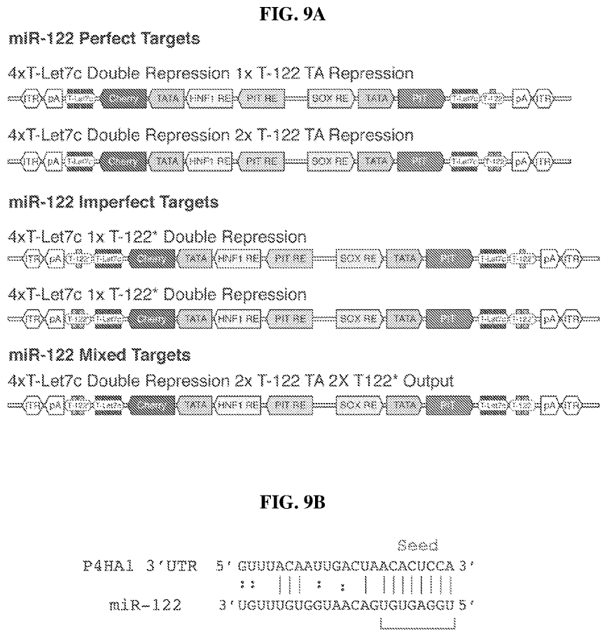

From these data, a HCC.V3 circuit that combines the miR-Let7c targets from HCC.V2 with weaker miR-122 repression (FIG. 9A) is expected to outperform both the HCC.V3 circuit and the HCC.V2 circuit. The repression strength elicited by miR-122 can be tuned by changing the number and positioning of T-122 targets, by introducing imperfectly complementary targets or by a combination of the two approaches. Imperfectly complementary target can be obtained by introducing random mutations in the sequence flanking the miRNA seed sequence or by using miR-122 targets derived from conserved 3′ UTR of genes regulated by the miRNA (FIG. 9B). The candidate that maximize the desired combination of liver protection and efficacy against HCC cells (HUH-7 in particular) can be selected.

It is expected that HCC.V3 will exhibit generalized miRNA detargeting from major organs (Let-7c) and benefit from combined protection (Let7c and miR-122) in the liver without significant reductions in its efficacy both in HepG2 and HUH-7. Being the organ with the highest biodistribution for most viral vectors, achieving the tightest possible liver detargeting is particularly desirable and might lead to further increases in the therapeutic window.

### Example 8. Discussion

This disclosure shows a path to the clinical translation of logic gene circuit approaches. Three underlying pillars are necessary to support such a translation, namely: (1) the knowledge of the molecular make up of a disease; (2) the availability of a platform that enables taking advantage of this knowledge; and (3) the translatability of this platform to a clinically-relevant therapeutic modality come together to deliver a viable therapeutic candidate with promising in vitro and in vivo efficacy and safety profile. The extensive mechanistic characterization described herein highlights the unique properties of multi-input cell classifiers, constructed in rational bottom-up fashion following a systematic procedure, compared to its individual components. Importantly, it is demonstrated herein that targeting specificity as gauged by reporter outputs tightly correlates with both efficacy and adverse effects in vivo.

Specific expression and other modalities of therapeutic control, such as timing and dosage, are the next frontier of gene therapy not only for cancer but also for other indications. A large effort has been invested into the development of novel capsids with preferential tissue targeting, as well as promoter elements for specific tissue expression. Notably, both lines of work rely on extensive screening of large libraries and they do not guarantee success; moreover, the claim of specificity can only be made in the presence of large panel of counter samples. For human therapy, these samples must be of human origin. Due to the large diversity of human tissues, superimposed on the large library sizes for capsid and/or promoter screen, will make this effort prohibitively complex. The bottom-up approach described herein uses rational design to create combinatorial specificity from multiple individual inputs. Narrowing down the candidate input space by profiling puts the engineering of complex programs able to address heterogeneous cell populations (as in our example of Huh-7 and HepG2 cells) on a rational, forward design background. This approach does not exclude the use of targeted capsids or specific promoters: they can be applied as needed. However, for a disseminated disease such as cancer, broad tropism capsid may be preferential; the burden of specific expression is then shifted to the classified program encoded in the genetic payload of the therapy. In other cases, capsid specificity and the classifier program can be used synergistically to achieve the best desired effect.

Efficient penetration of large multifocal tumors in the liver was achieved in vivo following a single systemic injection (FIGS. 5C-5D and FIGS. 8A-8C), and this provides strong evidence that even a single injection is capable of delivering a payload to disseminated and well-vascularized tumors, such as HCC. An output with a bystander effect is then able to efficaciously treat these tumors.

### Example 9. Materials and Method for Examples 1-8

Cell lines: HuH-7 cells were purchased from the Health Science Research Resources bank of the Japan Health Sciences Foundation (Cat-#JCRB0403) and cultured at 37° C., 5% CO2 in DMEM, low glucose, GlutaMAX (Life technologies, Cat #21885-025), supplemented with 10% FBS (Sigma-Aldrich, Cat #F9665 or Life technologies, Cat #10270106) and 1% Penicillin/Streptomycin solution (Sigma-Aldrich, P4333). Hep G2 cells were purchased from ATCC (Cat#HB-8065) and cultured at 37° C., 5% CO2 in RPMI (Gibco A10491-01) supplemented with 10% FBS (Sigma-Aldrich, Cat #F9665 or Life Technologies, Cat #10270106) and 1% Penicillin/Streptomycin solution (Sigma-Aldrich, P4333). HeLa cells were purchased from ATCC (Cat #CCL-2) and cultured at 37° C., 5% CO2 in DMEM, high glucose (Life technologies, Cat #41966), supplemented with 10% FBS (Sigma-Aldrich, Cat #F9665 or Life Technologies, Cat #10270106) and 1% Penicillin/Streptomycin solution (Sigma-Aldrich, P4333). Hep3B cells were purchased from ATCC (Cat#HB-8064) and cultured at 37° C., 5% CO2 in DMEM, low glucose, GlutaMAX (Life technologies, Cat #21885-025), supplemented with 10% FBS (Sigma-Aldrich, Cat #F9665 or Life technologies, Cat #10270106) and 1% Penicillin/Streptomycin solution (Sigma-Aldrich, P4333). HCT-116 cells were purchased from Deutsche Sammlung Von Microorganismen and Zellkulturen (DMZ), DMZ No ACC-581 and cultured at 37° C., 5% CO2 in DMEM GlutaMAX (Life technologies, Cat #31966-021), supplemented with 10% FBS (Sigma-Aldrich, Cat #F9665 or Life technologies, Cat #10270106) and 1% Penicillin/Streptomycin solution (Sigma-Aldrich, P4333). SW-620 cells were purchased from ATCC (Cat #CCL-227) and cultured at 37° C., 5% CO2 in DMEM GlutaMAX (Life technologies, Cat #31966-021), supplemented with 10% FBS (Sigma-Aldrich, Cat #F9665 or Life Technologies, Cat #10270106) and 1% Penicillin/Streptomycin solution (Sigma-Aldrich, P4333). LoVo cells were purchased from ATCC (Cat #CCL-229) and cultured at 37° C., 5% CO2 in DMEM GlutaMAX (Life technologies, Cat #31966-021), supplemented with 10% FBS (Sigma-Aldrich, Cat #F9665 or Life Technologies, Cat #10270106) and 1% Penicillin/Streptomycin solution (Sigma-Aldrich, P4333). A549 cells were purchased from ATCC (Cat #CCL-185) and cultured at 37° C., 5% CO2 in DMEM GlutaMAX (Life technologies, Cat #31966-021), supplemented with 10% FBS (Sigma-Aldrich, Cat #F9665 or Life Technologies, Cat #10270106) and 1% Penicillin/Streptomycin solution (Sigma-Aldrich, P4333). SH4 cells were purchased from ATCC (Cat #CCL-185) and cultured at 37° C., 5% CO2 in DMEM GlutaMAX (Life technologies, Cat #31966-021), supplemented with 10% FBS (Sigma-Aldrich, Cat #F9665 or Life Technologies, Cat #10270106) and 1% Penicillin/Streptomycin solution (Sigma-Aldrich, P4333). IGROV1 cells are part of the NCI-60 panel and were obtained by NCI (NIH). The cells were cultured at 37° C., 5% CO2 in RPMI (Gibco A10491-01) supplemented with 10% FBS (Sigma-Aldrich, Cat #F9665 or Life Technologies, Cat #10270106) and 1% Penicillin/Streptomycin solution (Sigma-Aldrich, P4333).

Creation of Luciferase and mCitrine Stable Cell Line (HepG2 LC): An HepG2 cell line stably expressing mCitrine and Luciferase (HepG2 LC) was created via TALEN editing of the AAVS locus. 4×10⁵ HepG2 cells were seeded in a 6-well plate and transfected after 24 h with a total of 2 μg DNA with Lipofectamine 2000. The transfection mix was composed as follows: 500 ng hAAVS1 1 L TALEN (pIK11), 500 ng hAAVS1 1R TALEN (pIK12) and 1 μg of Luciferase 2A Citrine under the control of a EF1A Promoter (pIK014). Transformed cells were expanded and kept in culture for 3 weeks in order to dilute the expression arising from transient transfection. After 3 weeks the mCitrine⁺ bulk population (<1%) was sorted using a BD FACS Aria III. The resulting 20.000 cells were seeded in a 24-Well plate in RPMI supplemented with 20% FBS for the first week to facilitate the initial recovery. The cells were cultured and expanded for 2 weeks to select for cells with stable transgene expression and avoid clones prone to be silences. Single mCitrine⁺ clones were sorted in a 96-well plate, cultured in RPMI supplemented with 20% FBS and expanded. Three different high expressing clones were selected and the best was used for successive experiments. Bioluminescence of the clone was measured for 5 min using the PhotonIMAGER RT (Biospace Laboratories) to confirm Luciferase expression.

Viral vector plasmid and virus production: Single-stranded (ss) AAV vectors were produced and purified as previously described. (Paterna 2004, Conway 1999) Briefly, human embryonic kidney cells (HEK293) expressing the simian virus large T-antigen (293T) were cotransfected with polyethylenimine (PEI)-mediated AAV vector plasmids (providing the to-be packaged AAV vector genome), AAV helper plasmids (providing the AAV serotype 2 rep proteins and the cap proteins of the AAV serotype of interest) and adenovirus (AV) helper plasmids pBS-E2A-VA-E4 (Glatzel 2000) in a 1:1:1 molar ratio. 96 to 120 h post transfection HEK293T cells were collected and separated from their supernatant by low-speed centrifugation (15 min at 1500 g/4° C.). AAV vectors released into the supernatant were PEG-precipitated overnight at 4° C. by adding PEG 8000 solution (final: 8% v/v) and NaCl (final: 0.5 M). PEG-precipitation was completed by low-speed centrifugation (60 min at 3488 g/4° C.). Cleared supernatant was discarded and the pelleted AAV vectors resuspended in AAV resuspension buffer (150 mM NaCl, 50 mM Tris-HCl, pH 8.5). HEK293T cells were resuspended in AAV resuspension buffer and lysed by Bertin's Minilys Homogenizer in combination with 7 mL soft tissue homogenizing CK14 tubes (two 1 min cycles at 5000 rpm/RT, intermitted by >4 min cooling at −20° C.). The crude cell lysate was treated with the BitNuclease endonuclease (75 U/mL, 30 to 90 min at 37° C.) and cleared by centrifugation (10 min at 17 000 g/4° C.). The PEG-pelleted AAV vectors were combined with the cleared lysate and subjected to discontinuous density iodixanol (OptiPrep, Axis-Shield) gradient (isopycnic) ultracentrifugation (2 h 15 min at 365 929 g/15° C.). Subsequently, the iodixanol was removed from the AAV vector containing fraction by three rounds of diafiltration (ultrafiltration) using Vivaspin 20 ultrafiltration devices (100 000 MWCO, PES membrane, Sartorius) and 1× phosphate buffered saline (PBS) supplemented with 1 mM MgCl₂ and 2.5 mM KCl according to the manufacturer's instructions. The AAV vectors were stored aliquoted at −80° C. Encapsidated viral vector genomes (vg) were quantified using the Qubit 3.0 fluorometer in combination with the Qubit dsDNA HS Assay Kit (both Life Technologies). Briefly, 5 μL of undiluted (or 1:10 diluted) AAV vectors were prepared in duplicate. One sample was heat-denatured (5 min at 95° C.) and the untreated and heat-denatured samples were quantified according to the manufacturer's instructions. Intraviral (encapsidated) vg/mL were calculated by subtracting the extraviral (nonencapsidated; untreated sample) from the total intra- and extraviral (encapsidated and nonencapsidated; heat-denatured sample).

Cell preparation for in vivo injection: HepG2 LC cells were cultured and passaged until 70-80% confluence in T-75 or T-150 flasks. For in vivo injection we used cells with low passage number (passage 12 or less) to minimize silencing of the reporter gene. Cells were detached by removing the growth medium, washing with PBS (10 ml for T-75 or 20 ml for T-150), and dissociating the cells with Trypsin (Gibco, 25200056) (2 ml for T-75 or 6 ml for T-150 Flask) for 5 min at 37° C. The cell suspension was diluted with 8 mL (T-75) or 24 ml (T-150) of PBS, gently resuspended by pipetting, and subsequently filtered in a 50 ml Falcon tube using a 100 μm filter to obtain a single cell suspension. Additional PBS was used to wash the filter 10 ml (T-75) or 20 ml for T-150 further diluting the cells to a total volume of 20 ml (T-75) or 50 ml (T-150). The cell suspension was centrifuged at 498 rpm at 4° C. for 9 min. The cell pellet was washed with 20 ml of PBS and centrifuged at 498 rpm at 4° C. for 6 min two more times to remove any trace of trypsin. The procedure is carried out with one or more flasks and tubes depending on the number of cells needed for the experiment. Each pellet is resuspended in a small amount of PBS (250-300 ul for each pellet) and a small aliquot is diluted (1:50 and 1:100) for manual counting of live cells using Neubauer chamber and trypan blue. At least four independent counts were taken per cell suspension and the average value was used to determine the number of cells to be injected. Cell suspension was inspected visually under the microscope to verify the absence of large clumps. At the end the volume was adjusted with PBS to about 2×10⁷ cells/mL. The cell suspension was kept on ice for the duration of the surgeries, given the high cell concentration the cells require resuspension before each injection. In order to minimize manipulation and improve viability the cells are divided in multiple stocks (2-3 tubes). We note that both the presence of cell clumps and the presence of residual trypsin or other cell-dissociation reagents is toxic and potentially life-threatening to the animals.

Xenograft mouse liver mouse model: All animal procedures were performed in accordance with the Swiss federal law and institutional guidelines of Eidgenössische Technische Hochschule (ETH) Zurich, and approved by the animal ethics committee of canton Basel-Stadt. Eight to ten-week-old immunodeficient NSG mice (NOD.Cg-Prkdcscid Il2rgtm1Wjl/SzJ, Charles River, Sulzfeld, Germany) were housed in a specific-pathogen-free facility. To generate the mouse liver tumors derived from human tumor cells, NSG mice were anesthetized with inhalational isoflurane. Using aseptic surgical technique, a left subcostal incision of 1-1.5 cm was made and the spleen was exposed. 10⁵ HepG2 cells in 50 μl PBS were injected into the lower lobe of spleen using a 27-gauge needle. Immediately upon removal of the needle the lower pole of the spleen was ligated. A 10-minute draining was allowed for the majority of cells to reach the liver for colonization before the major splenic vasculature was ligated and the spleen is removed. The abdominal incision was then closed with sutures. The tumor growth in mice was monitored by bioluminescence imaging 2-3 times per week (PhotonIMAGER RT, Biospace Lab).

In vivo delivery of reporter AAVs and gene expression analysis by fluorescent microscopy and flow cytometry: To visualize circuit output expression in vivo, 2×10¹² vg (viral genomes) of AAVs encoding mCherry output or PBS were administered as a single dose through tail vein 2 weeks after tumor cell transplantation. After 3 weeks mice were euthanized and immediately perfused transcardially with 50-70 mL HBSS containing 10 or 25 U/mL heparin (Sigma-Aldrich) to remove autofluorescent red blood cells. The organs and tissues (liver, lungs, brain, pancreases, skeletal muscles, heart and kidneys) were harvested and fresh tissue slices were prepared and kept on ice in PBS. The expression of mCherry was analyzed immediately by fluorescent microscopy.

In vivo delivery of therapeutic AAVs and prodrug treatment: Two weeks after tumor cell inoculation, tumor-bearing mice were first stratified based on tumor burden reflected by bioluminescence intensity (high vs low) and then randomized into various treatment groups to ensure tumor load comparability among groups. 4×10¹² vg (viral genomes) of AAV-circuit constructs or PBS were administered intravenously via two separate injections one week apart. Prodrug GCV (50 mg/kg, InvivoGen) or saline treatment was initiated on day 3 post first AAV injection, mice were injected intraperitoneally once per day for a 2-week duration. Tumor growth was assessed with bioluminescent imaging 2-3 times per week. Mice were monitored with score sheet and euthanized if endpoints were achieved. All mice were terminated after 14 days of prodrug treatment. The livers were harvested for ex vivo bioluminescent imaging analysis of tumor loads. Two weeks after tumor cell inoculation, tumor-bearing mice were first stratified based on tumor burden reflected by bioluminescence intensity (high vs low) and then randomized into various treatment groups to ensure tumor load comparability among groups. 4×10¹² vg (viral genomes) of AAV-circuit constructs or PBS were administered intravenously via two separate injections one week apart. Prodrug GCV (50 mg/kg, InvivoGen) or saline treatment was initiated on day 3 post first AAV injection, mice were injected intraperitoneally once per day for a 2-week duration. Tumor growth was assessed with bioluminescent imaging 2-3 times per week. Mice were monitored with score sheet and euthanized if endpoints were achieved. All mice were terminated after 14 days of prodrug treatment. The livers were harvested for ex vivo bioluminescent imaging analysis of tumor loads.

### REFERENCES

1. Al-Zaidy, S., Pickard, A. S., Kotha, K., Alfano, L. N., Lowes, L., Paul, G., Church, K., Lehman, K., Sproule, D. M., Dabbous, O., et al. (2019). Health outcomes in spinal muscular atrophy type 1 following AVXS-101 gene replacement therapy. Pediatric Pulmonology 54, 179-185.
2. Angelici, B., Mailand, E., Haefliger, B., and Benenson, Y. (2016). Synthetic Biology Platform for Sensing and Integrating Endogenous Transcriptional Inputs in Mammalian Cells. Cell Reports 16, 2525-2537.
3. Auslander, D., Auslander, S., Charpin-El Hamri, G., Sedlmayer, F., Muller, M., Frey, O., Hierlemann, A., Stelling, J., and Fussenegger, M. (2014). A Synthetic Multifunctional Mammalian pH Sensor and CO2 Transgene-Control Device. Molecular Cell 55, 397-408.
4. Benenson, Y. (2012). Biomolecular computing systems: principles, progress and potential. Nature Reviews Genetics 13, 455-468.
5. Benenson, Y., Gil, B., Ben-Dor, U., Adar, R., and Shapiro, E. (2004). An autonomous molecular computer for logical control of gene expression. Nature 429, 423-429.
6. Cho, J. H., Collins, J. J., and Wong, W. W. (2018). Universal Chimeric Antigen Receptors for Multiplexed and Logical Control of T Cell Responses. Cell 173, 1426-+.
7. Choudhury, S. R., Fitzpatrick, Z., Harris, A. F., Maitland, S. A., Ferreira, J. S., Zhang, Y. F., Ma, S., Sharma, R. B., Gray-Edwards, H. L., Johnson, J. A., et al. (2016). In Vivo Selection Yields AAV-B1 Capsid for Central Nervous System and Muscle Gene Therapy. Molecular Therapy 24, 1247-1257.
8. Coulouarn, C., Factor, V. M., Andersen, J. B., Durkin, M. E., and Thorgeirsson, S. S. (2009). Loss of miR-122 expression in liver cancer correlates with suppression of the hepatic phenotype and gain of metastatic properties. Oncogene 28, 3526-3536.
9. Dagogo-Jack, I., and Shaw, A. T. (2018). Tumour heterogeneity and resistance to cancer therapies. Nature Reviews Clinical Oncology 15, 81-94.
10. Dastor, M., Schreiber, J., Prochazka, L., Angelici, B., Kleinert, J., Klebba, I., Doshi, J., Shen, L., and Benenson, Y. (2018). A Workflow for In Vivo Evaluation of Candidate Inputs and Outputs for Cell Classifier Gene Circuits. Acs Synthetic Biology 7, 474-489.
11. Della Peruta, M., Badar, A., Rosales, C., Chokshi, S., Kia, A., Nathwani, D., Galante, E., Yan, R., Arstad, E., Davidoff, A. M., et al. (2015). Preferential Targeting of Disseminated Liver Tumors Using a Recombinant Adeno-Associated Viral Vector. Human Gene Therapy 26, 94-103.
12. Duan, D. S., Yue, Y. P., and Engelhardt, J. F. (2003). Consequences of DNA-dependent protein kinase catalytic subunit deficiency on recombinant adeno-associated virus genome circularization and heterodimerization in muscle tissue. J Virol 77, 4751-4759.
13. Freeman, S. M., Abboud, C. N., Whartenby, K. A., Packman, C. H., Koeplin, D. S., Moolten, F. L., and Abraham, G. N. (1993). The bystander effect—tumor regression when a fraction of the tumor mass is genetically modified. Cancer Res 53, 5274-5283.
14. Fussenegger, M., Morris, R. P., Fux, C., Rimann, M., von Stockar, B., Thompson, C. J., and Bailey, J. E. (2000). Streptogramin-based gene regulation systems for mammalian cells. Nat Biotechnol 18, 1203-1208.
15. Grimm, D., Lee, J. S., Wang, L., Desai, T., Akache, B., Storm, T. A., and Kay, M. A. (2008). In vitro and in vivo gene therapy vector evolution via multispecies interbreeding and retargeting of adeno-associated viruses. J Virol 82, 5887-5911.
16. Harries, L. W., Brown, J. E., and Gloyn, A. L. (2009). Species-Specific Differences in the Expression of the HNF1A, HNF1B and HNF4A Genes. Plos One 4.
17. Huang, H. Y., Liu, Y. Q., Liao, W. X., Cao, Y. B., Liu, Q., Guo, Y. K., Lu, Y. Y., and Xie, Z. (2019). Oncolytic adenovirus programmed by synthetic gene circuit for cancer immunotherapy. Nature Communications 10.
18. June, C. H., O'Connor, R. S., Kawalekar, O. U., Ghassemi, S., and Milone, M. C. (2018). CAR T cell immunotherapy for human cancer. Science 359, 1361-1365.
19. Juttner, J., Szabo, A., Gross-Scherf, B., Morikawa, R. K., Rompani, S. B., Hantz, P., Szikra, T., Esposti, F., Cowan, C. S., Bharioke, A., et al. (2019). Targeting neuronal and glial cell types with synthetic promoter AAVs in mice, non-human primates and humans. Nature Neuroscience 22, 1345-+.
20. Keeler, A. M., and Flotte, T. R. (2019). Recombinant Adeno-Associated Virus Gene Therapy in Light of Luxturna (and Zolgensma and Glybera): Where Are We, and How Did We Get Here? In Annual Review of Virology, Vol 6, 2019, L. Enquist, D. DiMaio, and T. Demody, eds., pp. 601-621.
21. Kloss, C. C., Condomines, M., Cartellieri, M., Bachmann, M., and Sadelain, M. (2013). Combinatorial antigen recognition with balanced signaling promotes selective tumor eradication by engineered T cells. Nat Biotechnol 31, 71-+.
22. Kota, J., Chivukula, R. R., O'Donnell, K. A., Wentzel, E. A., Montgomery, C. L., Hwang, H. W., Chang, T. C., Vivekanandan, P., Torbenson, M., Clark, K. R., et al. (2009). Therapeutic microRNA Delivery Suppresses Tumorigenesis in a Murine Liver Cancer Model. Cell 137, 1005-1017.
23. Landegger, L. D., Pan, B. F., Askew, C., Wassmer, S. J., Gluck, S. D., Galvin, A., Taylor, R., Forge, A., Stankovic, K. M., Holt, J. R., et al. (2017). A synthetic AAV vector enables safe and efficient gene transfer to the mammalian inner ear. Nat Biotechnol 35, 280-+.
24. Liao, Y. L., Sun, Y. M., Chau, G. Y., Chau, Y. P., Lai, T. C., Wang, J. L., Horng, J. T., Hsiao, M., and Tsou, A. P. (2008). Identification of SOX4 target genes using phylogenetic footprinting-based prediction from expression microarrays suggests that overexpression of SOX4 potentiates metastasis in hepatocellular carcinoma. Oncogene 27, 5578-5589.
25. Llovet, J. M., and Lencioni, R. (2020). mRECIST for HCC: Performance and novel refinements. Journal of Hepatology 72, 288-306.
26. Nelson, C. E., Hakim, C. H., Ousterout, D. G., Thakore, P. I., Moreb, E. A., Rivera, R. M. C., Madhavan, S., Pan, X. F., Ran, F. A., Yan, W. X., et al. (2016). In vivo genome editing improves muscle function in a mouse model of Duchenne muscular dystrophy. Science 351, 403-407.
27. Nissim, L., Wu, M. R., Pery, E., Binder-Nissim, A., Suzuki, H. I., Stupp, D., Wehrspaun, C., Tabach, Y., Sharp, P. A., and Lu, T. K. (2017). Synthetic RNA-Based Immunomodulatory Gene Circuits for Cancer Immunotherapy. Cell 171, 1138-+.
28. Richtig, G., Aigelsreiter, A., Schwarzenbacher, D., Ress, A. L., Adiprasito, J. B., Stiegelbauer, V., Hoefler, G., Schauer, S., Kiesslich, T., Kornprat, P., et al. (2017). SOX9 is a proliferation and stem cell factor in hepatocellular carcinoma and possess widespread prognostic significance in different cancer types. Plos One 12.
29. Roybal, K. T., Williams, J. Z., Morsut, L., Rupp, L. J., Kolinko, I., Choe, J. H., Walker, W. J., McNally, K. A., and Lim, W. A. (2016). Engineering T Cells with Customized Therapeutic Response Programs Using Synthetic Notch Receptors. Cell 167, 419-+.
30. Scholl, H. P. N., Strauss, R. W., Singh, M. S., Dalkara, D., Roska, B., Picaud, S., and Sahel, J. A. (2016). Emerging therapies for inherited retinal degeneration. Science Translational Medicine 8, 10.
31. Tastanova, A., Folcher, M., Muller, M., Camenisch, G., Ponti, A., Horn, T., Tikhomirova, M. S., and Fussenegger, M. (2018). Synthetic biology-based cellular biomedical tattoo for detection of hypercalcemia associated with. Science Translational Medicine 10.
32. Uhlen, M., Zhang, C., Lee, S., Sjostedt, E., Fagerberg, L., Bidkhori, G., Benfeitas, R., Arif, M., Liu, Z. T., Edfors, F., et al. (2017). A pathology atlas of the human cancer transcriptome. Science 357, 660-+.
33. Weber, W., and Fussenegger, M. (2012). Emerging biomedical applications of synthetic biology. Nature Reviews Genetics 13, 21-35.
34. Xie, Z., Wroblewska, L., Prochazka, L., Weiss, R., and Benenson, Y. (2011). Multi-Input RNAi-Based Logic Circuit for Identification of Specific Cancer Cells. Science 333, 1307-1311.
35. Ye, H. F., Xie, M. Q., Xue, S., Charpin-El Hamri, G., Yin, J. L., Zulewski, H., and Fussenegger, M. (2017). Self-adjusting synthetic gene circuit for correcting insulin resistance. Nature Biomedical Engineering 1.
36. Zah, E., Lin, M. Y., Silva-Benedict, A., Jensen, M. C., and Chen, Y. Y. (2016). T Cells Expressing CD19/CD20 Bispecific Chimeric Antigen Receptors Prevent Antigen Escape by Malignant B Cells. Cancer Immunology Research 4, 498-508.
37. Paterna, J. C., Feldon, J., and Bueler, H. (2004). Transduction profiles of recombinant adeno-associated virus vectors derived from serotypes 2 and 5 in the nigrostriatal system of rats. J Virol 78, 6808-6817.
38. Conway, J. E., Rhys, C. M., Zolotukhin, I., Zolotukhin, S., Muzyczka, N., Hayward, G. S., and Byrne, B. J. (1999). High-titer recombinant adeno-associated virus production utilizing a recombinant herpes simplex virus type I vector expressing AAV-2 Rep and Cap. Gene Ther 6, 986-993.
39. Glatzel, M., Flechsig, E., Navarro, B., Klein, M. A., Paterna, J. C., Bueler, H., and Aguzzi, A. (2000). Adenoviral and adeno-associated viral transfer of genes to the peripheral nervous system. Proc Natl Acad Sci USA 97, 442-447.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A contiguous polynucleic acid molecule comprising:
a) a first cassette encoding a first RNA whose expression is operably linked to a transactivator response element, wherein the first RNA comprises: (i) a nucleic acid sequence of an output; and (ii) a target site for a miRNA listed in TABLE 1 or a combination thereof; and
b) a second cassette encoding a second RNA, wherein the second RNA comprises a nucleic acid sequence of a transactivator;
wherein the transactivator of the second cassette, when expressed as a protein, binds and transactivates the transactivator response element of the first cassette.

2. The contiguous polynucleic acid molecule of claim 1, wherein the first RNA comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

3. The contiguous polynucleic acid molecule of claim 1 or claim 2, wherein the first RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

4. The contiguous polynucleic acid molecule of any one of claims 1-3, wherein the first RNA comprises a 5′ UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

5. The contiguous polynucleic acid molecule of any one of claims 1-4, wherein the second RNA further comprises a target site for a microRNA listed in TABLE 1 or a combination thereof.

6. The contiguous polynucleic acid molecule of any one of claims 1-5, wherein the second RNA further comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

7. The contiguous polynucleic acid molecule of claim 6, wherein the second RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

8. The contiguous polynucleic acid molecule of claim 6 or claim 7, wherein the second RNA comprises a 5′ UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

9. The contiguous polynucleic acid molecule of any one of claims 6-8, wherein at least one miRNA target site of the first cassette and at least one miRNA target site of the second cassette are identical nucleic acid sequences or are different sequences regulated by the same miRNA.

10. The contiguous polynucleic acid molecule of any one of claims 6-9, wherein the first RNA and the second RNA each comprises a let-7c target site.

11. The contiguous polynucleic acid molecule of any one of claims 1-10, wherein the transactivator response element comprises a nucleic acid sequence listed in TABLE 3 or a combination thereof.

12. The contiguous polynucleic acid molecule of any one of claims 1-10, wherein expression of the second RNA is operably linked to a transcription factor response element.

13. The contiguous polynucleic acid molecule of claim 12, wherein the transcription factor response element comprises a nucleic acid sequence listed in TABLE 4 or a combination thereof.

14. The contiguous polynucleic acid molecule of any one of claims 1-13, wherein the transactivator binds and transactivates the transactivator response element independently.

15. The contiguous polynucleic acid molecule of any one of claims 1-13, wherein expression of the first RNA is operably linked to a transcription factor response element.

16. The contiguous polynucleic acid molecule of claim 15, wherein the transcription factor response element comprises a nucleic acid sequence listed in TABLE 4 or a combination thereof.

17. The contiguous polynucleic acid molecule of any one of claims 12, 13, or 16, wherein the transactivator binds and transactivates the transactivator response element only in the presence of a transcription factor bound to the transcription factor response element.

18. The contiguous polynucleic acid molecule of any one of claim 1-17, wherein the first cassette and/or the second cassette comprises a promoter element.

19. The contiguous polynucleic acid molecule of claim 18, wherein the promoter element comprises a nucleic acid sequence listed in TABLE 5 or a combination thereof.

20. The contiguous polynucleic acid molecule of claim 18, wherein the promoter element comprises a mammalian promoter or promoter fragment.

21. The contiguous polynucleic acid molecule of any one of claims 15-17, wherein:
the first cassette comprises, from 5′ to 3′: (i) an upstream regulatory component comprising the transactivator response element and the transcription factor response element; (ii) the nucleic acid sequence encoding the output; and (iii) a downstream component comprising a let-7c target site; and
the second cassette comprises, from 5′ to 3′: (i) an upstream regulatory component comprising a transcription factor response element; (ii) the nucleic acid sequence encoding the transactivator; and (iii) a downstream component comprising a let-7c target site.

22. The contiguous polynucleic acid molecule of claim 21, wherein the transcription factor response element of the first cassette and the transcription factor response element of the second cassette consist of identical nucleic acid sequences.

23. The contiguous polynucleic acid molecule of claim 21, wherein the transcription factor response element of the first cassette and the transcription factor response element of the second cassette consist of different nucleic acid sequences.

24. The contiguous polynucleic acid molecule of any one of claims 15-23, wherein the first cassette and/or the second cassette comprises two or more transcription factor response elements.

25. The contiguous polynucleic acid molecule of claim 24, wherein the first cassette and/or the second cassette comprises two different transcription factor response elements.

26. The contiguous polynucleic acid molecule of any one of claims 21-25, wherein the upstream regulatory component of the first cassette comprises a promoter element.

27. The contiguous polynucleic acid molecule of claim 26, wherein the promoter element comprises a mammalian promoter or promoter fragment.

28. The contiguous polynucleic acid molecule of any one of claims 21-27, wherein the upstream regulatory component of the second cassette comprises a promoter element.

29. The contiguous polynucleic acid molecule of claim 28, wherein the promoter element comprises a mammalian promoter or promoter fragment.

30. The contiguous polynucleic acid molecule of any one of claims 1-29, wherein the first cassette and the second cassette are in a convergent orientation.

31. The contiguous polynucleic acid molecule of any one of claims 1-29, wherein the first cassette and the second cassette are in a divergent orientation.

32. The contiguous polynucleic acid molecule of any one of claims 1-29, wherein the first cassette and the second cassette are in a head-to-tail orientation.

33. The contiguous polynucleic acid molecule of any one of claims 1-32, wherein the first cassette and/or the second cassette is flanked by an insulator.

34. The contiguous polynucleic acid molecule of any one of claims 1-33, wherein the transactivator of the second cassette is tTA, rtTA, PIT-RelA, PIT-VP16, ET-VP16, ET-RelA, NarLc-VP16, or NarLc-RelA.

35. The contiguous polynucleic acid molecule of any one of claims 1-33, wherein the transactivator of the second cassette comprises a nucleic acid sequence listed in TABLE 2.

36. The contiguous polynucleic acid molecule of any one of claims 1-35, wherein the output is a protein or an RNA molecule.

37. The contiguous polynucleic acid molecule of any one of claims 1-36, wherein the output is a therapeutic.

38. The contiguous polynucleic acid molecule of claim 36 or claim 37, wherein the output is a fluorescent protein, a cytotoxin, an enzyme catalyzing a prodrug activation, an immunomodulatory protein and/or RNA, a DNA-modifying factor, cell-surface receptor, a gene expression-regulating factor, a kinase, an epigenetic modifier, and/or a factor necessary for vector replication, and/or a sequence encoding an antigen polypeptide of a pathogen.

39. The contiguous polynucleic acid molecule of claim 36 or claim 37, wherein the output is the thymidine kinase enzyme from human simplex herpes virus 1 (HSV-TK).

40. The contiguous polynucleic acid molecule of claim 38, wherein the immunomodulatory protein and/or RNA is a cytokine or a colony stimulating factor.

41. The contiguous polynucleic acid molecule of claim 38, wherein the DNA-modifying factor is a gene encoding a protein intended to correct a genetic defect, a DNA-modifying enzyme, and/or a component of a DNA-modifying system.

42. The contiguous polynucleic acid molecule of claim 41, wherein the DNA-modifying enzyme is a site-specific recombinase, homing endonuclease, or a protein component of a CRISPR/Cas DNA modification system.

43. The contiguous polynucleic acid molecule of claim 38, wherein the gene expression-regulating factor is a protein capable of regulating gene expression or a component of a multi-component system capable of regulating gene expression.

44. A contiguous polynucleic acid molecule comprising a nucleic acid sequence listed in TABLE 6.

45. A contiguous polynucleic acid molecule comprising a cassette encoding an RNA whose expression is operably linked to a transactivator response element, wherein the RNA comprises: (i) a nucleic acid sequence of an output; (ii) a nucleic acid sequence of a transactivator; and (iii) a target site for a miRNA listed in TABLE 1 or a combination thereof; wherein the transactivator, when expressed as a protein, binds and transactivates the transactivator response element.

46. The contiguous polynucleic acid molecule of claim 45, wherein the first RNA comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

47. The contiguous polynucleic acid molecule of claim 45 or claim 46, wherein the RNA further comprises a nucleic acid sequence of a polycistronic expression element separating the nucleic acid sequences of the output and the transactivator.

48. The contiguous polynucleic acid molecule of any one of claims 45-47, wherein the RNA comprises a 3′ UTR, and wherein the 3′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

49. The contiguous polynucleic acid molecule of any one of claims 45-48, wherein the RNA comprises a 5′UTR, and wherein the 5′ UTR comprises a let-7c target site, a let-7a target site, a let-7b target site, a let-7d target site, a let-7e target site, a let-7f target site, a let-7g target site, a let-7i target site, a miR-22 target site, a miR-26b target site, a miR-122 target site, a miR-208a target site, a miR-208b target site, a miR-1 target site, a miR-217 target site, a miR-216a target site, or a combination thereof.

50. The contiguous polynucleic acid molecule of any one of claim 45-49, wherein the RNA comprises a let-7c target site.

51. The contiguous polynucleic acid molecule of any one of claims 45-50, wherein the transactivator response element comprises a nucleic acid sequence listed in TABLE 3 or a combination thereof.

52. The contiguous polynucleic acid molecule of any one of claims 45-50, wherein the transactivator binds and transactivates the transactivator response element independently.

53. The contiguous polynucleic acid molecule of any one of claims 45-52, wherein the expression of the RNA is operably linked to a transactivator response element and a transcription factor response element.

54. The contiguous polynucleic acid molecule of claim 53, wherein the transcription factor response element comprises a nucleic acid sequence listed in TABLE 4 or a combination thereof.

55. The contiguous polynucleic acid molecule of claim 53, wherein the transactivator binds and transactivates the transactivator response element only in the presence of a transcription factor bound to the transcription factor response element.

56. The contiguous polynucleic acid molecule of any one of claim 45-55, wherein the cassette comprises a promoter element.

57. The contiguous polynucleic acid molecule of claim 56, wherein the promoter element comprises a nucleic acid sequence listed in TABLE 5 or a combination thereof.

58. The contiguous polynucleic acid molecule of claim 56, wherein the promoter element comprises a mammalian promoter or promoter fragment.

59. The contiguous polynucleic acid molecule of claim 53 or claim 55, wherein the contiguous polynucleic acid molecule comprises, from 5′ to 3′: (i) an upstream regulatory component comprising the transactivator response element and the transcription factor response element; (ii) the nucleic acid sequence encoding the output and the transactivator; and (iii) a downstream component comprising a let-7c target site.

60. The contiguous polynucleic acid molecule of claim 59, wherein the upstream regulatory component in (i) comprises a promoter element.

61. The contiguous polynucleic acid molecule of claim 60, wherein the promoter element comprises a mammalian promoter or promoter fragment.

62. The contiguous polynucleic acid molecule of any one of claims 45-61, wherein the transactivator of at least one cassette is tTA, rtTA, PIT-RelA, PIT-VP16, ET-VP16, ET-RelA, NarLc-VP16, or NarLc-RelA.

63. The contiguous polynucleic acid molecule of any one of claims 45-61, wherein the transactivator of the second cassette comprises a nucleic acid sequence listed in TABLE 2.

64. The contiguous polynucleic acid molecule of any one of claims 45-62, wherein the output is a protein or an RNA molecule.

65. The contiguous polynucleic acid molecule of any one of claims 45-64, wherein the output is a therapeutic protein or RNA molecule.

66. The contiguous polynucleic acid molecule of claim 64 or claim 65, wherein the output is a fluorescent protein, a cytotoxin, an enzyme catalyzing a prodrug activation, an immunomodulatory protein and/or RNA, a DNA-modifying factor, cell-surface receptor, a gene expression-regulating factor, a kinase, an epigenetic modifier, and/or a factor necessary for vector replication, and/or a sequence encoding an antigen polypeptide of a pathogen.

67. The contiguous polynucleic acid molecule of claim 64 or claim 65, wherein the output is the thymidine kinase enzyme from human simplex herpes virus 1 (HSV-TK).

68. The contiguous polynucleic acid molecule of claim 66, wherein the immunomodulatory protein and/or RNA is a cytokine or a colony stimulating factor.

69. The contiguous polynucleic acid molecule of claim 66, wherein the DNA-modifying factor is a gene encoding a protein intended to correct a genetic defect, a DNA-modifying enzyme, and/or a component of a DNA-modifying system.

70. The contiguous polynucleic acid molecule of claim 69, wherein the DNA-modifying enzyme is a site-specific recombinase, homing endonuclease, or a protein component of the CRISPR/Cas system.

71. The contiguous polynucleic acid molecule of claim 66, wherein the gene expression-regulating factor is a protein capable of regulating gene expression or a component of a multi-component system capable of regulating gene expression.

72. A vector comprising the contiguous polynucleic acid molecule of any one of claims 1-44 or claims 45-71.

73. An engineered viral genome comprising the contiguous polynucleic acid molecule of any one of claims 1-44 or claims 45-71.

74. The engineered viral genome of claim 73, wherein the viral genome is an adeno-associated virus (AAV) genome, a lentivirus genome, an adenovirus genome, a herpes simplex virus (HSV) genome, a Vaccinia virus genome, a poxvirus genome, a Newcastle Disease virus (NDV) genome, a Coxsackievirus genome, a rheovirus genome, a measles virus genome, a Vesicular Stomatitis virus (VSV) genome, a Parvovirus genome, a Seneca valley viral genome, a Maraba virus genome or a common cold virus genome.

75. A virion comprising the engineered viral genome of claim 73 or claim 74.

76. The virion of claim 75, further comprising an AAV-DJ, AAV8, AAV6, or AAV-B1 capsid.

77. A method of stimulating a cell-specific event in a population of cells comprising contacting a population of cells with the contiguous polynucleic acid molecule of any one of claims 1-44 or claims 45-71, the vector of claim 72, the engineered viral genome of claim 73 or claim 74, or the virion of claim 75 or claim 76, wherein the population of cells comprises at least one target cell type and one or more non-target cell types, wherein the target cell type(s) and the non-target cell types differ in levels and/or activity of one or more endogenous miRNAs, such that the levels and/or activity of the one or more endogenous miRNAs are at least two times higher in each of the two or more non-target cells relative to each of the target cells; and wherein the cell-specific event is regulated by expression levels of the output in the cells of the population of cells.

78. The method of claim 77, wherein at least a subset of the target cells and at least a subset of the non-target cells differ in levels or activity of an endogenous transcription factor, wherein the contiguous nucleic acid molecule further comprises a transcription factor response element corresponding to the endogenous transcription factor.

79. The method of claim 77, wherein at least a subset of the target cells and at least a subset of the non-target cells differ in levels or activity of a promoter fragment, wherein the contiguous nucleic acid molecule further comprises this promoter fragment.

80. A method of diagnosing a disease or a condition comprising administering a contiguous polynucleic acid molecule of any one of 1-44 or claims 45-71, the vector of claim 72, the engineered viral genome of claim 73 or claim 74, or the virion of claim 75 or claim 76 to a subject exhibiting one or more signs or symptoms associated with a disease or condition, wherein the levels of the output indicates the presence or absence of the disease and or condition.

81. The method of claim 80, wherein the disease is cancer.

82. The method of claim 81, wherein the cancer is hepatocellular carcinoma (HCC), metastatic colorectal cancer, a metastatic tumor in the liver, breast cancer, lung cancer, retinoblastoma, and glioblastoma.

83. A method of treating a disease or a condition comprising administering a contiguous polynucleic acid molecule of any one of 1-44 or claims 45-71, the vector of claim 72, the engineered viral genome of claim 73 or claim 74, or the virion of claim 75 or claim 76 to a subject having the disease or condition.

84. The method of claim 83, further comprising administering a prodrug, optionally wherein the prodrug is ganciclovir, optionally wherein the contiguous polynucleic acid molecule comprises a nucleic acid sequence listed in TABLE 6.

85. The method of claim 83, wherein the disease is cancer.

86. The method of claim 85, wherein the cancer is hepatocellular carcinoma (HCC)), metastatic colorectal cancer, a metastatic tumor in the liver, breast cancer, lung cancer, retinoblastoma, and glioblastoma.

87. A composition for use in a method of stimulating a cell-specific event in a population of cells comprising contacting a population of cells with the contiguous polynucleic acid molecule of any one of claims 1-44 or claims 45-71, the vector of claim 72, the engineered viral genome of claim 73 or claim 74, or the virion of claim 75 or claim 76, wherein the population of cells comprises at least one target cell type and one or more non-target cell types, wherein the target cell type(s) and the non-target cell types differ in levels and/or activity of one or more endogenous miRNAs, such that the levels and/or activity of the one or more endogenous miRNAs are at least two times higher in each of the two or more non-target cells relative to each of the target cells; and wherein the cell-specific event is regulated by expression levels of the output in the cells of the population of cells.

88. The method of claim 87, wherein at least a subset of the target cells and at least a subset of the non-target cells differ in levels or activity of an endogenous transcription factor, wherein the contiguous nucleic acid molecule further comprises a transcription factor response element corresponding to the endogenous transcription factor.

89. The method of claim 87, wherein at least a subset of the target cells and at least a subset of the non-target cells differ in levels or activity of a promoter fragment, wherein the contiguous nucleic acid molecule further comprises this promoter fragment.

90. A composition for use in a method of diagnosing a disease or a condition comprising administering a contiguous polynucleic acid molecule of any one of 1-44 or claims 45-71, the vector of claim 72, the engineered viral genome of claim 73 or claim 74, or the virion of claim 75 or claim 76 to a subject exhibiting one or more signs or symptoms associated with a disease or condition, wherein the levels of the output indicates the presence or absence of the disease and or condition.

91. The composition for use according to claim 90, wherein the disease is cancer.

92. The composition for use according to claim 91 wherein the cancer is hepatocellular carcinoma (HCC), metastatic colorectal cancer, a metastatic tumor in the liver, breast cancer, lung cancer, retinoblastoma, and glioblastoma.

93. A composition for use in a method of treating a disease or a condition comprising administering a contiguous polynucleic acid molecule of any one of 1-44 or claims 45-71, the vector of claim 72, the engineered viral genome of claim 73 or claim 74, or the virion of claim 75 or claim 76 to a subject having the disease or condition.

94. The method of claim 93, further comprising administering a prodrug, optionally wherein the prodrug is ganciclovir, optionally wherein the contiguous polynucleic acid molecule comprises a nucleic acid sequence listed in TABLE 6.

95. The composition for use according to claim 93, wherein the disease is cancer.

96. The composition for use according to claim 95, wherein the cancer is hepatocellular carcinoma (HCC), metastatic colorectal cancer, a metastatic tumor in the liver, breast cancer, lung cancer, retinoblastoma, and glioblastoma.

97. A method of stimulating a cell-specific event in a population of cells comprising contacting the population of cells with the contiguous polynucleic acid molecule or a composition comprising said contiguous polynucleic aid molecule, wherein:
a) the population of cells comprises at least one target cell type and two or more non-target cell types, wherein the target cell type(s) and the non-target cell types differ in levels of one or more endogenous miRNAs, such that the levels of the one or more endogenous miRNAs are at least two times higher in at least a subset of the non-target cells, such as at least two and optionally each of the two or more non-target cells, relative to each of the target cells; and
b) the contiguous polynucleic acid molecule comprises:
(i) a first cassette encoding a RNA whose expression is operably linked to a transactivator response element, wherein the first RNA comprises: a nucleic acid sequence of an output; and one or more miRNA target sites corresponding to the one or more endogenous miRNAs; and
(ii) a second cassette encoding a second RNA, wherein the second RNA comprises a nucleic acid sequence of a transactivator;
wherein the transactivator of the second cassette, when expressed as a protein, binds and transactivates the transactivator response element of the first cassette; and
wherein the cell-specific event is regulated by expression levels of the output in the cells of the population of cells.

98. The method of claim 97, wherein the contiguous polynucleic acid molecule comprises a nucleic acid sequence listed in TABLE 6.

99. A method of stimulating a cell-specific event in a population of cells comprising contacting the population of cells with the contiguous polynucleic acid molecule or a composition comprising said contiguous polynucleic aid molecule, wherein:
a) the population of cells comprises at least one target cell type and two or more non-target cell types, wherein the target cell type(s) and the non-target cell types differ in levels of one or more endogenous miRNAs, such that the levels of the one or more endogenous miRNAs are at least two times higher in at least a subset of the non-target cells, such as at least two and optionally each of the two or more non-target cells, relative to each of the target cells; and
b) the contiguous polynucleic acid molecule comprises a cassette encoding a mRNA whose expression is operably linked to a transactivator response element, wherein the RNA comprises: a nucleic acid sequence of an output; a nucleic acid sequence of a transactivator; and one or more miRNA target sites corresponding to the one or more endogenous miRNAs; and
wherein the transactivator, when expressed as a protein, binds and transactivates the transactivator response element of the cassette; and
wherein the cell-specific event is regulated by expression levels of the output in the cells of the population of cells.

100. The method of claim 97 or 99, wherein the composition comprising the contiguous polynucleic aid molecule comprises a vector comprising the contiguous polynucleic acid, an engineered viral genome comprising the contiguous polynucleic acid, or a virion comprising the polynucleic acid.

101. The method of any one of claims 97-100, wherein the endogenous miRNA is selected from the miRNAs listed in TABLE 1 or a combination of miRNAs listed in TABLE 1.

102. The method of any one of claims 97-101, wherein the endogenous miRNA is selected from the group consisting of let-7c, let-7a, let-7b, let-7d, let-7e, let-7f, let-7g, let-7i, miR-22, miR-26b, miR-122, miR-208a, miR-208b, miR-1, miR-217, miR-216a, or a combination thereof.

103. The method of any one of claims 97-101, wherein at least a subset of the target cells and at least a subset of the non-target cells differ in levels or activity of an endogenous transcription factor, wherein the contiguous nucleic acid molecule further comprises a transcription factor response element corresponding to the endogenous transcription factor.

104. The method of any one of claims 97-101, wherein at least a subset of the target cells and at least a subset of the non-target cells differ in levels or activity of a promoter fragment, wherein the contiguous nucleic acid molecule further comprises this promoter fragment.

105. The method of any one of claims 97-103, wherein the target cells are tumor cells and the cell-specific event is tumor cell death.

106. The method of claim 105, wherein the tumor cell death is mediated by immune targeting through the expression of activating receptor ligands, specific antigens, stimulating cytokines or any combination thereof.

107. The method of any one of claims 97-103, wherein the target cells are senescent cells and the cell-specific event is senescent cell death.

108. The method of any one of claims 97-107, further comprising contacting the population of cells with prodrug or a non-toxic precursor compound that is metabolized by the output into a therapeutic or a toxic compound.

109. The method of any one of claims 97-103, wherein output expression ensures the survival of the target cell population while the non-target cells are eliminated due to lack of output expression and in the presence of an unrelated and unspecific cell death-inducing agent.

110. The method of any one of claims 97-103, wherein the target cells comprise a particular phenotype of interest such that output expression is limited to the cells of this particular phenotype.

111. The method of any one of claims 97-102, wherein the target cells are a cell type of choice and the cell-specific event is the encoding of a novel function, through the expression of a gene naturally absent or inactive in the cell type of choice.

112. The method of any one of claims 97-111, wherein the population of cells comprises a multicellular organism.

113. The method of claim 112, wherein the multicellular organism is an animal.

114. The method of claim 113, wherein the animal is a human.

115. The method of any one of claims 97-114, wherein the population of cells is contacted ex-vivo.

116. The method of any one of claims 97-114, wherein the population of cells is contacted in-vivo.

117. A contiguous polynucleic acid molecule comprising:
a) a first cassette encoding a first RNA whose expression is operably linked to a transactivator response element, wherein the first RNA comprises: (i) a nucleic acid sequence of an output; and (ii) a target site for a miRNA, wherein said miRNA is highly expressed and/or active in at least two different healthy tissues of a mammal and is expressed at low level in one or more types of target cells;
b) a second cassette encoding a second RNA, wherein the second RNA comprises a nucleic acid sequence of a
wherein the transactivator of the second cassette, when expressed as a protein, binds and transactivates the transactivator response element of the first cassette.


SEQUENCE LISTING (306 sequences)

SEQ ID NO: 1 - RNA, 22 nt, Artificial Sequence
  Synthetic
ugagguaguagguuguaugguu

SEQ ID NO: 2 - RNA, 22 nt, Artificial Sequence
  Synthetic
aagcugccaguugaagaacugu

SEQ ID NO: 3 - RNA, 21 nt, Artificial Sequence
  Synthetic
uucaaguaauucaggauaggu

SEQ ID NO: 4 - RNA, 21 nt, Artificial Sequence
  Synthetic
cauuauuacuuuugguacgcg

SEQ ID NO: 5 - RNA, 22 nt, Artificial Sequence
  Synthetic
uggagugugacaaugguguuug

SEQ ID NO: 6 - RNA, 22 nt, Artificial Sequence
  Synthetic
cagcagcaauucauguuuugga

SEQ ID NO: 7 - RNA, 22 nt, Artificial Sequence
  Synthetic
cagcagcaauucauguuuugaa

SEQ ID NO: 8 - RNA, 22 nt, Artificial Sequence
  Synthetic
auaagacgagcaaaaagcuugu

SEQ ID NO: 9 - RNA, 22 nt, Artificial Sequence
  Synthetic
auaagacgaacaaaagguuugu

SEQ ID NO: 10 - RNA, 22 nt, Artificial Sequence
  Synthetic
uaaucucagcuggcaacuguga

SEQ ID NO: 11 - RNA, 23 nt, Artificial Sequence
  Synthetic
uacugcaucaggaacugacugga

SEQ ID NO: 12 - RNA, 23 nt, Artificial Sequence
  Synthetic
uacugcaucaggaacugauugga

SEQ ID NO: 13 - RNA, 22 nt, Artificial Sequence
  Synthetic
uuuguucguucggcucgcguga

SEQ ID NO: 14 - RNA, 22 nt, Artificial Sequence
  Synthetic
uaaggcacgcggugaaugccaa

SEQ ID NO: 15 - RNA, 22 nt, Artificial Sequence
  Synthetic
uggaauguaaagaaguauguau

SEQ ID NO: 16 - RNA, 22 nt, Artificial Sequence
  Synthetic
uuugguccccuucaaccagcug

SEQ ID NO: 17 - RNA, 22 nt, Artificial Sequence
  Synthetic
uuugguccccuucaaccagcua

SEQ ID NO: 18 - RNA, 23 nt, Artificial Sequence
  Synthetic
ucuuugguuaucuagcuguauga

SEQ ID NO: 19 - RNA, 22 nt, Artificial Sequence
  Synthetic
uccagcaucagugauuuuguug

SEQ ID NO: 20 - RNA, 21 nt, Artificial Sequence
  Synthetic
ugauuguccaaacgcaauucu

SEQ ID NO: 21 - RNA, 21 nt, Artificial Sequence
  Synthetic
uuuugcaccuuuuggagugaa

SEQ ID NO: 22 - RNA, 21 nt, Artificial Sequence
  Synthetic
auugacacuucugugaguaga

SEQ ID NO: 23 - RNA, 21 nt, Artificial Sequence
  Synthetic
uacugcagacaguggcaauca

SEQ ID NO: 24 - RNA, 24 nt, Artificial Sequence
  Synthetic
uggaagacuagugauuuuguuguu

SEQ ID NO: 25 - RNA, 22 nt, Artificial Sequence
  Synthetic
uccuucauuccaccggagucug

SEQ ID NO: 26 - RNA, 23 nt, Artificial Sequence
  Synthetic
uguaguguuuccuacuuuaugga

SEQ ID NO: 27 - RNA, 22 nt, Artificial Sequence
  Synthetic
acaguagucugcacauugguua

SEQ ID NO: 28 - RNA, 22 nt, Artificial Sequence
  Synthetic
uaacacugucugguaacgaugu

SEQ ID NO: 29 - RNA, 22 nt, Artificial Sequence
  Synthetic
uaauacugccugguaaugauga

SEQ ID NO: 30 - RNA, 21 nt, Artificial Sequence
  Synthetic
cugaccuaugaauugacagcc

SEQ ID NO: 31 - RNA, 22 nt, Artificial Sequence
  Synthetic
uguaacagcaacuccaugugga

SEQ ID NO: 32 - RNA, 22 nt, Artificial Sequence
  Synthetic
uggcaguguauuguuagcuggu

SEQ ID NO: 33 - RNA, 22 nt, Artificial Sequence
  Synthetic
ugagguaguagguuguauaguu

SEQ ID NO: 34 - RNA, 22 nt, Artificial Sequence
  Synthetic
ugagguaguagguugugugguu

SEQ ID NO: 35 - RNA, 22 nt, Artificial Sequence
  Synthetic
agagguaguagguugcauaguu

SEQ ID NO: 36 - RNA, 22 nt, Artificial Sequence
  Synthetic
ugagguaggagguuguauaguu

SEQ ID NO: 37 - RNA, 22 nt, Artificial Sequence
  Synthetic
ugagguaguagauuguauaguu

SEQ ID NO: 38 - RNA, 22 nt, Artificial Sequence
  Synthetic
ugagguaguaguuuguacaguu

SEQ ID NO: 39 - RNA, 22 nt, Artificial Sequence
  Synthetic
ugagguaguaguuugugcuguu

SEQ ID NO: 40 - RNA, 21 nt, Artificial Sequence
  Synthetic
ugagaugaagcacuguagcuc

SEQ ID NO: 41 - RNA, 22 nt, Artificial Sequence
  Synthetic
ucagugcacuacagaacuuugu

SEQ ID NO: 42 - DNA, 22 bp, Artificial Sequence
  Synthetic
aaccatacaacctactacctca

SEQ ID NO: 43 - DNA, 22 bp, Artificial Sequence
  Synthetic
acagttcttcaactggcagctt

SEQ ID NO: 44 - DNA, 21 bp, Artificial Sequence
  Synthetic
acctatcctgaattacttgaa

SEQ ID NO: 45 - DNA, 21 bp, Artificial Sequence
  Synthetic
cgcgtaccaaaagtaataatg

SEQ ID NO: 46 - DNA, 22 bp, Artificial Sequence
  Synthetic
caaacaccattgtcacactcca

SEQ ID NO: 47 - DNA, 22 bp, Artificial Sequence
  Synthetic
gtccaaaacatgaattgctgct

SEQ ID NO: 48 - DNA, 22 bp, Artificial Sequence
  Synthetic
gtccaaaacatgaattgctgct

SEQ ID NO: 49 - DNA, 22 bp, Artificial Sequence
  Synthetic
acaagctttttgctcgtcttat

SEQ ID NO: 50 - DNA, 22 bp, Artificial Sequence
  Synthetic
acaaaccttttgttcgtcttat

SEQ ID NO: 51 - DNA, 22 bp, Artificial Sequence
  Synthetic
tcacagttgccagctgagatta

SEQ ID NO: 52 - DNA, 23 bp, Artificial Sequence
  Synthetic
tccagtcagttcctgatgcagta

SEQ ID NO: 53 - DNA, 23 bp, Artificial Sequence
  Synthetic
tccaatcagttcctgatgcagta

SEQ ID NO: 54 - DNA, 22 bp, Artificial Sequence
  Synthetic
tcacgcgagccgaacgaacaaa

SEQ ID NO: 55 - DNA, 22 bp, Artificial Sequence
  Synthetic
ttggcattcaccgcgtgcctta

SEQ ID NO: 56 - DNA, 22 bp, Artificial Sequence
  Synthetic
atacatacttctttacattcca

SEQ ID NO: 57 - DNA, 22 bp, Artificial Sequence
  Synthetic
cagctggttgaaggggaccaaa

SEQ ID NO: 58 - DNA, 22 bp, Artificial Sequence
  Synthetic
tagctggttgaaggggaccaaa

SEQ ID NO: 59 - DNA, 23 bp, Artificial Sequence
  Synthetic
tcatacagctagataaccaaaga

SEQ ID NO: 60 - DNA, 22 bp, Artificial Sequence
  Synthetic
tccagcatcagtgattttgttg

SEQ ID NO: 61 - DNA, 21 bp, Artificial Sequence
  Synthetic
tgattgtccaaacgcaattct

SEQ ID NO: 62 - DNA, 21 bp, Artificial Sequence
  Synthetic
ttcactccaaaaggtgcaaaa

SEQ ID NO: 63 - DNA, 21 bp, Artificial Sequence
  Synthetic
attgacacttctgtgagtaga

SEQ ID NO: 64 - DNA, 21 bp, Artificial Sequence
  Synthetic
tactgcagacagtggcaatca

SEQ ID NO: 65 - DNA, 24 bp, Artificial Sequence
  Synthetic
aacaacaaaatcactagtcttcca

SEQ ID NO: 66 - DNA, 22 bp, Artificial Sequence
  Synthetic
cagactccggtggaatgaagga

SEQ ID NO: 67 - DNA, 23 bp, Artificial Sequence
  Synthetic
tccataaagtaggaaacactaca

SEQ ID NO: 68 - DNA, 22 bp, Artificial Sequence
  Synthetic
taaccaatgtgcagactactgt

SEQ ID NO: 69 - DNA, 22 bp, Artificial Sequence
  Synthetic
acatcgttaccagacagtgtta

SEQ ID NO: 70 - DNA, 22 bp, Artificial Sequence
  Synthetic
tcatcattaccaggcagtatta

SEQ ID NO: 71 - DNA, 21 bp, Artificial Sequence
  Synthetic
ggctgtcaattcataggtcag

SEQ ID NO: 72 - DNA, 22 bp, Artificial Sequence
  Synthetic
tccacatggagttgctgttaca

SEQ ID NO: 73 - DNA, 22 bp, Artificial Sequence
  Synthetic
accagctaacaatacactgcca

SEQ ID NO: 74 - DNA, 22 bp, Artificial Sequence
  Synthetic
aactatacaacctactacctca

SEQ ID NO: 75 - DNA, 22 bp, Artificial Sequence
  Synthetic
aaccacacaacctactacctca

SEQ ID NO: 76 - DNA, 22 bp, Artificial Sequence
  Synthetic
aactatgcaacctactacctct

SEQ ID NO: 77 - DNA, 22 bp, Artificial Sequence
  Synthetic
aactatacaacctcctacctca

SEQ ID NO: 78 - DNA, 22 bp, Artificial Sequence
  Synthetic
aactatacaatctactacctca

SEQ ID NO: 79 - DNA, 22 bp, Artificial Sequence
  Synthetic
aactgtacaaactactacctca

SEQ ID NO: 80 - DNA, 22 bp, Artificial Sequence
  Synthetic
aacagcacaaactactacctca

SEQ ID NO: 81 - DNA, 22 bp, Artificial Sequence
  Synthetic
gagctacagtgcttcatctcat

SEQ ID NO: 82 - DNA, 22 bp, Artificial Sequence
  Synthetic
acaaagttctgtagtgcactga

SEQ ID NO: 83 - DNA, 578 bp, Artificial Sequence
  Synthetic
atgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 84 - protein, 192 aa, Artificial Sequence
  Synthetic
HDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 85 - DNA, 366 bp, Artificial Sequence
  Synthetic
caggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggcgcacaacgcccacctgatccggcaccagcaccccttggagctcccggtctccccaatggcctcctttcaggagatgaagacttctcctccattgcggacatggacttctcagccctgctgagtcagatcagctcc

SEQ ID NO: 86 - protein, 122 aa, Artificial Sequence
  Synthetic
QAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 87 - DNA, 630 bp, Artificial Sequence
  Synthetic
cccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 88 - protein, 210 aa, Artificial Sequence
  Synthetic
PKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 89 - DNA, 234 bp, Artificial Sequence
  Synthetic
gcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 90 - protein, 78 aa, Artificial Sequence
  Synthetic
APPTDVSLGDELHLDGEDVAMAHADALDDFDLDMLGDGDSPGPGFTPHDSAPYGALDMADFEFEQMFTDALGIDEYGG

SEQ ID NO: 91 - DNA, 123 bp, Artificial Sequence
  Synthetic
ccggcagatgcccttgatgacttcgatttggacatgctcccagcggatgccttggacgattttgatctcgatatgcttcccgccgacgcactcgatgatttcgatctggatatgctcccgggt

SEQ ID NO: 92 - protein, 41 aa, Artificial Sequence
  Synthetic
PADALDDFDLDMLPADALDDFDLDMLPADALDDFDLDMLPG

SEQ ID NO: 93 - DNA, 126 bp, Artificial Sequence
  Synthetic
ggtccggcagatgcccttgatgacttcgatttggacatgctcccagcggatgccttggacgattttgatctcgatatgcttcccgccgacgcactcgatgatttcgatctggatatgctcccgggt

SEQ ID NO: 94 - protein, 42 aa, Artificial Sequence
  Synthetic
GPADALDDFDLDMLPADALDDFDLDMLPADALDDFDLDMLPG

SEQ ID NO: 95 - DNA, 1374 bp, Artificial Sequence
  Synthetic
atgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 96 - protein, 457 aa, Artificial Sequence
  Synthetic
MSRGEVRMAKAGREGPRDSVWLSGEGRRGGRRGGQPSGLDRDRITGVTVRLLDTEGLTGFSMRRLAAELNVTAMSVYWYVDTKDQLLELALDAVFGELRHPDPDAGLDWREELRALARENRALLVRHPWSSRLVGTYLNIGPHSLAFSRAVQNVVRRSGLPAHRLTGAISAVFQFVYGYGTIEGRFLARVADTGLSPEEYFQDSMTAVTEVPDTAGVIEDAQDIMAARGGDTVAEMLDRDFEFALDLLVAGIDAMVEQASAYSRAHDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 97 - DNA, 1164 bp, Artificial Sequence
  Synthetic
atgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 98 - protein, 388 aa, Artificial Sequence
  Synthetic
MSRGEVRMAKAGREGPRDSVWLSGEGRRGGRRGGQPSGLDRDRITGVTVRLLDTEGLTGFSMRRLAAELNVTAMSVYWYVDTKDQLLELALDAVFGELRHPDPDAGLDWREELRALARENRALLVRHPWSSRLVGTYLNIGPHSLAFSRAVQNVVRRSGLPAHRLTGAISAVFQFVYGYGTIEGRFLARVADTGLSPEEYFQDSMTAVTEVPDTAGVIEDAQDIMAARGGDTVAEMLDRDFEFALDLLVAGIDAMVEQASAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 99 - DNA, 1179 bp, Artificial Sequence
  Synthetic
atgccccgccccaagctcaagtccgatgacgaggtactcgaggccgccaccgtagtgctgaagcgttgcggtcccatagagttcacgctcagcggagtagcaaaggaggtggggctctcccgcgcagcgttaatccagcgcttcaccaaccgcgatacgctgctggtgaggatgatggagcgcggcgtcgagcaggtgcggcattacctgaatgcgataccgataggcgcagggccgcaagggctctgggaatttttgcaggtgctcgttcggagcatgaacactcgcaacgacttctcggtgaactatctcatctcctggtacgagctccaggtgccggagctacgcacgcttgcgatccagcggaaccgcgcggtggtggaggggatccgcaagcgactgcccccaggtgctcctgcggcagctgagttgctcctgcactcggtcatcgctggcgcgacgatgcagtgggccgtcgatccggatggtgagctagctgatcatgtgctggctcagatcgctgccatcctgtgtttaatgtttcccgaacacgacgatttccaactcctccaggcacatgcgtccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 100 - protein, 392 aa, Artificial Sequence
  Synthetic
MPRPKLKSDDEVLEAATVVLKRCGPIEFTLSGVAKEVGLSRAALIQRFTNRDTLLVRMMERGVEQVRHYLNAIPIGAGPQGLWEFLQVLVRSMNTRNDFSVNYLISWYELQVPELRTLAIQRNRAVVEGIRKRLPPGAPAAAELLLHSVIAGATMQWAVDPDGELADHVLAQIAAILCLMFPEHDDFQLLQAHASAYSRAHDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 101 - DNA, 969 bp, Artificial Sequence
  Synthetic
atgccccgccccaagctcaagtccgatgacgaggtactcgaggccgccaccgtagtgctgaagcgttgcggtcccatagagttcacgctcagcggagtagcaaaggaggtggggctctcccgcgcagcgttaatccagcgcttcaccaaccgcgatacgctgctggtgaggatgatggagcgcggcgtcgagcaggtgcggcattacctgaatgcgataccgataggcgcagggccgcaagggctctgggaatttttgcaggtgctcgttcggagcatgaacactcgcaacgacttctcggtgaactatctcatctcctggtacgagctccaggtgccggagctacgcacgcttgcgatccagcggaaccgcgcggtggtggaggggatccgcaagcgactgcccccaggtgctcctgcggcagctgagttgctcctgcactcggtcatcgctggcgcgacgatgcagtgggccgtcgatccggatggtgagctagctgatcatgtgctggctcagatcgctgccatcctgtgtttaatgtttcccgaacacgacgatttccaactcctccaggcacatgcgtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 102 - protein, 323 aa, Artificial Sequence
  Synthetic
MPRPKLKSDDEVLEAATVVLKRCGPIEFTLSGVAKEVGLSRAALIQRFTNRDTLLVRMMERGVEQVRHYLNAIPIGAGPQGLWEFLQVLVRSMNTRNDFSVNYLISWYELQVPELRTLAIQRNRAVVEGIRKRLPPGAPAAAELLLHSVIAGATMQWAVDPDGELADHVLAQIAAILCLMFPEHDDFQLLQAHASAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 103 - DNA, 896 bp, Artificial Sequence
  Synthetic
  misc_feature (128)..(128): n is a, c, g, or t
atgaaagcgttaacggccaggcaacaagaggtgtttgatctcatccgtgatcacatcagccagacaggtatgccgccgacgcgtgcggaaatcgcgcagcgtttggggttccgttccccaaacgcggntgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggtgacttgctggcagtgcataaaactcaggatgtacgtaacggtcaggtcgttgtcgcacgtattgatgacgaagttaccgttaagcgcctgaaaaaacagggcaataaagtcgaactgttgccagaaaatagcgagtttaaaccaattgtcgttgaccttcgtcagcagagcttcaccattgaaggtctggcggttggggttattcgcaacggcgactggctgtctagctatccttatgacgtgcctgactatgccagcctgggaggatctagagcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagtg

SEQ ID NO: 104 - protein, 296 aa, Artificial Sequence
  Synthetic
MKALTARQQEVFDLIRDHISQTGMPPTRAEIAQRLGFRSPNAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLSSYPYDVPDYASLGGSRAPPTDVSLGDELHLDGEDVAMAHADALDDFDLDMLGDGDSPGPGFTPHDSAPYGALDMADFEFEQMFTDALGIDEYGG

SEQ ID NO: 105 - DNA, 339 bp, Escherichia coli
atggctacgaccgagcgggacgtaaaccagcttactccgagagagagggacattttgaagctgattgcgcaggggcttcccaataagatgattgccagacgccttgatatcacggaaagcactgtgaaagtccacgtgaaacacatgctcaaaaagatgaaactcaagtcccgcgtggaagctgcggtctgggtacatcaggagcgaatctttggtccggcagatgcccttgatgacttcgatttggacatgctcccagcggatgccttggacgattttgatctcgatatgcttcccgccgacgcactcgatgatttcgatctggatatgctcccgggt

SEQ ID NO: 106 - protein, 113 aa, Escherichia coli
MATTERDVNQLTPRERDILKLIAQGLPNKMIARRLDITESTVKVHVKHMLKKMKLKSRVEAAVWVHQERIFGPADALDDFDLDMLPADALDDFDLDMLPADALDDFDLDMLPG

SEQ ID NO: 107 - DNA, 456 bp, Escherichia coli
atggctacgaccgagcgggacgtaaaccagcttactccgagagagagggacattttgaagctgattgcgcaggggcttcccaataagatgattgccagacgccttgatatcacggaaagcactgtgaaagtccacgtgaaacacatgctcaaaaagatgaaactcaagtcccgcgtggaagctgcggtctgggtacatcaggagcgaatctttgccagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtga

SEQ ID NO: 108 - protein, 151 aa, Escherichia coli
MATTERDVNQLTPRERDILKLIAQGLPNKMIARRLDITESTVKVHVKHMLKKMKLKSRVEAAVWVHQERIFASAPPTDVSLGDELHLDGEDVAMAHADALDDFDLDMLGDGDSPGPGFTPHDSAPYGALDMADFEFEQMFTDALGIDEYGG

SEQ ID NO: 109 - DNA, 348 bp, Escherichia coli
atggctacgaccgagcgggacgtaaaccagcttactccgagagagagggacattttgaagctgattgcgcaggggcttcccaataagatgattgccagacgccttgatatcacggaaagcactgtgaaagtccacgtgaaacacatgctcaaaaagatgaaactcaagtcccgcgtggaagctgcggtctgggtacatcaggagcgaatctttgccagcggtccggcagatgcccttgatgacttcgatttggacatgctcccagcggatgccttggacgattttgatctcgatatgcttcccgccgacgcactcgatgatttcgatctggatatgctcccgggttga

SEQ ID NO: 110 - protein, 115 aa, Escherichia coli
MATTERDVNQLTPRERDILKLIAQGLPNKMIARRLDITESTVKVHVKHMLKKMKLKSRVEAAVWVHQERIFASGPADALDDFDLDMLPADALDDFDLDMLPADALDDFDLDMLPG

SEQ ID NO: 111 - DNA, 843 bp, Artificial Sequence
  Synthetic
atgcaagaaaactacaagattctcgtggtggatgatgacatgcgacttcgcgcattgctcgaaagatatctgaccgagcagggatttcaagtgcgctccgtggccaatgccgagcagatggataggctcttgacgagggagtcgttccatctgatggtgctggaattgatgcttcccggtgaggacggattgtccatttgccggagacttaggtcgcagtcaaaccccatgccgatcatcatggtcacagcgaagggagaggaggtcgatagaattgtaggtcttgagattggggcagacgactacatccccaagccgttcaatccccgggaacttcttgcgcgaatccgagccgtgctcaggcgacaggccaacgagctgcccggagctccatcgcaagaggaagcggtcatcgcgttcgggaagttcaagttgaacctcggcacgagagagatgtttcgggaagatgaacctatgccgctcacatcgggggagtttgcggtcttgaaagcacttgtctcacacccgagagaacctctgtcgcgggataaactcatgaatctggcgagaggcagagagtatagcgcgatggaaaggtccatcgatgtccagattagccgcctccgccgcatggtggaggaagatccagcccaccctcggtacatccagactgtatggggattggggtatgtgttcgtaccggatgggtcaaaagcaggaccggcggacgcactggatgactttgacttggatatgctcccagcggatgcgttggacgattttgaccttgacatgttgcctgccgacgcgcttgacgacttcgacttggacatgctgcccggt

SEQ ID NO: 112 - protein, 281 aa, Artificial Sequence
  Synthetic
MQENYKILVVDDDMRLRALLERYLTEQGFQVRSVANAEQMDRLLTRESFHLMVLELMLPGEDGLSICRRLRSQSNPMPIIMVTAKGEEVDRIVGLEIGADDYIPKPFNPRELLARIRAVLRRQANELPGAPSQEEAVIAFGKFKLNLGTREMFREDEPMPLTSGEFAVLKALVSHPREPLSRDKLMNLARGREYSAMERSIDVQISRLRRMVEEDPAHPRYIQTVWGLGYVFVPDGSKAGPADALDDFDLDMLPADALDDFDLDMLPADALDDFDLDMLPG

SEQ ID NO: 113 - protein, 141 aa, Escherichia coli
MVESYKFNGWELDINSRSLIGPDGEQYKLPRSEFRAMLHFCENPGKIQSRAELLKKMTGRELKPHDRTVDVTIRRIRKHFESTPDTPEIIATIHGEGYRFCGPADALDDFDLDMLPADALDDFDLDMLPADALDDFDLDML

SEQ ID NO: 114 - protein, 381 aa, Escherichia coli
MQLQSMKKEIRHLHQALSTSWQWGHILTNSPAMMDICKDTAKIALSQASVLISGESGTGKELIARAIHYNSRRAKGPFIKVNCAALPESLLESELFGHEKGAFTGAQTLRQGLFERANEGTLLLDEIGEMPLVLQAKLLRILQEREFERIGGHQTIKVDIRIIAATNRDLQAMVKEGTFREDLFYRLNVIHLILPPLRDRREDISLLANHFLQKFSSENQRDIIDIDPMAMSLLTAWSWPGNIRELSNVIERAVVMNSGPIIFSEDLPPQIRQPVCNAGEVKTAPVGERNLKEEIKRVEKRIIMEVLEQQEGNRTRTALMLGISRRALMYKLQEYGIDPADVPADALDDFDLDMLPADALDDFDLDMLPADALDDFDLDML

SEQ ID NO: 115 - protein, 144 aa, Escherichia coli
MQRELQQQDAESPLIIDEGRFQASWRGKMLDLTPAEFRLLKTLSHEPGKVFSREQLLNHLYDDYRVVTDRTIDSHIKNLRRKLESLDAEQSFIRAVYGVGYRWEAPADALDDFDLDMLPADALDDFDLDMLPADALDDFDLDML

SEQ ID NO: 116 - protein, 139 aa, Escherichia coli
MEEVIEMQGLSLDPTSHRVMAGEEPLEMGPTEFKLLHFFMTHPERVYSREQLLNHVWGTNVYVEDRTVDVHIRRLRKALEPGGHDRMVQTVRGTGYRFSTPADALDDFDLDMLPADALDDFDLDMLPADALDDFDLDML

SEQ ID NO: 117 - protein, 127 aa, Escherichia coli
MNGYCYFPFSLNRFVGSLTSDQQKLDSLSKQEISVMRYILDGKDNNDIAEKMFISNKTVSTYKSRLMEKLECKSLMDLYTFAQRNKIGPADALDDFDLDMLPADALDDFDLDMLPADALDDFDLDML

SEQ ID NO: 118 - protein, 390 aa, Escherichia coli
MSHYQEQQQPRNVQLNGPTTDIIGEAPAMQDVFRIIGRLSRSSISVLINGESGTGKELVAHALHRHSPRAKAPFIALNMAAIPKDLIESELFGHEKGAFTGANTIRQGRFEQADGGTLFLDEIGDMPLDVQTRLLRVLADGQFYRVGGYAPVKVDVRIIAATHQNLEQRVQEGKFREDLFHRLNVIRVHLPPLRERREDIPRLARHFLQVAARELGVEAKLLHPETEAALTRLAWPGNVRQLENTCRWLTVMAAGQEVLIQDLPGELFESTVAESTSQMQPDSWATLLAQWADRALRSGHQNLLSEAQPELERTLLTTALRHTQGHKQEAARLLGWGRNTLTRKLKELGMEPADALDDFDLDMLPADALDDFDLDMLPADALDDFDLDML

SEQ ID NO: 119 - protein, 130 aa, Escherichia coli
MGSKVFSERVNQYLREREMFGAEEDPFSVLTERELDVLHELAQGLSNKQIASVLNISEQTVKVHIRNLLRKLNVRSRVAATILFLQQRGAQPADALDDFDLDMLPADALDDFDLDMLPADALDDFDLDML

SEQ ID NO: 120 - protein, 146 aa, Escherichia coli
MRRHNNQGESELIVGNLTLNMGRRQVWMGGEELILTPKEYALLSRLMLKAGSPVHREILYNDIYNWDNEPSTNTLEVHIHNLRDKVGKARIRTVRGFGYMLVANEENPADALDDFDLDMLPADALDDFDLDMLPADALDDFDLDML

SEQ ID NO: 121 - protein, 163 aa, Escherichia coli
MQERSKQDVSLLPENQQALKFIPCTGHSRIYLLQMKDVAFVSSRMSGVYVTSHEGKEGFTELTLRTLESRTPLLRCHRQYLVNLAHLQEIRLEDNGQAELILRNGLTVPVSRRYLKSLKEAIGLPADALDDFDLDMLPADALDDFDLDMLPADALDDFDLDML

SEQ ID NO: 122 - protein, 157 aa, Escherichia coli
MRRSHWSEQQQNNDNGSPTLEVDALVLNPGRQEASFDGQTLELTGTEFTLLYLLAQHLGQVVSREHLSQEVLGKRLTPFDRAIDMHISNLRRKLPDRKDGHPWFKTLRGRGYLMVSASPADALDDFDLDMLPADALDDFDLDMLPADALDDFDLDML

SEQ ID NO: 123 - protein, 150 aa, Escherichia coli
MRRVKKFSTPSPVIRIGHFELNEPAAQISWFDTPLALTRYEFLLLKTLLKSPGRVWSRQQLMDSVWEDAQDTYDRTVDTHIKTLRAKLRAINPDLSPINTHRGMGYSLRGLPADALDDFDLDMLPADALDDFDLDMLPADALDDFDLDML

SEQ ID NO: 124 - protein, 151 aa, Escherichia coli
MRRGAAVIIESQFQVADLMVDLVSRKVTRSGTRITLTSKEFTLLEFFLRHQGEVLPRSLIASQVWDMNFDSDTNAIDVAVKRLRGKIDNDFEPKLIQTVRGVGYMLEVPDGQPADALDDFDLDMLPADALDDFDLDMLPADALDDFDLDML

SEQ ID NO: 125 - protein, 158 aa, Escherichia coli
MQKKMALEKHQYYDQAELDQLIHGSSSNEQDPRRLPKGLTPQTLRTLCQWIDAHQDYEFSTDELANEVNISRVSCRKYLIWLVNCHILFTSIHYGVTGRPVYRYRIQAEHYSLLKQYCQPADALDDFDLDMLPADALDDFDLDMLPADALDDFDLDML

SEQ ID NO: 126 - protein, 144 aa, Escherichia coli
MQRKHMLESIDSASQKQIDEMFNAYARGEPKDELPTGIDPLTLNAVRKLFKEPGVQHTAETVAQALTISRTTARRYLEYCASRHLIIAEIVHGKVGRPQRIYHSGPADALDDFDLDMLPADALDDFDLDMLPADALDDFDLDML

SEQ ID NO: 127 - protein, 362 aa, Escherichia coli
MQSAPATDERWREAIVTRSPLMLRLLEQARLVAQSDVSVLINGQSGTGKEIFAQAIHNASPRNSKPFIAINCGALPEQLLESELFGHARGAFTGAVSNREGLFQAAEGGTLFLDEIGDMPAPLQVKLLRVLQERKVRPLGSNRDIDINVRIISATHRDLPKAMARGEFREDLYYRLNVVSLKIPALAERTEDIPLLANHLLRQAAERHKPFVRAFSTDAMKRLMTASWPGNVRQLVNVIEQCVALTSSPVISDALVEQALEGENTALPTFVEARNQFELNYLRKLLQITKGNVTHAARMAGRNRTEFYKLLSRHELDANDFKEPADALDDFDLDMLPADALDDFDLDMLPADALDDFDLDML

SEQ ID NO: 128 - protein, 147 aa, Escherichia coli
MQHHALNSTLEISGLRMDSVSHSVSRDNISITLTRKEFQLLWLLASRAGEIIPRTVIASEIWGINFDSDTNTVDVAIRRLRAKVDDPFPEKLIATIRGMGYSFVAVKKPADALDDFDLDMLPADALDDFDLDMLPADALDDFDLDML

SEQ ID NO: 129 - protein, 147 aa, Escherichia coli
MRRNSGLASQVISLPPFQVDLSRRELSINDEVIKLTAFEYTIMETLIRNNGKVVSKDSLMLQLYPDAELRESHTIDVLMGRLRKKIQAQYPQEVITTVRGQGYLFELRPADALDDFDLDMLPADALDDFDLDMLPADALDDFDLDML

SEQ ID NO: 130 - protein, 142 aa, Escherichia coli
MRTNGQASNELRHGNVMLDPGKRIATLAGEPLTLKPKEFALLELLMRNAGRVLSRKLIEEKLYTWDEEVTSNAVEVHVHHLRRKLGSDFIRTVHGIGYTLGEKPADALDDFDLDMLPADALDDFDLDMLPADALDDFDLDML

SEQ ID NO: 131 - protein, 131 aa, Escherichia coli
MGKKFTPESVSRLLEKISAGGYGDKRLSPKESEVLRLFAEGFLVTEIAKKLNRSIKTISSQKKSAMMKLGVENDIALLNYLSSVTLSPADKDPADALDDFDLDMLPADALDDFDLDMLPADALDDFDLDML

SEQ ID NO: 132 - protein, 163 aa, Escherichia coli
MRQNEQATLTKGLQETSLTPYKALHFGTLTIDPINRVVTLANTEISLSTADFELLWELATHAGQIMDRDALLKNLRGVSYDGLDRSVDVAISRLRKKLLDNAAEPYRIKTVRNKGYLFAPHAWEPADALDDFDLDMLPADALDDFDLDMLPADALDDFDLDML

SEQ ID NO: 133 - protein, 120 aa, Escherichia coli
MTGGCYLTPDIAIKLASGRQDPLTKRERQVAEKLAQGMAVKEIAAELGLSPKTVHVHRANLMEKLGVSNDVELARRMFDGWPADALDDFDLDMLPADALDDFDLDMLPADALDDFDLDML

SEQ ID NO: 134 - protein, 168 aa, Escherichia coli
MAAWQQQQTSSTPAATVTRENDTINLVKDERIIVTPINDIYYAEAHEKMTFVYTRRESYVMPMNITEFCSKLPPSHFFRCHRSFCVNLNKIREIEPWFNNTYILRLKDLDFEVPVSRSKVKEFRQLMHLPADALDDFDLDMLPADALDDFDLDMLPADALDDFDLDML

SEQ ID NO: 135 - protein, 359 aa, Escherichia coli
MHTHSIDAETPAVTASQFGMVGKSPAMQHLLSEIALVAPSEATVLIHGDSGTGKELVARAIHASSARSEKPLVTLNCAALNESLLESELFGHEKGAFTGADKRREGRFVEADGGTLFLDEIGDISPMMQVRLLRAIQEREVQRVGSNQIISVDVRLIAATHRDLAAEVNAGRFRQDLYYRLNVVAIEVPSLRQRREDIPLLAGHFLQRFAERNRKAVKGFTPQAMDLLIHYDWPGNIRELENAVERAVVLLTGEYISERELPLAIASTPIPLGQSQDIQPLVEVEKEVILAALEKTGGNKTEAARQLGITRKTLLAKLSRPADALDDFDLDMLPADALDDFDLDMLPADALDDFDLDML

SEQ ID NO: 136 - protein, 401 aa, Homo sapiens
MAHVSSETQDVSPKDELTASEASTRSPLCEHTFPGDSDLRSMIEEHAFQVLSQGSLLESPSYTVCVSEPDKDDDFLSLNFPRKLWKIVESDQFKSISWDENGTCIVINEELFKKEILETKAPYRIFQTDAIKSFVRQLNLYGFSKIQQNFQRSAFLATFLSEEKESSVLSKLKFYYNPNFKRGYPQLLVRVKRRIGVKNASPISTLFNEDFNKKHFRAGANMENHNSALAAEASEESLFSASKNLNMPLTRESSVRQIIANSSVPIRSGFPPPSPSTSVGPSEQIATDQHAILNQLTTIHMHSHSTYMQARGHIVNFITTTTSQYHIISPLQNGYFGLTVEPSAVPTRYPLVSVNEAPYRNMLPAGNPWLQMPTIADRSAAPHSRLALQPSPLDKYHPNYN

SEQ ID NO: 137 - protein, 272 aa, Homo sapiens
MNSDSSSVSSRASSPDMDEMYLRDHHHRHHHHQESRLNSVSSTQGDMMQKMPGESLSRAGAKAAGESSKYKIKKQLSEQDLQQLRLKINGRERKRMHDLNLAMDGLREVMPYAHGPSVRKLSKIATLLLARNYILMLTSSLEEMKRLVGEIYGGHHSAFHCGTVGHSAGHPAHAANSVHPVHPILGGALSSGNASSPLSAASLPAIGTIRPPHSLLKAPSTPPALQLGSGFQHWAGLPCPCTICQMPPPPHLSALSTANMARLSAESKDLLK

SEQ ID NO: 138 - protein, 193 aa, Homo sapiens
MDNLRETFLSLEDGLGSSDSPGLLSSWDWKDRAGPFELNQASPSQSLSPAPSLESYSSSPCPAVAGLPCEHGGASSGGSEGCSVGGASGLVEVDYNMLAFQPTHLQGGGGPKAQKGTKVRMSVQRRRKASEREKLRMRTLADALHTLRNYLPPVYSQRGQPLTKIQTLKYTIKYIGELTDLLNRGREPRAQSA

SEQ ID NO: 139 - DNA, 67 bp, Artificial Sequence
  Synthetic
gaaatagcgctgtacagcgtatgggaatctcttgtacggtgtacgagtatcttcccgtacaccgtac

SEQ ID NO: 140 - DNA, 42 bp, Artificial Sequence
  Synthetic
catgtgattgaatataaccgacgtgactgttacatttagggg

SEQ ID NO: 141 - DNA, 40 bp, Artificial Sequence
  Synthetic
tactgtatatatatacagtatactgtatatatatacagta

SEQ ID NO: 142 - DNA, 43 bp, Artificial Sequence
  Synthetic
tacccctataggggtatagcgccggctacccctataggggtat

SEQ ID NO: 143 - DNA, 85 bp, Artificial Sequence
  Synthetic
tacccctataggggtatagcgccggctacccctataggggtattacccctataggggtatagcgccggctacccctataggggta

SEQ ID NO: 144 - DNA, 8 bp, Artificial Sequence
  Synthetic
wakrrkta

SEQ ID NO: 145 - DNA, 21 bp, Artificial Sequence
  Synthetic
atttacattttgaaacatcta

SEQ ID NO: 146 - DNA, 19 bp, Artificial Sequence
  Synthetic
wahatgwwacmaarwdtww

SEQ ID NO: 147 - DNA, 10 bp, Artificial Sequence
  Synthetic
atgttaataa

SEQ ID NO: 148 - DNA, 32 bp, Artificial Sequence
  Synthetic
atgttaataatatgtggcataagcgttaaatg

SEQ ID NO: 149 - DNA, 15 bp, Artificial Sequence
  Synthetic
wamawwtwrttaama

SEQ ID NO: 150 - DNA, 20 bp, Artificial Sequence
  Synthetic
gctatgcagaaatttgcaca

SEQ ID NO: 151 - DNA, 17 bp, Artificial Sequence
  Synthetic
ttctycmydatyksyks

SEQ ID NO: 152 - DNA, 38 bp, Artificial Sequence
  Synthetic
tgtcataaaactgtcatattccttacatataactgtca

SEQ ID NO: 153 - DNA, 16 bp, Artificial Sequence
  Synthetic
ctgwcayaaawctgwm

SEQ ID NO: 154 - DNA, 27 bp, Artificial Sequence
  Synthetic
ttcttacgcctgtaggattagtaagaa

SEQ ID NO: 155 - DNA, 17 bp, Artificial Sequence
  Synthetic
tkcytacamctgtarga

SEQ ID NO: 156 - DNA, 17 bp, Artificial Sequence
  Synthetic
tgcaccawwwtggtgca

SEQ ID NO: 157 - DNA, 17 bp, Artificial Sequence
  Synthetic
tgcmcyaaaatsgtgca

SEQ ID NO: 158 - DNA, 9 bp, Artificial Sequence
  Synthetic
  misc_feature (1)..(1): n is a, c, g, or t
ntaccccta

SEQ ID NO: 159 - DNA, 8 bp, Artificial Sequence
  Synthetic
mtacyyct

SEQ ID NO: 160 - DNA, 20 bp, Artificial Sequence
  Synthetic
  misc_feature (10)..(11): n is a, c, g, or t
cttaaggttnncttaaggtt

SEQ ID NO: 161 - DNA, 11 bp, Artificial Sequence
  Synthetic
  misc_feature (2)..(2): n is a, c, g, or t
  misc_feature (4)..(4): n is a, c, g, or t
  misc_feature (10)..(10): n is a, c, g, or t
ancnctaaant

SEQ ID NO: 162 - DNA, 15 bp, Artificial Sequence
  Synthetic
  misc_feature (6)..(10): n is a, c, g, or t
gtaaannnnngtaaa

SEQ ID NO: 163 - DNA, 15 bp, Artificial Sequence
  Synthetic
gtaaarmwrygwaar

SEQ ID NO: 164 - DNA, 16 bp, Artificial Sequence
  Synthetic
  misc_feature (6)..(11): n is a, c, g, or t
ttcacnnnnnnttcac

SEQ ID NO: 165 - DNA, 23 bp, Artificial Sequence
  Synthetic
  misc_feature (11)..(12): n is a, c, g, or t
aaaatgacaannttgtcattttt

SEQ ID NO: 166 - DNA, 26 bp, Artificial Sequence
  Synthetic
tgattacaaaactttaaaaagtgctg

SEQ ID NO: 167 - DNA, 70 bp, Artificial Sequence
  Synthetic
tgattacaaaactttaaaaagtgctgcatagcgccggccgcgcctgattacaaaactttaaaaagtgctg

SEQ ID NO: 168 - DNA, 62 bp, Artificial Sequence
  Synthetic
tgattacaaaactttaaaaagtgctgtagcgccggctgattacaaaactttaaaaagtgctg

SEQ ID NO: 169 - DNA, 17 bp, Artificial Sequence
  Synthetic
tkwwttwaattwykwwa

SEQ ID NO: 170 - DNA, 13 bp, Artificial Sequence
  Synthetic
gatctattctttt

SEQ ID NO: 171 - DNA, 13 bp, Artificial Sequence
  Synthetic
tatctttttttat

SEQ ID NO: 172 - DNA, 18 bp, Artificial Sequence
  Synthetic
  misc_feature (5)..(14): n is a, c, g, or t
tgtcnnnnnnnnnngaca

SEQ ID NO: 173 - DNA, 17 bp, Artificial Sequence
  Synthetic
  misc_feature (9)..(9): n is a, c, g, or t
cattacaanttgtaatg

SEQ ID NO: 174 - DNA, 17 bp, Artificial Sequence
  Synthetic
  misc_feature (7)..(11): n is a, c, g, or t
catgaannnnntgttta

SEQ ID NO: 175 - DNA, 17 bp, Artificial Sequence
  Synthetic
wrtttakswwyygttta

SEQ ID NO: 176 - DNA, 17 bp, Artificial Sequence
  Synthetic
  misc_feature (7)..(11): n is a, c, g, or t
rttaamnnnnnrttaam

SEQ ID NO: 177 - DNA, 14 bp, Artificial Sequence
  Synthetic
taagaatattccta

SEQ ID NO: 178 - DNA, 14 bp, Artificial Sequence
  Synthetic
awymrgaykwwtyt

SEQ ID NO: 179 - DNA, 18 bp, Artificial Sequence
  Synthetic
  misc_feature (1)..(2): n is a, c, g, or t
  misc_feature (7)..(12): n is a, c, g, or t
  misc_feature (17)..(18): n is a, c, g, or t
nntacannnnnntactnn

SEQ ID NO: 180 - DNA, 14 bp, Artificial Sequence
  Synthetic
kwcwtwtvgttaca

SEQ ID NO: 181 - DNA, 31 bp, Artificial Sequence
  Synthetic
ggcaaaactaagaaattttccaggttttgcc

SEQ ID NO: 182 - DNA, 10 bp, Artificial Sequence
  Synthetic
ggcatttcat

SEQ ID NO: 183 - DNA, 18 bp, Artificial Sequence
  Synthetic
gcgagtcaaaaaaactca

SEQ ID NO: 184 - DNA, 15 bp, Artificial Sequence
  Synthetic
  misc_feature (7)..(9): n is a, c, g, or t
ttcgaannnttcgaa

SEQ ID NO: 185 - DNA, 19 bp, Artificial Sequence
  Synthetic
rcrttcgaaacrttcgaww

SEQ ID NO: 186 - DNA, 17 bp, Artificial Sequence
  Synthetic
rttcgaahsdttcgaay

SEQ ID NO: 187 - DNA, 19 bp, Artificial Sequence
  Synthetic
rcattcyaaacattcyahw

SEQ ID NO: 188 - DNA, 17 bp, Artificial Sequence
  Synthetic
rttcgaaysdttcgaay

SEQ ID NO: 189 - DNA, 10 bp, Artificial Sequence
  Synthetic
accatatgtt

SEQ ID NO: 190 - DNA, 10 bp, Artificial Sequence
  Synthetic
rccatatgkt

SEQ ID NO: 191 - DNA, 10 bp, Artificial Sequence
  Synthetic
avcakmtgtt

SEQ ID NO: 192 - DNA, 10 bp, Artificial Sequence
  Synthetic
rccatatgkt

SEQ ID NO: 193 - DNA, 10 bp, Artificial Sequence
  Synthetic
accatatgkt

SEQ ID NO: 194 - DNA, 10 bp, Artificial Sequence
  Synthetic
amcakmtgtt

SEQ ID NO: 195 - DNA, 10 bp, Artificial Sequence
  Synthetic
accatatgkt

SEQ ID NO: 196 - DNA, 10 bp, Artificial Sequence
  Synthetic
amcatatgby

SEQ ID NO: 197 - DNA, 12 bp, Artificial Sequence
  Synthetic
srccawwtgkys

SEQ ID NO: 198 - DNA, 12 bp, Artificial Sequence
  Synthetic
brccawwtgkyv

SEQ ID NO: 199 - DNA, 15 bp, Artificial Sequence
  Synthetic
agatcaaagggggta

SEQ ID NO: 200 - DNA, 45 bp, Artificial Sequence
  Synthetic
agatcaaagggggtaagatcaaagggggtaagatcaaagggggta

SEQ ID NO: 201 - DNA, 90 bp, Artificial Sequence
  Synthetic
agatcaaagggggtaagatcaaagggggtaagatcaaagggggtaagatcaaagggggtaagatcaaagggggtaagatcaaagggggta

SEQ ID NO: 202 - DNA, 20 bp, Artificial Sequence
  Synthetic
cgcgccgaccacgtggtcca

SEQ ID NO: 203 - DNA, 32 bp, Artificial Sequence
  Synthetic
cgcgccgaccacgtggtcgaccacgtggtcca

SEQ ID NO: 204 - DNA, 44 bp, Artificial Sequence
  Synthetic
cgcgccgaccacgtggtcgaccacgtggtcgaccacgtggtcca

SEQ ID NO: 205 - DNA, 31 bp, Artificial Sequence
  Synthetic
gaccttgagtacgtgcgtctctgcacgtatg

SEQ ID NO: 206 - DNA, 62 bp, Artificial Sequence
  Synthetic
gaccttgagtacgtgcgtctctgcacgtatggaccttgagtacgtgcgtctctgcacgtatg

SEQ ID NO: 207 - DNA, 93 bp, Artificial Sequence
  Synthetic
gaccttgagtacgtgcgtctctgcacgtatggaccttgagtacgtgcgtctctgcacgtatggaccttgagtacgtgcgtctctgcacgtatg

SEQ ID NO: 208 - DNA, 21 bp, Artificial Sequence
  Synthetic
tgtttattgtttattgtttat

SEQ ID NO: 209 - DNA, 42 bp, Artificial Sequence
  Synthetic
tgtttattgtttattgtttattgtttattgtttattgtttat

SEQ ID NO: 210 - DNA, 36 bp, Artificial Sequence
  Synthetic
gcaaagcaaacagcaaagcaaacagcaaagcaaaca

SEQ ID NO: 211 - DNA, 72 bp, Artificial Sequence
  Synthetic
gcaaagcaaacagcaaagcaaacagcaaagcaaacagcaaagcaaacagcaaagcaaacagcaaagcaaaca

SEQ ID NO: 212 - DNA, 36 bp, Artificial Sequence
  Synthetic
tgtttgctttgctgtttgctttgctgtttgctttgc

SEQ ID NO: 213 - DNA, 72 bp, Artificial Sequence
  Synthetic
tgtttgctttgctgtttgctttgctgtttgctttgctgtttgctttgctgtttgctttgctgtttgctttgc

SEQ ID NO: 214 - DNA, 72 bp, Artificial Sequence
  Synthetic
gaacacccagaacacccagaacacccagaacacccagaacacccagaacacccagaacacccagaacaccca

SEQ ID NO: 215 - DNA, 54 bp, Artificial Sequence
  Synthetic
gaacacccagaacacccagaacacccagaacacccagaacacccagaacaccca

SEQ ID NO: 216 - DNA, 15 bp, Artificial Sequence
  Synthetic
agttaataatttaac

SEQ ID NO: 217 - DNA, 30 bp, Artificial Sequence
  Synthetic
agttaataatttaacagttaataatttaac

SEQ ID NO: 218 - DNA, 45 bp, Artificial Sequence
  Synthetic
agttaataatttaacagttaataatttaacagttaataatttaac

SEQ ID NO: 219 - DNA, 60 bp, Artificial Sequence
  Synthetic
agttaataatttaacagttaataatttaacagttaataatttaacagttaataatttaac

SEQ ID NO: 220 - DNA, 48 bp, Artificial Sequence
  Synthetic
ctacacaaagccctctgtgtaagactacacaaagccctctgtgtaaga

SEQ ID NO: 221 - DNA, 72 bp, Artificial Sequence
  Synthetic
ctacacaaagccctctgtgtaagactacacaaagccctctgtgtaagactacacaaagccctctgtgtaaga

SEQ ID NO: 222 - DNA, 48 bp, Artificial Sequence
  Synthetic
ctacacaaagccctctttgtgagactacacaaagccctctttgtgaga

SEQ ID NO: 223 - DNA, 72 bp, Artificial Sequence
  Synthetic
ctacacaaagccctctttgtgagactacacaaagccctctttgtgagactacacaaagccctctttgtgaga

SEQ ID NO: 224 - DNA, 30 bp, Artificial Sequence
  Synthetic
ccattgttctccattgttctccattgttct

SEQ ID NO: 225 - DNA, 60 bp, Artificial Sequence
  Synthetic
ccattgttctccattgttctccattgttctccattgttctccattgttctccattgttct

SEQ ID NO: 226 - DNA, 28 bp, Artificial Sequence
  Synthetic
aacaaagaacaaagaacaaagaacaaag

SEQ ID NO: 227 - DNA, 45 bp, Artificial Sequence
  Synthetic
aaccgttaaacggttaaccgttaaacggttaaccgttaaacggtt

SEQ ID NO: 228 - DNA, 69 bp, Artificial Sequence
  Synthetic
agagatatttagtgaatcagcaagtggaaccaaaaagacttgaggactgattggatgaggagaggttag

SEQ ID NO: 229 - DNA, 138 bp, Artificial Sequence
  Synthetic
agagatatttagtgaatcagcaagtggaaccaaaaagacttgaggactgattggatgaggagaggttagagagatatttagtgaatcagcaagtggaaccaaaaagacttgaggactgattggatgaggagaggttag

SEQ ID NO: 230 - DNA, 91 bp, Artificial Sequence
  Synthetic
actggtgccctcctcaactcccacctgcatctggggcccatactggttggctcccgcggtgccatgtctgcagtgtgccccccagccccgg

SEQ ID NO: 231 - DNA, 182 bp, Artificial Sequence
  Synthetic
actggtgccctcctcaactcccacctgcatctggggcccatactggttggctcccgcggtgccatgtctgcagtgtgccccccagccccggactggtgccctcctcaactcccacctgcatctggggcccatactggttggctcccgcggtgccatgtctgcagtgtgccccccagccccgg

SEQ ID NO: 232 - DNA, 128 bp, Artificial Sequence
  Synthetic
cgcgccgaccacgtggtcgaccacgtggtccacgcgccgaccacgtggtcgaccacgtggtccacgcgccgaccacgtggtcgaccacgtggtccacgcgccgaccacgtggtcgaccacgtggtcca

SEQ ID NO: 233 - DNA, 49 bp, Artificial Sequence
  Synthetic
gtcacgtggctcagtcacgtggctcagtcacgtggctcagtcacgtggc

SEQ ID NO: 234 - DNA, 98 bp, Artificial Sequence
  Synthetic
gtcacgtggctcagtcacgtggctcagtcacgtggctcagtcacgtggcgtcacgtggctcagtcacgtggctcagtcacgtggctcagtcacgtggc

SEQ ID NO: 235 - DNA, 48 bp, Artificial Sequence
  Synthetic
gaccacgtggtcgaccacgtggtcgaccacgtggtcgaccacgtggtc

SEQ ID NO: 236 - DNA, 96 bp, Artificial Sequence
  Synthetic
gaccacgtggtcgaccacgtggtcgaccacgtggtcgaccacgtggtcgaccacgtggtcgaccacgtggtcgaccacgtggtcgaccacgtggtc

SEQ ID NO: 237 - DNA, 124 bp, Artificial Sequence
  Synthetic
cctctaccccctttgatcttaccccctttgatcttaccccctttgatcttaccccctttgatcttaccccctttgatcttaccccctttgatcttaccccctttgatcttaccccctttgatct

SEQ ID NO: 238 - DNA, 514 bp, Artificial Sequence
  Synthetic
ggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactgcagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacac

SEQ ID NO: 239 - DNA, 199 bp, Artificial Sequence
  Synthetic
ctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacac

SEQ ID NO: 240 - DNA, 1150 bp, Artificial Sequence
  Synthetic
aaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaataactgtctgcaagcttatgattcccaaatatctatctctagcctcaatcttgttccagaagataaaaagtagtattcaaatgcacatcaacgtctccacttggagggcttaaagacgtttcaacatacaaaccggggagttttgcctggaatgtttcctaaaatgtgtcctgtagcacatagggtcctcttgttccttaaaatctaattacttttagcccagtgctcatcccacctatggggagatgagagtgaaaagggagcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctaactggtctccttatcatagtcttatccccttttgaaacaaaagagacagtttcaaaatacaaatatgatttttattagctcccttttgttgtctataatagtcccagaaggagttataaactccatttaaaaagtctttgagatgtggcccttgccaactttgccaggctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttcccaagtttgcttatttatgaaaagttatcgataatttctttagttttgtat

SEQ ID NO: 241 - DNA, 609 bp, Artificial Sequence
  Synthetic
tccctgcccacccgcggaaaccgccccaggtgggccgcgccccctccccagcagccagcagggcgccagggctgagccggccgtggaggggagcgggtcccgcgggttatacaggcgccggggctccgcggcaggcaagagaagctgaggcctgagaacggcccgggccttggcgtacggcaggggacgacctgggatgggggcagcgggcggcggcgcagggagtgggccgggggccggtgtgcgcgggcgggacggggcccggggtcgggagaccaccgctcggaagatggggccgggagaggccgccgtcgcagcgcagagggcaccggcggggagacgcgaggacgcggggccgggaacacggacgccggagtagaagcgcggggggcgcgggctggagcgggggcggggacgccggggtcgggggcggtgcgggtttgaggggagggggcggggcgggtccttccctgggggggtggggagagggggcgggggcccatgtgaccggctcagaccggttctggagacaaaaggggccgcggcggccggagcgggacgggcccggcgcgggagggagcgaagcagcgcgggcagcgagcgagtgag

SEQ ID NO: 242 - DNA, 319 bp, Artificial Sequence
  Synthetic
accaccgctcggaagatggggccgggagaggccgccgtcgcagcgcagagggcaccggcggggagacgcgaggacgcggggccgggaacacggacgccggagtagaagcgcggggggcgcgggctggagcgggggcggggacgccggggtcgggggcggtgcgggtttgaggggagggggcggggcgggtccttccctgggggggtggggagagggggcgggggcccatgtgaccggctcagaccggttctggagacaaaaggggccgcggcggccggagcgggacgggcccggcgcgggagggagcgaagcagcgcgg

SEQ ID NO: 243 - DNA, 70 bp, Artificial Sequence
  Synthetic
ccgcggcggccggagcgggacgggcccggcgcgggagggagcgaagcagcgcgggcagcgagcgagtgag

SEQ ID NO: 244 - DNA, 1511 bp, Artificial Sequence
  Synthetic
ggagtctcactctgtcgcccaagctggagtgcagtagtgcgatctcagctcactgcaacctctgccctctgagttcaagtgattctcctgcctcagcctcccgagtagctgggattacaggcgcctgccaccgcgcccagctaattttttgtatttttggtagagacggggtttcaccatcttggccaggctggtcttgaactcctgacctcatgatccacccgcctcggcttcccaaagtgctgggattacaggcgtgagccaccgtgcctggcctaaagaactggatttctaatggtgaaatctaagcaggagaggtgggatttgggtgtaggatacctttcaaatagccttctactccatctatgaaataggctagctttggctcagtaaatttgctgtgtaatgattttctaatgagttaggctggctttaagcccctggttatttcgttgtaaccagttaggctttgcctcttgaagggccacctgggactgtcgtgcagtagattttcttttaacgccccagaatcaggtgctttctctgactttgtgtggctctactgaatcaaatctagcaagccacagaggctttcagacttttaagatacaatattcaaaggtgaggcaggctgtgaaaagcccagcggtccctggctgtccctgaacgcgactatttgcaggttggctttgagaacccggtcagagctgcgttaggaaaacggttcccgggaagctcctcagagagtagaatgaggaggtggattttgtgtgaaggaacaccttgtgtggctctggtggccaggaaagagctggcacaagctgaaagaaggcctgtggcgaagcggagggggacctaagtcagggacccccacctgcccccaggaaggatgaaaaggagacaaaaatcctaaagggaaaagccctccaggctgtaggccaatgagcggcgggaaggaggagtgaggctggggaacttctcccagagccagtcagagcggacggctgctgggaagccaatcagcgcgctcgagcctgcagcccctctgcagtagttatgccagagcgccctgtgtagagcggctgcgagcgggcagctgggctcggctgccgggagccaccgcgcgggctccgcaccctcctctcgcactgccttcgcccggtccccgcgccgcggtgccccagtggcccccgccgcgctccacgccgcgcccccgcaccccgccggctaccggccgcacaaccgccaccgccccctggccgcgcggctcgcctcgccccgccccgtccctcctcgccccgccccaccccagtcagccccgccctgccccgcgccgccaagcggttcccgccctcgcccagcgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 245 - DNA, 1200 bp, Artificial Sequence
  Synthetic
ggagaggtgggatttgggtgtaggatacctttcaaatagccttctactccatctatgaaataggctagctttggctcagtaaatttgctgtgtaatgattttctaatgagttaggctggctttaagcccctggttatttcgttgtaaccagttaggctttgcctcttgaagggccacctgggactgtcgtgcagtagattttcttttaacgccccagaatcaggtgctttctctgactttgtgtggctctactgaatcaaatctagcaagccacagaggctttcagacttttaagatacaatattcaaaggtgaggcaggctgtgaaaagcccagcggtccctggctgtccctgaacgcgactatttgcaggttggctttgagaacccggtcagagctgcgttaggaaaacggttcccgggaagctcctcagagagtagaatgaggaggtggattttgtgtgaaggaacaccttgtgtggctctggtggccaggaaagagctggcacaagctgaaagaaggcctgtggcgaagcggagggggacctaagtcagggacccccacctgcccccaggaaggatgaaaaggagacaaaaatcctaaagggaaaagccctccaggctgtaggccaatgagcggcgggaaggaggagtgaggctggggaacttctcccagagccagtcagagcggacggctgctgggaagccaatcagcgcgctcgagcctgcagcccctctgcagtagttatgccagagcgccctgtgtagagcggctgcgagcgggcagctgggctcggctgccgggagccaccgcgcgggctccgcaccctcctctcgcactgccttcgcccggtccccgcgccgcggtgccccagtggcccccgccgcgctccacgccgcgcccccgcaccccgccggctaccggccgcacaaccgccaccgccccctggccgcgcggctcgcctcgccccgccccgtccctcctcgccccgccccaccccagtcagccccgccctgccccgcgccgccaagcggttcccgccctcgcccagcgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 246 - DNA, 600 bp, Artificial Sequence
  Synthetic
aaagggaaaagccctccaggctgtaggccaatgagcggcgggaaggaggagtgaggctggggaacttctcccagagccagtcagagcggacggctgctgggaagccaatcagcgcgctcgagcctgcagcccctctgcagtagttatgccagagcgccctgtgtagagcggctgcgagcgggcagctgggctcggctgccgggagccaccgcgcgggctccgcaccctcctctcgcactgccttcgcccggtccccgcgccgcggtgccccagtggcccccgccgcgctccacgccgcgcccccgcaccccgccggctaccggccgcacaaccgccaccgccccctggccgcgcggctcgcctcgccccgccccgtccctcctcgccccgccccaccccagtcagccccgccctgccccgcgccgccaagcggttcccgccctcgcccagcgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 247 - DNA, 300 bp, Artificial Sequence
  Synthetic
ccccgcaccccgccggctaccggccgcacaaccgccaccgccccctggccgcgcggctcgcctcgccccgccccgtccctcctcgccccgccccaccccagtcagccccgccctgccccgcgccgccaagcggttcccgccctcgcccagcgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 248 - DNA, 200 bp, Artificial Sequence
  Synthetic
gtcagccccgccctgccccgcgccgccaagcggttcccgccctcgcccagcgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 249 - DNA, 150 bp, Artificial Sequence
  Synthetic
cgcccaggtagctgcgaggaaacttttgcagcggctgggtagcagcacgtctcttgctcctcagggccactgccaggcttgccgagtcctgggactgctctcgctccggctgccactctcccgcgctctcctagctccctgcgaagcagg

SEQ ID NO: 250 - DNA, 455 bp, Artificial Sequence
  Synthetic
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 251 - DNA, 258 bp, Artificial Sequence
  Synthetic
ccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 252 - DNA, 159 bp, Artificial Sequence
  Synthetic
cgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 253 - DNA, 108 bp, Artificial Sequence
  Synthetic
cccctccccttcctttccgcggccccgccctctcctcgcggcgcgagtttcaggcagcgctgcgtcctgctgcgcacgtgggaagccctggccccggccacccccgcg

SEQ ID NO: 254 - DNA, 83 bp, Artificial Sequence
  Synthetic
cccgggtccccggcccagccccctccgggccctcccagcccctccccttcctttccgcggccccgccctctcctcgcggcgcg

SEQ ID NO: 255 - DNA, 976 bp, Artificial Sequence
  Synthetic
ccatagaaccagagaagtgagtggatgtgatgcccagctccagaagtgactccagaacaccctgttccaaagcagaggacacactgattttttttttaataggctgcaggacttactgttggtgggacgccctgctttgcgaagggaaaggaggagtttgccctgagcacaggcccccaccctccactgggctttccccagctcccttgtcttcttatcacggtagtggcccagtccctggcccctgactccagaaggtggccctcctggaaacccaggtcgtgcagtcaacgatgtactcgccgggacagcgatgtctgctgcactccatccctcccctgttcatttgtccttcatgcccgtctggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtgg

SEQ ID NO: 256 - DNA, 500 bp, Artificial Sequence
  Synthetic
caatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtgg

SEQ ID NO: 257 - DNA, 250 bp, Artificial Sequence
  Synthetic
ttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtgg

SEQ ID NO: 258 - DNA, 150 bp, Artificial Sequence
  Synthetic
tacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtgg

SEQ ID NO: 259 - DNA, 85 bp, Artificial Sequence
  Synthetic
cctaagagggcgtgcgctcccgacatgccccgcggcgcgccattaaccgccagatttgaatcgcgggacccgttggcagaggtgg

SEQ ID NO: 260 - DNA, 860 bp, Artificial Sequence
  Synthetic
aattctagtttggtcctagatgaccacatatccattgttccttcaacgagcacatggtaaagagcctagaacacagagacacagaacacagtggagaaaagggagtgaaatgtctttaatgacacttactatatatgggattttgtgacaatatacaaggatggttaagacatataaggtgatgcaaaaaaacatattaacaattatagtgacaaaaaatgaggagcatataattatacattgatttatacagagtaccagaggaacacagcattgagagccgtaacaccacctgagggagtggagaaaggcttcagagagaaagtgttttttggaatggatcactgtttccaaaagaactaaagtacagtttgagaaatgcatacttaattcattacttttttcccctcaactttaataataaatttacccaacaaaaaagtttatttttgacttgtaaatctcttaaaatcataaaaaagtaaaattagcttttaaaaacaggtagtcaccatagcattgaatgtgtagtttataatacagcaaagttaaatacaatttcaaattacctattaagttagttgctcatttctttgatttcatttagcattgatctaactcaatgtggaagaaggttacattcgtgcaagttaacacggcttaatgattaactatgttcacctaccaaccttaccttttctgggcaaatattggtatatatagagttaagaagtctaggtctgcttccagaagaaaacagttccacgttgcttgaaattgaaaatcaagataaaaatgttcacaattaagctccttctttttattgttcctctagttatttcctccagaattgatcaaga

SEQ ID NO: 261 - DNA, 347 bp, Artificial Sequence
  Synthetic
atagcattgaatgtgtagtttataatacagcaaagttaaatacaatttcaaattacctattaagttagttgctcatttctttgatttcatttagcattgatctaactcaatgtggaagaaggttacattcgtgcaagttaacacggcttaatgattaactatgttcacctaccaaccttaccttttctgggcaaatattggtatatatagagttaagaagtctaggtctgcttccagaagaaaacagttccacgttgcttgaaattgaaaatcaagataaaaatgttcacaattaagctccttctttttattgttcctctagttatttcctccagaattgatcaaga

SEQ ID NO: 262 - DNA, 241 bp, Artificial Sequence
  Synthetic
tcaatgtggaagaaggttacattcgtgcaagttaacacggcttaatgattaactatgttcacctaccaaccttaccttttctgggcaaatattggtatatatagagttaagaagtctaggtctgcttccagaagaaaacagttccacgttgcttgaaattgaaaatcaagataaaaatgttcacaattaagctccttctttttattgttcctctagttatttcctccagaattgatcaaga

SEQ ID NO: 263 - DNA, 404 bp, Artificial Sequence
  Synthetic
tgggatgtttcgagcagtcctgctgaagtccttttatatcctgtttaagggatgcctgttaactagtaaccttcagtgagcaaacatatgactctatttccttacgttgaagttaggcaatttgccaataattaacagagcaggggtcacttgtatcctatgttcaaggacaaagaccacttcagagtggaaaaaaaatctaaactgttcaaatagattatttcccctgaagaataattcattcatctcaacataagacatagatatagccataaagaaaaggtagcagacttactatgtaactccaaatacaagttcaggctattcattagtggatatatttcttgattatccagttatagtatattttattttatttagtgtatcgcatctggtttaacata

SEQ ID NO: 264 - DNA, 470 bp, Artificial Sequence
  Synthetic
atgagggaagcgggtgtgatccacttgaaaactgctggttccttcaccgcaggcagtgctggaagtgggatgtttcgagcagtcctgctgaagtccttttatatcctgtttaagggatgcctgttaactagtaaccttcagtgagcaaacatatgactctatttccttacgttgaagttaggcaatttgccaataattaacagagcaggggtcacttgtatcctatgttcaaggacaaagaccacttcagagtggaaaaaaaatctaaactgttcaaatagattatttcccctgaagaataattcattcatctcaacataagacatagatatagccataaagaaaaggtagcagacttactatgtaactccaaatacaagttcaggctattcattagtggatatatttcttgattatccagttatagtatattttattttatttagtgtatcgcatctggtttaacatag

SEQ ID NO: 265 - DNA, 800 bp, Artificial Sequence
  Synthetic
atgagggaagcgggtgtgatccacttgaaaactgctggttccttcaccgcaggcagtgctggaagtgggatgtttcgagcagtcctgctgaagtccttttatatcctgtttaagggatgcctgttaactagtaaccttcagtgagcaaacatatgactctatttccttacgttgaagttaggcaatttgccaataattaacagagcaggggtcacttgtatcctatgttcaaggacaaagaccacttcagagtggaaaaaaaatctaaactgttcaaatagattatttcccctgaagaataattcattcatctcaacataagacatagatatagccataaagaaaaggtagcagacttactatgtaactccaaatacaagttcaggctattcattagtggatatatttcttgattatccagttatagtatattttattttatttagtgtatcgcatctggtttaacatagaaaacttacagcacaaaacctgatgagccagctcccattctaattttatgtgccaaagaataattccatatgtatgtcacaggtgcatgggtcagctgcaacatcctctcaagccctaagatgatgatgctaacagcaacaaatgggcactgatagtttccatttctctacacattagagttgatggaaaacttttaaaacttcccagtgcgtatcgaaactagaactcagacgttggcgtgtcagagtctgtgtgtctagaggtccagacatgtttgctaaggcttcatatgtagttgagtttattttttatttttttaaattcatggc

SEQ ID NO: 266 - DNA, 1104 bp, Artificial Sequence
  Synthetic
atgagggaagcgggtgtgatccacttgaaaactgctggttccttcaccgcaggcagtgctggaagtgggatgtttcgagcagtcctgctgaagtccttttatatcctgtttaagggatgcctgttaactagtaaccttcagtgagcaaacatatgactctatttccttacgttgaagttaggcaatttgccaataattaacagagcaggggtcacttgtatcctatgttcaaggacaaagaccacttcagagtggaaaaaaaatctaaactgttcaaatagattatttcccctgaagaataattcattcatctcaacataagacatagatatagccataaagaaaaggtagcagacttactatgtaactccaaatacaagttcaggctattcattagtggatatatttcttgattatccagttatagtatattttattttatttagtgtatcgcatctggtttaacatagaaaacttacagcacaaaacctgatgagccagctcccattctaattttatgtgccaaagaataattccatatgtatgtcacaggtgcatgggtcagctgcaacatcctctcaagccctaagatgatgatgctaacagcaacaaatgggcactgatagtttccatttctctacacattagagttgatggaaaacttttaaaacttcccagtgcgtatcgaaactagaactcagacgttggcgtgtcagagtctgtgtgtctagaggtccagacatgtttgctaaggcttcatatgtagttgagtttattttttatttttttaaattcaggcgactgggtttgaattttgccctctccgttatctgccacatgactttgtgtgaggtttctaataccaactgcaaacaaccctaagcccacgtgtgctgttgctcaaagctttgtcgcaaatactgagctcacaccacatacctctcatagctctatgtctggttctgtttgtcacttcctgagcccatgaaacctctcagaagcaatatggttaaacaaactggactttagtctatgaaaggctctacccttgactattcaaactgtcagccagatgacaaaaactcaaaccagctttattctggc

SEQ ID NO: 267 - DNA, 942 bp, Artificial Sequence
  Synthetic
atgagggaagcgggtgtgatccacttgaaaactgctggttccttcaccgcaggcagtgctggaagtgggatgtttcgagcagtcctgctgaagtccttttatatcctgtttaagggatgcctgttaactagtaaccttcagtgagcaaacatatgactctatttccttacgttgaagttaggcaatttgccaataattaacagagcaggggtcacttgtatcctatgttcaaggacaaagaccacttcagagtggaaaaaaatcttgcaaatgctgcaaatgttcttcaccatctaaactgttcaaatagattatttcccctgaagaataattcattcatctcaacataagacatagatatagccataaagaaaaggtagcagacttactatgtaactccaaatacattctttttgaaagaaataataaaatgcacaccatatgctaggcactgaacaaattgtttcagtagttcaggctattcattagtggatatatttcttgattatccagttattatttcgctcaaaaccatcggtcaagtatattttattttatttagtgtatcgcatctggtttaacatagaaaacttacagcacaaaacctgatgagccagctcccattctaattttatgtgccaaagaataattccatatgtatgtcacaggtgcatgggtcagctgcaacatcctctcaagccctaagatgatgatgctaacagcaacaaatgggcactgacatacttctgaccctaagagtgcttcactcataccttcaccctcaatgccgtagagtctatgatagtttccatttctctacacattagagttgatggaaaacttttaaaacttcccagtgcgtatcgaaactagaactcagacgttggcgtgtcagagtctgtgtgtctagaggtccagacatgtttgctaaggcttcatatg

SEQ ID NO: 268 - DNA, 1097 bp, Artificial Sequence
  Synthetic
tagcccgacagagcaagagaggagccgctacccagccgccgcaaaagtttcctcgcagctacctgggcgctgggcgagggcgggaacagcttggcggtgcggggcggcccggggcggagccttgtgggcgtggcgaggagggacggggcggggcgaggcaaggcgagccgcgctgcctggaggacggcgtggggtcgtgtagctgctggcctgcgggatgcggggcgtggcaaggagcttagctgggagattgggtttaccaaggtggcgggcaagccttggtgggagaggcgcgggaagaggataaggagcgtgtgcggtggctcccggcaatcctgccctgacactcgctcgccgctgctctacactgggcgctctggcataactactgcagaggggctgcaggctcaggcacgctgattggcttcccagcagcagtcccctctgactggctctgggagaagttccccagcctcactcctcctttccgcctccctttggcctacagccgggagggcttttccttttcagcctttgcaagctctccatcttccttggagtggagtggaggtctgcggtttaggtacccgactcgaccctaggccttctcccacccagatctggctccttctggccaccagagcccacacaaggtttcctaagcacaaaatccctctccttgctgttttctgagaaaggtttcttgggaaccctttcccaatgcagctgtggccaagccctcaaagcctacccacaaatagtcacgttccagagcgctggggacctctggatttcacagcctggctcatctttgtacctaaaaggtctggaagcccgtgtagcttgctgggtttcattcaatagaaccacacaaagtaaatgtgtgcaaatttaggcacttgatcctgattcctaggtgaatcatatcatctacaggataatcacgggcgaccctcataaagcaaagtgtagctggtgagagtaactcattcaggaaatcattttacagatgaaattcattaagtcatggttagtctgtttcatacctggagtagagccctatttagaagatttcctggatgtcaatccacgtttct

SEQ ID NO: 269 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccaagcactctgatttgacaattaaagcactctgatttgacaattaaagcactctgatttgacaattaaagcactctgatttgacaattagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 270 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccaaccatacaacctactacctcaaaccatacaacctactacctcaaaccatacaacctactacctcaaaccatacaacctactacctcagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 271 - DNA, 3782 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 272 - DNA, 3940 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggtgaggtagtaggttgtatggtttgaggtagtaggttgtatggtttgaggtagtaggttgtatggtttgaggtagtaggttgtatggttatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtaccaaccatacaacctactacctcaaaccatacaacctactacctcaaaccatacaacctactacctcaaaccatacaacctactacctcaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 273 - DNA, 4107 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctagggggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggaattcgaagcttacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggaagagccgagggcaggggaagtcttctaacatgcggggacgtggaggaaaatcccgggcccagatctatgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggaagcttggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 274 - DNA, 4134 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctagggggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggaattcgaagcttacgacggacatgtgaaatagcgctgtacagcgtatgggaatctcttgtacggtgtacgagtatcttcccgtacaccgtacggcgcgccctacacaaagccctctttgtgagactacacaaagccctctttgtgagactacacaaagccctctttgtgagacatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggaagagccgagggcaggggaagtcttctaacatgcggggacgtggaggaaaatcccgggcccagatctatgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggaagcttggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 275 - DNA, 3789 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacctatcctgaattacttgaaacctatcctgaattacttgaaacctatcctgaattacttgaaacctatcctgaattacttgaagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 276 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacagttcttcaactggcagcttacagttcttcaactggcagcttacagttcttcaactggcagcttacagttcttcaactggcagcttgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 277 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcccgtgttcacagcggaccttgatcgtgttcacagcggaccttgatcgtgttcacagcggaccttgatcgtgttcacagcggaccttgatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 278 - DNA, 3792 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatctccaaaacatgaattgctgctgtccaaaacatgaattgctgctgtccaaaacatgaattgctgctgtccaaaacatgaattgctgctggtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 279 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcccaaacaccattgtcacactccacaaacaccattgtcacactccacaaacaccattgtcacactccacaaacaccattgtcacactccagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 280 - DNA, 3797 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcctccagtcagttcctgatgcagtatccagtcagttcctgatgcagtatccagtcagttcctgatgcagtatccagtcagttcctgatgcagtagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 281 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcctcacagttgccagctgagattatcacagttgccagctgagattatcacagttgccagctgagattatcacagttgccagctgagattagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 282 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacaagctttttgctcgtcttatacaagctttttgctcgtcttatacaagctttttgctcgtcttatacaagctttttgctcgtcttatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 283 - DNA, 3793 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacaaaccttttgttcgtcttatacaaaccttttgttcgtcttatacaaaccttttgttcgtcttatacaaaccttttgttcgtcttatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 284 - DNA, 3792 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtacccaaacaccattgtcacactccaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 285 - DNA, 4213 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatcccgccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgaggtacccaaacaccattgtcacactccaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 286 - DNA, 4361 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacttgtggactaagtttgttcacatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggtgaggtagtaggttgtatggtttgaggtagtaggttgtatggtttgaggtagtaggttgtatggtttgaggtagtaggttgtatggttatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatcccgccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgaggtaccaaccatacaacctactacctcaaaccatacaacctactacctcaaaccatacaacctactacctcaaaccatacaacctactacctcaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 287 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccaagcactctgatttgacaattaaagcactctgatttgacaattaaagcactctgatttgacaattaaagcactctgatttgacaattagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 288 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccaaccatacaacctactacctcaaaccatacaacctactacctcaaaccatacaacctactacctcaaaccatacaacctactacctcagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 289 - DNA, 3347 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 290 - DNA, 3505 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggtgaggtagtaggttgtatggtttgaggtagtaggttgtatggtttgaggtagtaggttgtatggtttgaggtagtaggttgtatggttatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtaccaaccatacaacctactacctcaaaccatacaacctactacctcaaaccatacaacctactacctcaaaccatacaacctactacctcaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 291 - DNA, 3672 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctagggggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggaattcgaagcttacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggaagagccgagggcaggggaagtcttctaacatgcggggacgtggaggaaaatcccgggcccagatctatgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggaagcttggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 292 - DNA, 3699 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctagggggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggaattcgaagcttacgacggacatgtgaaatagcgctgtacagcgtatgggaatctcttgtacggtgtacgagtatcttcccgtacaccgtacggcgcgccctacacaaagccctctttgtgagactacacaaagccctctttgtgagactacacaaagccctctttgtgagacatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggaagagccgagggcaggggaagtcttctaacatgcggggacgtggaggaaaatcccgggcccagatctatgagtcgaggagaggtgcgcatggcgaaggcagggcgggaggggccgcgggacagcgtgtggctgtcgggggaggggcggcgcggcggtcgccgtggggggcagccgtccgggctcgaccgggaccggatcaccggggtcaccgtccggctgctggacacggagggcctgacggggttctcgatgcgccgcctggccgccgagctgaacgtcaccgcgatgtccgtgtactggtacgtcgacaccaaggaccagttgctcgagctcgccctggacgccgtcttcggcgagctgcgccacccggacccggacgccgggctcgactggcgcgaggaactgcgggccctggcccgggagaaccgggcgctgctggtgcgccacccctggtcgtcccggctggtcggcacctacctcaacatcggcccgcactcgctggccttctcccgcgcggtgcagaacgtcgtgcgccgcagcgggctgcccgcgcaccgcctgaccggcgccatctcggccgtcttccagttcgtctacggctacggcaccatcgagggccgcttcctcgcccgggtggcggacaccgggctgagtccggaggagtacttccaggactcgatgaccgcggtgaccgaggtgccggacaccgcgggcgtcatcgaggacgcgcaggacatcatggcggcccggggcggcgacaccgtggcggagatgctggaccgggacttcgagttcgccctcgacctgctcgtcgcgggcatcgacgcgatggtcgaacaggcctccgcgtacagccgcgcgcatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggaagcttggtaccgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 293 - DNA, 3354 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacctatcctgaattacttgaaacctatcctgaattacttgaaacctatcctgaattacttgaaacctatcctgaattacttgaagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 294 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacagttcttcaactggcagcttacagttcttcaactggcagcttacagttcttcaactggcagcttacagttcttcaactggcagcttgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 295 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcccgtgttcacagcggaccttgatcgtgttcacagcggaccttgatcgtgttcacagcggaccttgatcgtgttcacagcggaccttgatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 296 - DNA, 3357 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatctccaaaacatgaattgctgctgtccaaaacatgaattgctgctgtccaaaacatgaattgctgctgtccaaaacatgaattgctgctggtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 297 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcccaaacaccattgtcacactccacaaacaccattgtcacactccacaaacaccattgtcacactccacaaacaccattgtcacactccagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 298 - DNA, 3362 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcctccagtcagttcctgatgcagtatccagtcagttcctgatgcagtatccagtcagttcctgatgcagtatccagtcagttcctgatgcagtagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 299 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatcctcacagttgccagctgagattatcacagttgccagctgagattatcacagttgccagctgagattatcacagttgccagctgagattagtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 300 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacaagctttttgctcgtcttatacaagctttttgctcgtcttatacaagctttttgctcgtcttatacaagctttttgctcgtcttatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 301 - DNA, 3358 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccccgcggcatatgttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagttggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttcgtacgttcgaagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagacgcggatccacaaaccttttgttcgtcttatacaaaccttttgttcgtcttatacaaaccttttgttcgtcttatacaaaccttttgttcgtcttatgtcgacctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 302 - DNA, 3357 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagcggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggtacccaaacaccattgtcacactccaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 303 - DNA, 3778 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggcttcgaatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatcccgccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgaggtacccaaacaccattgtcacactccaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 304 - DNA, 3926 bp, Artificial Sequence
  Synthetic
cagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctcctaggtgaggtagtaggttgtatggtttgaggtagtaggttgtatggtttgaggtagtaggttgtatggtttgaggtagtaggttgtatggttatcgatgaattcgaagcttctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggaggcctgttcgaccatcgcgtcgatgcccgcgacgagcaggtcgagggcgaactcgaagtcccggtccagcatctccgccacggtgtcgccgccccgggccgccatgatgtcctgcgcgtcctcgatgacgcccgcggtgtccggcacctcggtcaccgcggtcatcgagtcctggaagtactcctccggactcagcccggtgtccgccacccgggcgaggaagcggccctcgatggtgccgtagccgtagacgaactggaagacggccgagatggcgccggtcaggcggtgcgcgggcagcccgctgcggcgcacgacgttctgcaccgcgcgggagaaggccagcgagtgcgggccgatgttgaggtaggtgccgaccagccgggacgaccaggggtggcgcaccagcagcgcccggttctcccgggccagggcccgcagttcctcgcgccagtcgagcccggcgtccgggtccgggtggcgcagctcgccgaagacggcgtccagggcgagctcgagcaactggtccttggtgtcgacgtaccagtacacggacatcgcggtgacgttcagctcggcggccaggcggcgcatcgagaaccccgtcaggccctccgtgtccagcagccggacggtgaccccggtgatccggtcccggtcgagcccggacggctgccccccacggcgaccgccgcgccgcccctcccccgacagccacacgctgtcccgcggcccctcccgccctgccttcgccatgcgcacctctcctcgactcataccggtagcgctagcgatgagctctggtagtagactagtggcccccattatataccctctagagcatatgtctcacaaagagggctttgtgtagtctcacaaagagggctttgtgtagtctcacaaagagggctttgtgtagggcgcgcccccgtagcttggcgtaatcacatgtccgtcgttttacaacgtcgtgactgggaaaaccctggcctgcaaggcgattaagttgggtaacgccagggttttcccagtcacgacgttgtaaaacgacggacatgtgaaatagcgctgtacagcgtatgggaatctcttgtacggtgtacgagtatcttcccgtacaccgtacggcgcgccagttaataattaactagttaataattaactagttaataattaactcatatgctctagagggtatataatgggggccactagtctactaccagagctcatcgctagcgctggatcccgccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactgaggtaccaaccatacaacctactacctcaaaccatacaacctactacctcaaaccatacaacctactacctcaaaccatacaacctactacctcaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 305 - RNA, 23 nt, Artificial Sequence
  Synthetic
guuuacaauugacuaacacucca

SEQ ID NO: 306 - RNA, 23 nt, Artificial Sequence
  Synthetic
uggagugugacaaugguguuugu